(19) United States
(12) Patent Application Publication
(10) Pub. No.: US 2025/0041227 A1
(43) Pub. Date: Feb. 6, 2025

(54) TABLET FORMULATIONS OF RBP4 INHIBITORS AND METHODS OF USE

(71) Applicant: BELITE BIO, INC, San Diego, CA (US)

(72) Inventors: Irene Cheng-Chi WANG, San Diego, CA (US); Tom Yu-Hsin WANG, San Diego, CA (US)

(21) Appl. No.: 18/711,434

(22) PCT Filed: Nov. 22, 2022
(86) PCT No.: PCT/US2022/080335

§ 371 Date: May 17, 2024

Related U.S. Application Data
(60) Provisional application No. 63/282,540, filed on Nov. 23, 2021.

(51) Int. Cl. A61K 9/20; A61K 31/4545; A61P 27/02; C07D 471/04

(52) U.S. Cl. CPC A61K 9/2054; A61K 9/2018; A61K 31/4545; A61P 27/02; C07D 471/04

(57) ABSTRACT

Provided herein are heterocyclic derivative compounds and tablet pharmaceutical compositions comprising said compounds that are useful for the treatment of retinal binding protein (RBP4) related diseases, such as macular degeneration and the like.

### CROSS-REFERENCE

This application claims the benefit of U.S. Provisional Patent Application No. 63/282,540 filed on Nov. 23, 2021, which is incorporated by reference in its entirety.

### BACKGROUND

A need exists in the medicinal arts for the effective treatment of visual diseases and disorders associated with retinol-binding protein 4 (RBP4).

### BRIEF SUMMARY OF THE INVENTION

Provided herein are formulations for lowering serum or plasma concentrations of RBP4.

Provided herein are tablet pharmaceutical compositions comprising:
(i) a compound of Formula (I), a pharmaceutically acceptable salt, crystalline, solvate, polymorph, prodrug, metabolite, N-oxide, stereoisomer, or isomer thereof:

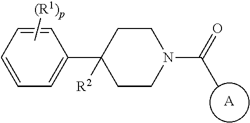

Formula (I)
wherein:
each R¹ is independently halogen, haloalkyl, or alkyl;
R² is —H, —OH, or halogen;
p is 0, 1, 2, 3, 4, or 5;
A has the structure:

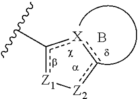

wherein:
α, β, χ, and 6 are each independently absent or present, and when present each is a bond;
X is C;
Z₁ is S, O, or N;
Z₂ is S, O, N, or NR³;
R³ is H, C₁-C₄ alkyl, or oxetane; and
B is a substituted or unsubstituted fused 5-, 6-, or 7-membered ring structure; and
(ii) at least one excipient, wherein the excipient comprises one or more of a disintegrant, dispersion polymer, diluent, glidant, and lubricant. Further provided herein are pharmaceutical compositions wherein A has the structure

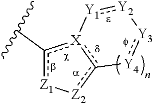

wherein:
n is 0, 1, or 2;
α, β, χ, δ, ε, and ϕ are each independently absent or present, and when present each is a bond;
Z₁ is S, O, or N;
Z₂ is S, O, N or NR³,
wherein R³ is H, C₁-C₄ alkyl, or oxetane;
X is C;
Y₁, Y₂, Y₃ and each occurrence of Y₄ are each independently CR⁴, C(R⁵)₂, NR⁶, O, N, SO₂, or —(C=O)—, wherein:
R⁴ is H, halogen, C₁-C₁₀ alkyl, C₁-C₁₀ cycloalkyl, —O(C₁-C₁₀ alkyl), —C(O)OH, —C(O)O(C₁-C₁₀ alkyl), —C(O)NH₂, —C(O)NH(C₁-C₄ alkyl), —C(O)N(C₁-C₄ alkyl)₂, —NHC(O)NH(C₁-C₁₀ alkyl), —NHC(O)N(C₁-C₄ alkyl)₂, —SO₂NH(C₁-C₁₀ alkyl), —SO₂N(C₁-C₁₀ alkyl)₂, —CN, or —CF₃;
R⁵ is H or C₁-C₁₀ alkyl; and
R⁶ is H, C₁-C₁₀ alkyl, C₃-C₆ cycloalkyl, —(C₁-C₁₀ alkylene)CF₃, —(C₁-C₁₀ alkylene)OCH₃, —(C₁-C₁₀ alkylene)-halogen, —SO₂(C₁-C₁₀ alkyl), —SO₂(C₁-C₁₀ alkylene)-CF₃, —SO₂(C₁-C₁₀ alkylene)OCH₃, —SO₂(C₁-C₁₀ alkylene)-halogen, —C(O)(C₁-C₁₀ alkyl), —C(O)(C₁-C₁₀ alkylene)CF₃, —C(O)(C₁-C₁₀ alkylene)OCH₃, —C(O)(C₁-C₁₀ alkylene)-halogen, —C(O)NH(C₁-C₁₀ alkyl), —C(O)N(C₁-C₁₀ alkyl)₂, —(C₁-C₁₀ alkyl)C(O)OH, —C(O)NH₂, or oxetane. Further provided herein are pharmaceutical compositions wherein A has the structure

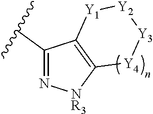

wherein:
n is 0;
R³ is H, C₁-C₄ alkyl, or oxetane;
Y₁ and Y₃ are each CH₂ or C(CH₃)₂;
Y₂ is O, SO₂, or NR⁶; and
R⁶ is H, C₁-C₄ alkyl, C₃-C₆ cycloalkyl, —(C₁-C₄ alkylene)CF₃, —(C₁-C₄ alkylene)OCH₃, —(C₁-C₄ alkylene)-halogen, —SO₂(C₁-C₄ alkyl), —SO₂(C₁-C₄ alkylene)CF₃, —SO₂(C₁-C₄ alkylene)OCH₃, —SO₂(C₁-C₄ alkylene)-halogen, —C(O)(C₁-C₄ alkyl), —C(O)(C₁-C₄ alkylene)CF₃, —C(O)(C₁-C₄ alkylene)OCH₃, —C(O)(C₁-C₄ alkylene)-halogen, —C(O)NH(C₁-C₄ alkyl), —C(O)N(C₁-C₄ alkyl)₂, —(C₁-C₄ alkylene)C(O)OH, —C(O)NH₂, or oxetane. Further provided herein are pharmaceutical compositions wherein the compound of Formula (I) has the structure:

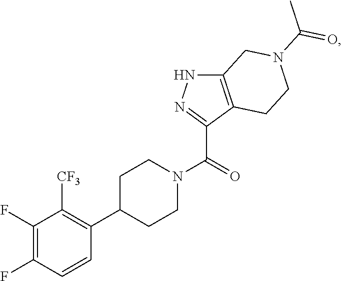 [SMILES: CC(=O)N1CCc2c(C(=O)N3CCC(c4ccc(F)c(F)c4C(F)(F)F)CC3)n[nH]c2C1]

or a pharmaceutically acceptable salt, crystalline, solvate, polymorph, prodrug, metabolite, N-oxide, stereoisomer, or isomer thereof. Further provided herein are pharmaceutical compositions wherein the compound of Formula (I) is effective to reduce RBP4 concentrations in dosage forms of about 1 to about 200 mg or about 5 to about 25 mg. Further provided herein are pharmaceutical compositions wherein the compound of Formula (I) is a micronized crystalline. Further provided herein are pharmaceutical compositions wherein the compound of Formula (I) is a polymorph exhibiting an x-ray powder diffraction pattern having at least three characteristic peaks expressed in degrees two theta (+/−0.5 degree theta) at 6.7, 9.3, 14.1, 17.2, 23.5, 27.1, and/or 29.0. Further provided herein are pharmaceutical compositions wherein the compound of Formula (I) is amorphous. Further provided herein are pharmaceutical compositions wherein the at least one excipient comprises a disintegrant. Further provided herein are pharmaceutical compositions wherein the disintegrant is selected from the group comprising agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof. Further provided herein are pharmaceutical compositions wherein the disintegrant comprises CCNa, MCC, crospovidone, tapioca starch, or a combination thereof. Further provided herein are pharmaceutical compositions wherein the disintegrant is about 0.01-99% by weight, 1-50% by weight, or 2-20% by weight of the pharmaceutical composition. Further provided herein are pharmaceutical compositions wherein the CCNa or MCC is about 0.75% or 3% by weight of the pharmaceutical composition. Further provided herein are pharmaceutical compositions wherein the at least one excipient comprises a dispersion polymer. Further provided herein are pharmaceutical compositions wherein the dispersion polymer is selected from the group comprising hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, and combinations thereof. Further provided herein are pharmaceutical compositions wherein the compound of Formula (I) is amorphous and/or molecularly dispersed in the dispersion polymer. Further provided herein are pharmaceutical compositions wherein the dispersion polymer comprises HPC, HPMC, or both. Further provided herein are pharmaceutical compositions wherein the dispersion polymer comprises HPMC, HPMC-AS, PVP, or a combination thereof. Further provided herein are pharmaceutical compositions wherein the dispersion polymer is about 0.01-99% by weight, 1-50% by weight, or 2-20% by weight of the pharmaceutical composition. Further provided herein are pharmaceutical compositions wherein the HPC or HPMC is about 6% by weight of the pharmaceutical composition. Further provided herein are pharmaceutical compositions wherein the HPMC, HPMC-AS, or PVP is about 10% or 20% by weight of the pharmaceutical composition. Further provided herein are pharmaceutical compositions wherein the at least one excipient comprises about 0.75% of CCNa, MCC, crospovidone, or tapioca starch by weight of the pharmaceutical composition and about 6% of HPC by weight of the of the pharmaceutical composition. Further provided herein are pharmaceutical compositions wherein the at least one excipient comprises about 0.75% of CCNa, MCC, crospovidone, or tapioca starch by weight of the pharmaceutical composition and about 10% of HPMC by weight of the pharmaceutical composition. Further provided herein are pharmaceutical compositions wherein the at least one excipient comprises about 0.75% of CCNa, MCC, crospovidone, or tapioca starch by weight of the pharmaceutical composition and about 20% of HPMC by weight of the pharmaceutical composition. Further provided herein are pharmaceutical compositions wherein the compound of Formula (I) is

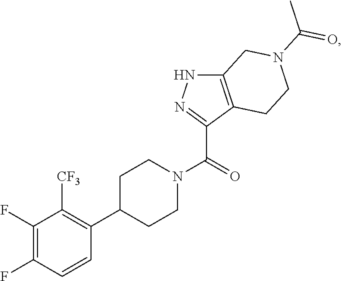 [SMILES: CC(=O)N1CCc2c(C(=O)N3CCC(c4ccc(F)c(F)c4C(F)(F)F)CC3)n[nH]c2C1]

or a pharmaceutically acceptable salt, crystalline solvate, polymorph, prodrug, metabolite, N-oxide, stereoisomer, or isomer thereof. Further provided herein are pharmaceutical compositions wherein the at least one excipient comprises a diluent, a binder, an anti-adherent, a filler, a sweetener, a wetting agent, a glidant, a lubricant, or a surfactant, or any combinations thereof. Further provided herein are pharmaceutical compositions wherein the compound of Formula (I) is not formulated for delayed release. Further provided herein are pharmaceutical compositions wherein the at least one excipient is not formulated for delayed release. Further provided herein are pharmaceutical compositions further comprising an external coating. Further provided herein are pharmaceutical compositions wherein the external coating is about 0.01-99% by weight, 1-50% by weight, or 2-20% by weight of the pharmaceutical composition. Further provided herein are pharmaceutical compositions wherein the external coating does not cause delayed release of the compound of Formula (I) and/or the at least one excipient. Further provided herein are pharmaceutical compositions wherein the diluent comprises MCC, lactose monohydrate (LMH), or both. Further provided herein are pharmaceutical compositions wherein the binder comprises MCC, HPC, or both. Further provided herein are pharmaceutical compositions wherein the anti-adherent comprises colloidal silicon dioxide (CSD), magnesium stearate, or both. Further provided herein are pharmaceutical compositions wherein the glidant comprise CSD. Further provided herein are pharmaceutical compositions wherein the lubricant comprises magnesium stearate. Further provided herein are pharmaceutical compositions wherein the at least one excipient comprises one or more of MCC, LMH, HPC, CCNa, CSD and magnesium stearate. Further provided herein are pharmaceutical compositions wherein the at least one excipient comprises two or more of MCC, LMH, HPC, CCNa, CSD and magnesium stearate. Further provided herein are pharmaceutical compositions wherein the diluent functions as a binder and/or a disintegrant. Further provided herein are pharmaceutical compositions wherein the anti-adherent functions as a gliant or a lubricant. Further provided herein are pharmaceutical compositions wherein the dry weight of the pharmaceutical composition is from about 0.1 mg to about 400 mg. Further provided herein are pharmaceutical compositions wherein the compound of Formula (I) is about 1-99.99% by weight, about 10-80% by weight, or about 20-60% by weight of the pharmaceutical composition. Further provided herein are pharmaceutical compositions comprising 1-10% by weight of the compound of Formula (I). Further provided herein are pharmaceutical compositions comprising 0.1-1.5% by weight of CCNa. Further provided herein are pharmaceutical compositions comprising 1-10% by weight of HPC. Further provided herein are pharmaceutical compositions comprising 15-60% by weight of MCC. Further provided herein are pharmaceutical compositions comprising 15-60% by weight of LMH. Further provided herein are pharmaceutical compositions comprising 0.1-2% by weight of Magnesium Stearate. Further provided herein are pharmaceutical compositions wherein the pharmaceutical composition does not exhibit substantial loss of chemical stability, degradation, and/or decomposition after about 1 week, 2 weeks, 3 weeks, 4 weeks, 10 weeks, 20 weeks, 30 weeks, 40 weeks, one year, two years, or three years at 25° C. when stored in otherwise atmospheric conditions. Further provided herein are pharmaceutical compositions wherein the pharmaceutical composition does not exhibit substantial loss of chemical stability, degradation, and/or decomposition of the compound of Formula (I), a pharmaceutically acceptable salt, crystalline, solvate, polymorph, prodrug, metabolite, N-oxide, stereoisomer, or isomer thereof, or the at least one excipient after about 1 week, 2 weeks, 3 weeks, 4 weeks, 10 weeks, 20 weeks, 30 weeks, 40 weeks, one year, two years, or three years at 25° C. when stored in otherwise atmospheric conditions. Further provided herein are pharmaceutical compositions wherein the pharmaceutical composition does not exhibit substantial loss of chemical stability, degradation, and/or decomposition after about 1 week, 2 weeks, 3 weeks, 4 weeks, 10 weeks, 20 weeks, 30 weeks, 40 weeks, or one year at 40° C. when stored in otherwise atmospheric conditions. Further provided herein are pharmaceutical compositions wherein the pharmaceutical composition does not exhibit substantial loss of chemical stability, degradation, and/or decomposition of the compound of Formula (I), a pharmaceutically acceptable salt, crystalline, solvate, polymorph, prodrug, metabolite, N-oxide, stereoisomer, or isomer thereof, or the at least one excipient after about 1 week, 2 weeks, 3 weeks, 4 weeks, 10 weeks, 20 weeks, 30 weeks, 40 weeks, or one year at 40° C. when stored in otherwise atmospheric conditions. Further provided herein are pharmaceutical compositions wherein at least about 5%, 10%, 25%, 50%, 75%, 80%, 90%, 95%, or 100% of the pharmaceutical composition by weight has dissociated or dissolved after about 5 minutes, 10 minutes, 20 minutes, 30 minutes, 40 minutes, 50 minutes, or one hour when placed in an aqueous medium at about pH 7 and at 25° C. Further provided herein are pharmaceutical compositions wherein at least about 5%, 10%, 25%, 50%, 75%, 80%, 90%, 95%, or 100% of the pharmaceutical composition by weight has dissociated or dissolved after about 5 minutes, 10 minutes, 20 minutes, 30 minutes, 40 minutes, 50 minutes, or one hour when placed in a simulated gastric fluid at 37° C. Further provided herein are pharmaceutical compositions wherein the aqueous medium comprises water. Further provided herein are pharmaceutical compositions wherein the pharmaceutical composition is placed in USP Apparatus Type II at 50 rpm in 900 mL of simulated gastric fluid medium at 37° C. Further provided herein are pharmaceutical compositions wherein the at least one excipient is about 0.01-99% by weight, 1-50% by weight, or 2-20% by weight of the pharmaceutical composition.

Provided herein are methods of treating an eye disease comprising administering a therapeutically effective amount of a pharmaceutical composition described herein to a subject in need thereof. Further provided herein are pharmaceutical compositions wherein the eye disease is a disease characterized by excessive lipofuscin accumulation in the retina. Further provided herein are pharmaceutical compositions wherein the disease characterized by excessive lipofuscin accumulation comprises Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or an ABCA4 gene associated retinal disease.

Provided herein are methods for lowering the serum or plasma concentration of RBP4 in a subject, comprising administering to the subject a therapeutically effective amount of a pharmaceutical composition herein. Further provided herein are pharmaceutical compositions wherein the therapeutically effective amount of the pharmaceutical composition comprises about 0.1 mg to about 400 mg of the compound of Formula (I). T Further provided herein are pharmaceutical compositions wherein the therapeutically effective amount of the pharmaceutical composition comprises about 0.5 mg to about 50 mg of the compound of Formula (I). Further provided herein are pharmaceutical compositions wherein the therapeutically effective amount of the pharmaceutical composition comprises about 0.1 mg, about 0.5 mg, about 1 mg, about 5 mg, about 10 mg, about 25 mg, about 50 mg, about 100 mg, about 200 mg, or about 400 mg of the compound of Formula (I). Further provided herein are pharmaceutical compositions wherein the pharmaceutical composition is administered one, two, three, or four times daily. Further provided herein are pharmaceutical compositions wherein the pharmaceutical composition is administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. Further provided herein are pharmaceutical compositions wherein the pharmaceutical composition is administered once daily. Further provided herein are pharmaceutical compositions wherein the pharmaceutical composition is administered orally. Further provided herein are pharmaceutical compositions wherein the serum or plasma RBP4 concentration of the subject is reduced to below 1 μM after treatment. Further provided herein are methods of manufacturing a tablet pharmaceutical composition comprising the steps: (i) co-sifting a compound of Formula (I) described herein or its pharmaceutically acceptable salt, a disintegrant, a dispersion polymer, two diluents, a glidant and a lubricant through a 30-mesh screen; (ii) loading the sifted materials from step (i) to a blender and blending for a first period of time; (iii) unloading the blended materials from step (ii) and sifting through a 30-mesh screen; (iv) adding the sifted materials from step (iii) to a blender and blending for a second period of time; (v) co-sifting a glidant and the lubricant through a 60-mesh screen and lubricating the blended materials from the step (iv) with a blender for a third period of time; (vi) granulating the lubricated materials from step (v) by roller compaction; (vii) sifting the lubricant through a 60-mesh screen and lubricating with granulated materials from the step (vi) for a fourth period of time; and (viii) compressing the materials from the step (vii) into the tablet pharmaceutical composition. Further provided herein are methods further comprising packaging the manufactured tablet pharmaceutical composition into a bottle. Further provided herein are methods wherein the disintegrant comprises agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, or combination thereof. Further provided herein are methods wherein the dispersion polymer comprises hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, poly oxyethylene-poly oxypropylene block copolymers, or combination thereof. Further provided herein are methods wherein the diluent comprises CCNa, HPC, MCC, LMH, CSD, magnesium stearate, or a combination thereof. Further provided herein are methods wherein the glidant comprises colloidal silicon dioxide, talk, corn starch, metal silicates, higher fatty acid metal salts, metal oxides, alkaline earth metal salts, metal hydroxides, or combination thereof. Further provided herein are methods wherein the lubricant comprises magnesium stearate, calcium stearate, zinc stearate, sodium stearate, stearic acid, aluminum stearate, leucine, glyceryl behenate, hydrogenated vegetable oil, sodium stearyl fumarate, or any combination thereof. Further provided herein are methods wherein the blending in step (ii) is carried out for 15 min at 15 rpm. Further provided herein are methods wherein the blending in step (iv) is carried out for 15 min at 15 rpm. Further provided herein are methods wherein the blending in step (v) is carried out for 5 min at 15 rpm. Further provided herein are methods wherein the blending in step (vii) is carried out for 5 min at 15 rpm. Further provided herein are methods wherein the step (vi) is carried out at a roll pressure from about 2 to about 5 MPa, a roll gap from about 0.3 to about 4 mm, a roll speed from about 3 to about 7 rpm, and/or a feed screw speed from about 18 to about 81 rpm. Further provided herein are methods wherein the step (vi) is carried out at a roll pressure of about 3 MPa, a roll gap of about 2 mm, a roll speed of about 4 rpm, and/or a feed screw speed of about 20 rpm. Further provided herein are methods wherein the step (viii) is carried out using a round-shaped punch of about 6.0 mm at a rotary speed from about 20 to about 30 rpm, a thickness scale from about 1.0 to about 3.5 mm, a fill depth scale from about 3.0 to about 10.0 mm, and/or a main compression force from about 3.0 to about 10.0 kN. Further provided herein are methods wherein the step (viii) is carried out at a rotary speed of about 30 rpm, a thickness scale of about 2.0 mm, a fill depth scale of about 6.0 mm, and/or a main compression force of about 7.0 kN. Further provided herein are methods wherein the bulk density of the lubricated materials of step (vi) is from about 0.45 to about 0.6 g/ml. Further provided herein are methods wherein the bulk density of the lubricated materials of step (vi) is from about 0.5 g/ml. Further provided herein are methods wherein the tablet pharmaceutical composition manufactured according to the method is from about 92.5 to about 107.5 mg. Further provided herein are methods wherein the tablet pharmaceutical composition manufactured according to the method is about 100 mg. Further provided herein are methods wherein the tablet pharmaceutical composition manufactured according to the method comprises a thickness from about 2.7 to about 3.3 mm. Further provided herein are methods wherein the tablet pharmaceutical composition manufactured according to the method comprises a thickness of about 3.0 mm. Further provided herein are methods wherein the tablet pharmaceutical composition manufactured according to the method comprises a hardness from about 45 to about 95N. Further provided herein are methods wherein the tablet pharmaceutical composition manufactured according to the method comprises a hardness of about 70N. Further provided herein are methods wherein the tablet pharmaceutical composition manufactured according to the method comprises a friability of no more than 1.0% by weight. Further provided herein are methods wherein the tablet pharmaceutical composition manufactured according to the method comprises a disintegration time of no more than 15 min. Further provided herein are methods wherein at least one of the two diluent functions as a binder and/or disintegrant. Further provided herein are methods wherein the glidant functions as an anti-adherent. Further provided herein are methods wherein the lubricant functions as an anti-adherent. Further provided herein are methods wherein at least one of the blenders used in steps (ii), (iv) and (v) is a bin blender of about 100 L. Further provided herein are methods wherein the step of packaging is carried out using a HDPE bottle of about 45 ml and the HDPE bottle contains 30 of the manufactured tablet pharmaceutical composition. Further provided herein are methods further comprising after the step (iv), taking a sample of the blended materials from about 98.5 to about 295.5 mg from each of about 10 locations of the blender. Further provided herein are methods wherein the sample is about 197 mg. Further provided herein are methods wherein the taking is carried out so that all the individual assays are within mean±10% (absolute) and RSD % and the NMT is about 5%. Further provided herein are methods wherein after step (v), testing BD and/or TD of the lubricated materials from step (v) so that the resulting density is around 0.5 g/ml or from about 0.45 to about 0.6 g/ml. Further provided herein are methods wherein the lubricant used in step (v) is intragranular. Further provided herein are methods wherein the lubricant used in step (vii) is extragranular. Further provided herein are methods wherein the disintegrant is selected from the group comprising agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof. Further provided herein are methods wherein the dispersion polymer selected from the group comprising hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, and combinations thereof. Further provided herein are methods wherein the compound of Formula (I) has the structure:

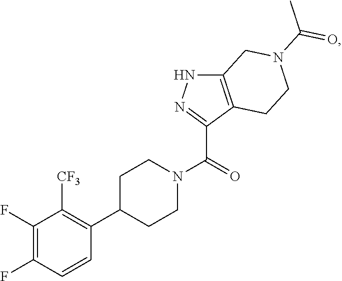 [SMILES: CC(=O)N1CCc2c(C(=O)N3CCC(c4ccc(F)c(F)c4C(F)(F)F)CC3)n[nH]c2C1]

or a pharmaceutically acceptable salt, crystalline solvate, polymorph, prodrug, metabolite, N-oxide, stereoisomer, or isomer thereof. Further provided herein are methods wherein each of the disintegrant, dispersion polymer, two diluents, glidant and lubricant is about 0.01-99% by weight, 1-50% by weight, or 2-20% by weight of the tablet pharmaceutical composition. Further provided herein are methods wherein the compound of Formula (I) is about 1-99.99% by weight, about 10-80% by weight, or about 20-60% by weight of the tablet pharmaceutical composition. Further provided herein are methods wherein the dry weight of the tablet pharmaceutical composition is from about 0.1 mg to about 400 mg.

Provided herein are methods for lowering the serum or plasma concentration of RBP4 in a subject, comprising administering to the subject a tablet pharmaceutical composition comprising a therapeutically effective amount of a compound having the structure

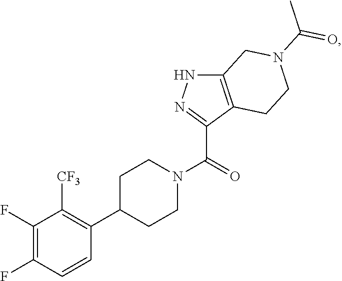 [SMILES: CC(=O)N1CCc2c(C(=O)N3CCC(c4ccc(F)c(F)c4C(F)(F)F)CC3)n[nH]c2C1]

or a pharmaceutically acceptable salt, crystalline, solvate, polymorph, prodrug, metabolite, N-oxide, stereoisomer, or isomer thereof and at least one excipient, wherein the pharmaceutical composition is administered in an amount of about 5 mg; wherein the pharmaceutical composition is administered daily; wherein the serum or plasma levels of RBP4 of the subject are reduced to below 1 μM.

Provided herein are pharmaceutical compositions comprising any one of formulas 1-140 from Tables 31-38. Provided herein are methods for lowering the serum or plasma concentration of RBP4 in a subject, comprising administering to the subject a tablet pharmaceutical described herein.

Provided herein are methods for treating Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or ABCA4 gene associated retinal diseases in a subject, comprising administering to the subject a tablet pharmaceutical provided herein.

### INCORPORATION BY REFERENCE

All publications, patents, and patent applications mentioned in this specification are herein incorporated by reference for the specific purposes identified herein.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the pharmacokinetic profiles of five different tablet formulations and the API-in-Capsule formulation of a compound of Formula (I). The y-axis is labeled Concentration of the Compound of Formula (I) (ng/ml, from 1-1000 at log 10 intervals); the x-axis is labeled Time (hours, from 0-24 at 4 hour intervals). Legend: Phase 1: API in Capsule (filled diamonds); Phase 2: 3% CCNa (open squares); Phase 3: 0.75% CCNa (filled circles); Phase 4: 0.75% CCNa+6% HPC (open triangles); Phase 5: 0.75% CCNa+10% HPMC (X's); Phase 6: 0.75% CCNa+20% HPMC (open circles).

FIG. 2 illustrates the pharmacodynamic profiles of the five tablet formulations and the API-in-Capsule formulation of a compound of Formula (I). The y-axis is labeled Mean RBP4 Concentration (ng/ml, from 1-25000 at 5000 unit intervals); the x-axis is labeled Time (hours, from 0-24 at 4 hour intervals). Legend: Phase 1: API in Capsule (filled diamonds); Phase 2: 3% CCNa (open squares); Phase 3: 0.75% CCNa (filled circles); Phase 4: 0.75% CCNa+6% HPC (open triangles); Phase 5: 0.75% CCNa+10% HPMC (X's); Phase 6: 0.75% CCNa+20% HPMC (open circles).

FIG. 3 illustrates the serum RBP4 reduction profiles of the five tablet formulations and the API-in-Capsule formulation of a compound of Formula (I). The y-axis is labeled Mean RBP4 Level (%, from 0-100 at 10% intervals); the x-axis is labeled Time (hours, from 0-24 at 4 hour intervals). Legend: Phase 1: API in Capsule (filled diamonds); Phase 2: 3% CCNa (open squares); Phase 3: 0.75% CCNa (filled circles); Phase 4: 0.75% CCNa+6% HPC (open triangles); Phase 5: 0.75% CCNa+10% HPMC (X's); Phase 6: 0.75% CCNa+200% HPMC (open circles).

### DETAILED DESCRIPTION OF THE INVENTION

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

### Definitions

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the =O radical.

“Thioxo” refers to the =S radical.

“Imino” refers to the =N—H radical.

“Oximo” refers to the =N—OH radical.

“Hydrazino” refers to the =N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), alkyl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl(optionally substituted with halogen, hydroxy, methoxy, or trifluoromethyl), carbocyclylalkyl (optionally substituted with halogen, hydroxy, methoxy, or trifluoromethyl), aryl(optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₈ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), alkyl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₅ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene). Unless stated otherwise specifically in the specification, an alkenylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atom (e.g., C₂ alkylene). In other embodiments, an alkynylene comprises five to eight carbon atoms (e.g., C₅-C₈ alkynylene). In other embodiments, an alkynylene comprises three to five carbon atoms (e.g., C₃-C₅ alkynylene). Unless stated otherwise specifically in the specification, an alkynylene chain is optionally substituted by one or more of the following substituents: halo, cyano, nitro, oxo, thioxo, imino, oximo, trimethylsilanyl, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl(optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R^{c}-aryl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Aralkenyl” refers to a radical of the formula —R^{d}-aryl where R^{d} is an alkenylene chain as defined above. The aryl part of the aralkenyl radical is optionally substituted as described above for an aryl group. The alkenylene chain part of the aralkenyl radical is optionally substituted as defined above for an alkenylene group.

“Aralkynyl” refers to a radical of the formula —R^{e}-aryl, where R^{e} is an alkynylene chain as defined above. The aryl part of the aralkynyl radical is optionally substituted as described above for an aryl group. The alkynylene chain part of the aralkynyl radical is optionally substituted as defined above for an alkynylene chain.

“Aralkoxy” refers to a radical bonded through an oxygen atom of the formula —O—R^{c}-ayl where R^{c} is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl(optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Carbocyclylalkynyl” refers to a radical of the formula —R^{c}-carbocyclyl where R^{c} is an alkynylene chain as defined above. The alkynylene chain and the carbocyclyl radical are optionally substituted as defined above.

“Carbocyclylalkoxy” refers to a radical bonded through an oxygen atom of the formula —O—R^{c}-carbocyclyl where R^{c} is an alkylene chain as defined above. The alkylene chain and the carbocyclyl radical are optionally substituted as defined above.

As used herein, “carboxylic acid bioisostere” refers to a functional group or moiety that exhibits similar physical, biological and/or chemical properties as a carboxylic acid moiety. Examples of carboxylic acid bioisosteres include, but are not limited to,

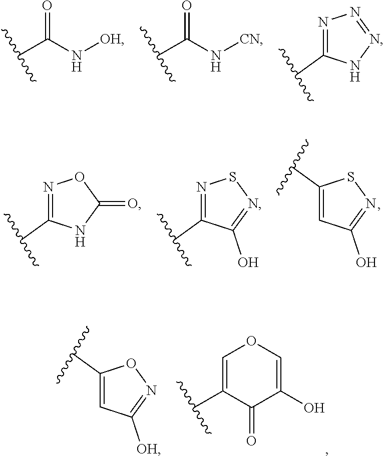

and the like.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclylradical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heterocyclylalkyl” refers to a radical of the formula —R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkyl radical is optionally substituted as defined above for an alkylene chain. The heterocyclyl part of the heterocyclylalkyl radical is optionally substituted as defined above for a heterocyclyl group.

“Heterocyclylalkoxy” refers to a radical bonded through an oxygen atom of the formula —O—R^{c}-heterocyclyl where R^{c} is an alkylene chain as defined above. If the heterocyclyl is a nitrogen-containing heterocyclyl, the heterocyclyl is optionally attached to the alkyl radical at the nitrogen atom. The alkylene chain of the heterocyclylalkoxy radical is optionally substituted as defined above for an alkylene chain. The heterocyclyl part of the heterocyclylalkoxy radical is optionally substituted as defined above for a heterocyclyl group.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

“Heteroarylalkyl” refers to a radical of the formula —R-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkyl radical is optionally substituted as defined above for an alkylene chain. The heteroaryl part of the heteroarylalkyl radical is optionally substituted as defined above for a heteroaryl group.

“Heteroarylalkoxy” refers to a radical bonded through an oxygen atom of the formula —O—R^{c}-heteroaryl, where R^{c} is an alkylene chain as defined above. If the heteroaryl is a nitrogen-containing heteroaryl, the heteroaryl is optionally attached to the alkyl radical at the nitrogen atom. The alkylene chain of the heteroarylalkoxy radical is optionally substituted as defined above for an alkylene chain. The heteroaryl part of the heteroarylalkoxy radical is optionally substituted as defined above for a heteroaryl group.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, itis intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

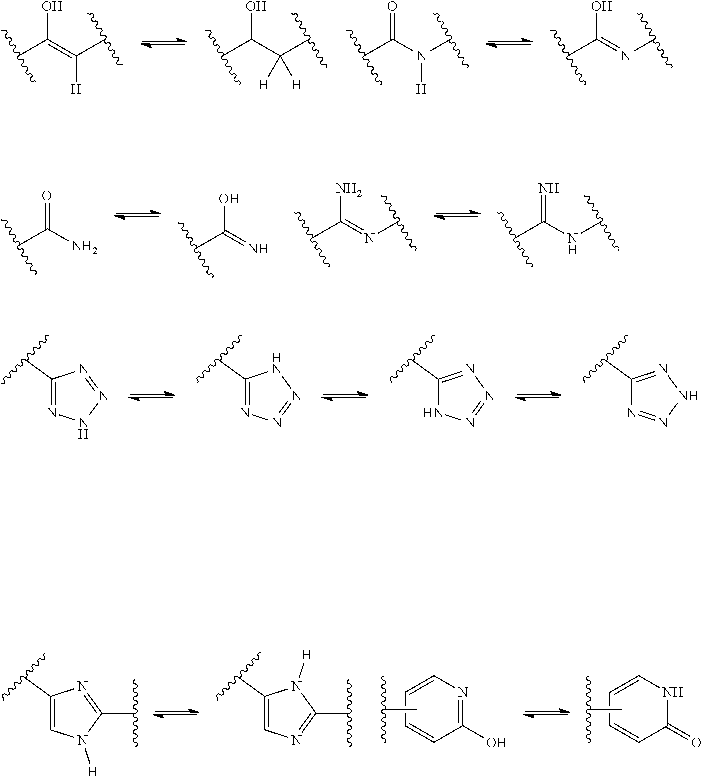

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Deuterium-transfer reagents suitable for use in nucleophilic substitution reactions, such as iodomethane-d₃ (CD₃I), are readily available and may be employed to transfer a deuterium-substituted carbon atom under nucleophilic substitution reaction conditions to the reaction substrate. The use of CD₃I is illustrated, by way of example only, in the reaction schemes below.

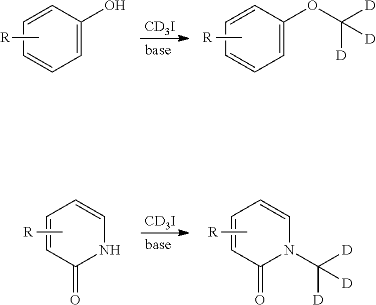

Deuterium-transfer reagents, such as lithium aluminum deuteride (LiAlD₄), are employed to transfer deuterium under reducing conditions to the reaction substrate. The use of LiAlD₄ is illustrated, by way of example only, in the reaction schemes below.

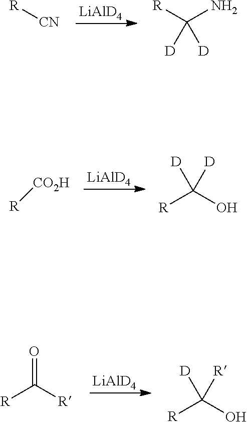

Deuterium gas and palladium catalyst are employed to reduce unsaturated carbon-carbon linkages and to perform a reductive substitution of aryl carbon-halogen bonds as illustrated, by way of example only, in the reaction schemes below.

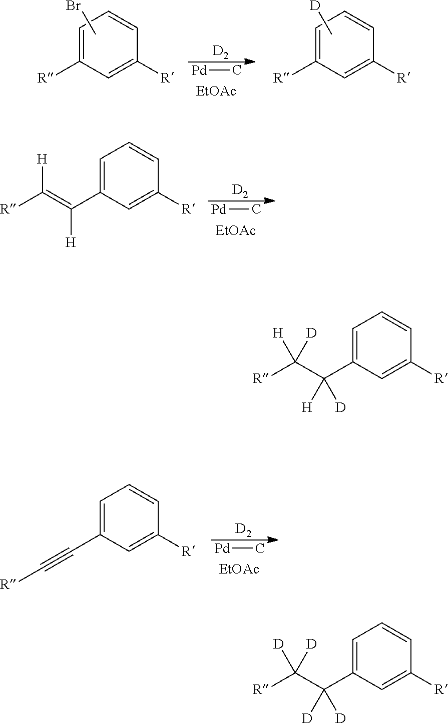

In one embodiment, the compounds disclosed herein contain one deuterium atom. In another embodiment, the compounds disclosed herein contain two deuterium atoms. In another embodiment, the compounds disclosed herein contain three deuterium atoms. In another embodiment, the compounds disclosed herein contain four deuterium atoms. In another embodiment, the compounds disclosed herein contain five deuterium atoms. In another embodiment, the compounds disclosed herein contain six deuterium atoms. In another embodiment, the compounds disclosed herein contain more than six deuterium atoms. In another embodiment, the compound disclosed herein is fully substituted with deuterium atoms and contains no non-exchangeable ¹H hydrogen atoms. In one embodiment, the level of deuterium incorporation is determined by synthetic methods in which a deuterated synthetic building block is used as a starting material.

Throughout the specification, examples, and claims, various components are expressed as being present in ratios, e.g. 1:2, 1:3, 1:4, or 1:5 and the like. Unless otherwise specified, such ratios refer to the ratio of each component by weight.

“Pharmaceutically acceptable salt” includes both acid and base addition salts. A pharmaceutically acceptable salt of any one of the heterocyclic RBP4 inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

“Prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam).

A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987.

The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like

Throughout the specification and claims, x-ray powder diffraction (XRPD) peaks are described. XRPD peak values in the application refer to those obtained using a copper source with a wavelength of 1.5406 angstrom unless otherwise noted.

As used herein, “Compound 1” or “CMPD-1” refers to Compound No. 1 as indicated in Table 1. Compound 1 has the structure

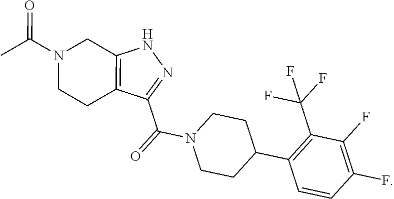 [SMILES: CC(=O)N1CCc2c(C(=O)N3CCC(c4ccc(F)c(F)c4C(F)(F)F)CC3)n[nH]c2C1]

Compound 1 is also referred to by its full chemical name of 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)ethan-1-one.

### RBP4 Inhibitory Compounds

Provided herein in some embodiments are RBP4 inhibitory compounds and pharmaceutical compositions comprising said compounds. The subject compounds and compositions are useful for inhibiting RPB4 and for the treatment of eye diseases or disorders, such as Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or ABCA4 gene associated retinal diseases.

Some embodiments provided herein describe a compound, or a pharmaceutically acceptable salt, solvate, polymorph, prodrug, metabolite, N-oxide, stereoisomer, or isomer thereof, for use in treating a metabolic disease or disorder, having the structure of Formula (I):

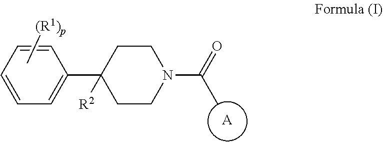

wherein:
   each R¹ is independently halogen, optionally substituted alkyl, optionally substituted cycloalkyl, optionally substituted heterocyclyl, optionally substituted heterocycloalkyl, —COR⁷, —CON(R⁷)₂, optionally substituted (C₀-C₄ alkylene)-CN, optionally substituted (C₀-C₄ alkylene)-OR⁷, optionally substituted (C₀-C₄ alkylene)-N(R⁷)₂, optionally substituted (C₀-C₄ alkylene)N(R⁸)—COR⁷, optionally substituted (C₀-C₄ alkylene)-SO₂N(R⁷)₂, optionally substituted (C₀-C₄ alkylene)-SO₂R⁷, optionally substituted (C₀-C₄ alkylene)N(R⁸)—SO₂N(R⁷)₂, or optionally substituted (C₀-C₄ alkylene)N(R⁸)—SO₂R⁷;
      each R⁷ is independently selected from H, optionally substituted alkyl, optionally substituted alkenyl, optionally substituted carbocyclyl, optionally substituted carbocyclylalkyl, optionally substituted aryl, optionally substituted aralkyl, optionally substituted heterocyclyl, optionally substituted heterocyclylalkyl, optionally substituted heteroaryl, or optionally substituted heteroarylalkyl; or two R¹¹ groups together with the nitrogen to which they are attached join to form an optionally substituted N-heterocyclyl;
      each R⁸ is independently selected from H or optionally substituted alkyl;
   R² is —H, —OH, optionally substituted alkyl, or halogen;
   p is 0, 1, 2, 3, 4, or 5;
   A has the structure:

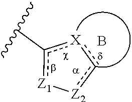

wherein:
   α, β, χ, and δ are each independently absent or present, and when present each is a bond;
   X is C;
   Z₁ is S, O, or N;
   Z₂ is S, O, N, or NR³;
   R³ is H, optionally substituted alkyl, or oxetane; and
   B is a substituted or unsubstituted fused 5-, 6-, or 7-membered ring structure.

Some embodiments provided herein describe a compound, or a pharmaceutically acceptable salt, solvate, polymorph, prodrug, metabolite, N-oxide, stereoisomer, or isomer thereof, having the structure of Formula (I) wherein:
each R¹ is independently halogen, optionally substituted C₁₋₆ alkyl, optionally substituted C₃₋₆ cycloalkyl, optionally substituted C₂₋₆ heterocyclyl, optionally substituted C₃₋₁₀ heterocycloalkyl, —COR⁷, —CON(R⁷)₂, optionally substituted (C₀-C₄ alkylene)-CN, optionally substituted (C₀-C₄ alkylene)-OR⁷, optionally substituted (C₀-C₄ alkylene)-N(R⁷)₂, optionally substituted (C₀-C₄ alkylene)N(R⁸)—COR⁷, optionally substituted (C₀-C₄ alkylene)-SO₂N(R⁷)₂, optionally substituted (C₀-C₄ alkylene)-SO₂R⁷, optionally substituted (C₀-C₄ alkylene)N(R⁸)—SO₂N(R⁷)₂, or optionally substituted (C₀-C₄ alkylene)N(R⁸)—SO₂R⁷;
each R⁷ is independently selected from H, optionally substituted C₁₋₆ alkyl, optionally substituted C₃₋₆ carbocyclyl, optionally substituted C₃₋₁₀ carbocyclylalkyl, optionally substituted C₂₋₆ heterocyclyl, optionally substituted C₂₋₁₀ heterocyclylalkyl; or two R¹¹ groups together with the nitrogen to which they are attached join to form an optionally substituted C₂-6 N-heterocyclyl;
each R⁸ is independently selected from H or optionally substituted C₁₋₆ alkyl;
R² is —H, —OH, optionally substituted C₁₋₆ alkyl, or halogen;
p is 0, 1, 2, 3, 4, or 5;
A has the structure:

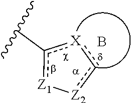

wherein:
α, β, χ, and δ are each independently absent or present, and when present each is a bond;
X is C;
Z₁ is S, O, or N;
Z₂ is S, O, N, or NR³;
R³ is H, optionally substituted C₁₋₆ alkyl, or oxetane; and
B is a substituted or unsubstituted fused 5-, 6-, or 7-membered ring structure.

Certain embodiments provided herein describe a compound, or a pharmaceutically acceptable salt, solvate, polymorph, prodrug, metabolite, N-oxide, stereoisomer, or isomer thereof, having the structure of Formula (I) wherein:
each R¹ is independently halogen, C₁₋₆ alkyl, C₁₋₆ haloalkyl, C₃₋₆ cycloalkyl, C₂₋₆ heterocyclyl, C₃₋₁₀ heterocycloalkyl, —COR⁷, —CON(R⁷)₂, (C₀-C₄ alkylene)-CN, (C₀-C₄ alkylene)-OR⁷, (C₀-C₄ alkylene)-N(R⁷)₂, (C₀-C₄ alkylene)N(R⁸)—COR⁷, (C₀-C₄ alkylene)-SO₂N(R⁷)₂, (C₀-C₄ alkylene)-SO₂R⁷, (C₀-C₄ alkylene)N(R⁸)—SO₂N(R⁷)₂, or (C₀-C₄ alkylene)N(R⁸)—SO₂R⁷;
   each R⁷ is independently selected from H, C₁₋₆ alkyl, C₃₋₆ carbocyclyl, C₃₋₁₀ carbocyclylalkyl, C₂₋₆ heterocyclyl, C₂₋₁₀ heterocyclylalkyl; or two R¹¹ groups together with the nitrogen to which they are attached join to form a C₂-6 N-heterocyclyl;
   each R⁸ is independently selected from H or C₁₋₆ alkyl;
R² is —H, —OH, C₁₋₆ alkyl, or halogen;
p is 0, 1, 2, 3, 4, or 5; A has the structure:

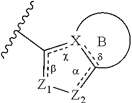

wherein:
   α, β, χ, and δ are each independently absent or present, and when present each is a bond;
   X is C;
   Z₁ is S, O, or N;
   Z₂ is S, O, N, or NR³;
   R³ is H, C₁₋₆ alkyl, or oxetane; and
   B is a substituted or unsubstituted fused 5-, 6-, or 7-membered ring structure.

Some embodiments provided herein describe a compound, or a pharmaceutically acceptable salt, solvate, polymorph, prodrug, metabolite, N-oxide, stereoisomer, or isomer thereof, having the structure of Formula (I) wherein:
each R¹ is independently halogen, haloalkyl, or alkyl;
R² is —H, —OH, or halogen;
p is 0, 1, 2, 3, 4, or 5;
A has the structure:

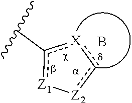

wherein:
   α, β, χ, and δ are each independently absent or present, and when present each is a bond;
   X is C;
   Z₁ is S, O, or N;
   Z₂ is S, O, N, or NR³;
   R³ is H, C₁-C₄ alkyl, or oxetane; and
   B is a substituted or unsubstituted fused 5-, 6-, or 7-membered ring structure.

Certain embodiments provided herein describe a compound, or a pharmaceutically acceptable salt, solvate, polymorph, prodrug, metabolite, N-oxide, stereoisomer, or isomer thereof, having the structure of Formula (I) wherein:
each R¹ is independently Br, Cl, F, C₁₋₆ fluoroalkyl, or C₁₋₆ alkyl;
R² is —H, —OH, Br, Cl, or F;
p is 0, 1, 2, 3, 4, or 5;
A has the structure:

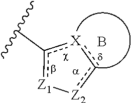

wherein:
   α, β, χ, and δ are each independently absent or present, and when present each is a bond;
   X is C;
   Z₁ is S, O, or N;
   Z₂ is S, O, N, or NR³;
   R³ is H, C₁-C₄ alkyl, or oxetane; and
   B is a substituted or unsubstituted fused 5-, 6-, or 7-membered ring structure.

For any and all of the embodiments of Formula (I), substituents are selected from among a subset of the listed alternatives.

In some embodiments, each R¹ is independently halogen, optionally substituted C₁₋₆ alkyl, optionally substituted C₃₋₆ cycloalkyl, optionally substituted C₂₋₆ heterocyclyl, optionally substituted C₃₋₁₀ heterocycloalkyl, —COR⁷, —CON(R⁷)₂, optionally substituted (C₀-C₄ alkylene)-CN, optionally substituted (C₀-C₄ alkylene)-OR⁷, optionally substituted (C₀-C₄ alkylene)-N(R⁷, optionally substituted (C₀-C₄ alkylene)N(R⁸)—COR⁷, optionally substituted (C₀-C₄ alkylene)-SO₂N(R⁷)₂, optionally substituted (C₀-C₄ alkylene)-SO₂R⁷, optionally substituted (C₀-C₄ alkylene)N(R⁸)—SO₂N(R⁷)₂, or optionally substituted (C₀-C₄ alkylene)N(R⁸)—SO₂R⁷. In certain embodiments, each R¹ is independently halogen, C₁₋₆ alkyl, C₁₋₆ haloalkyl, C₃₋₆ cycloalkyl, C₂₋₆ heterocyclyl, C₃₋₁₀ heterocycloalkyl, —COR⁷, —CON(R⁷)₂, (C₀-C₄ alkylene)-CN, (C₀-C₄ alkylene)-OR⁷, (C₀-C₄ alkylene)-N(R⁷)₂, (C₀-C₄ alkylene)N(R⁸)—COR⁷, (C₀-C₄ alkylene)-SO₂N(R⁷)₂, (C₀-C₄ alkylene)-SO₂R⁷, (C₀-C₄ alkylene)N(R⁸)—SO₂N(R⁷)₂, or (C₀-C₄ alkylene)N(R⁸)—SO₂R⁷. In some embodiments, each R¹ is independently halogen, C₁₋₆ alkyl, C₁₋₆ haloalkyl, —COR⁷, —CON(R⁷)₂, (C₀-C₄ alkylene)-CN, (C₀-C₄ alkylene)-OR⁷, or (C₀-C₄ alkylene)-N(R⁷)₂. In other embodiments, each R¹ is independently (C₀-C₄ alkylene)N(R⁸)—COR⁷, (C₀-C₄ alkylene)-SO₂N(R⁷)₂, (C₀-C₄ alkylene)-SO₂R⁷, (C₀-C₄ alkylene)N(R⁸)—SO₂N(R⁷)₂, or (C₀-C₄ alkylene)N(R⁸)—SO₂R⁷. In some embodiments, each R¹ is independently halogen, C₁₋₆ alkyl, C₁₋₆ haloalkyl, —COR⁷, —CON(R⁷)₂, —CN, (C₀-C₄ alkylene)-OR⁷, or (C₀-C₄ alkylene)-N(R⁷)₂. In some embodiments, each R¹ is independently halogen, C₁₋₆ alkyl, C₁₋₆ haloalkyl, or —CN. In certain embodiments, each R¹ is independently F, Br, Cl, C₁₋₆ haloalkyl, or C₁₋₆ alkyl. In specific embodiments, each R¹ is independently F or CF₃.

In some embodiments, each R⁷ is independently selected from H, optionally substituted C₁₋₆ alkyl, optionally substituted C₃₋₆ carbocyclyl, optionally substituted C₃₋₁₀ carbocyclylalkyl, optionally substituted C₂₋₆ heterocyclyl, optionally substituted C₂₋₁₀ heterocyclylalkyl; or two R¹¹ groups together with the nitrogen to which they are attached join to form an optionally substituted C₂₋₆ N-heterocyclyl. In some embodiments, each R⁷ is independently selected from H, C₁₋₆ alkyl, C₃₋₆ carbocyclyl, C₃₋₁₀ carbocyclylalkyl, C₂₋₆ heterocyclyl, C₂₋₁₀ heterocyclylalkyl; or two R¹¹ groups together with the nitrogen to which they are attached join to form a C₂₋₆ N-heterocyclyl. In some embodiments, each R⁷ is independently selected from H, C₁₋₆ alkyl, or C₃₋₆ carbocyclyl. In certain embodiments, two R¹¹ groups together with the nitrogen to which they are attached join to form an optionally substituted C₂₋₆ N-heterocyclyl. In some embodiments, each R⁷ is independently selected from H or C₁₋₆ alkyl. In some embodiments, each R⁷ is H or Me.

In some embodiments, each R⁸ is independently selected from H, C₁₋₆ alkyl, or C₁₋₆ haloalkyl. In some embodiments, each R⁸ is independently selected from H or C₁₋₆ alkyl. In some embodiments, each R⁸ is independently selected from H or Me. In some embodiments, each R⁸ is H.

In some embodiments, p is 0, 1, 2, 3, or 4. In some embodiments, p is 0, 1, 2, or 3. In some embodiments, p is 0, 1 or 2. In some embodiments, p is 0 or 1. In some embodiments, p is 1, 2, or 3. In some embodiments, p is 1 or 2. In some embodiments, p is 1, 2, 3, or 4. In some embodiments, p is 2, 3, or 4. In some embodiments, p is 2 or 3. In some embodiments, p is 0. In some embodiments, p is 1. In some embodiments, p is 2. In some embodiments, p is 3. In some embodiments, p is 4. In some embodiments, p is 5. In some embodiments, p is 1.

In some embodiments, R² is —H, —OH, optionally substituted alkyl, or halogen. In some embodiments, R² is —H, —OH, alkyl, haloalkyl, or halogen. In some embodiments, R² is —H, —OH, C₁₋₆ alkyl, C₁₋₆ haloalkyl, or halogen. In some embodiments, R² is —H, —OH, Me, CF₃, or halogen. In some embodiments, R² is —H, —OH, Me, CF₃, Cl, or F. In some embodiments, R² is —H, —OH, Me, CF₃, or F. In some embodiments, R² is —H, —OH, or halogen. In some embodiments, R² is —H, —OH, or F. In some embodiments, R² is —H. In some embodiments, R² is —OH. In some embodiments, R² is F. In some embodiments, R² is Cl.

In some embodiments, when a is present, then Z₁ is O or S, Z₂ is N, X is C, χ is present, and β and δ are absent. In other embodiments, when a is absent, then Z₁ is N, Z₂ is NR³, X is C, β and δ are present, and χ is absent. In certain embodiments, when a is absent, then Z₁ is N, Z₂ is O or S, X is C, β and δ are present, and χ is absent.

In some embodiments, A has the structure

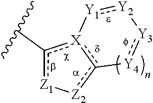

wherein:
   n is 0, 1, or 2;
   α, β, λ, S, ε, and ϕ are each independently absent or present, and when present each is a bond;
   Z₁ is S, O, or N;
   Z₂ is S, O, N or NR³,
      wherein R³ is H, C₁-C₄ alkyl, or oxetane;
   X is C;
   Y₁, Y₂, Y₃ and each occurrence of Y₄ are each independently CR⁴, C(R⁵)₂, NR⁶, O, N, SO₂, or —(C=O)—, wherein:
      R⁴ is H, halogen, C₁-C₁₀ alkyl, C₁-C₁₀ cycloalkyl, —O(C₁-C₁₀ alkyl), —C(O)OH, —C(O)O(C₁-C₁₀ alkyl), —C(O)NH₂, —C(O)NH (C₁-C₄ alkyl), —C(O)N(C₁-C₄ alkyl)₂, —NHC(O)NH(C₁-C₁₀ alkyl), —NHC(O)N(C₁-C₄ alkyl)₂, —SO₂NH(C₁-C₁₀ alkyl), —SO₂N(C₁-C₁₀ alkyl)₂, —CN, or —CF₃;
      R⁵ is H or C₁-C₁₀ alkyl; and
      R⁶ is H, C₁-C₁₀ alkyl, C₃-C₆ cycloalkyl, —(C₁-C₁₀ alkylene)CF₃, —(C₁-C₁₀ alkylene)OCH₃, —(C₁-C₁₀ alkylene)-halogen, —SO₂(C₁-C₁₀ alkyl), —SO₂(C₁-C₁₀ alkylene)-CF₃, —SO₂(C₁-C₁₀ alkylene)OCH₃, —SO₂(C₁-C₁₀ alkylene)-halogen, —C(O)(C₁-C₁₀ alkyl), —C(O)(C₁-C₁₀ alkylene)CF₃, —C(O)(C₁-C₁₀ alkylene)OCH₃, —C(O)(C₁-C₁₀ alkylene)-halogen, —C(O)NH(C₁-C₁₀ alkyl), —C(O)N(C₁-C₁₀ alkyl)₂, —(C₁-C₁₀ alkylene)C(O)OH, —C(O)NH₂, or oxetane.

In some embodiments, when a is present, then Z₁ is O or S, Z₂ is N, X is C, χ is present, and β and δ are absent. In other embodiments, when a is absent, then Z₁ is N, Z₂ is N, X is C, β and δ are present, and χ is absent. In certain embodiments, when a is absent, then Z₁ is N, Z₂ is O or S, X is C, β and δ are present, and χ is absent. In further or additional embodiments, when ε and ϕ are each present, then n=1, and each of Y₁, Y₂, Y₃, and Y₄, are independently —CR⁴— or N. In other embodiments, when ε and ϕ are each absent, then n=0, 1 or 2, each of Y₁, Y₂, Y₃, and each occurrence of Y₄ are independently C(R⁵)₂, NR⁶, O, or SO₂.

In some embodiments, β and δ are present. In some embodiments, α, χ, ε, and ϕ are absent. In some embodiments, Z₁ is N. In some embodiments, Z₂ is O, S, or NR³; wherein R³ is H, C₁-C₄ alkyl, or oxetane. In some embodiments, X is C. In certain embodiments, β and δ are present; α, χ, ε, and ϕ are absent; Z₁ is N; Z₂ is O, S, or NR³; R³ is H, C₁-C₄ alkyl, or oxetane; and X is C.

In some embodiments, β, δ, ε, and ϕ are present. In some embodiments, α, and χ are absent. In some embodiments, Z₁ is N. In some embodiments, Z₂ is O or NR₃, wherein R³ is H, C₁-C₄ alkyl, or oxetane. In some embodiments, X is C. In certain embodiments, β, δ, ε, and ϕ are present; α, and χ are absent; Z₁ is N; Z₂ is O or NR₃; R³ is H, C₁-C₄ alkyl, or oxetane; and X is C.

In some embodiments, A has the structure:

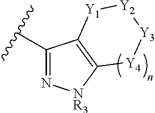

wherein
   n is 0;
   R³ is H, C₁-C₄ alkyl, or oxetane;
   Y₁ and Y₃ are each CH₂ or C(CH₃)₂;
   Y₂ is O, SO₂, or NR⁶; and
      R⁶ is H, C₁-C₄ alkyl, C₃-C₆ cycloalkyl, —(C₁-C₄ alkylene)CF₃, —(C₁-C₄ alkylene)OCH₃, —(C₁-C₄ alkylene)-halogen, —SO₂(C₁-C₄ alkyl), —SO₂(C₁-C₄ alkylene)CF₃, —SO₂(C₁-C₄ alkylene)OCH₃, —SO₂(C₁-C₄ alkylene)-halogen, —C(O)(C₁-C₄ alkyl), —C(O)(C₁-C₄ alkylene)CF₃, —C(O)(C₁-C₄ alkylene)OCH₃, —C(O)(C₁-C₄ alkylene)-halogen, —C(O)NH(C₁-C₄ alkyl), —C(O)N(C₁-C₄ alkyl)₂, —(C₁-C₄ alkylene)C(O)OH, —C(O)NH₂, or oxetane.

In some embodiments, A has the structure:

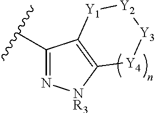

n is 1;
R³ is H, C₁-C₄ alkyl, or oxetane;
Y₁ and Y₄ are CH₂ or C(CH₃)₂;
Y₂ and Y₃ are each CH₂ or C(CH₃)₂, O, SO₂, or NR⁶; and
   R⁶ is H, C₁-C₄ alkyl, C₃-C₆ cycloalkyl, —(C₁-C₄ alkylene)CF₃, —(C₁-C₄ alkylene)OCH₃, —(C₁-C₄ alkylene)-halogen, —SO₂(C₁-C₄ alkyl), —SO₂(C₁-C₄ alkylene)CF₃, —SO₂(C₁-C₄ alkylene)OCH₃, —SO₂(C₁-C₄ alkylene)-halogen, —C(O)(C₁-C₄ alkyl), —C(O)(C₁-C₄ alkylene)CF₃, —C(O)(C₁-C₄ alkylene)OCH₃, —C(O)(C₁-C₄ alkylene)-halogen, —C(O)NH(C₁-C₄ alkyl), —C(O)N(C₁-C₄ alkyl)₂, —(C₁-C₄ alkylene)C(O)OH, —C(O)NH₂, or oxetane.

In some embodiments, A has the structure:

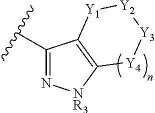

n is 2;
R³ is H, C₁-C₄ alkyl, or oxetane;
Y₁ and Y₄ are CH₂ or C(CH₃)₂;
Y₂ and Y₃ are each CH₂ or C(CH₃)₂, O, SO₂, or NR⁶; and
   R⁶ is H, C₁-C₄ alkyl, C₃-C₆ cycloalkyl, —(C₁-C₄ alkylene)CF₃, —(C₁-C₄ alkylene)OCH₃, —(C₁-C₄ alkylene)-halogen, —SO₂(C₁-C₄ alkyl), —SO₂(C₁-C₄ alkylene)CF₃, —SO₂(C₁-C₄ alkylene)OCH₃, —SO₂(C₁-C₄ alkylene)-halogen, —C(O)(C₁-C₄ alkyl), —C(O)(C₁-C₄ alkylene)CF₃, —C(O)(C₁-C₄ alkylene)OCH₃, —C(O)(C₁-C₄ alkylene)-halogen, —C(O)NH(C₁-C₄ alkyl), —C(O)N(C₁-C₄ alkyl)₂, —(C₁-C₄ alkylene)C(O)OH, —C(O)NH₂, or oxetane.

In some embodiments, A has the structure:

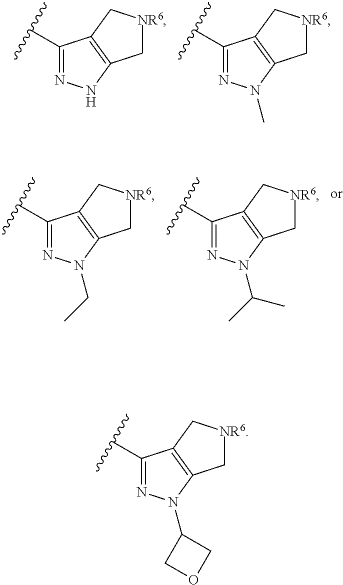

In other embodiments, A has the structure:

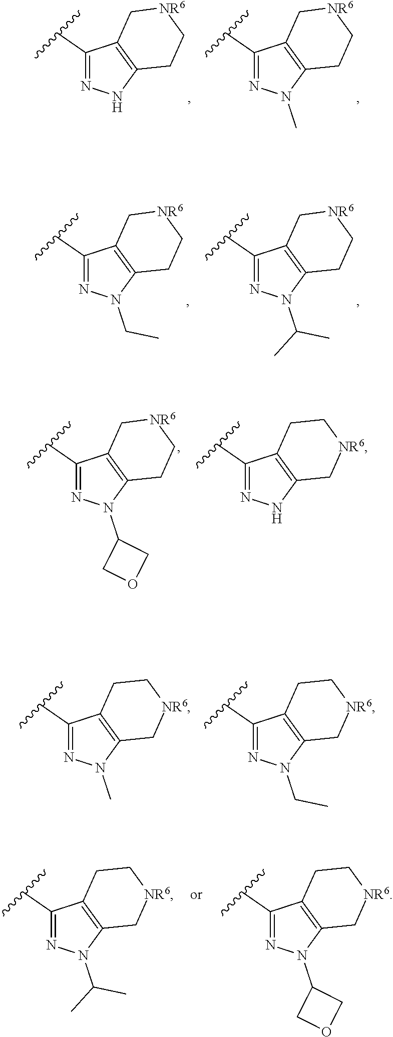

In certain embodiments, A has the structure:

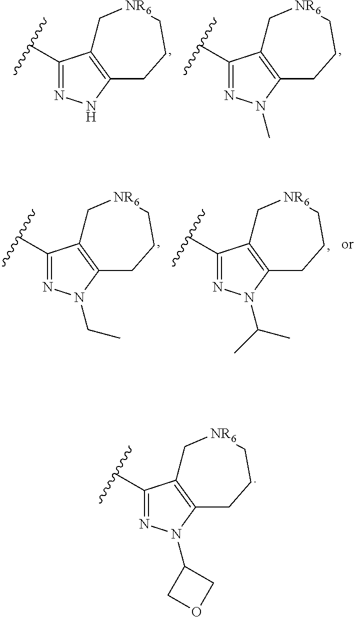

In certain embodiments, A has the structure:

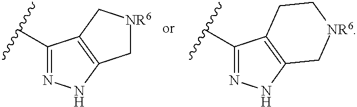

In certain embodiments, A has the structure:

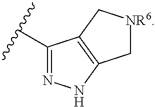

In certain embodiments, A has the structure:

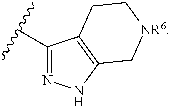

In some embodiments, R⁶ is H, C₁-C₆ alkyl, C₃-C₆ cycloalkyl, —(C₁-C₆ alkylene)CF₃, —(C₁-C₆ alkylene)OCH₃, —(C₁-C₆ alkylene)-halogen, —SO₂—C₁-C₆ alkyl, —SO₂(C₁-C₆ alkylene)-CF₃, —SO₂(C₁-C₆ alkylene)OCH₃, —SO₂(C₁-C₆ alkylene)-halogen, —C(O)(C₁-C₆ alkyl), —C(O)(C₁-C₆ alkylene)CF₃, —C(O)(C₁-C₆ alkylene)OCH₃, —C(O)(C₁-C₆ alkylene)-halogen, —C(O)NH(C₁-C₆ alkyl), —C(O)N(C₁-C₆ alkyl)₂, —(C₁-C₆ alkylene)C(O)OH, —C(O)NH₂, or oxetane. In some embodiments, R⁶ is C₁-C₆ alkyl, C₃-C₆ cycloalkyl, —(C₁-C₆ alkylene)CF₃, —(C₁-C₆ alkylene)OCH₃, —(C₁-C₆ alkylene)-halogen, —SO₂—C₁-C₆ alkyl, —SO₂(C₁-C₆ alkylene)-CF₃, —SO₂(C₁-C₆ alkylene)OCH₃, —SO₂(C₁-C₆ alkylene)-halogen, —C(O)(C₁-C₆ alkyl), —C(O)(C₁-C₆ alkylene)CF₃, —C(O)(C₁-C₆ alkylene)OCH₃, —C(O)(C₁-C₆ alkylene)-halogen, —C(O)NH(C₁-C₆ alkyl), —C(O)N(C₁-C₆ alkyl)₂, —(C₁-C₆ alkylene)C(O)OH, —C(O)NH₂, or oxetane. In some embodiments, R⁶ is —C(O)(C₁-C₆ alkyl). In some embodiments, R⁶ is H, C₁-C₄ alkyl, —CH₂CH₂CH₃, —CH(CH₃)₂, —CH₂CH(CH₃)₂, t-Bu, —CH₂OCH₃, —CH₂CF₃, —CH₂Cl, —CH₂F, —CH₂CH₂OCH₃, —CH₂CH₂CF₃, —CH₂CH₂Cl, —CH₂CH₂F,

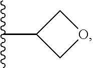 [SMILES: *C1COC1]

—SO₂CH₃, —SO₂CH₂CH₃, —SO₂CH₂CH₂CH₃, —SO₂CH(CH₃)₂, —SO₂CH₂CH(CH₃)₂, —SO₂(t-Bu), —SO₂CH₂OCH₃, —SO₂CH₂CF₃, —SO₂CH₂Cl, —SO₂CH₂F, —SO₂CH₂CH₂OCH₃, —SO₂CH₂CH₂CF₃, —SO₂CH₂CH₂C₁, —SO₂CH₂CH₂F,

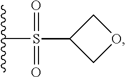 [SMILES: *S(=O)(=O)C1COC1]

C(O)CH₃, C(O)CH₂CH₃, —C(O)CH₂CH₂CH₃, —C(O)CH(CH₃)₂, —C(O)CH₂CH(CH₃)₂, —C(O)t-Bu, —C(O)CH₂OCH₃, —C(O)CH₂CF₃, —C(O)CH₂Cl, —C(O)CH₂F, —C(O)CH₂CH₂OCH₃, —C(O)CH₂CH₂CF₃, —C(O)CH₂CH₂Cl, —C(O)CH₂CH₂F,

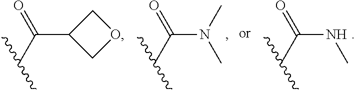

In some embodiments, R⁶ is —C(O)(C₁-C₆ alkyl). In some embodiments, R⁶ is H, C₁-C₄ alkyl, —CH₂CH₂CH₃, —CH(CH₃)₂, —CH₂CH(CH₃)₂, t-Bu, —CH₂OCH₃, —CH₂CF₃, —CH₂Cl, —CH₂F, —CH₂CH₂OCH₃, —CH₂CH₂CF₃, —CH₂CH₂Cl, —CH₂CH₂F, or

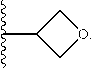 [SMILES: *C1COC1]

In other embodiments, R⁶ is —SO₂CH₃, —SO₂CH₂CH₃, —SO₂CH₂CH₂CH₃, —SO₂CH(CH₃)₂, —SO₂CH₂CH(CH₃)₂, —SO₂(t-Bu), —SO₂CH₂OCH₃, —SO₂CH₂CF₃, —SO₂CH₂Cl, —SO₂CH₂F, —SO₂CH₂CH₂OCH₃, —SO₂CH₂CH₂CF₃, —SO₂CH₂CH₂Cl, —SO₂CH₂CH₂F, or

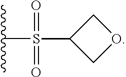 [SMILES: *S(=O)(=O)C1COC1]

In certain embodiments, R⁶ is C(O)CH₃, C(O)CH₂CH₃, —C(O)CH₂CH₂CH₃, —C(O)CH(CH₃)₂, —C(O)CH₂CH(CH₃)₂, —C(O)t-Bu, —C(O)CH₂OCH₃, —C(O)CH₂CF₃, —C(O)CH₂Cl, —C(O)CH₂F, C(O)CH₂CH₂OCH₃, —C(O)CH₂CH₂CF₃, —C(O)CH₂CH₂Cl, —C(O)CH₂CH₂F,

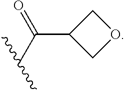 [SMILES: *C(=O)C1COC1]

In some embodiments, A has the structure:

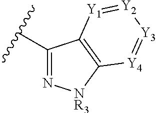

wherein:
   Y₁, Y₂, Y₃ and each occurrence of Y₄ are each independently CR⁴, or N;
   wherein:
      R³ is H, halogen, C₁-C₁₀ alkyl, C₁-C₁₀ cycloalkyl, —O(C₁-C₁₀ alkyl), —C(O)OH, —C(O)O(C₁-C₁₀ alkyl), —C(O)NH₂, —C(O)NH (C₁-C₄ alkyl), —C(O)N(C₁-C₄ alkyl)₂, —NHC(O)NH(C₁-C₁₀ alkyl), —NHC(O)N(C₁-C₄ alkyl)₂, —SO₂NH(C₁-C₁₀ alkyl), —SO₂N(C₁-C₁₀ alkyl)₂, —CN, or —CF₃.

In some embodiments, Y₁, Y₂, Y₃ and Y₄ are CH. In some embodiments, Y₁, Y₂, Y₃ are CH and Y₄ is N. In some embodiments, Y₁, Y₂, Y₄ are CH and Y₃ is N. In some embodiments, Y₁, Y₃, Y₄ are CH and Y₂ is N. In some embodiments, Y₂, Y₃, Y₄ are CH and Y₁ is N.

In certain embodiments, A has the structure:

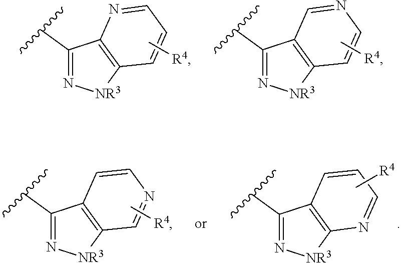

In some embodiments, R³ is H, halogen, C₁-C₁₀ alkyl, C₁-C₁₀ cycloalkyl, —O(C₁-C₁₀ alkyl), —C(O)OH, —C(O)O(C₁-C₁₀ alkyl), —C(O)NH₂, —C(O)NH (C₁-C₄ alkyl), —C(O)N(C₁-C₄ alkyl)₂, —NHC(O)NH(C₁-C₁₀ alkyl), —NHC(O)N(C₁-C₄ alkyl)₂, —SO₂NH(C₁-C₁₀ alkyl), —SO₂N(C₁-C₁₀ alkyl)₂, —CN, or —CF₃. In some embodiments, R³ is H, halogen, C₁-C₆ alkyl, C₁-C₆ cycloalkyl, —O(C₁-C₆ alkyl), —C(O)OH, —C(O)O(C₁-C₆ alkyl), —C(O)NH₂, —C(O)NH (C₁-C₄ alkyl), —C(O)N(C₁-C₄ alkyl)₂, —NHC(O)NH(C₁-C₆ alkyl), —NHC(O)N(C₁-C₄ alkyl)₂, —SO₂NH(C₁-C₆ alkyl), —SO₂N(C₁-C₆ alkyl)₂, —CN, or —CF₃. In some embodiments, R₄ is H, halogen, C₁-C₄ alkyl, C₃-C₆ cycloalkyl, —O(C₁-C₄ alkyl), —CN, —CF₃, —C(O)OH, —C(O)NH₂, —C(O)N(CH₃)₂, —C(O)NHCH₃, or —NHC(O)N(CH₃)₂. In some embodiments, R₄ is H, halogen, methyl, methoxy, —CN, —CF₃, —C(O)N(CH₃)₂, —C(O)NHCH₃, or —C(O)Me.

In some embodiments, the heterocyclic compounds of Formula (I) are provided in Table 1.

TABLE 1

| Compound | | |
|---|---|---|
| No. | Name | Structure |
| 1 | 1-(3-(4-(3,4-difluoro-2-(trifluoromethyl)phen- yl)piperidine-1-car- bonyl)-1,4,5,7-tetra- hydro-6H-pyrazolo [3,4-c]pyridin-6-yl)- ethan-1-one | 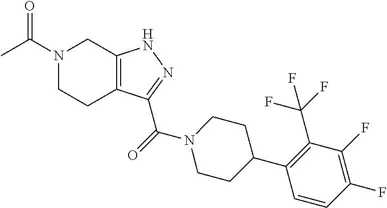 [SMILES: CC(=O)N1CCc2c(C(=O)N3CCC(c4ccc(F)c(F)c4C(F)(F)F)CC3)n[nH]c2C1] |
| 2 | 1-(3-(4-(3,4-difluoro-2- (trifluoromethyl)phen- yl)piperidine-1-carbon- yl)-4,6-dihydropyrrolo [3,4-c]pyrazol-5(1H)- yl)ethan-1-one | 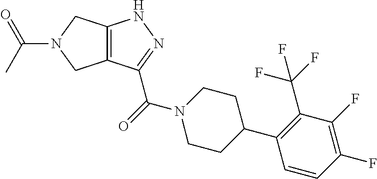 [SMILES: CC(=O)N1Cc2[nH]nc(C(=O)N3CCC(c4ccc(F)c(F)c4C(F)(F)F)CC3)c2C1] |
| 12 | (4-(3,4-difluoro-2- (trifluoromethyl)phen- yl)piperidin-1-yl)(6- ethyl-4,5,6,7-tetra- hydro-1H-pyrazolo [3,4-c]pyridin-3-yl)- methanone | 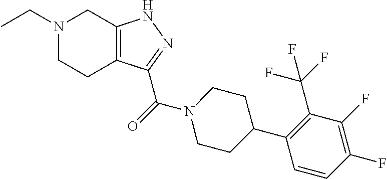 [SMILES: CCN1CCc2c(C(=O)N3CCC(c4ccc(F)c(F)c4C(F)(F)F)CC3)n[nH]c2C1] |
| 13 | (4-(4-fluoro-2-(tri- fluoromethyl)phenyl)- piperidin-1-yl)(5- (methylsulfonyl)- 4,5,6,7-tetrahydro- 1H-pyrazolo[4,3-c] pyridin-3-yl)meth- anone | 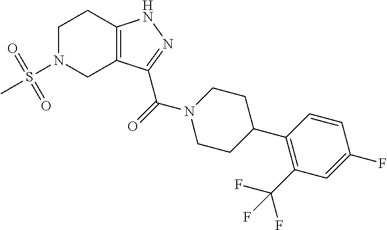 [SMILES: CS(=O)(=O)N1CCc2[nH]nc(C(=O)N3CCC(c4ccc(F)cc4C(F)(F)F)CC3)c2C1] |
| 14 | 1-(3-(4-(2-fluoro-6- (trifluoromethyl)phen- yl)piperidine-1-car- bonyl)-1,4,5,7-tetra- hydro-6H-pyrazolo [3,4-c]pyridin-6-yl)- ethan-1-one | 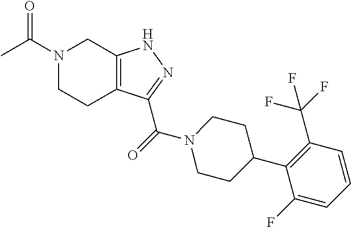 [SMILES: CC(=O)N1CCc2c(C(=O)N3CCC(c4c(F)cccc4C(F)(F)F)CC3)n[nH]c2C1] |
| 17 | (4-(3,5-bis(trifluoro- methyl)phenyl)piper- idin-1-yl)(5-(methyl- sulfonyl)-4,5,6,7-tetra- hydro-1H-pyrazolo [4,3-c]pyridin-3-yl)- methanone | 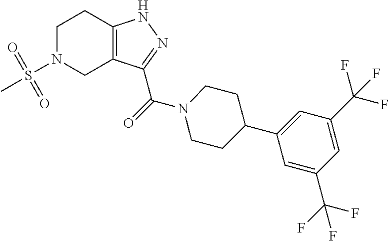 [SMILES: CS(=O)(=O)N1CCc2[nH]nc(C(=O)N3CCC(c4cc(C(F)(F)F)cc(C(F)(F)F)c4)CC3)c2C1] |
| 22 | 3-(4-(3,5-difluoro-2- (trifluoromethyl)phen- yl)piperidine-1-car- bonyl)-N-methyl- 1,4,5,7-tetrahydro-6H- pyrazolo[3,4-c]pyr- idine-6-carboxamide | 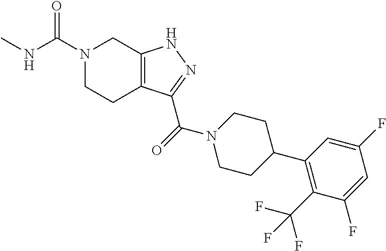 [SMILES: CNC(=O)N1CCc2c(C(=O)N3CCC(c4cc(F)cc(F)c4C(F)(F)F)CC3)n[nH]c2C1] |
| 23 | (6-(cyclopropylmeth- yl)-4,5,6,7-tetrahydro- 1H-pyrazolo[3,4-c] pyridin-3-yl)(4-(3,4- difluoro-2-(trifluoro- methyl)phenyl)piper- idin-1-yl)methanone | 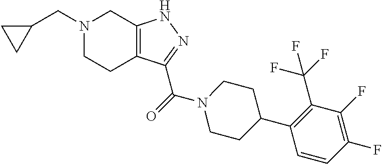 [SMILES: O=C(c1n[nH]c2c1CCN(CC1CC1)C2)N1CCC(c2ccc(F)c(F)c2C(F)(F)F)CC1] |
| 26 | (4-(3,4-difluoro-2- (trifluoromethyl)phen- yl)piperidin-1-yl)(5- neopentyl-4,5,6,7- tetrahydro-1H-pyr- azolo[4,3-c]pyridin- 3-yl)methanone | 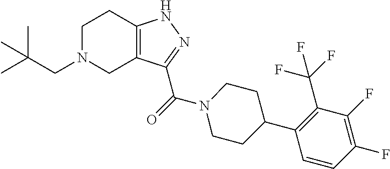 [SMILES: CC(C)(C)CN1CCc2[nH]nc(C(=O)N3CCC(c4ccc(F)c(F)c4C(F)(F)F)CC3)c2C1] |
| 27 | (4-(2-chloro-5-fluoro- phenyl)piperidin-1-yl) (5-(methylsulfonyl)- 4,5,6,7-tetrahydro-1H- pyrazolo[4,3-c]pyridin- 3-yl)methanone | 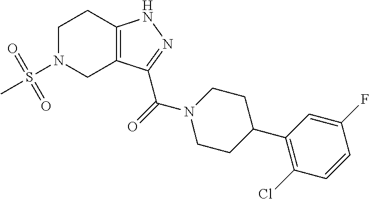 [SMILES: CS(=O)(=O)N1CCc2[nH]nc(C(=O)N3CCC(c4cc(F)ccc4Cl)CC3)c2C1] |
| 31 | 3-(4-(3,4-difluoro-2- (trifluoromethyl)phen- yl)piperidine-1-car- bonyl)-N-methyl- 1,4,5,7-tetrahydro- 6H-pyrazolo[3,4-c] pyridine-6-carbox- amide | 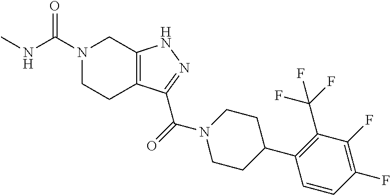 [SMILES: CNC(=O)N1CCc2c(C(=O)N3CCC(c4ccc(F)c(F)c4C(F)(F)F)CC3)n[nH]c2C1] |
| 32 | (4-(3,4-difluoro-2- (trifluoromethyl)phen- yl)piperidin-1-yl)(6- neopentyl-4,5,6,7- tetrahydro-1H-pyr- azolo[3,4-c]pyridin- 3-yl)methanone | 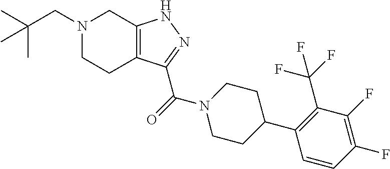 [SMILES: CC(C)(C)CN1CCc2c(C(=O)N3CCC(c4ccc(F)c(F)c4C(F)(F)F)CC3)n[nH]c2C1] |
| 35 | 1-(3-(4-(3,5-difluoro- 2-(trifluoromethyl)- phenyl)piperidine-1- carbonyl)-1,4,5,7- tetrahydro-6H-pyr- azolo[3,4-c]pyridin- 6-yl)ethan-1-one | 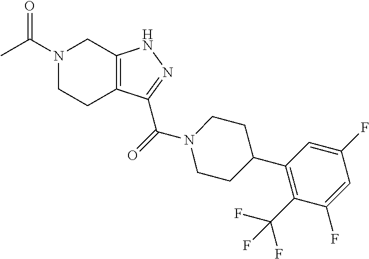 [SMILES: CC(=O)N1CCc2c(C(=O)N3CCC(c4cc(F)cc(F)c4C(F)(F)F)CC3)n[nH]c2C1] |
| 38 | 1-(3-(4-(3,4-difluoro- 2-(trifluoromethyl)- phenyl)piperidine-1- carbonyl)-1,4,5,7- tetrahydro-6H-pyr- azolo[3,4-c]pyridin- 6-yl)-3-methylbutan- 1-one | 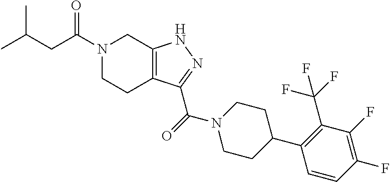 [SMILES: CC(C)CC(=O)N1CCc2c(C(=O)N3CCC(c4ccc(F)c(F)c4C(F)(F)F)CC3)n[nH]c2C1] |
| 39 | 1-(3-(4-(3,4-difluoro- 2-(trifluoromethyl)- phenyl)piperidine-1- carbonyl)-1,4,5,7- tetrahydro-6H-pyr- azolo[3,4-c]pyridin- 6-yl)propan-1-one | 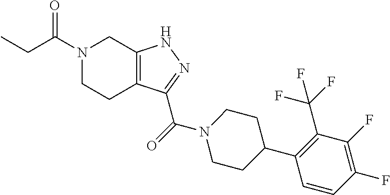 [SMILES: CCC(=O)N1CCc2c(C(=O)N3CCC(c4ccc(F)c(F)c4C(F)(F)F)CC3)n[nH]c2C1] |
| 41 | 3-(4-(3,4-difluoro- 2-(trifluoromethyl)- phenyl)piperidine-1- carbonyl)-1,4,5,7- tetrahydro-6H-pyr- azolo[3,4-c]pyridine- 6-carbonitrile | 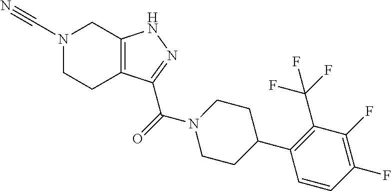 [SMILES: N#CN1CCc2c(C(=O)N3CCC(c4ccc(F)c(F)c4C(F)(F)F)CC3)n[nH]c2C1] |
| 44 | (4-(3,4-difluoro-2- (trifluoromethyl)- phenyl)piperidin- 1-yl)(5-(3,3,3-tri- fluoropropyl)- 4,5,6,7-tetrahydro- 1H-pyrazolo[4,3- c]pyridin-3-yl)- methanone | 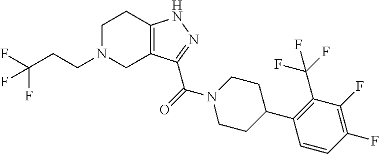 [SMILES: O=C(c1n[nH]c2c1CN(CCC(F)(F)F)CC2)N1CCC(c2ccc(F)c(F)c2C(F)(F)F)CC1] |
| 52 | 3-(4-(3-fluoro-2- (trifluoromethyl)- phenyl)piperidine- 1-carbonyl)-N- methyl-1,4,6,7- tetrahydro-5H- pyrazolo[4,3-c] pyridine-5-carbox- amide | 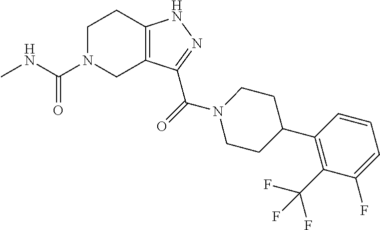 [SMILES: CNC(=O)N1CCc2[nH]nc(C(=O)N3CCC(c4cccc(F)c4C(F)(F)F)CC3)c2C1] |
| 57 | 3-(4-(3,4-difluoro-2- (trifluoromethyl)- phenyl)piperidine-1- carbonyl)-1,4,6,7- tetrahydro-5H-pyr- azolo[4,3-c]pyridine- 5-carbonitrile | 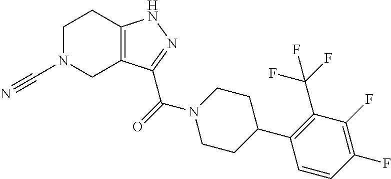 [SMILES: N#CN1CCc2[nH]nc(C(=O)N3CCC(c4ccc(F)c(F)c4C(F)(F)F)CC3)c2C1] |
| 63 | (4-(3-fluoro-2- (trifluoromethyl)- phenyl)piperidin- 1-yl)(5-(3,3,3- trifluoropropyl)- 4,5,6,7-tetrahydro- 1H-pyrazolo[4,3- c]pyridin-3-yl)- methanone | 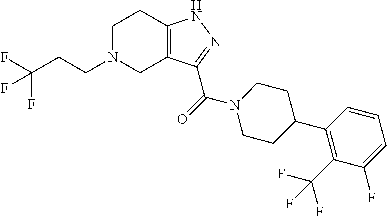 [SMILES: O=C(c1n[nH]c2c1CN(CCC(F)(F)F)CC2)N1CCC(c2cccc(F)c2C(F)(F)F)CC1] |
| 67 | 1-(3-(4-(3,4-difluoro- 2-(trifluoromethyl)- phenyl)piperidine-1- carbonyl)-1,4,6,7- tetrahydro-5H-pyr- azolo[4,3-c]pyridin- 5-yl)ethan-1-one | 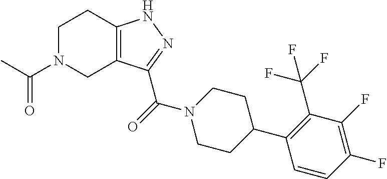 [SMILES: CC(=O)N1CCc2[nH]nc(C(=O)N3CCC(c4ccc(F)c(F)c4C(F)(F)F)CC3)c2C1] |
| 69 | 3-(4-(3-fluoro-2- (trifluoromethyl)- phenyl)piperidine-1- carbonyl)-N-methyl- 1,4,5,7-tetrahydro- 6H-pyrazolo[3,4-c] pyridine-6-carbox- amide | 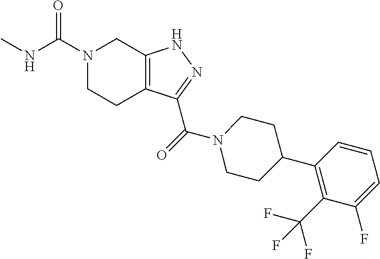 [SMILES: CNC(=O)N1CCc2c(C(=O)N3CCC(c4cccc(F)c4C(F)(F)F)CC3)n[nH]c2C1] |
| 70 | (4-(3-fluoro-2- (trifluoromethyl)- phenyl)piperidin-1- yl)(5-(methylsulfon- yl)-4,5,6,7-tetra- hydro-1H-pyrazolo [4,3-c]pyridin-3-yl)- methanone | 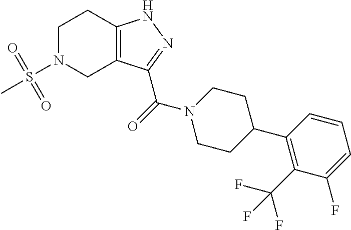 [SMILES: CS(=O)(=O)N1CCc2[nH]nc(C(=O)N3CCC(c4cccc(F)c4C(F)(F)F)CC3)c2C1] |
| 75 | (4-(3,4-difluoro-2- (trifluoromethyl)- phenyl)piperidin-1- yl)(4,5,6,7-tetra- hydro-1H-pyrazolo [4,3-c]pyridin-3- yl)methanone | 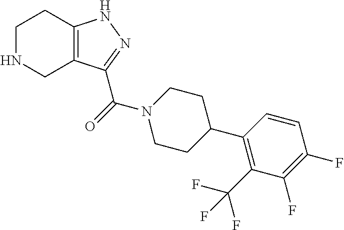 [SMILES: O=C(c1n[nH]c2c1CNCC2)N1CCC(c2ccc(F)c(F)c2C(F)(F)F)CC1] |
| 76 | (4-(3,4-difluoro-2- (trifluoromethyl) phenyl)piperidin-1- yl)(4,5,6,7-tetra- hydro-1H-pyrazolo [3,4-c]pyridin-3-yl)- methanone | 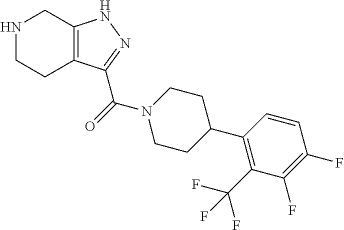 [SMILES: O=C(c1n[nH]c2c1CCNC2)N1CCC(c2ccc(F)c(F)c2C(F)(F)F)CC1] |
| 82 | 1-(3-(4-(3-fluoro-2- (trifluoromethyl)- phenyl)piperidine-1- carbonyl)-1,4,5,7- tetrahydro-6H-pyr- azolo[3,4-c]pyridin- 6-yl)ethan-1-one | 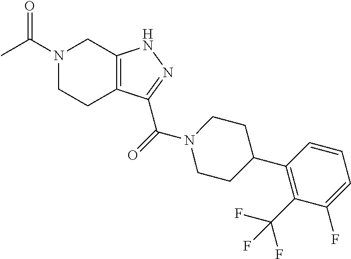 [SMILES: CC(=O)N1CCc2c(C(=O)N3CCC(c4cccc(F)c4C(F)(F)F)CC3)n[nH]c2C1] |
| 84 | 1-(3-(4-(3,5-bis(tri- fluoromethyl)phen- yl)piperidine-1- carbonyl)-1,4,5,7- tetrahydro-6H-pyr- azolo[3,4-c]pyridin- 6-yl)ethan-1-one | 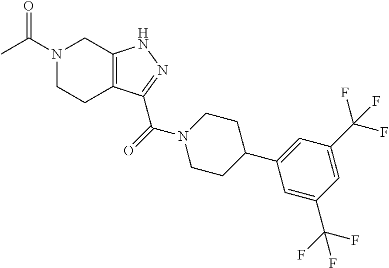 [SMILES: CC(=O)N1CCc2c(C(=O)N3CCC(c4cc(C(F)(F)F)cc(C(F)(F)F)c4)CC3)n[nH]c2C1] |
| 85 | 1-(3-(4-(4-fluoro-2- (trifluoromethyl)- phenyl)piperidine- 1-carbonyl)-1,4,5,7- tetrahydro-6H-pyr- azolo[3,4-c]pyridin- 6-yl)ethan-1-one | 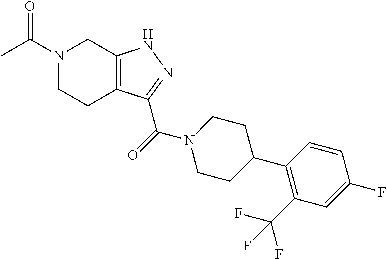 [SMILES: CC(=O)N1CCc2c(C(=O)N3CCC(c4ccc(F)cc4C(F)(F)F)CC3)n[nH]c2C1] |
| 88 | (5-(cyclopropylmeth- yl)-4,5,6,7-tetrahydro- 1H-pyrazolo[4,3- c]pyridin-3-yl)(4-(3- fluoro-2-(trifluoro- methyl)phenyl)piper- idin-1-yl)methanone | 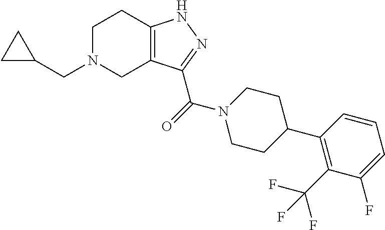 [SMILES: O=C(c1n[nH]c2c1CN(CC1CC1)CC2)N1CCC(c2cccc(F)c2C(F)(F)F)CC1] |

In certain embodiments, the heterocyclic compound of Formula (I) is 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(3,4-difluoro-2-(trifluoromethyl)phenyl)piperidine-1-carbonyl)-4,6-dihydropyrrolo[3,4-c]pyrazol-5(1H)-yl)ethan-1-one; (4-(3-fluoro-2,5-bis(trifluoromethyl)phenyl)piperidin-1-yl)(4,5,6,7-tetrahydro-1H-pyrazolo[4,3-c]pyridin-3-yl)methanone; (4-(2-chloro-3-fluorophenyl)piperidin-1-yl)(5-(2-methoxyethyl)-4,5,6,7-tetrahydro-1H-pyrazolo[4,3-c]pyridin-3-yl)methanone; (4-(2-chloro-3-fluorophenyl)piperidin-1-yl)(5-(3,3,3-trifluoropropyl)-4,5,6,7-tetrahydro-1H-pyrazolo[4,3-c]pyridin-3-yl)methanone; (4-(2-chloro-3-fluorophenyl)piperidin-1-yl)(5-(2,2,2-trifluoroethyl)-4,5,6,7-tetrahydro-1H-pyrazolo[4,3-c]pyridin-3-yl)methanone; (4-(2-chloro-3-fluorophenyl)piperidin-1-yl)(5-(oxetan-3-yl)-4,5,6,7-tetrahydro-1H-pyrazolo[4,3-c]pyridin-3-yl)methanone; (4-(4-fluoro-2-(trifluoromethyl)phenyl)piperidin-1-yl)(4,5,6,7-tetrahydro-1H-pyrazolo[4,3-c]pyridin-3-yl)methanone; (4-(4-fluoro-2-(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(5-(cy clopropylmethyl)-4,5,6,7-tetrahydro-1H-pyrazolo[4,3-c]pyridin-3-yl)methanone; (4-(2-chloro-3-fluorophenyl)piperidin-1-yl)(5-ethyl-4,5,6,7-tetrahydro-1H-pyrazolo[4,3-c]pyridin-3-yl)methanone; (4-(3,4-difluoro-2-(trifluoromethyl)phenyl)piperidin-1-yl)(6-ethyl-4,5,6,7-tetrahydro-1H-pyrazolo[3,4-c]pyridin-3-yl)methanone; (4-(4-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; 1-(3-(4-(2-fluoro-6-(trifluoromethyl)phenyl)piperidine-1-carbonyl)-1,4,5,7-tetrahydro-6H-pyrazolo[3,4-c]pyridin-6-yl)ethan-1-one; (4-(3-fluoro-2,5-bis(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(6-(cyclopropylmethyl)-4,5,6,7-tetrahydro-1H-pyrazolo[3,4-c]pyridin-3-yl)methanone; (4-(3,5-bis(trifluoromethyl)phenyl)piperidin-1-yl)(5-(methylsulfonyl)-4,5,6,7-tetrahydro-1H-pyrazolo[4,3-c]pyridin-3-yl)methanone; (4-(2-chloro-3-fluorophenyl)piperidin-1-yl)(5-(methylsulfonyl)-4,5,6,7-tetrahydro-1H-pyrazolo[4,3-c]pyridin-3-yl)methanone; (4-(2-chloro-3-fluorophenyl)piperidin-1-yl)(6-(oxetan-3-yl)-4,5,6,7-tetrahydro-1H-pyrazolo[3,4-c]pyridin-3-yl)methanone; 3-(4-(2-chloro-3-fluorophenyl)piperidine-1-carbonyl)-1,4,5,7-tetrahydro-6H-pyrazolo[3,4-c]pyridine-6-carbonitrile(4-(5-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; 3-(4-(3,5-difluoro-2-(trifluoromethyl)phenyl)piperidine-1-carbonyl)-N-methyl-1,4,5,7-tetrahydro-6H-pyrazolo[3,4-c]pyridine-6-carboxamide(6-(cyclopropylmethyl)-4,5,6,7-tetrahydro-1H-pyrazolo[3,4-c]pyridin-3-yl)(4-(3,4-difluoro-2-(trifluoromethyl)phenyl)piperidin-1-yl)methanone; methyl 3-(4-(3,5-difluoro-2-(trifluoromethyl)phenyl)piperidine-1-carbonyl)-1,4,5,7-tetrahydro-6H-pyrazolo[3,4-c]pyridine-6-carboxylate; (4-(3,5-difluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; (4-(3,4-difluoro-2-(trifluoromethyl)phenyl)piperidin-1-yl)(5-neopentyl-4,5,6,7-tetrahydro-1H-pyrazolo[4,3-c]pyridin-3-yl)methanone; (4-(2-chloro-5-fluorophenyl)piperidin-1-yl)(5-(methylsulfonyl)-4,5,6,7-tetrahydro-1H-pyrazolo[4,3-c]pyridin-3-yl)methanone; (4-(3,5-difluoro-2-(trifluoromethyl)phenyl)piperidin-1-yl)(6-(2,2,2-trifluoroethyl)-4,5,6,7-tetrahydro-1H-pyrazolo[3,4-c]pyridin-3-yl)methanone; 3-(4-(3,5-difluoro-2-(trifluoromethyl)phenyl)piperidine-1-carbonyl)-1,4,5,7-tetrahydro-6H-pyrazolo[3,4-c]pyridine-6-carbonitrile; (4-(3,5-difluoro-2-(trifluoromethyl)phenyl)piperidin-1-yl)(6-(oxetan-3-yl)-4,5,6,7-tetrahydro-1H-pyrazolo[3,4-c]pyridin-3-yl)methanone; 3-(4-(3,4-difluoro-2-(trifluoromethyl)phenyl)piperidine-1-carbonyl)-N-methyl-1,4,5,7-tetrahydro-6H-pyrazolo[3,4-c]pyridine-6-carboxamide; (4-(3,4-difluoro-2-(trifluoromethyl)phenyl)piperidin-1-yl)(6-neopentyl-4,5,6,7-tetrahydro-1H-pyrazolo[3,4-c]pyridin-3-yl)methanone; (4-(3,4-difluoro-2-(trifluoromethyl)phenyl)piperidin-1-yl)(6-(2,2,2-trifluoroethyl)-4,5,6,7-tetrahydro-1H-pyrazolo[3,4-c]pyridin-3-yl)methanone; methyl 3-(4-(2-chloro-3-fluorophenyl)piperidine-1-carbonyl)-1,4,5,7-tetrahydro-6H-pyrazolo[3,4-c]pyridine-6-carboxylate; 1-(3-(4-(3,5-difluoro-2-(trifluoromethyl)phenyl)piperidine-1-carbonyl)-1,4,5,7-tetrahydro-6H-pyrazolo[3,4-c]pyridin-6-yl)ethan-1-one; (6-(cyclopropylmethyl)-4,5,6,7-tetrahydro-1H-pyrazolo[3,4-c]pyridin-3-yl)(4-(3,5-difluoro-2-(trifluoromethyl)phenyl)piperidin-1-yl)methanone; (4-(3,4-difluoro-2-(trifluoromethyl)phenyl)piperidin-1-yl)(6-(2-methoxyethyl)-4,5,6,7-tetrahydro-1H-pyrazolo[3,4-c]pyridin-3-yl)methanone; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)-3-methylbutan-1-one; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)propan-1-one; 1-(3-(4-(3,4-difluoro-2-(trifluoromethyl)phenyl)piperidine-1-carbonyl)-1,4,5,7-tetrahydro-6H-pyrazolo[3,4-c]pyridin-6-yl)-2-methylpropan-1-one; 3-(4-(3,4-difluoro-2-(trifluoromethyl)phenyl)piperidine-1-carbonyl)-1,4,5,7-tetrahydro-6H-pyrazolo[3,4-c]pyridine-6-carbonitrile; (4-(3,4-difluoro-2-(trifluoromethyl)phenyl)piperidin-1-yl)(6-(3,3,3-trifluoropropyl)-4,5,6,7-tetrahydro-1H-pyrazolo[3,4-c]pyridin-3-yl)methanone; (4-(3,4-difluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; (4-(3,4-difluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; 3-(4-(3,4-difluoro-2-(trifluoromethyl)phenyl)piperidine-1-carbonyl)-N-methyl-1,4,6,7-tetrahydro-5H-pyrazolo[4,3-c]pyridine-5-carboxamide; (4-(3,4-difluoro-2-(trifluoromethyl)phenyl)piperidin-1-yl)(6-(oxetan-3-yl)-4,5,6,7-tetrahydro-1H-pyrazolo[3,4-c]pyridin-3-yl)methanone; methyl 3-(4-(3,4-difluoro-2-(trifluoromethyl)phenyl)piperidine-1-carbonyl)-1,4,5,7-tetrahydro-6H-pyrazolo[3,4-c]pyridine-6-carboxylate; 2-(3-(4-(3,4-difluoro-2-(trifluoromethyl)phenyl)piperidine-1-carbonyl)-1,4,5,7-tetrahydro-6H-pyrazolo[3,4-c]pyridin-6-yl)acetic acid; (4-(3,4-difluoro-2-(trifluoromethyl)phenyl)piperidin-1-yl)(5-(oxetan-3-yl)-4,5,6,7-tetrahydro-1H-pyrazolo[4,3-c]pyridin-3-yl)methanone; (5-(cyclopropylmethyl)-4,5,6,7-tetrahydro-1H-pyrazolo[4,3-c]pyridin-3-yl)(4-(3,4-difluoro-2-(trifluoromethyl)phenyl)piperidin-1-yl)methanone; (4-(3,4-difluoro-2-(trifluoromethyl)phenyl)piperidin-1-yl)(5-ethyl-4,5,6,7-tetrahydro-1H-pyrazolo[4,3-c]pyridin-3-yl)methanone; 3-(4-(3-fluoro-2-(trifluoromethyl)phenyl)piperidine-1-carbonyl)-N-methyl-1,4,6,7-tetrahydro-5H-pyrazolo[4,3-c]pyridine-5-carboxamide; (4-(3,4-difluoro-2-(trifluoromethyl)phenyl)piperidin-1-yl)(6-methyl-4,5,6,7-tetrahydro-1H-pyrazolo[3,4-c]pyridin-3-yl)methanone; methyl 3-(4-(3,5-difluoro-2-(trifluoromethyl)phenyl)piperidine-1-carbonyl)-1,4,6,7-tetrahydro-5H-pyrazolo[4,3-c]pyridine-5-carboxylate; (4-(3,5-difluoro-2-(trifluoromethyl)phenyl)piperidin-1-yl)(5-ethyl-4,5,6,7-tetrahydro-1H-pyrazolo[4,3-c]pyridin-3-yl)methanone; (4-(3,5-difluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; 3-(4-(3,4-difluoro-2-(trifluoromethyl)phenyl)piperidine-1-carbonyl)-1,4,6,7-tetrahydro-5H-pyrazolo[4,3-c]pyridine-5-carbonitrile; methyl 3-(4-(3,4-difluoro-2-(trifluoromethyl)phenyl)piperidine-1-carbonyl)-1,4,6,7-tetrahydro-5H-pyrazolo[4,3-c]pyridine-5-carboxylate; (4-(3-fluoro-2-(trifluoromethyl)phenyl)piperidin-1-yl)(6-(3,3,3-trifluoropropyl)-4,5,6,7-tetrahydro-1H-pyrazolo[3,4-c]pyridin-3-yl)methanone; (6-ethyl-4,5,6,7-tetrahydro-1H-pyrazolo[3,4-c]pyridin-3-yl)(4-(3-fluoro-2-(trifluoromethyl)phenyl)piperidin-1-yl)methanone; (4-(3-fluoro-2-(trifluoromethyl)phenyl)piperidin-1-yl)(5-(oxetan-3-yl)-4,5,6,7-tetrahydro-1H-pyrazolo[4,3-c]pyridin-3-yl)methanone; (4-(3-fluoro-2-(trifluoromethyl)phenyl)piperidin-1-yl)(5-(2-methoxyethyl)-4,5,6,7-tetrahydro-1H-pyrazolo[4,3-c]pyridin-3-yl)methanone; (4-(3-fluoro-2-(trifluoromethyl)phenyl)piperidin-1-yl)(5-(3,3,3-trifluoropropyl)-4,5,6,7-tetrahydro-1H-pyrazolo[4,3-c]pyridin-3-yl)methanone; (4-(2-chloro-5-fluorophenyl)piperidin-1-yl)(4,5,6,7-tetrahydro-1H-pyrazolo[3,4-c]pyridin-3-yl)methanone; imidazo[1,2-a]pyridin-2-yl(4-(2-(trifluoromethyl)phenyl)piperidin-1-yl)methanone; (4-(5-fluoro-2-(trifluoromethyl)phenyl)piperidin-1-yl)(4,5,6,7-tetrahydro-1H-pyrazolo[4,3-c]pyridin-3-yl)methanone; 1-(3-(4-(3,4-difluoro-2-(trifluoromethyl)phenyl)piperidine-1-carbonyl)-1,4,6,7-tetrahydro-5H-pyrazolo[4,3-c]pyridin-5-yl)ethan-1-one; (5-ethyl-4,5,6,7-tetrahydro-1H-pyrazolo[4,3-c]pyridin-3-yl)(4-(3-fluoro-2-(trifluoromethyl)phenyl)piperidin-1-yl)methanone; 3-(4-(3-fluoro-2-(trifluoromethyl)phenyl)piperidine-1-carbonyl)-N-methyl-1,4,5,7-tetrahydro-6H-pyrazolo[3,4-c]pyridine-6-carboxamide; (4-(3-fluoro-2-(trifluoromethyl)phenyl)piperidin-1-yl)(5-(methylsulfonyl)-4,5,6,7-tetrahydro-1H-pyrazolo[4,3-c]pyridin-3-yl)methanone; (4-(3,5-difluoro-2-(trifluoromethyl)phenyl)piperidin-1-yl)(4,5,6,7-tetrahydro-1H-pyrazolo[4,3-c]pyridin-3-yl)methanone; (4-(3,5-difluoro-2-(trifluoromethyl)phenyl)piperidin-1-yl)(4,5,6,7-tetrahydro-1H-pyrazolo[3,4-c]pyridin-3-yl)methanone; (4-(2-chloro-3-fluorophenyl)piperidin-1-yl)(4,5,6,7-tetrahydro-1H-pyrazolo[4,3-c]pyridin-3-yl)methanone; (4-(2-chloro-3-fluorophenyl)piperidin-1-yl)(4,5,6,7-tetrahydro-1H-pyrazolo[3,4-c]pyridin-3-yl)methanone; (4-(3,4-difluoro-2-(trifluoromethyl)phenyl)piperidin-1-yl)(4,5,6,7-tetrahydro-1H-pyrazolo[4,3-c]pyridin-3-yl)methanone; (4-(3,4-difluoro-2-(trifluoromethyl)phenyl)piperidin-1-yl)(4,5,6,7-tetrahydro-1H-pyrazolo[3,4-c]pyridin-3-yl)methanone; (4-(3,5-bis(trifluoromethyl)phenyl)piperidin-1-yl)(4,5,6,7-tetrahydro-1H-pyrazolo[4,3-c]pyridin-3-yl)methanone; (4-(3,5-bis(trifluoromethyl)phenyl)piperidin-1-yl)(4,5,6,7-tetrahydro-1H-pyrazolo[3,4-c]pyridin-3-yl)methanone; (4-(3-fluoro-2-(trifluoromethyl)phenyl)piperidin-1-yl)(4,5,6,7-tetrahydro-1H-pyrazolo[4,3-c]pyridin-3-yl)methanone; (4-(3-fluoro-2-(trifluoromethyl)phenyl)piperidin-1-yl)(4,5,6,7-tetrahydro-1H-pyrazolo[3,4-c]pyridin-3-yl)methanone; 1-(3-(4-(2-chloro-3-fluorophenyl)piperidine-1-carbonyl)-1,4,5,7-tetrahydro-6H-pyrazolo[3,4-c]pyridin-6-yl)ethan-1-one; 1-(3-(4-(3-fluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(5-fluoro-2-(trifluoromethyl)phenyl)piperidine-1-carbonyl)-1,4,5,7-tetrahydro-6H-pyrazolo[3,4-c]pyridin-6-yl)ethan-1-one; 1-(3-(4-(3,5-bis(trifluoromethyl)phenyl)piperidine-1-carbonyl)-1,4,5,7-tetrahydro-6H-pyrazolo[3,4-c]pyridin-6-yl)ethan-1-one; 1-(3-(4-(4-fluoro-2-(trifluoromethyl)phenyl)piperidine-1-carbonyl)-1,4,5,7-tetrahydro-6H-pyrazolo[3,4-c]pyridin-6-yl)ethan-1-one; (4-(2-fluoro-6-(trifluoromethyl)phenyl)piperidin-1-yl)(4,5,6,7-tetrahydro-1H-pyrazolo[4,3-c]pyridin-3-yl)methanone; (4-(3-fluoro-2-(trifluoromethyl)phenyl)piperidin-1-yl)(5-(2,2,2-trifluoroethyl)-4,5,6,7-tetrahydro-1H-pyrazolo[4,3-c]pyridin-3-yl)methanone; (5-(cyclopropylmethyl)-4,5,6,7-tetrahydro-1H-pyrazolo[4,3-c]pyridin-3-yl)(4-(3-fluoro-2-(trifluoromethyl)phenyl)piperidin-1-yl)methanone; methyl 3-(4-(3-fluoro-2-(trifluoromethyl)phenyl)piperidine-1-carbonyl)-1,4,6,7-tetrahydro-5H-pyrazolo[4,3-c]pyridine-5-carboxylate; 3-(4-(3-fluoro-2-(trifluoromethyl)phenyl)piperidine-1-carbonyl)-1,4,6,7-tetrahydro-5H-pyrazolo[4,3-c]pyridine-5-carbonitrile; 1-(3-(4-(2-chloro-5-fluorophenyl)piperidine-1-carbonyl)-1,4,5,7-tetrahydro-6H-pyrazolo[3,4-c]pyridin-6-yl)ethan-1-one; (4-(2-fluoro-6-(trifluoromethyl)phenyl)piperidin-1-yl)(4,5,6,7-tetrahydro-1H-pyrazolo[3,4-c]pyridin-3-yl)methanone; tert-butyl 2-(3-(4-(3,4-difluoro-2-(trifluoromethyl)phenyl)piperidine-1-carbonyl)-1,4,5,7-tetrahydro-6H-pyrazolo[3,4-c]pyridin-6-yl)acetate; tert-butyl 3-(4-(3,5-difluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(3,5-difluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-fluoro-6-(trifluoromethyl)phenyl)piperidine-1-carbonyl)-1,4,6,7-tetrahydro-5H-pyrazolo[4,3-c]pyridine-5-carboxylate; tert-butyl 3-(4-(5-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(5-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-chloro-3-fluorophenyl)piperidine-1-carbonyl)-1,4,6,7-tetrahydro-5H-pyrazolo[4,3-c]pyridine-5-carboxylate; tert-butyl 3-(4-(2-fluoro-6-(trifluoromethyl)phenyl)piperidine-1-carbonyl)-1,4,5,7-tetrahydro-6H-pyrazolo[3,4-c]pyridine-6-carboxylatetert-butyl 3-(4-(3,5-bis(trifluoromethyl)phenyl)piperidine-1-carbonyl)-1,4,6,7-tetrahydro-5H-pyrazolo[4,3-c]pyridine-5-carboxylate; tert-butyl 3-(4-(3,5-bis(trifluoromethyl)phenyl)piperidine-1-carbonyl)-1,4,5,7-tetrahydro-6H-pyrazolo[3,4-c]pyridine-6-carboxylate; tert-butyl 3-(4-(2-chloro-5-fluorophenyl)piperidine-1-carbonyl)-1,4,6,7-tetrahydro-5H-pyrazolo[4,3-c]pyridine-5-carboxylate; tert-butyl 3-(4-(2-chloro-5-fluorophenyl)piperidine-1-carbonyl)-1,4,5,7-tetrahydro-6H-pyrazolo[3,4-c]pyridine-6-carboxylate; tert-butyl 3-(4-(4-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3-fluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3,4-difluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(3,4-difluoro-2-(trifluoromethyl)phenyl)piperidine-1-carbonyl)-1,4,5,7-tetrahydro-6H-pyrazolo[3,4-c]pyridine-6-carboxylate; tert-butyl 3-(4-(3-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(4-fluoro-2-(trifluoromethyl)phenyl)piperidine-1-carbonyl)-1,4,6,7-tetrahydro-5H-pyrazolo[4,3-c]pyridine-5-carboxylate; tert-butyl 3-(4-(2-chloro-3-fluorophenyl)piperidine-1-carbonyl)-1,4,5,7-tetrahydro-6H-pyrazolo[3,4-c]pyridine-6-carboxylate; (6,6-dimethyl-1,4,6,7-tetrahydropyrano[4,3-c]pyrazol-3-yl)(4-(2-(trifluoromethyl)phenyl)piperidin-1-yl)methanone; (6,6-dioxido-1,4,5,7-tetrahydrothiopyrano[3,4-c]pyrazol-3-yl)(4-(2-(trifluoromethyl)phenyl)piperidin-1-yl)methanone; (1,4,6,7-tetrahydropyrano[4,3-c]pyrazol-3-yl)(4-(2-(trifluoromethyl)phenyl)piperidin-1-yl)methanone; (1,4,5,7-tetrahydropyrano[3,4-c]pyrazol-3-yl)(4-(2-(trifluoromethyl)phenyl)piperidin-1-yl)methanone; (1-methyl-4,5,6,7-tetrahydro-1H-pyrazolo[4,3-c]pyridin-3-yl)(4-(2-(trifluoromethyl)phenyl)piperidin-1-yl)methanone; (1-methyl-4,5,6,7-tetrahydro-1H-pyrazolo[3,4-c]pyridin-3-yl)(4-(2-(trifluoromethyl)phenyl)piperidin-1-yl)methanone; 1-ethyl-N,N-dimethyl-3-(4-(2-(trifluoromethyl)phenyl)piperidine-1-carbonyl)-1,4,5,7-tetrahydro-6H-pyrazolo[3,4-c]pyridine-6-carboxamide; (5-(2,2,2-trifluoroethyl)-4,5,6,7-tetrahydro-1H-pyrazolo[4,3-c]pyridin-3-yl)(4-(2-(trifluoromethyl)phenyl)piperidin-1-yl)methanone; (6-(2,2,2-trifluoroethyl)-4,5,6,7-tetrahydro-1H-pyrazolo[3,4-c]pyridin-3-yl)(4-(2-(trifluoromethyl)phenyl)piperidin-1-yl)methanone; (5-chloro-1H-indazol-3-yl)(4-(2-(trifluoromethyl)phenyl)piperidin-1-yl)methanone; (1H-pyrazolo[3,4-b]pyridin-3-yl)(4-(2-(trifluoromethyl)phenyl)piperidin-1-yl)methanone; (6-chloro-1H-indazol-3-yl)(4-(2-(trifluoromethyl)phenyl)piperidin-1-yl)methanone; (5-(methylsulfonyl)-4,5,6,7-tetrahydro-1H-pyrazolo[4,3-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,6,7-tetrahydro-5H-pyrazolo[4,3-c]pyridin-5-yl)ethan-1-one; (4,5,6,7-tetrahydro-1H-pyrazolo[4,3-c]pyridin-3-yl)(4-(2-(trifluoromethyl)phenyl)piperidin-1-yl)methanone; (6-fluoro-1-(oxetan-3-yl)-1H-indazol-3-yl)(4-(2-(trifluoromethyl)phenyl)piperidin-1-yl)methanone; (1-ethyl-6-fluoro-1H-indazol-3-yl)(4-(2-(trifluoromethyl)phenyl)piperidin-1-yl)methanone; (6-fluoro-1-isopropyl-1H-indazol-3-yl)(4-(2-(trifluoromethyl)phenyl)piperidin-1-yl)methanone; 1-(3-(4-fluoro-4-(2-(trifluoromethyl)phenyl)piperidine-1-carbonyl)-1,4,6,7-tetrahydro-5H-pyrazolo[4,3-c]pyridin-5-yl)ethan-1-one; (5-fluoro-1-methyl-1H-indazol-3-yl)(4-(2-(trifluoromethyl)phenyl)piperidin-1-yl)methanone; (6-fluoro-1H-indazol-3-yl)(4-(2-(trifluoromethyl)phenyl)piperidin-1-yl)methanone; (6-methyl-4,5,6,7-tetrahydro-1H-pyrazolo[3,4-c]pyridin-3-yl)(4-(2-(trifluoromethyl)phenyl)piperidin-1-yl)methanone; (6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,5,7-tetrahydro-6H-pyrazolo[3,4-c]pyridin-6-yl)ethan-1-one; (5-fluoro-1H-indazol-3-yl)(4-(2-(trifluoromethyl)phenyl)piperidin-1-yl)methanone; (5-((chloromethyl)sulfonyl)-4,5,6,7-tetrahydro-1H-pyrazolo[4,3-c]pyridin-3-yl)(4-(2-(trifluoromethyl)phenyl)piperidin-1-yl)methanone; (5-(2-methoxy ethyl)-4,5,6,7-tetrahydro-1H-pyrazolo[4,3-c]pyridin-3-yl)(4-(2-(trifluoromethyl)phenyl)piperidin-1-yl)methanone; (4-fluoro-4-(2-(trifluoromethyl)phenyl)piperidin-1-yl)(4,5,6,7-tetrahydro-1H-pyrazolo[4,3-c]pyridin-3-yl)methanone; 1-(3-(4-(4-fluoro-2-(trifluoromethyl)phenyl)piperidine-1-carbonyl)-1,4,5,7-tetrahydro-6H-pyrazolo[3,4-c]pyridin-6-yl)ethan-1-one; (1-ethyl-5-fluoro-1H-indazol-3-yl)(4-(2-(trifluoromethyl)phenyl)piperidin-1-yl)methanone; (6-fluoro-1-methyl-1H-indazol-3-yl)(4-(2-(trifluoromethyl)phenyl)piperidin-1-yl)methanone; 3-(4-(2-(trifluoromethyl)phenyl)piperidine-1-carbonyl)-1,4,5,7-tetrahydro-6H-pyrazolo[4,3-c]pyridin-6-one; 3-(4-(2-(trifluoromethyl)phenyl)piperidine-1-carbonyl)-1,4,6,7-tetrahydro-5H-pyrazolo[3,4-c]pyridin-5-one; 6-methyl-3-(4-(2-(trifluoromethyl)phenyl)piperidine-1-carbonyl)-1,4,6,7-tetrahydro-5H-pyrazolo[3,4-c]pyridin-5-one; 5-methyl-3-(4-(2-(trifluoromethyl)phenyl)piperidine-1-carbonyl)-1,4,5,7-tetrahydro-6H-pyrazolo[4,3-c]pyridin-6-one; (5,5-dioxido-1,4,6,7-tetrahydrothiopyrano[4,3-c]pyrazol-3-yl)(4-(2-(trifluoromethyl)phenyl)piperidin-1-yl)methanone; (1-methyl-5-(methylsulfonyl)-4,5,6,7-tetrahydro-1H-pyrazolo[4,3-c]pyridin-3-yl)(4-(2-(trifluoromethyl)phenyl)piperidin-1-yl)methanone; (1-methyl-6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(1-ethyl-3-(4-(2-(trifluoromethyl)phenyl)piperidine-1-carbonyl)-1,4,5,7-tetrahydro-6H-pyrazolo[3,4-c]pyridin-6-yl)ethan-1-one; (5-(methoxymethyl)-4,5,6,7-tetrahydro-1H-pyrazolo[4,3-c]pyridin-3-yl)(4-(2-(trifluoromethyl)phenyl)piperidin-1-yl)methanone; (6-(methoxymethyl)-4,5,6,7-tetrahydro-1H-pyrazolo[3,4-c]pyridin-3-yl)(4-(2-(trifluoromethyl)phenyl)piperidin-1-yl)methanone; (5-methoxy-1H-indazol-3-yl)(4-(2-(trifluoromethyl)phenyl)piperidin-1-yl)methanone; (5-(oxetan-3-yl)-4,5,6,7-tetrahydro-1H-pyrazolo[4,3-c]pyridin-3-yl)(4-(2-(trifluoromethyl)phenyl)piperidin-1-yl)methanone; (5-isobutyl-4,5,6,7-tetrahydro-1H-pyrazolo[4,3-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,6,7-tetrahydro-5H-pyrazolo[4,3-c]pyridin-5-yl)propan-1-one; (5-ethyl-4,5,6,7-tetrahydro-1H-pyrazolo[4,3-c]pyridin-3-yl)(4-(2-(trifluoromethyl)phenyl)piperidin-1-yl)methanone; 3-methyl-1-(3-(4-(2-(trifluoromethyl)phenyl)piperidine-1-carbonyl)-1,4,6,7-tetrahydro-5H-pyrazolo[4,3-c]pyridin-5-yl)butan-1-one; 2-methyl-1-(3-(4-(2-(trifluoromethyl)phenyl)piperidine-1-carbonyl)-1,4,6,7-tetrahydro-5H-pyrazolo[4,3-c]pyridin-5-yl)propan-1-one; 2,2-dimethyl-1-(3-(4-(2-(trifluoromethyl)phenyl)piperidine-1-carbonyl)-1,4,6,7-tetrahydro-5H-pyrazolo[4,3-c]pyridin-5-yl)propan-1-one; (5-(isopropylsulfonyl)-4,5,6,7-tetrahydro-1H-pyrazolo[4,3-c]pyridin-3-yl)(4-(2-(trifluoromethyl)phenyl)piperidin-1-yl)methanone; (5-(isobutylsulfonyl)-4,5,6,7-tetrahydro-1H-pyrazolo[4,3-c]pyridin-3-yl)(4-(2-(trifluoromethyl)phenyl)piperidin-1-yl)methanone; (5-(ethylsulfonyl)-4,5,6,7-tetrahydro-1H-pyrazolo[4,3-c]pyridin-3-yl)(4-(2-(trifluoromethyl)phenyl)piperidin-1-yl)methanone; 3-(4-(2-(trifluoromethyl)phenyl)piperidine-1-carbonyl)-1H-indazole-5-carbonitrile; (7-chloro-1H-indazol-3-yl)(4-(2-(trifluoromethyl)phenyl)piperidin-1-yl)methanone; (5,6-difluoro-1H-indazol-3-yl)(4-(2-(trifluoromethyl)phenyl)piperidin-1-yl)methanone; (6-(2-methoxyethyl)-4,5,6,7-tetrahydro-1H-pyrazolo[3,4-c]pyridin-3-yl)(4-(2-(trifluoromethyl)phenyl)piperidin-1-yl)methanone; 3,3,3-trifluoro-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (5-(tert-butyl)-4,5,6,7-tetrahydro-1H-pyrazolo[4,3-c]pyridin-3-yl)(4-(2-(trifluoromethyl)phenyl)piperidin-1-yl)methanone; (5-isopropyl-4,5,6,7-tetrahydro-1H-pyrazolo[4,3-c]pyridin-3-yl)(4-(2-(trifluoromethyl)phenyl)piperidin-1-yl)methanone; N-methyl-3-(4-(2-(trifluoromethyl)phenyl)piperidine-1-carbonyl)-1,4,6,7-tetrahydro-5H-pyrazolo[4,3-c]pyridine-5-carboxamide; N-methyl-3-(4-(2-(trifluoromethyl)phenyl)piperidine-1-carbonyl)-1,4,5,7-tetrahydro-6H-pyrazolo[3,4-c]pyridine-6-carboxamide; (5-bromo-1H-indazol-3-yl)(4-(2-(trifluoromethyl)phenyl)piperidin-1-yl)methanone; tert-butyl 3-(4-(2-(trifluoromethyl)phenyl)piperidine-1-carbonyl)-1,4,5,7-tetrahydro-6H-pyrazolo[3,4-c]pyridine-6-carboxylate; tert-butyl 3-(4-(2-(trifluoromethyl)phenyl)piperidine-1-carbonyl)-1,4,6,7-tetrahydro-5H-pyrazolo[4,3-c]pyridine-5-carboxylate; (5-fluoro-1-isopropyl-1H-indazol-3-yl)(4-(2-(trifluoromethyl)phenyl)piperidin-1-yl)methanone; (7-fluoro-1H-indazol-3-yl)(4-(2-(trifluoromethyl)phenyl)piperidin-1-yl)methanone; (1H-pyrazolo[4,3-c]pyridin-3-yl)(4-(2-(trifluoromethyl)phenyl)piperidin-1-yl)methanone; (1H-pyrazolo[3,4-c]pyridin-3-yl)(4-(2-(trifluoromethyl)phenyl)piperidin-1-yl)methanone; (1H-pyrazolo[4,3-b]pyridin-3-yl)(4-(2-(trifluoromethyl)phenyl)piperidin-1-yl)methanone; (6-methoxy-1H-indazol-3-yl)(4-(2-(trifluoromethyl)phenyl)piperidin-1-yl)methanone; (5-fluoro-1-(oxetan-3-yl)-1H-indazol-3-yl)(4-(2-(trifluoromethyl)phenyl)piperidin-1-yl)methanone; (1-ethyl-5-(methylsulfonyl)-4,5,6,7-tetrahydro-1H-pyrazolo[4,3-c]pyridin-3-yl)(4-(2-(trifluoromethyl)phenyl)piperidin-1-yl)methanone; (1-ethyl-6-(methylsulfonyl)-4,5,6,7-tetrahydro-1H-pyrazolo[3,4-c]pyridin-3-yl)(4-(2-(trifluoromethyl)phenyl)piperidin-1-yl)methanone; 1-(1-methyl-3-(4-(2-(trifluoromethyl)phenyl)piperidine-1-carbonyl)-1,4,6,7-tetrahydro-5H-pyrazolo[4,3-c]pyridin-5-yl)ethan-1-one; 1-(1-methyl-3-(4-(2-(trifluoromethyl)phenyl)piperidine-1-carbonyl)-1,4,5,7-tetrahydro-6H-pyrazolo[3,4-c]pyridin-6-yl)ethan-1-one; N,N-dimethyl-3-(4-(2-(trifluoromethyl)phenyl)piperidine-1-carbonyl)-1,4,6,7-tetrahydro-5H-pyrazolo[4,3-c]pyridine-5-carboxamide; N,N-dimethyl-3-(4-(2-(trifluoromethyl)phenyl)piperidine-1-carbonyl)-1,4,5,7-tetrahydro-6H-pyrazolo[3,4-c]pyridine-6-carboxamide; (1-methyl-5,5-dioxido-1,4,6,7-tetrahydrothiopyrano[4,3-c]pyrazol-3-yl)(4-(2-(trifluoromethyl)phenyl)piperidin-1-yl)methanone; (4-(2-(trifluoromethyl)phenyl)piperidin-1-yl)(1,6,6-trimethyl-1,4,6,7-tetrahydropyrano[4,3-c]pyrazol-3-yl)methanone; (1-methyl-1,4,6,7-tetrahydropyrano[4,3-c]pyrazol-3-yl)(4-(2-(trifluoromethyl)phenyl)piperidin-1-yl)methanone; 2-methyl-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (6-(isopropylsulfonyl)-4,5,6,7-tetrahydro-1H-pyrazolo[3,4-c]pyridin-3-yl)(4-(2-(trifluoromethyl)phenyl)piperidin-1-yl)methanone; (6-(ethylsulfonyl)-4,5,6,7-tetrahydro-1H-pyrazolo[3,4-c]pyridin-3-yl)(4-(2-(trifluoromethyl)phenyl)piperidin-1-yl)methanone; 1-(3-(4-(2-(trifluoromethyl)phenyl)piperidine-1-carbonyl)-1,4,5,7-tetrahydro-6H-pyrazolo[3,4-c]pyridin-6-yl)propan-1-one; 2-methoxy-1-(3-(4-(2-(trifluoromethyl)phenyl)piperidine-1-carbonyl)-1,4,6,7-tetrahydro-5H-pyrazolo[4,3-c]pyridin-5-yl)ethan-1-one; 3,3,3-trifluoro-1-(3-(4-(2-(trifluoromethyl)phenyl)piperidine-1-carbonyl)-1,4,6,7-tetrahydro-5H-pyrazolo[4,3-c]pyridin-5-yl)propan-1-one; (1H-indazol-3-yl)(4-(2-(trifluoromethyl)phenyl)piperidin-1-yl)methanone; (1-methyl-1H-indazol-3-yl)(4-(2-(trifluoromethyl)phenyl)piperidin-1-yl)methanone; (6-(oxetan-3-yl)-4,5,6,7-tetrahydro-1H-pyrazolo[3,4-c]pyridin-3-yl)(4-(2-(trifluoromethyl)phenyl)piperidin-1-yl)methanone; (6-(tert-butylsulfonyl)-4,5,6,7-tetrahydro-1H-pyrazolo[3,4-c]pyridin-3-yl)(4-(2-(trifluoromethyl)phenyl)piperidin-1-yl)methanone; 2,2-dimethyl-1-(3-(4-(2-(trifluoromethyl)phenyl)piperidine-1-carbonyl)-1,4,5,7-tetrahydro-6H-pyrazolo[3,4-c]pyridin-6-yl)propan-1-one; (6-(tert-butyl)-4,5,6,7-tetrahydro-1H-pyrazolo[3,4-c]pyridin-3-yl)(4-(2-(trifluoromethyl)phenyl)piperidin-1-yl)methanone; (6-(isobutylsulfonyl)-4,5,6,7-tetrahydro-1H-pyrazolo[3,4-c]pyridin-3-yl)(4-(2-(trifluoromethyl)phenyl)piperidin-1-yl)methanone; 3-methyl-1-(3-(4-(2-(trifluoromethyl)phenyl)piperidine-1-carbonyl)-1,4,5,7-tetrahydro-6H-pyrazolo[3,4-c]pyridin-6-yl)butan-1-one; (6-isobutyl-4,5,6,7-tetrahydro-1H-pyrazolo[3,4-c]pyridin-3-yl)(4-(2-(trifluoromethyl)phenyl)piperidin-1-yl)methanone; (6-isopropyl-4,5,6,7-tetrahydro-1H-pyrazolo[3,4-c]pyridin-3-yl)(4-(2-(trifluoromethyl)phenyl)piperidin-1-yl)methanone; (6-ethyl-4,5,6,7-tetrahydro-1H-pyrazolo[3,4-c]pyridin-3-yl)(4-(2-(trifluoromethyl)phenyl)piperidin-1-yl)methanone; (5-(tert-butylsulfonyl)-4,5,6,7-tetrahydro-1H-pyrazolo[4,3-c]pyridin-3-yl)(4-(2-(trifluoromethyl)phenyl)piperidin-1-yl)methanone; tert-butyl 3-(4-fluoro-4-(2-(trifluoromethyl)phenyl)piperidine-1-carbonyl)-1,4,6,7-tetrahydro-5H-pyrazolo[4,3-c]pyridine-5-carboxylate; (4-hydroxy-4-(2-(trifluoromethyl)phenyl)piperidin-1-yl)(1-methyl-1H-indazol-3-yl)methanone; 1-(3-(4-hydroxy-4-(2-(trifluoromethyl)phenyl)piperidine-1-carbonyl)-1,4,6,7-tetrahydro-5H-pyrazolo[4,3-c]pyridin-5-yl)ethan-1-one; 3-(4-(3,4-difluoro-2-(trifluoromethyl)phenyl)piperidine-1-carbonyl)-N-methyl-4,6-dihydropyrrolo[3,4-c]pyrazole-5(1H)-carboxamide; (4-(3,4-difluoro-2-(trifluoromethyl)phenyl)piperidin-1-yl)(5-neopentyl-1,4,5,6-tetrahydropyrrolo[3,4-c]pyrazol-3-yl)methanone; (4-(3,4-difluoro-2-(trifluoromethyl)phenyl)piperidin-1-yl)(5-(oxetan-3-yl)-1,4,5,6-tetrahydropyrrolo[3,4-c]pyrazol-3-yl)methanone; (5-(cyclopropylmethyl)-1,4,5,6-tetrahydropyrrolo[3,4-c]pyrazol-3-yl)(4-(3,4-difluoro-2-(trifluoromethyl)phenyl)piperidin-1-yl)methanone; (4-(3,4-difluoro-2-(trifluoromethyl)phenyl)piperidin-1-yl)(5-ethyl-1,4,5,6-tetrahydropyrrolo[3,4-c]pyrazol-3-yl)methanone; 1-(3-(4-(3,4-difluoro-2-(trifluoromethyl)phenyl)piperidine-1-carbonyl)-4,6-dihydropyrrolo[3,4-c]pyrazol-5(1H)-yl)-3-methylbutan-1-one; 1-(3-(4-(3,4-difluoro-2-(trifluoromethyl)phenyl)piperidine-1-carbonyl)-4,6-dihydropyrrolo[3,4-c]pyrazol-5(1H)-yl)-2-methylpropan-1-one; (4-(3,4-difluoro-2-(trifluoromethyl)phenyl)piperidin-1-yl)(5-picolinoyl-1,4,5,6-tetrahydropyrrolo[3,4-c]pyrazol-3-yl)methanone; 3-(4-(3,4-difluoro-2-(trifluoromethyl)phenyl)piperidine-1-carbonyl)-4,6-dihydropyrrolo[3,4-c]pyrazole-5(1H)-carbonitrile; (4-(3,4-difluoro-2-(trifluoromethyl)phenyl)piperidin-1-yl)(5-(2-methoxyethyl)-1,4,5,6-tetrahydropyrrolo[3,4-c]pyrazol-3-yl)methanone; (4-(3,4-difluoro-2-(trifluoromethyl)phenyl)piperidin-1-yl)(5-(3,3,3-trifluoropropyl)-1,4,5,6-tetrahydropyrrolo[3,4-c]pyrazol-3-yl)methanone; (5-benzoyl-1,4,5,6-tetrahydropyrrolo[3,4-c]pyrazol-3-yl)(4-(3,4-difluoro-2-(trifluoromethyl)phenyl)piperidin-1-yl)methanone; methyl 3-(4-(3,4-difluoro-2-(trifluoromethyl)phenyl)piperidine-1-carbonyl)-4,6-dihydropyrrolo[3,4-c]pyrazole-5(1H)-carboxylate; (4-(3,4-difluoro-2-(trifluoromethyl)phenyl)piperidin-1-yl)(1,4,5,6-tetrahydropyrrolo[3,4-c]pyrazol-3-yl)methanone; (4-(3,4-difluoro-2-(trifluoromethyl)phenyl)piperidin-1-yl)(5-(2,2,2-trifluoroethyl)-1,4,5,6-tetrahydropyrrolo[3,4-c]pyrazol-3-yl)methanone; (4-(3,4-difluoro-2-(trifluoromethyl)phenyl)piperidin-1-yl)(5-(pyrrolidine-1-carbonyl)-1,4,5,6-tetrahydropyrrolo[3,4-c]pyrazol-3-yl)methanone; (4-(3,4-difluoro-2-(trifluoromethyl)phenyl)piperidin-1-yl)(5-isonicotinoyl-1,4,5,6-tetrahydropyrrolo[3,4-c]pyrazol-3-yl)methanone; (4-(3,4-difluoro-2-(trifluoromethyl)phenyl)piperidin-1-yl)(5-nicotinoyl-1,4,5,6-tetrahydropyrrolo[3,4-c]pyrazol-3-yl)methanone; (4-(3,4-difluoro-2-(trifluoromethyl)phenyl)piperidin-1-yl)(5-(piperidine-1-carbonyl)-1,4,5,6-tetrahydropyrrolo[3,4-c]pyrazol-3-yl)methanone; (4-(3,4-difluoro-2-(trifluoromethyl)phenyl)piperidin-1-yl)(5-(piperazine-1-carbonyl)-1,4,5,6-tetrahydropyrrolo[3,4-c]pyrazol-3-yl)methanone; 1-(3-(4-(3,4-difluoro-2-(trifluoromethyl)phenyl)piperidine-1-carbonyl)-4,6-dihydropyrrolo[3,4-c]pyrazol-5(1H)-yl)propan-1-one; (5,5-dioxido-4,6-dihydro-1H-thieno[3,4-c]pyrazol-3-yl)(4-(2-(trifluoromethyl)phenyl)piperidin-1-yl)methanone; (4,6-dihydro-1H-furo[3,4-c]pyrazol-3-yl)(4-(2-(trifluoromethyl)phenyl)piperidin-1-yl)methanone; (1-ethyl-5-(methylsulfonyl)-1,4,5,6-tetrahydropyrrolo[3,4-c]pyrazol-3-yl)(4-(2-(trifluoromethyl)phenyl)piperidin-1-yl)methanone; (1-methyl-5-(methylsulfonyl)-1,4,5,6-tetrahydropyrrolo[3,4-c]pyrazol-3-yl)(4-(2-(trifluoromethyl)phenyl)piperidin-1-yl)methanone; (1-methyl-1,4,5,6-tetrahydropyrrolo[3,4-c]pyrazol-3-yl)(4-(2-(trifluoromethyl)phenyl)piperidin-1-yl)methanone; 2-methoxy-1-(3-(4-(2-(trifluoromethyl)phenyl)piperidine-1-carbonyl)-4,6-dihydropyrrolo[3,4-c]pyrazol-5(1H)-yl)ethan-1-one; (5-(2-methoxyethyl)-1,4,5,6-tetrahydropyrrolo[3,4-c]pyrazol-3-yl)(4-(2-(trifluoromethyl)phenyl)piperidin-1-yl)methanone; 3,3,3-trifluoro-1-(3-(4-(2-(trifluoromethyl)phenyl)piperidine-1-carbonyl)-4,6-dihydropyrrolo[3,4-c]pyrazol-5(1H)-yl)propan-1-one; (5-(oxetan-3-yl)-1,4,5,6-tetrahydropyrrolo[3,4-c]pyrazol-3-yl)(4-(2-(trifluoromethyl)phenyl)piperidin-1-yl)methanone; (5-(isobutylsulfonyl)-1,4,5,6-tetrahydropyrrolo[3,4-c]pyrazol-3-yl)(4-(2-(trifluoromethyl)phenyl)piperidin-1-yl)methanone; (5-(isopropylsulfonyl)-1,4,5,6-tetrahydropyrrolo[3,4-c]pyrazol-3-yl)(4-(2-(trifluoromethyl)phenyl)piperidin-1-yl)methanone; (5-(ethylsulfonyl)-1,4,5,6-tetrahydropyrrolo[3,4-c]pyrazol-3-yl)(4-(2-(trifluoromethyl)phenyl)piperidin-1-yl)methanone; tert-butyl 3-(4-(2-(trifluoromethyl)phenyl)piperidine-1-carbonyl)-4,6-dihydropyrrolo[3,4-c]pyrazole-5(1H)-carboxylate; (5-methyl-1,4,5,6-tetrahydropyrrolo[3,4-c]pyrazol-3-yl)(4-(2-(trifluoromethyl)phenyl)piperidin-1-yl)methanone; (5-(methylsulfonyl)-1,4,5,6-tetrahydropyrrolo[3,4-c]pyrazol-3-yl)(4-(2-(trifluoromethyl)phenyl)piperidin-1-yl)methanone; 1-(3-(4-(2-(trifluoromethyl)phenyl)piperidine-1-carbonyl)-4,6-dihydropyrrolo[3,4-c]pyrazol-5(1H)-yl)ethan-1-one; (5-(tert-butylsulfonyl)-1,4,5,6-tetrahydropyrrolo[3,4-c]pyrazol-3-yl)(4-(2-(trifluoromethyl)phenyl)piperidin-1-yl)methanone; (5-(tert-butyl)-1,4,5,6-tetrahydropyrrolo[3,4-c]pyrazol-3-yl)(4-(2-(trifluoromethyl)phenyl)piperidin-1-yl)methanone; (5-isobutyl-1,4,5,6-tetrahydropyrrolo[3,4-c]pyrazol-3-yl)(4-(2-(trifluoromethyl)phenyl)piperidin-1-yl)methanone; (5-isopropyl-1,4,5,6-tetrahydropyrrolo[3,4-c]pyrazol-3-yl)(4-(2-(trifluoromethyl)phenyl)piperidin-1-yl)methanone; (5-ethyl-1,4,5,6-tetrahydropyrrolo[3,4-c]pyrazol-3-yl)(4-(2-(trifluoromethyl)phenyl)piperidin-1-yl)methanone; N-methyl-3-(4-(2-(trifluoromethyl)phenyl)piperidine-1-carbonyl)-4,6-dihydropyrrolo[3,4-c]pyrazole-5(1H)-carboxamide; 1-(1-methyl-3-(4-(2-(trifluoromethyl)phenyl)piperidine-1-carbonyl)-4,6-dihydropyrrolo[3,4-c]pyrazol-5(1H)-yl)ethan-1-one; 2,2-dimethyl-1-(3-(4-(2-(trifluoromethyl)phenyl)piperidine-1-carbonyl)-4,6-dihydropyrrolo[3,4-c]pyrazol-5(1H)-yl)propan-1-one; 3-methyl-1-(3-(4-(2-(trifluoromethyl)phenyl)piperidine-1-carbonyl)-4,6-dihydropyrrolo[3,4-c]pyrazol-5(1H)-yl)butan-1-one; 2-methyl-1-(3-(4-(2-(trifluoromethyl)phenyl)piperidine-1-carbonyl)-4,6-dihydropyrrolo[3,4-c]pyrazol-5(1H)-yl)propan-1-one; 1-(3-(4-(2-(trifluoromethyl)phenyl)piperidine-1-carbonyl)-4,6-dihydropyrrolo[3,4-c]pyrazol-5(1H)-yl)propan-1-one; N,N-dimethyl-3-(4-(2-(trifluoromethyl)phenyl)piperidine-1-carbonyl)-4,6-dihydropyrrolo[3,4-c]pyrazole-5(1H)-carboxamide; (5-(2,2,2-trifluoroethyl)-1,4,5,6-tetrahydropyrrolo[3,4-c]pyrazol-3-yl)(4-(2-(trifluoromethyl)phenyl)piperidin-1-yl)methanone; (5-(methoxymethyl)-1,4,5,6-tetrahydropyrrolo[3,4-c]pyrazol-3-yl)(4-(2-(trifluoromethyl)phenyl)piperidin-1-yl)methanone; (1,4,5,6-tetrahydropyrrolo[3,4-c]pyrazol-3-yl)(4-(2-(trifluoromethyl)phenyl)piperidin-1-yl)methanone; tert-butyl 4-(3-(4-(3,4-difluoro-2-(trifluoromethyl)phenyl)piperidine-1-carbonyl)-1,4,5,6-tetrahydropyrrolo[3,4-c]pyrazole-5-carbonyl)piperazine-1-carboxylate; tert-butyl 3-(4-(3,4-difluoro-2-(trifluoromethyl)phenyl)piperidine-1-carbonyl)-4,6-dihydropyrrolo[3,4-c]pyrazole-5(1H)-carboxylate; (5,5-dioxido-4,6,7,8-tetrahydro-1H-thiepino[4,3-c]pyrazol-3-yl)(4-(2-(trifluoromethyl)phenyl)piperidin-1-yl)methanone; (4,6,7,8-tetrahydro-1H-oxepino[4,3-c]pyrazol-3-yl)(4-(2-(trifluoromethyl)phenyl)piperidin-1-yl)methanone; N-methyl-3-(4-(2-(trifluoromethyl)phenyl)piperidine-1-carbonyl)-4,6,7,8-tetrahydropyrazolo[4,3-c]azepine-5(1H)-carboxamide; (5-(2,2,2-trifluoroethyl)-1,4,5,6,7,8-hexahydropyrazolo[4,3-c]azepin-3-yl)(4-(2-(trifluoromethyl)phenyl)piperidin-1-yl)methanone; (5-(tert-butylsulfonyl)-1,4,5,6,7,8-hexahydropyrazolo[4,3-c]azepin-3-yl)(4-(2-(trifluoromethyl)phenyl)piperidin-1-yl)methanone; 2,2-dimethyl-1-(3-(4-(2-(trifluoromethyl)phenyl)piperidine-1-carbonyl)-4,6,7,8-tetrahydropyrazolo[4,3-c]azepin-5(1H)-yl)propan-1-one; (5-(tert-butyl)-1,4,5,6,7,8-hexahydropyrazolo[4,3-c]azepin-3-yl)(4-(2-(trifluoromethyl)phenyl)piperidin-1-yl)methanone; (5-(isobutylsulfonyl)-1,4,5,6,7,8-hexahydropyrazolo[4,3-c]azepin-3-yl)(4-(2-(trifluoromethyl)phenyl)piperidin-1-yl)methanone; 3-methyl-1-(3-(4-(2-(trifluoromethyl)phenyl)piperidine-1-carbonyl)-4,6,7,8-tetrahydropyrazolo[4,3-c]azepin-5(1H)-yl)butan-1-one; (5-isobutyl-1,4,5,6,7,8-hexahydropyrazolo[4,3-c]azepin-3-yl)(4-(2-(trifluoromethyl)phenyl)piperidin-1-yl)methanone; (5-(isopropylsulfonyl)-1,4,5,6,7,8-hexahydropyrazolo[4,3-c]azepin-3-yl)(4-(2-(trifluoromethyl)phenyl)piperidin-1-yl)methanone; 2-methyl-1-(3-(4-(2-(trifluoromethyl)phenyl)piperidine-1-carbonyl)-4,6,7,8-tetrahydropyrazolo[4,3-c]azepin-5(1H)-yl)propan-1-one; (1-ethyl-5-(methylsulfonyl)-1,4,5,6,7,8-hexahydropyrazolo[4,3-c]azepin-3-yl)(4-(2-(trifluoromethyl)phenyl)piperidin-1-yl)methanone; 1-(1-methyl-3-(4-(2-(trifluoromethyl)phenyl)piperidine-1-carbonyl)-4,6,7,8-tetrahydropyrazolo[4,3-c]azepin-5(1H)-yl)ethan-1-one; (5-(methoxymethyl)-1,4,5,6,7,8-hexahydropyrazolo[4,3-c]azepin-3-yl)(4-(2-(trifluoromethyl)phenyl)piperidin-1-yl)methanone; (5-methyl-1,4,5,6,7,8-hexahydropyrazolo[4,3-c]azepin-3-yl)(4-(2-(trifluoromethyl)phenyl)piperidin-1-yl)methanone; (5-isopropyl-1,4,5,6,7,8-hexahydropyrazolo[4,3-c]azepin-3-yl)(4-(2-(trifluoromethyl)phenyl)piperidin-1-yl)methanone; (5-(ethylsulfonyl)-1,4,5,6,7,8-hexahydropyrazolo[4,3-c]azepin-3-yl)(4-(2-(trifluoromethyl)phenyl)piperidin-1-yl)methanone; 1-(3-(4-(2-(trifluoromethyl)phenyl)piperidine-1-carbonyl)-4,6,7,8-tetrahydropyrazolo[4,3-c]azepin-5(1H)-yl)propan-1-one; (5-ethyl-1,4,5,6,7,8-hexahydropyrazolo[4,3-c]azepin-3-yl)(4-(2-(trifluoromethyl)phenyl)piperidin-1-yl)methanone; (5-(methylsulfonyl)-1,4,5,6,7,8-hexahydropyrazolo[4,3-c]azepin-3-yl)(4-(2-(trifluoromethyl)phenyl)piperidin-1-yl)methanone; 1-(3-(4-(2-(trifluoromethyl)phenyl)piperidine-1-carbonyl)-4,6,7,8-tetrahydropyrazolo[4,3-c]azepin-5(1H)-yl)ethan-1-one; (1,4,5,6,7,8-hexahydropyrazolo[4,3-c]azepin-3-yl)(4-(2-(trifluoromethyl)phenyl)piperidin-1-yl)methanone; (1-methyl-5-(methylsulfonyl)-1,4,5,6,7,8-hexahydropyrazolo[4,3-c]azepin-3-yl)(4-(2-(trifluoromethyl)phenyl)piperidin-1-yl)methanone; (1-methyl-1,4,5,6,7,8-hexahydropyrazolo[4,3-c]azepin-3-yl)(4-(2-(trifluoromethyl)phenyl)piperidin-1-yl)methanone; N,N-dimethyl-3-(4-(2-(trifluoromethyl)phenyl)piperidine-1-carbonyl)-4,6,7,8-tetrahydropyrazolo[4,3-c]azepine-5(1H)-carboxamide; (5-(2-methoxyethyl)-1,4,5,6,7,8-hexahydropyrazolo[4,3-c]azepin-3-yl)(4-(2-(trifluoromethyl)phenyl)piperidin-1-yl)methanone; 2-methoxy-1-(3-(4-(2-(trifluoromethyl)phenyl)piperidine-1-carbonyl)-4,6,7,8-tetrahydropyrazolo[4,3-c]azepin-5(1H)-yl)ethan-1-one; 3,3,3-trifluoro-1-(3-(4-(2-(trifluoromethyl)phenyl)piperidine-1-carbonyl)-4,6,7,8-tetrahydropyrazolo[4,3-c]azepin-5(1H)-yl)propan-1-one; (5-(oxetan-3-yl)-1,4,5,6,7,8-hexahydropyrazolo[4,3-c]azepin-3-yl)(4-(2-(trifluoromethyl)phenyl)piperidin-1-yl)methanone; tert-butyl 3-(4-(2-(trifluoromethyl)phenyl)piperidine-1-carbonyl)-4,6,7,8-tetrahydropyrazolo[4,3-c]azepine-5(1H)-carboxylate; (6-(trifluoromethyl)imidazo[1,2-b]pyridazin-2-yl)(4-(2-(trifluoromethyl)phenyl)piperidin-1-yl)methanone; (6-fluoroimidazo[1,2-b]pyridazin-2-yl)(4-(2-(trifluoromethyl)phenyl)piperidin-1-yl)methanone; (6-(pyrrolidin-1-yl)imidazo[1,2-b]pyridazin-2-yl)(4-(2-(trifluoromethyl)phenyl)piperidin-1-yl)methanone; (6-cyclopropylimidazo[1,2-b]pyridazin-2-yl)(4-(2-(trifluoromethyl)phenyl)piperidin-1-yl)methanone; (6-methoxyimidazo[1,2-b]pyridazin-2-yl)(4-(2-(trifluoromethyl)phenyl)piperidin-1-yl)methanone; (6-methylimidazo[1,2-b]pyridazin-2-yl)(4-(2-(trifluoromethyl)phenyl)piperidin-1-yl)methanone; (6-chloroimidazo[1,2-b]pyridazin-2-yl)(4-(2-(trifluoromethyl)phenyl)piperidin-1-yl)methanone; imidazo[1,2-b]pyridazin-2-yl(4-(2-(trifluoromethyl)phenyl)piperidin-1-yl)methanone; (6-chloro-2-methylimidazo[1,2-b]pyridazin-3-yl)(4-(2-(trifluoromethyl)phenyl)piperidin-1-yl)methanone; (1H-benzo[d]imidazol-2-yl)(4-(2-(trifluoromethyl)phenyl)piperidin-1-yl)methanone; (1H-imidazo[4,5-b]pyridin-2-yl)(4-(2-(trifluoromethyl)phenyl)piperidin-1-yl)methanone; (4-(5-fluoro-2-(trifluoromethyl)phenyl)piperidin-1-yl)(4,5,6,7-tetrahydro-1H-pyrazolo[3,4-c]pyridin-3-yl)methanone; (4-(3-fluoro-2-(trifluoromethyl)phenyl)piperidin-1-yl)(6-(2-methoxyethyl)-4,5,6,7-tetrahydro-1H-pyrazolo[3,4-c]pyridin-3-yl)methanone; 6-methyl-2-(4-(2-(trifluoromethyl)phenyl)piperidine-1-carbonyl)pyrimidine-4-carboxylic acid; methyl 6-methyl-2-(4-(2-(trifluoromethyl)phenyl)piperidine-1-carbonyl)pyrimidine-4-carboxylate; N-(cyclopropylsulfonyl)-2-(4-(2-(trifluoromethyl)phenyl)piperidine-1-carbonyl)benzamide; N-(phenylsulfonyl)-2-(4-(2-(trifluoromethyl)phenyl)piperidine-1-carbonyl)benzamide; N-(methylsulfonyl)-2-(4-(2-(trifluoromethyl)phenyl)piperidine-1-carbonyl)benzamide; 3-(4-(2-(trifluoromethyl)phenyl)piperidine-1-carbonyl)benzamide; 2-(4-(2-(trifluoromethyl)phenyl)piperidine-1-carbonyl)benzamide; 4-(4-(2-(trifluoromethyl)phenyl)piperidine-1-carbonyl)benzoic acid; 3-(4-(2-(trifluoromethyl)phenyl)piperidine-1-carbonyl)benzoic acid; 2-(4-(2-(trifluoromethyl)phenyl)piperidine-1-carbonyl)benzoic acid; 4-(4-(2-(tert-butyl)phenyl)piperidine-1-carbonyl)benzoic acid; 2-(4-(2-(tert-butyl)phenyl)piperidine-1-carbonyl)benzoic acid; 3-(4-(2-(tert-butyl)phenyl)piperidine-1-carbonyl)benzoic acid; 4-(4-(2-(trifluoromethyl)phenyl)piperidine-1-carbonyl)benzamide; 1-(3-(4-(2-(trifluoromethyl)phenyl)piperidine-1-carbonyl)-4,7-dihydroisothiazolo[5,4-c]pyridin-6(5H)-yl)ethan-1-one; (4,5,6,7-tetrahydroisothiazolo[5,4-c]pyridin-3-yl)(4-(2-(trifluoromethyl)phenyl)piperidin-1-yl)methanone; (4,5,6,7-tetrahydroisothiazolo[4,5-c]pyridin-3-yl)(4-(2-(trifluoromethyl)phenyl)piperidin-1-yl)methanone; 1-(3-(4-(2-(trifluoromethyl)phenyl)piperidine-1-carbonyl)-6,7-dihydroisoxazolo[4,5-c]pyridin-5(4H)-yl)ethan-1-one; 1-(3-(4-(2-(trifluoromethyl)phenyl)piperidine-1-carbonyl)-4,7-dihydroisoxazolo[5,4-c]pyridin-6(5H)-yl)ethan-1-one; (4,5,6,7-tetrahydroisoxazolo[4,5-c]pyridin-3-yl)(4-(2-(trifluoromethyl)phenyl)piperidin-1-yl)methanone; benzo[c]isothiazol-3-yl(4-(2-(trifluoromethyl)phenyl)piperidin-1-yl)methanone; benzo[d]thiazol-2-yl(4-(2-(trifluoromethyl)phenyl)piperidin-1-yl)methanone; benzo[d]isoxazol-3-yl(4-(2-(trifluoromethyl)phenyl)piperidin-1-yl)methanone; 1-(3-(4-(2-(trifluoromethyl)phenyl)piperidine-1-carbonyl)-6,7-dihydroisothiazolo[4,5-c]pyridin-5(4H)-yl)ethan-1-one; benzo[d]oxazol-2-yl(4-(2-(trifluoromethyl)phenyl)piperidin-1-yl)methanone; (3-methyloxetan-3-yl)(4-(2-(trifluoromethyl)phenyl)piperidin-1-yl)methanone; oxetan-3-yl(4-(2-(trifluoromethyl)phenyl)piperidin-1-yl)methanone; 2-(2-hydroxyphenyl)-1-(4-(2-(trifluoromethyl)phenyl)piperidin-1-yl)ethan-1-one; (4-(2-(tert-butyl)phenyl)piperidin-1-yl)(tetrahydrothiophen-2-yl)methanone; rac-tert-butyl (2R,3R)-2-(4-(2-(tert-butyl)phenyl)piperidine-1-carbonyl)-3-hydroxypyrrolidine-1-carboxylate; (2R,4R)-2-(4-(2-(tert-butyl)phenyl)piperidine-1-carbonyl)-4-hydroxypyrrolidine-1-carboxylate 2-(2-oxo-2-(4-(2-(trifluoromethyl)phenyl)piperidin-1-yl)ethyl)phenyl sulfamate; (4-(2-(tert-butyl)phenyl)piperidin-1-yl)(1,1-dioxidotetrahydrothiophen-2-yl)methanone; rac-(4-(2-(tert-butyl)phenyl)piperidin-1-yl)((2R,3R)-3-hydroxypyrrolidin-2-yl)methanone; rac-(4-(2-(tert-butyl)phenyl)piperidin-1-yl)((2R,4R)-4-hydroxypyrrolidin-2-yl)methanone; rac-(R)-1-(2-(4-(2-(tert-butyl)phenyl)piperidine-1-carbonyl)pyrrolidin-1-yl)ethan-1-one; (6-bromo-1H-pyrrolo[3,2-b]pyridin-2-yl)(4-(2-(trifluoromethyl)phenyl)piperidin-1-yl)methanone; (5-morpholino-1H-pyrrolo[3,2-b]pyridin-2-yl)(4-(2-(trifluoromethyl)phenyl)piperidin-1-yl)methanone; (5-(1H-imidazol-1-yl)-1H-pyrrolo[3,2-b]pyridin-2-yl)(4-(2-(trifluoromethyl)phenyl)piperidin-1-yl)methanone; (5-chloro-1H-pyrrolo[3,2-b]pyridin-2-yl)(4-(2-(trifluoromethyl)phenyl)piperidin-1-yl)methanone; (5-fluoro-1H-pyrrolo[3,2-b]pyridin-2-yl)(4-(2-(trifluoromethyl)phenyl)piperidin-1-yl)methanone; (5-methyl-1H-pyrrolo[3,2-b]pyridin-2-yl)(4-(2-(trifluoromethyl)phenyl)piperidin-1-yl)methanone; (6-methoxy-1H-pyrrolo[3,2-b]pyridin-2-yl)(4-(2-(trifluoromethyl)phenyl)piperidin-1-yl)methanone; imidazo[1,2-a]pyridin-2-yl(4-(2-(trifluoromethyl)phenyl)piperidin-1-yl)methanone; (6-chloro-2-methylimidazo[1,2-b]pyridazin-3-yl)(4-(2-(trifluoromethyl)phenyl)piperidin-1-yl)methanone; imidazo[1,2-b]pyridazin-6-yl(4-(2-(trifluoromethyl)phenyl)piperidin-1-yl)methanone; (1H-pyrrolo[2,3-b]pyridin-2-yl)(4-(2-(trifluoromethyl)phenyl)piperidin-1-yl)methanone; (1H-pyrrolo[3,2-c]pyridin-2-yl)(4-(2-(trifluoromethyl)phenyl)piperidin-1-yl)methanone; (6-chloro-1H-pyrrolo[3,2-b]pyridin-2-yl)(4-(2-(trifluoromethyl)phenyl)piperidin-1-yl)methanone; (6-morpholino-1H-pyrrolo[3,2-b]pyridin-2-yl)(4-(2-(trifluoromethyl)phenyl)piperidin-1-yl)methanone; (6-(1H-imidazol-1-yl)-1H-pyrrolo[3,2-b]pyridin-2-yl)(4-(2-(trifluoromethyl)phenyl)piperidin-1-yl)methanone; (1-methyl-1H-pyrrolo[3,2-b]pyridin-2-yl)(4-(2-(trifluoromethyl)phenyl)piperidin-1-yl)methanone; (5-methoxy-1H-pyrrolo[3,2-b]pyridin-2-yl)(4-(2-(trifluoromethyl)phenyl)piperidin-1-yl)methanone; (6-fluoro-1H-pyrrolo[3,2-b]pyridin-2-yl)(4-(2-(trifluoromethyl)phenyl)piperidin-1-yl)methanone; (1H-imidazo[4,5-b]pyridin-2-yl)(4-(2-(trifluoromethyl)phenyl)piperidin-1-yl)methanone; (6-methyl-1H-pyrrolo[3,2-b]pyridin-2-yl)(4-(2-(trifluoromethyl)phenyl)piperidin-1-yl)methanone; (1H-indol-2-yl)(4-(2-(trifluoromethyl)phenyl)piperidin-1-yl)methanone; (1H-pyrrolo[3,2-b]pyridin-2-yl)(4-(2-(trifluoromethyl)phenyl)piperidin-1-yl)methanone; (1H-pyrrolo[2,3-c]pyridin-2-yl)(4-(2-(trifluoromethyl)phenyl)piperidin-1-yl)methanone; (1H-pyrazol-3-yl)(4-(2-(trifluoromethyl)phenyl)piperidin-1-yl)methanone; (1H-1,2,3-triazol-5-yl)(4-(2-(trifluoromethyl)phenyl)piperidin-1-yl)methanone; pyrazin-2-yl(4-(2-(trifluoromethyl)phenyl)piperidin-1-yl)methanone; (6-methoxypyridazin-3-yl)(4-(2-(trifluoromethyl)phenyl)piperidin-1-yl)methanone; (6-methylpyridazin-3-yl)(4-(2-(trifluoromethyl)phenyl)piperidin-1-yl)methanone; (4-methyl-1,2,3-thiadiazol-5-yl)(4-(2-(trifluoromethyl)phenyl)piperidin-1-yl)methanone; (6-chloropyridazin-3-yl)(4-(2-(trifluoromethyl)phenyl)piperidin-1-yl)methanone; pyridazin-3-yl(4-(2-(trifluoromethyl)phenyl)piperidin-1-yl)methanone; pyridazin-4-yl(4-(2-(trifluoromethyl)phenyl)piperidin-1-yl)methanone; 4-(2-(trifluoromethyl)phenyl)piperidine-1-carboxylic acid; or 3-oxo-3-(4-(2-(trifluoromethyl)phenyl)piperidin-1-yl)propanoic acid.

### Preparation of Compounds

The compounds used in the chemical reactions described herein are made according to organic synthesis techniques known to those skilled in this art, starting from commercially available chemicals and/or from compounds described in the chemical literature. “Commercially available chemicals” are obtained from standard commercial sources including Acros Organics (Pittsburgh, PA), Aldrich Chemical (Milwaukee, WI, including Sigma Chemical and Fluka), Apin Chemicals Ltd. (Milton Park, UK), Avocado Research (Lancashire, U.K.), BDH Inc. (Toronto, Canada), Bionet (Cornwall, U.K.), Chemservice Inc. (West Chester, PA), Crescent Chemical Co. (Hauppauge, NY), Eastman Organic Chemicals, Eastman Kodak Company (Rochester, NY), Fisher Scientific Co. (Pittsburgh, PA), Fisons Chemicals (Leicestershire, UK), Frontier Scientific (Logan, UT), ICN Biomedicals, Inc. (Costa Mesa, CA), Key Organics (Cornwall, U.K.), Lancaster Synthesis (Windham, NH), Maybridge Chemical Co. Ltd. (Cornwall, U.K.), Parish Chemical Co. (Orem, UT), Pfaltz & Bauer, Inc. (Waterbury, CN), Polyorganix (Houston, TX), Pierce Chemical Co. (Rockford, IL), Riedel de Haen AG (Hanover, Germany), Spectrum Quality Product, Inc. (New Brunswick, NJ), TCI America (Portland, OR), Trans World Chemicals, Inc. (Rockville, MD), and Wako Chemicals USA, Inc. (Richmond, VA).

Suitable reference books and treatise that detail the synthesis of reactants useful in the preparation of compounds described herein, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modem Synthetic Reactions”, 2nd Ed., W. A. Benjamin, Inc. Menlo Park, Calif 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Additional suitable reference books and treatise that detail the synthesis of reactants useful in the preparation of compounds described herein, or provide references to articles that describe the preparation, include for example, Fuhrhop, J. and Penzlin G. “Organic Synthesis: Concepts, Methods, Starting Materials”, Second, Revised and Enlarged Edition (1994) John Wiley & Sons ISBN: 3-527-29074-5; Hoffman, R. V. “Organic Chemistry, An Intermediate Text” (1996) Oxford University Press, ISBN 0-19-509618-5; Larock, R. C. “Comprehensive Organic Transformations: A Guide to Functional Group Preparations” 2nd Edition (1999) Wiley-VCH, ISBN: 0-471-19031-4; March, J. “Advanced Organic Chemistry: Reactions, Mechanisms, and Structure” 4th Edition (1992) John Wiley & Sons, ISBN: 0-471-60180-2; Otera, J. (editor) “Modern Carbonyl Chemistry” (2000) Wiley-VCH, ISBN: 3-527-29871-1; Patai, S. “Patai's 1992 Guide to the Chemistry of Functional Groups” (1992) Interscience ISBN: 0-471-93022-9; Solomons, T. W. G. “Organic Chemistry” 7th Edition (2000) John Wiley & Sons, ISBN: 0-471-19095-0; Stowell, J. C., “Intermediate Organic Chemistry” 2nd Edition (1993) Wiley-Interscience, ISBN: 0-471-57456-2; “Industrial Organic Chemicals: Starting Materials and Intermediates: An Ullmann's Encyclopedia” (1999) John Wiley & Sons, ISBN: 3-527-29645-X, in 8 volumes; “Organic Reactions” (1942-2000) John Wiley & Sons, in over 55 volumes; and “Chemistry of Functional Groups” John Wiley & Sons, in 73 volumes.

Alternatively, specific and analogous reactants can be identified through the indices of known chemicals and reactions prepared by the Chemical Abstract Service of the American Chemical Society, which are available in most public and university libraries, as well as through on-line databases (contact the American Chemical Society, Washington, D.C. for more details). Chemicals that are known but not commercially available in catalogs are optionally prepared by custom chemical synthesis houses, where many of the standard chemical supply houses (e.g., those listed above) provide custom synthesis services. A reference for the preparation and selection of pharmaceutical salts of the heterocyclic RBP4 inhibitory compound described herein is P. H. Stahl & C. G. Wermuth “Handbook of Pharmaceutical Salts”, Verlag Helvetica Chimica Acta, Zurich, 2002.

### Retinol Binding Protein 4 (RBP4)

Retinol-binding protein 4 (RBP4), the sole retinol transporter in blood, is secreted from adipocytes and the liver. Lowering levels of RBP4 can lead to reduction in the accumulation of lipofuscin that leads to vision loss in diseases like Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or ABCA4 gene associated retinal diseases. In some instances, lowering RBP4 reduces the accumulation of lipofuscin in the retina. In some embodiments, compounds and formulations described herein lower serum or plasma RBP4 and thus delay or stop vision loss from excessive accumulation of lipofuscin in the retina.

In some embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 300% from baseline. In some embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 300% from baseline. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 300% from baseline. In some embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 300% from baseline. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 20% from baseline. In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 25% from baseline. In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 300% from baseline. In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 40% from baseline. In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 50% from baseline. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 65% from baseline. In certain embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 80% from baseline. In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 85% from baseline.

In some embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 48 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 1 mg/dL. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 2 mg/dL. In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 5 mg/dL. In certain embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 10 mg/dL. In some embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced by at least 15 mg/dL.

In some embodiments, after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 μM. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 μM. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 μM. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 μM. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 μM.

In some embodiments, after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 μM. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of are reduced to below 1.5 μM. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 μM. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 μM. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 μM.

In some embodiments, after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 μM. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of are reduced to below 2 μM. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 μM. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 μM. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 μM.

In some embodiments, after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 mM. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 mM. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 mM. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 mM. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1 mM.

In some embodiments, after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 mM. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of are reduced to below 1.5 mM. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 mM. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 mM. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 1.5 mM.

In some embodiments, after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 mM. In other embodiments, 6 hours after administration of a compound of Formula (I), a pharmaceutically acceptable salt, solvate, polymorph, prodrug, metabolite, N-oxide, stereoisomer, or isomer thereof, the serum or plasma levels of are reduced to below 2 mM. In some embodiments, 12 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 mM. In certain embodiments, 24 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 mM. In some embodiments, 36 hours after administration of a compound of Formula (I), a pharmaceutically acceptable salt, solvate, polymorph, prodrug, metabolite, N-oxide, stereoisomer, or isomer thereof, the serum or plasma levels of RBP4 are reduced to below 2 mM.

### Methods of Treatment

In some embodiments, a compound disclosed herein is used to treat or ameliorate a disease associated with altered RBP4 pathways when administered to a subject in need thereof. In some cases, a compound disclosed herein is used to treat or ameliorate the effects of a disease associated with altered RBP4 pathway when administered to a subject in need thereof. Exemplary diseases associated with altered RBP4 include eye diseases. In some instances, the eye disease is characterized by excessive lipofuscin accumulation in the retina. In some instances, a compound disclosed herein is used to treat or ameliorate an eye disease when administered to a subject in need thereof. Exemplary eye disease includes Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or an ABCA4 gene associated retinal disease.

### Age-Related Macular Degeneration

Age-related macular degeneration (AMD) is a common eye condition and a leading cause of vision loss among people aged 50 and older. It causes damage to the macula, a small spot near the center of the retina and the part of the eye needed for sharp, central vision. As AMD progresses, a blurred area near the center of vision is a common symptom. Over time, the blurred area may grow larger and the subject may develop blank spots in his or her central vision.

Some embodiments provided herein describe the use of the compounds of Formula (I) described herein for treating AMD in a subject in need thereof. In some embodiments, the compounds of Formula (I) inhibit AMD. In certain embodiments, the compounds of Formula (1) arrest development of AMD or its clinical symptoms. In certain embodiments, the compounds of Formula (I) reduce development of AMD or its clinical symptoms. In certain embodiments, the compounds of Formula (I) relieve the subject of AMD. In certain embodiments, the compounds of Formula (I) cause regression, reversal, or amelioration of AMD. In certain embodiments, the compounds of Formula (I) reduce the number, frequency, duration, or severity of AMD clinical symptoms.

In some embodiments, the compounds of Formula (I) are used prophylactically. In certain embodiments, the compounds of Formula (I) are used to prevent or reduce the risk of developing AMD. In certain embodiments, the compounds of Formula (I) cause the clinical symptoms of AMD to not develop in a subject who may be predisposed to AMD but who does not yet experience or display symptoms of AMD.

### Dry (Atrophic) Age-Related Macular Degeneration

Approximately 85% to 90% of the cases of macular degeneration are the “dry” (atrophic) type. It is estimated that 62.9 million individuals worldwide have this form of AMD; 8 million of them are Americans. Due to increasing life expectancy and current demographics this number is expected to triple by 2020. There is currently no FDA-approved treatment for dry AMD. Given the lack of treatment and high prevalence, development of drugs for dry AMD is of upmost importance. Clinically, atrophic AMD represents a slowly progressing neurodegenerative disorder in which specialized neurons (rod and cone photoreceptors) die in the central part of the retina called the macula. Histopathological and clinical imaging studies indicate that photoreceptor degeneration in dry AMD is triggered by abnormalities in the retinal pigment epithelium (RPE) that lies beneath photoreceptors and provides critical metabolic support to these light-sensing neuronal cells. Experimental and clinical data indicate that excessive accumulation of cytotoxic autofluorescent lipid-protein-retinoid aggregates (lipofuscin) in the RPE is a major trigger of dry AMD. The major cytotoxic component of RPE lipofuscin is pyridinium bisretinoid A2E. Additional cytotoxic bisretinoids are isoA2E, atRAL di-PE, and A2-DHP-PE. Formation of A2E and other lipofuscin bisretinoids, such as A2-DHP-PE (A2-dihydropyridine-phosphatidylethanolamine) and atRALdi-PE (all-trans-retinal dimer-phosphatidylethanolamine), begins in photoreceptor cells in a non-enzymatic manner and can be considered as a by-product of the properly functioning visual cycle.

Some embodiments provided herein describe the use of the compounds of Formula (I) described herein for treating dry (atrophic) AMD in a subject in need thereof. In some embodiments, the compounds of Formula (I)inhibit dry (atrophic) AMD. In certain embodiments, the compounds of Formula (I) arrest development of dry (atrophic) AMD or its clinical symptoms. In certain embodiments, the compounds of Formula (I) reduce development of dry (atrophic) AMD or its clinical symptoms. In certain embodiments, the compounds of Formula (I) relieve the subject of dry (atrophic) AMD. In certain embodiments, the compounds of Formula (I) cause regression, reversal, or amelioration of dry (atrophic) AMD. In certain embodiments, the compounds of Formula (I) reduce the number, frequency, duration, or severity of dry (atrophic) AMD clinical symptoms.

In some embodiments, the compounds of Formula (I) are used prophylactically. In certain embodiments, the compounds of Formula (I) are used to prevent or reduce the risk of developing dry (atrophic) AMD. In certain embodiments, the compounds of Formula (I) cause the clinical symptoms of dry (atrophic) AMD to not develop in a subject who may be predisposed to dry (atrophic) AMD but who does not yet experience or display symptoms of dry (atrophic) AMD. Juvenile Macular Degeneration (Stargardt Disease)

Stargardt Disease (STGD) is an inherited form of juvenile-onset macular degeneration. STGD is characterized by the dramatic accumulation of lipofuscin in the retina. STGD is linked to defects in the ABCA4 gene. Excessive production of lipofuscin bisretinoids is thought to be the sole biochemical trigger of monogenic STGD caused by recessive mutations in the ABCA4 gene. Symptoms include wavy vision, blind spots, blurriness, loss of depth perception, sensitivity to glare, impaired color vision, and difficulty adapting to dim lighting. Symptoms typically develop before age 20.

Some embodiments provided herein describe the use of the compounds of Formula (I) described herein for treating STGD in a subject in need thereof. In some embodiments, the compounds of Formula (I) inhibit STGD. In certain embodiments, the compounds of Formula (I) arrest development of STGD or its clinical symptoms. In certain embodiments, the compounds of Formula (I) reduce development of STGD or its clinical symptoms. In certain embodiments, the compounds of Formula (I) relieve the subject of STGD. In certain embodiments, the compounds of Formula (I) cause regression, reversal, or amelioration of STGD. In certain embodiments, the compounds of Formula (I) reduce the number, frequency, duration, or severity of STGD clinical symptoms.

In some embodiments, the compounds of Formula (I) are used prophylactically. In certain embodiments, the compounds of Formula (I) are used to prevent or reduce the risk of developing STGD. In certain embodiments, the compounds of Formula (I) cause the clinical symptoms of STGD to not develop in a subject who may be predisposed to STGD but who does not yet experience or display symptoms of STGD.

### Best Disease

Vitelliform dystrophy, or Best disease, is a hereditary retinal dystrophy involving the retinal pigment epithelium (RPE), and leads to a characteristic bilateral yellow “egg-yolk” appearance of the macula. This disease tends to present itself in childhood or early adulthood. Best disease is caused by mutations in the BEST1 gene, which encodes the transmembrane protein bestrophin 1. The mutations lead to a buildup of lipofuscin between the outer retina and the retinal pigment epithelium.

Some embodiments provided herein describe the use of the compounds of Formula (I) described herein for treating Best disease in a subject in need thereof. In some embodiments, the compounds of Formula (I) inhibit Best disease. In certain embodiments, the compounds of Formula (I) arrest development of Best disease or its clinical symptoms. In certain embodiments, the compounds of Formula (I) reduce development of Best disease or its clinical symptoms. In certain embodiments, the compounds of Formula (I) relieve the subject of Best disease. In certain embodiments, the compounds of Formula (I) cause regression, reversal, or amelioration of Best disease. In certain embodiments, the compounds of Formula (I) reduce the number, frequency, duration, or severity of Best disease clinical symptoms.

In some embodiments, the compounds of Formula (I) are used prophylactically. In certain embodiments, the compounds of Formula (I) are used to prevent or reduce the risk of developing Best disease. In certain embodiments, the compounds of Formula (I) cause the clinical symptoms of Best disease to not develop in a subject who may be predisposed to Best disease but who does not yet experience or display symptoms of Best disease.

### Geographic Atrophy

Geographic atrophy is a chronic progressive degeneration of the macula and can be seen as part of late-stage age-related macular degeneration (AMD). The condition leads to central scotomas and permanent loss of visual acuity. The disease is characterized by localized sharply demarcated atrophy of outer retinal tissue, retinal pigment epithelium and choriocapillaris.

Some embodiments provided herein describe the use of the compounds of Formula (I) described herein for treating geographic atrophy in a subject in need thereof. In some embodiments, the compounds of Formula (I) inhibit geographic atrophy. In certain embodiments, the compounds of Formula (I) arrest development of geographic atrophy or its clinical symptoms. In certain embodiments, the compounds of Formula (I) reduce development of geographic atrophy or its clinical symptoms. In certain embodiments, the compounds of Formula (I) relieve the subject of geographic atrophy. In certain embodiments, the compounds of Formula (I) cause regression, reversal, or amelioration of geographic atrophy. In certain embodiments, the compounds of Formula (I) reduce the number, frequency, duration, or severity of geographic atrophy clinical symptoms.

In some embodiments, the compounds of Formula (I) are used prophylactically. In certain embodiments, the compounds of Formula (I) are used to prevent or reduce the risk of developing geographic atrophy. In certain embodiments, the compounds of Formula (I) cause the clinical symptoms of geographic atrophy to not develop in a subject who may be predisposed to geographic atrophy but who does not yet experience or display symptoms of geographic atrophy.

### Adult Vitelliform Maculopathy

Adult vitelliform maculopathy is an eye disorder that can cause progressive vision loss. The condition causes the accumulation of lipofuscin in the cells underlying the macula. The condition typically manifests after the age of 40. The condition can be caused by mutations in the RDS and VMD2 genes.

Some embodiments provided herein describe the use of the compounds of Formula (I) described herein for treating adult vitelliform maculopathyin a subject in need thereof. In some embodiments, the compounds of Formula (I) inhibit adult vitelliform maculopathy. In certain embodiments, the compounds of Formula (I) arrest development of adult vitelliform maculopathy or its clinical symptoms. In certain embodiments, the compounds of Formula (I) reduce development of adult vitelliform maculopathy clinical symptoms or its clinical symptoms. In certain embodiments, the compounds of Formula (I) relieve the subject of adult vitelliform maculopathy. In certain embodiments, the compounds of Formula (I) cause regression, reversal, or amelioration of adult vitelliform maculopathy. In certain embodiments, the compounds of Formula (I) reduce the number, frequency, duration, or severity of adult vitelliform maculopathy clinical symptoms.

In some embodiments, the compounds of Formula (I) are used prophylactically. In certain embodiments, the compounds of Formula (I) are used to prevent or reduce the risk of developing adult vitelliform maculopathy. In certain embodiments, the compounds of Formula (I) cause the clinical symptoms of adult vitelliform maculopathy to not develop in a subject who may be predisposed to adult vitelliform maculopathy but who does not yet experience or display symptoms of adult vitelliform maculopathy.

### Stargardt-Like Macular Dystrophy

Stargardt-like macular dystrophy is similar in symptoms and presentation to Stargardt disease, but typically presents later in childhood than Stargardt disease. Stargardt-like macular dystrophy is linked with mutations in the EVOVL4 gene.

Some embodiments provided herein describe the use of the compounds of Formula (I) described herein for treating Stargardt-like macular dystrophy in a subject in need thereof. In some embodiments, the compounds of Formula (I) inhibit Stargardt-like macular dystrophy. In certain embodiments, the compounds of Formula (I) arrest development Stargardt-like macular dystrophy or its clinical symptoms. In certain embodiments, the compounds of Formula (I) reduce development of Stargardt-like macular dystrophy or its clinical symptoms. In certain embodiments, the compounds of Formula (I) relieve the subject of Stargardt-like macular dystrophy. In certain embodiments, the compounds of Formula (I) cause regression, reversal, or amelioration of Stargardt-like macular dystrophy. In certain embodiments, the compounds of Formula (I) reduce the number, frequency, duration, or severity of Stargardt-like macular dystrophy clinical symptoms.

In some embodiments, the compounds of Formula (I) are used prophylactically. In certain embodiments, the compounds of Formula (I) are used to prevent or reduce the risk of developing Stargardt-like macular dystrophy. In certain embodiments, the compounds of Formula (I) cause the clinical symptoms of Stargardt-like macular dystrophy to not develop in a subject who may be predisposed to Stargardt-like macular dystrophy but who does not yet experience or display symptoms of Stargardt-like macular dystrophy.

### Diabetic Retinopathy

Diabetic retinopathy is a diabetes complication that affects the eyes. It may be caused by damage to the blood vessels of the light sensitive tissue at the back of the eye, and can eventually cause blindness. Diabetic retinopathy can be caused when new blood vessels in the retina fail to grow. Diabetic retinopathy may also result from blood vessels becoming damaged and closing off, causing the growth of new, abnormal blood vessels in the retina.

Some embodiments provided herein describe the use of the compounds of Formula (I) described herein for treating diabetic retinopathy in a subject in need thereof. In some embodiments, the compounds of Formula (I)inhibit diabetic retinopathy. In certain embodiments, the compounds of Formula (I) arrest development diabetic retinopathy or its clinical symptoms. In certain embodiments, the compounds of Formula (I) reduce development of diabetic retinopathy or its clinical symptoms. In certain embodiments, the compounds of Formula (I) relieve the subject's diabetic retinopathy. In certain embodiments, the compounds of Formula (I) cause regression, reversal, or amelioration of diabetic retinopathy. In certain embodiments, the compounds of Formula (I) reduce the number, frequency, duration, or severity of diabetic retinopathy clinical symptoms.

In some embodiments, the compounds of Formula (I) are used prophylactically. In certain embodiments, the compounds of Formula (I) are used to prevent or reduce the risk of developing diabetic retinopathy. In certain embodiments, the compounds of Formula (I) cause the clinical symptoms of diabetic retinopathy to not develop in a subject who may be predisposed to diabetic retinopathy but who does not yet experience or display symptoms of diabetic retinopathy.

### ABCA4 Gene Associated Retinal Diseases

ATP-binding cassette, subfamily A, member 4 (ABCA4) is a protein encoded by the ABCA4 gene in humans and other eukaryotes. The ABCA4 protein is expressed almost exclusively in the retina and is implicated in Stargardt and other eye diseases, including but not limited to fundus flavimaculatus, cone-rod dystrophy, retinitis pigmentosa, and age-related macular degeneration. Diminished ABCA4 activity is linked with excessive accumulation of toxic retinoids and lipofuscin. Such mutations in some instances are detected by sequencing a subject's DNA or RNA.

Some embodiments provided herein describe the use of the compounds of Formula (I) described herein for treating ABCA4 gene associated retinal diseases in a subject in need thereof. In some embodiments, the compounds of Formula (I) inhibit ABCA4 gene associated retinal diseases. In certain embodiments, the compounds of Formula (I) arrest development ABCA4 gene associated retinal diseases or their clinical symptoms. In certain embodiments, the compounds of Formula (I) reduce development of ABCA4 gene associated retinal diseases or their clinical symptoms. In certain embodiments, the compounds of Formula (I) relieve the subject ABCA4 gene associated retinal diseases. In certain embodiments, the compounds of Formula (I) cause regression, reversal, or amelioration ABCA4 gene associated retinal diseases. In certain embodiments, the compounds of Formula (I) reduce the number, frequency, duration, or severity of ABCA4 gene associated retinal disease clinical symptoms.

In some embodiments, the compounds of Formula (I) are used prophylactically. In certain embodiments, the compounds of Formula (I) are used to prevent or reduce the risk of developing ABCA4 gene associated retinal diseases. In certain embodiments, the compounds of Formula (I) cause the clinical symptoms ABCA4 gene associated retinal diseases to not develop in a subject who may be predisposed to ABCA4 gene associated retinal diseases but who does not yet experience or display symptoms of ABCA4 gene associated retinal diseases.

### Pharmaceutical Compositions

In certain embodiments, a compound of Formula (I) as described herein may be combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Provided herein is a pharmaceutical composition comprising at least one heterocyclic RBP4 inhibitory compound, or a pharmaceutically acceptable salt, crystalline, solvate, polymorph, prodrug, metabolite, N-oxide, stereoisomer, or isomer thereof, together with one or more pharmaceutically acceptable carriers. The carrier(s)(or excipient(s)) is acceptable or suitable if the carrier is compatible with the other ingredients of the composition and not deleterious to the recipient (i.e., the subject or patient) of the composition.

One embodiment provides a pharmaceutical composition comprising a compound of Formula (I), or a pharmaceutically acceptable salt, crystalline, solvate, polymorph, prodrug metabolite, N-oxide, stereoisomer, or isomer thereof, and at least one excipient. In one embodiment, the pharmaceutical composition is provided in a dosage form for oral administration, which comprise a compound provided herein, and one or more pharmaceutically acceptable excipients or carriers.

In certain embodiments, the pharmaceutical composition disclosed herein may be in the form of a tablet, pill, granule, capsule, or a variant thereof. In certain embodiments, a tablet pharmaceutical composition is disclosed herein. In certain embodiments, the tablet pharmaceutical composition may include a compound of Formula (I), a pharmaceutically acceptable salt, solvate, polymorph, prodrug, metabolite, N-oxide, stereoisomer, or isomer thereof. In certain embodiments, the compound of Formula (I) in the tablet pharmaceutical composition may include a compound of Formula (I) disclosed herein. In certain embodiments, the tablet pharmaceutical composition may include at least one excipient.

Although embodiments of present disclosure may recite limitations, amounts, percentages, properties, compositions, process, and/or methods related to a tablet pharmaceutical composition, it is not intended to limit the scope of the present disclosure. One skilled in the art will understand that the limitations, amounts, percentages, properties, compositions, process, and/or methods related to a tablet pharmaceutical composition may be applicable to other forms of pharmaceutical composition, such as a pill pharmaceutical composition, a granule pharmaceutical composition, a capsule pharmaceutical composition, or a variant thereof.

In certain embodiments, the heterocyclic RBP4 inhibitory compound as described by Formula (I) is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable dosage forms include, for example, tablets, pills, granules, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, PA (2005)).

In some embodiments, the pharmaceutical compositions provided herein are formulated for oral administration in tablet, pill, granule, or capsule form. In some embodiments, the pharmaceutical formulation is formulated as a tablet. In some embodiments, the pharmaceutical formulation is formulated as a capsule. In some embodiments, a tablet comprises a solid carrier or an adjuvant. In some embodiments, physiological saline solution, dextrose or other saccharide solution, or glycols are optionally included. In some embodiments, a capsule comprises a solid carrier such as gelatin.

In another embodiment, the pharmaceutical compositions are provided in a dosage form for parenteral administration, which comprise a compound provided herein, and one or more pharmaceutically acceptable excipients or carriers. In some embodiments, preservatives, stabilizers, buffers, antioxidants, and/or other additives are included.

### Compositions

In certain embodiments, a tablet pharmaceutical composition is disclosed herein. In certain embodiments, the tablet pharmaceutical composition may include a compound of Formula (I), a pharmaceutically acceptable salt, solvate, polymorph, prodrug, metabolite, N-oxide, stereoisomer, or isomer thereof. In certain embodiments, the compound of Formula (I) in the tablet pharmaceutical composition may include a compound of Formula (I) disclosed herein. In certain embodiments, the tablet pharmaceutical composition may include at least one excipient.

In certain embodiments, the compound of Formula (I) of the tablet pharmaceutical composition may be effective to reduce RBP4 concentrations in dosages forms from about 1 to about 200 mg, e.g., about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, or about 200 mg, or any amount therebetween.

In certain embodiments, the compound of Formula (I) of the tablet pharmaceutical composition may be effective to reduce RBP4 concentrations in dosages forms of from about 2 to about 100 mg, e.g., 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg, 10.5 mg, 11 mg, 11.5 mg 12 mg, 12.5 mg, 13 mg, 13.5 mg, 14 mg, 14.5 mg, 15 mg, 15.5 mg, 16 mg, 16.5 mg, 17 mg, 17.5 mg, 18 mg, 18.5 mg, 19 mg, 19.5 mg, 20 mg, 20.5 mg, 21 mg, 21.5 mg, 22 mg, 22.5 mg, 23 mg, 23.5 mg, 24 mg, 24.5 mg, 25 mg, 25.5 mg, 26 mg, 26.5 mg, 27 mg, 27.5 mg, 28 mg, 28.5 mg, 29 mg, 29.5 mg, 30 mg, 30.5 mg, 31 mg, 31.5 mg, 32 mg, 32.5 mg, 33 mg, 33.5 mg, 34 mg, 34.5 mg, 35 mg, 35.5 mg, 36 mg, 36.5 mg, 37 mg, 37.5 mg, 38 mg, 38.5 mg, 39 mg, 39.5 mg, 40 mg, 40.5 mg, 41 mg, 41.5 mg, 42 mg, 42.5 mg, 43 mg, 43.5 mg, 44 mg, 44.5 mg, 45 mg, 45.5 mg, 46 mg, 46.5 mg, 47 mg, 47.5 mg, 48 mg, 48.5 mg, 49 mg, 49.5 mg, 50 mg, 50.5 mg, 51 mg, 51.5 mg, 52 mg, 52.5 mg, 53 mg, 53.5 mg, 54 mg, 54.5 mg, 55 mg, 55.5 mg, 56 mg, 56.5 mg, 57 mg, 57.5 mg, 58 mg, 58.5 mg, 59 mg, 59.5 mg, 60 mg, 60.5 mg, 61 mg, 61.5 mg, 62 mg, 62.5 mg, 63 mg, 63.5 mg, 64 mg, 64.5 mg, 65 mg, 65.5 mg, 66 mg, 66.5 mg, 67 mg, 67.5 mg, 68 mg, 68.5 mg, 69 mg, 69.5 mg, 70 mg, 70.5 mg, 71 mg, 71.5 mg, 72 mg, 72.5 mg, 73 mg, 73.5 mg, 74 mg, 74.5 mg, 75 mg, 75.5 mg, 76 mg, 76.5 mg, 77 mg, 77.5 mg, 78 mg, 78.5 mg, 79 mg, 79.5 mg, 80 mg, 80.5 mg, 81 mg, 81.5 mg, 82 mg, 82.5 mg, 83 mg, 83.5 mg, 84 mg, 84.5 mg, 85 mg, 85.5 mg, 86 mg, 86.5 mg, 87 mg, 87.5 mg, 88 mg, 88.5 mg, 89 mg, 89.5 mg, 90 mg, 90.5 mg, 91 mg, 91.5 mg, 92 mg, 92.5 mg, 93 mg, 93.5 mg, 94 mg, 94.5 mg, 95 mg, 95.5 mg, 96 mg, 96.5 mg, 97 mg, 97.5 mg, 98 mg, 98.5 mg, 99 mg, 99.5 mg, or 100 mg, or any amount therebetween.

In certain embodiments, the compound of Formula (I) of the tablet pharmaceutical composition may be effective to reduce RBP4 concentrations in dosages forms of from about 1 to about 50 mg, e.g., about 1 mg, about 1.5 mg, about 2 mg, about 2.5 mg, about 3 mg, about 3.5 mg, about 4 mg, 4.2 mg, 4.4 mg, 4.6 mg, 4.8 mg, 5 mg, 5.2 mg, 5.4 mg, 5.6 mg, 5.8 mg, 6 mg, 6.2 mg, 6.4 mg, 6.6 mg, 6.8 mg, 7 mg, 7.2 mg, 7.4 mg, 7.6 mg, 7.8 mg, 8 mg, 8.2 mg, 8.4 mg, 8.6 mg, 8.8 mg, 9 mg, 9.2 mg, 9.4 mg, 9.6 mg, 9.8 mg, 10 mg, 10.2 mg, 10.4 mg, 10.6 mg, 10.8 mg, 11 mg, 11.2 mg, 11.4 mg, 11.6 mg, 11.8 mg, 12 mg, 12.2 mg, 12.4 mg, 12.6 mg, 12.8 mg, 13 mg, 13.2 mg, 13.4 mg, 13.6 mg, 13.8 mg, 14 mg, 14.2 mg, 14.4 mg, 14.6 mg, 14.8 mg, 15 mg, 15.2 mg, 15.4 mg, 15.6 mg, 15.8 mg, 16 mg, 16.2 mg, 16.4 mg, 16.6 mg, 16.8 mg, 17 mg, 17.2 mg, 17.4 mg, 17.6 mg, 17.8 mg, 18 mg, 18.2 mg, 18.4 mg, 18.6 mg, 18.8 mg, 19 mg, 19.2 mg, 19.4 mg, 19.6 mg, 19.8 mg, 20 mg, 20.2 mg, 20.4 mg, 20.6 mg, 20.8 mg, 21 mg, 21.2 mg, 21.4 mg, 21.6 mg, 21.8 mg, 22 mg, 22.2 mg, 22.4 mg, 22.6 mg, 22.8 mg, 23 mg, 23.2 mg, 23.4 mg, 23.6 mg, 23.8 mg, 24 mg, 24.2 mg, 24.4 mg, 24.6 mg, 24.8 mg, 25 mg, 25.2 mg, 25.4 mg, 25.6 mg, 25.8 mg, 26 mg, 26.2 mg, 26.4 mg, 26.6 mg, 26.8 mg, 27 mg, 27.2 mg, 27.4 mg, 27.6 mg, 27.8 mg, 28 mg, 28.2 mg, 28.4 mg, 28.6 mg, 28.8 mg, 29 mg, 29.2 mg, 29.4 mg, 29.6 mg, 29.8 mg, 30 mg, 30.2 mg, 30.4 mg, 30.6 mg, 30.8 mg, 31 mg, 31.2 mg, 31.4 mg, 31.6 mg, 31.8 mg, 32 mg, 32.2 mg, 32.4 mg, 32.6 mg, 32.8 mg, 33 mg, 33.2 mg, 33.4 mg, 33.6 mg, 33.8 mg, 34 mg, 34.2 mg, 34.4 mg, 34.6 mg, 34.8 mg, 35 mg, 35.2 mg, 35.4 mg, 35.6 mg, 35.8 mg, 36 mg, 36.2 mg, 36.4 mg, 36.6 mg, 36.8 mg, 37 mg, 37.2 mg, 37.4 mg, 37.6 mg, 37.8 mg, 38 mg, 38.2 mg, 38.4 mg, 38.6 mg, 38.8 mg, 39 mg, 39.2 mg, 39.4 mg, 39.6 mg, 39.8 mg, 40 mg, 40.2 mg, 40.4 mg, 40.6 mg, 40.8 mg, 41 mg, 41.2 mg, 41.4 mg, 41.6 mg, 41.8 mg, 42 mg, 42.2 mg, 42.4 mg, 42.6 mg, 42.8 mg, 43 mg, 43.2 mg, 43.4 mg, 43.6 mg, 43.8 mg, 44 mg, 44.2 mg, 44.4 mg, 44.6 mg, 44.8 mg, 45 mg, 45.2 mg, 45.4 mg, 45.6 mg, 45.8 mg, 46 mg, 46.2 mg, 46.4 mg, 46.6 mg, 46.8 mg, 47 mg, 47.2 mg, 47.4 mg, 47.6 mg, 47.8 mg, 48 mg, 48.2 mg, 48.4 mg, 48.6 mg, 48.8 mg, 49 mg, 49.2 mg, 49.4 mg, 49.6 mg, 49.8 mg, or 50 mg, or any amount therebetween.

In certain embodiments, the compound of Formula (I) of the tablet pharmaceutical composition may be effective to reduce RBP4 concentrations in dosages forms of from about 1 to about 25 mg, e.g., about 1 mg, 1.2 mg, 1.4 mg, 1.5 mg, 1.7 mg, 2 mg, 2.2 mg, 2.4 mg, 2.5 mg, 2.7 mg, 2.9 mg, 3.0 mg, 3.2 mg, 3.4 mg, 3.5 mg, 3.7 mg, 3.9 mg, 4.0 mg, 4.2 mg, 4.4 mg, 4.6 mg, 4.8 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg, 20 mg, 20.1 mg, 20.2 mg, 20.3 mg, 20.4 mg, 20.5 mg, 20.6 mg, 20.7 mg, 20.8 mg, 20.9 mg, 21 mg, 21.1 mg, 21.2 mg, 21.3 mg, 21.4 mg, 21.5 mg, 21.6 mg, 21.7 mg, 21.8 mg, 21.9 mg, 22 mg, 22.1 mg, 22.2 mg, 22.3 mg, 22.4 mg, 22.5 mg, 22.6 mg, 22.7 mg, 22.8 mg, 22.9 mg, 23 mg, 23.1 mg, 23.2 mg, 23.3 mg, 23.4 mg, 23.5 mg, 23.6 mg, 23.7 mg, 23.8 mg, 23.9 mg, 24 mg, 24.1 mg, 24.2 mg, 24.3 mg, 24.4 mg, 24.5 mg, 24.6 mg, 24.7 mg, 24.8 mg, 24.9 mg, or 25 mg, or any amount therebetween.

In certain embodiments, the compound of Formula (I) of a tablet pharmaceutical composition disclosed herein may be a micronized crystalline. In certain embodiments, the compound of Formula (I) of a tablet pharmaceutical composition disclosed herein may be a polymorph exhibiting an x-ray powder diffraction pattern having at least three characteristic peaks expressed in degrees two theta (+/−0.5 degree theta) at 6.7, 9.3, 14.1, 17.2, 23.5, 27.1, and/or 29.0. In certain embodiments, the compound of Formula (I) of a tablet pharmaceutical composition disclosed herein may be amorphous.

In certain embodiments, the at least one excipient of a tablet pharmaceutical composition disclosed herein may include a disintegrant. In embodiments, the disintegrant may be selected from the group comprising agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof. In embodiments, the disintegrant may include CCNa, MCC, or both.

In embodiments, a disintegrant of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%0, 0.44%, 0.45%, 0.46%, 0.47%, 0.48%, 0.49%, 0.50%, 0.51%, 0.52%, 0.53%, 0.54%, 0.55%0, 0.56%, 0.57%, 0.58%, 0.59%, 0.60%, 0.61%, 0.62%, 0.63%, 0.64%, 0.65%, 0.66%, 0.67%0, 0.68%, 0.69%, 0.70%, 0.71%, 0.72%, 0.73%, 0.74%, 0.75%, 0.76%, 0.77%, 0.78%, 0.79%0, 0.80%, 0.81%, 0.82%, 0.83%, 0.84%, 0.85%, 0.86%, 0.87%, 0.88%, 0.89%, 0.90%, 0.91%0, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. The various percentages listed above are applicable to agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof.

In embodiments, a disintegrant of a tablet pharmaceutical composition disclosed herein is from about 0.01% to about 99% by weight of the tablet pharmaceutical composition, e.g., about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. The various ranges listed above are applicable to agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof.

In embodiments, a disintegrant of a tablet pharmaceutical composition disclosed herein may comprise CCNa. In embodiments, the CCNa may be about 0.75% by weight of the tablet pharmaceutical composition. In embodiments, the CCNa may be about 3% by weight of the tablet pharmaceutical composition. In embodiments, the CCNa may be about any other percentage disclosed herein by weight of the tablet pharmaceutical composition.

In embodiments, the at least one excipient of a tablet pharmaceutical composition disclosed herein may comprise a dispersion polymer. In embodiments, the dispersion polymer may comprise hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, or combinations thereof. In embodiments, the compound of Formula (I) of a tablet pharmaceutical composition disclosed herein may be amorphous and molecularly dispersed in the dispersion polymer. In embodiments, a dispersion polymer may comprise HPC. In embodiments, a dispersion polymer may comprise HPMC.

In embodiments, a dispersion polymer of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. The various percentages listed above are applicable to hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, or combinations thereof.

In embodiments, a dispersion polymer of a tablet pharmaceutical composition disclosed herein may be from about 0.01% to about 99% by weight of the tablet pharmaceutical composition, e.g., about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 22% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. The various ranges listed above are applicable to hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, poly oxyethylene-poly oxypropylene block copolymers, or combinations thereof.

In certain embodiments, a dispersion polymer may comprise HPC. In embodiments, the HPC may be about 6% by weight of a tablet pharmaceutical composition disclosed herein. In certain embodiments, a dispersion polymer may comprise HPMC. In certain embodiments, HPMC may be about 10% by weight of a tablet pharmaceutical composition disclosed herein. In certain embodiments, HPMC may be about 20% by weight of a tablet pharmaceutical composition disclosed herein. In certain embodiments, the at least one excipient may comprise both about 0.75% of CCNa and about 6% of HPC by weight of a tablet pharmaceutical composition disclosed herein. In certain embodiments, the at least one excipient may comprise both about 0.75% of CCNa and about 10% of HPMC by weight of a tablet pharmaceutical composition disclosed herein. In certain embodiments, the at least one excipient may comprise both about 0.75% of CCNa and about 20% of HPMC by weight of a tablet pharmaceutical composition disclosed herein.

In certain embodiments, a dispersion polymer may comprise PVP. In certain embodiments, HPMC may be about 10% by weight of a tablet pharmaceutical composition disclosed herein. In certain embodiments, PVP may be about 20% by weight of a tablet pharmaceutical composition disclosed herein. In certain embodiments, the at least one excipient may comprise both about 0.75% of CCNa and about 6% of PVP by weight of a tablet pharmaceutical composition disclosed herein. In certain embodiments, the at least one excipient may comprise both about 0.75% of CCNa and about 10% of PVP by weight of a tablet pharmaceutical composition disclosed herein. In certain embodiments, the at least one excipient may comprise both about 0.75% of CCNa and about 20% of PVP by weight of a tablet pharmaceutical composition disclosed herein.

In certain embodiments, the at least one excipient may comprise a disintegrant, a dispersion polymer, a diluent, a binder, an anti-adherent, a filler, a sweetener, a wetting agent, a glidant, a lubricant, or a surfactant, or any combinations thereof. In certain embodiments, a diluent may comprise microcrystalline cellulose (MCC), lactose monohydrate (LMH), or both. In embodiments, a binder may comprise MCC, HPC, or both. In certain embodiments, an anti-adherent may comprise colloidal silicon dioxide (CSD), magnesium stearate, or both. In certain embodiments, a glidant may comprise CSD. In certain embodiments, a lubricant may comprise magnesium stearate. In certain embodiments, a lubricant may be ingragranular or extragranular. In certain embodiments, the at least one excipient may comprise MCC, LMH, HPC, CCNa, CSD, and magnesium stearate. In certain embodiments, a diluent may function as a binder and/or a disintegrant. In certain embodiments, MCC may function as a binder and/or a disintegrant. In certain embodiments, an anti-adherent may function as a gliant or a lubricant. In certain embodiments, CSD may function as a gliant. In certain embodiments, magnesium stearate may function as a lubricant. In certain embodiments, the compound of Formula (I) of a tablet pharmaceutical composition may not be formulated for delayed release. In certain embodiments, at least one of the at least one excipient may not be formulated for delayed release. In certain embodiments, each of the at least one excipient may not be formulated for delayed release.

In embodiments, a diluent of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%0, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%5, 5.90%, 6.00%, 6.10%, 6.20%, 6.30%, 6.40%, 6.50%, 6.60%, 6.70%, 6.80%, 6.90%, 7.00%7, 7.10%, 7.20%, 7.30%, 7.40%, 7.50%7, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. The various percentages listed above are applicable to microcrystalline cellulose (MCC), lactose monohydrate (LMH), or both. In embodiments, the diluent may comprise about 43.25% of MCC by weight of the tablet pharmaceutical composition. In embodiments, the diluent may comprise about 43.50% of LMH by weight of the tablet pharmaceutical composition. In embodiments, the diluent may comprise about 43.25% of MCC by weight of the tablet pharmaceutical composition and about 43.50% of LMH by weight of the tablet pharmaceutical composition.

In embodiments, a diluent of a tablet pharmaceutical composition disclosed herein may be from about 0.010% to about 99% by weight of the tablet pharmaceutical composition, e.g, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.10% to about 3%, about 0.10% to about 4%, about 0.10% to about 5%, about 0.10% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to ab out 90%, about 80% to about 99%, about 90% to about 99%. The various ranges listed above are applicable to microcrystalline cellulose (MCC), lactose monohydrate (LMH), or both.

In embodiments, a binder of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. The various percentages listed above are applicable to MCC, HPC, or both. In embodiments, a binder may comprise about 43.25% of MCC by weight of the tablet pharmaceutical composition. In embodiments, a binder may comprise about 66% of HPC by weight of the tablet pharmaceutical composition. In embodiments, a binder may comprise about 43.25% of MCC by weight of the tablet pharmaceutical composition and about 6% of HPC by weight of the tablet pharmaceutical composition.

In embodiments, a binder of a tablet pharmaceutical composition disclosed herein may be from about 0.01I % to about 99% by weight of the tablet pharmaceutical composition, e.g, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.10% to about 3%, about 0.10% to about 4%, about 0.10% to about 5%, about 0.10% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 10% to about 70%, about 10% to about 80%, about 10% to about 90%, about 1% to about 99%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. The various ranges listed above are applicable to MCC, HPC, or both.

In embodiments, an anti-adherent of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. The various percentages listed above are applicable to CSD, magnesium stearate, or both. In embodiments, an anti-adherent may comprise about 0.5% of CSD by weight of the tablet pharmaceutical composition. In embodiments, an anti-adherent may comprise about 1.0% of magnesium stearate by weight of the tablet pharmaceutical composition. In embodiments, an anti-adherent may comprise about 0.5% of CSD by weight of the tablet pharmaceutical composition and about 1.0% of magnesium stearate by weight of the tablet pharmaceutical composition.

In embodiments, an anti-adherent of a tablet pharmaceutical composition disclosed herein may be from about 0.01% to about 99% by weight of the tablet pharmaceutical composition, e.g., about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. The various ranges listed above are applicable to CSD, magnesium stearate, or both.

In embodiments, a glidant of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. The various percentages listed above are applicable to CSD. In embodiments, a glidant may comprise about 0.5% of CSD by weight of the tablet pharmaceutical composition.

In embodiments, a glidant of a tablet pharmaceutical composition disclosed herein may be from about 0.010% to about 99% by weight of the tablet pharmaceutical composition, e.g, about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.10% to about 3%, about 0.10% to about 4%, about 0.10% to about 5%, about 0.10% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 2% to about 30%, about 2% to about 40%, about 2% to about 50%, about 2% to about 60%, about 22% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. The various ranges listed above are applicable to CSD.

In embodiments, a lubricant of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. The various percentages listed above are applicable to magnesium stearate. In embodiments, a lubricant may comprise about 1.0% of magnesium stearate by weight of the tablet pharmaceutical composition.

In embodiments, a lubricant of a tablet pharmaceutical composition disclosed herein may be from about 0.01% to about 99% by weight of the tablet pharmaceutical composition, e.g., about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, aboutO0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 22%, about 0.1% to about 3%, about 0.1% to about 4%, about 0.1% to about 55%, about 0.1% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 22% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. The various ranges listed above are applicable to magnesium stearate.

In certain embodiments, a tablet pharmaceutical composition disclosed herein may comprise an external coating. In embodiments, the external coating of a tablet pharmaceutical composition disclosed herein may be from about 0.01% to about 99% by weight of the tablet pharmaceutical composition, e.g., about 0.01% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 20%, ab out 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. In embodiments, the external coating of a tablet pharmaceutical composition may not cause delayed release of the compound of Formula (I) and/or the at least one excipient.

In certain embodiments, the dry weight of a tablet pharmaceutical composition disclosed herein may be from about 0.1 mg to about 400 mg, e.g., about 0.1 mg, 0.2 mg, 0.3 mg 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 102 mg, 104 mg, 106 mg, 108 mg, 110 mg, 112 mg, 114 mg, 116 mg, 118 mg, 120 mg, 122 mg, 124 mg, 126 mg, 128 mg, 130 mg, 132 mg, 134 mg, 136 mg 138 mg, 140 mg, 142 mg, 144 mg, 146 mg, 148 mg, 150 mg, 152 mg, 154 mg, 156 mg, 158 mg 160 mg, 162 mg, 164 mg, 166 mg, 168 mg, 170 mg, 172 mg, 174 mg, 176 mg, 178 mg, 180 mg 182 mg, 184 mg, 186 mg, 188 mg, 190 mg, 192 mg, 194 mg, 196 mg, 198 mg, 200 mg, 202 mg 204 mg, 206 mg, 208 mg, 210 mg, 212 mg, 214 mg, 216 mg, 218 mg, 220 mg, 222 mg, 224 mg 226 mg, 228 mg, 230 mg, 232 mg, 234 mg, 236 mg, 238 mg, 240 mg, 242 mg, 244 mg, 246 mg 248 mg, 250 mg, 252 mg, 254 mg, 256 mg, 258 mg, 260 mg, 262 mg, 264 mg, 266 mg, 268 mg 270 mg, 272 mg, 274 mg, 276 mg, 278 mg, 280 mg, 282 mg, 284 mg, 286 mg, 288 mg, 290 mg 292 mg, 294 mg, 296 mg, 298 mg, 300 mg, 302 mg, 304 mg, 306 mg, 308 mg, 310 mg, 312 mg 314 mg, 316 mg, 318 mg, 320 mg, 322 mg, 324 mg, 326 mg, 328 mg, 330 mg, 332 mg, 334 mg 336 mg, 338 mg, 340 mg, 342 mg, 344 mg, 346 mg, 348 mg, 350 mg, 352 mg, 354 mg, 356 mg 358 mg, 360 mg, 362 mg, 364 mg, 366 mg, 368 mg, 370 mg, 372 mg, 374 mg, 376 mg, 378 mg 380 mg, 382 mg, 384 mg, 386 mg, 388 mg, 390 mg, 392 mg, 394 mg, 396 mg, 398 mg, or 400 mg or any amount therebetween.

In embodiments, the compound of Formula (I) may be from about 1% to about 99.99% by weight of the tablet pharmaceutical composition, e.g., about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 1% to about 99.99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 10% to about 99.99%, about 20% to about 30%, about 20% to about 40%, about 20% to about 50%, about 20% to about 60%, about 20% to about 70%, about 20% to about 80%, about 20% to about 90%, about 20% to about 99%, about 20% to about 99.99%, about 30% to about 40%, about 30% to about 50%, about 30% to about 60%, about 30% to about 70%, about 30% to about 80%, about 30% to about 90%, about 30% to about 99%, about 30% to about 99.99%, about 40% to about 50%, about 40% to about 60%, about 40% to about 70%, about 40% to about 80%, about 40% to about 90%, about 40% to about 99%, about 40% to about 99.99%, about 50% to about 60%, about 50% to about 70%, about 50% to about 80%, about 50% to about 90%, about 50% to about 99%, about 50% to about 99.99%, about 60% to about 70%, about 60% to about 80%, about 60% to about 90%, about 60% to about 99%, about 60% to about 99.99%, about 70% to about 80%, about 70% to about 90%, about 70% to about 99%, about 70% to about 99.99%, about 80% to about 90%, about 80% to about 99%, about 80% to about 99.99%, about 90% to about 99%, about 90% to about 99.99%, or about 99% to about 99.99%,

In certain embodiments, a tablet pharmaceutical composition disclosed herein may not exhibit oxidation, loss of chemical stability, substantial degradation, and/or decomposition after about 1 week, 2 weeks, 3 weeks, 4 weeks, 10 weeks, 20 weeks, 30 weeks, 40 weeks, one year, two years, or three years, or any time period therebetween, at about 25° C. when stored in otherwise atmospheric conditions. In certain embodiments, the storage conditions of the tablet pharmaceutical composition may include a relative humidity. In certain embodiments, the storage conditions of the tablet pharmaceutical composition may include a relative humidity of about 60% or about 75%.

In certain embodiments, a tablet pharmaceutical composition disclosed herein may not exhibit substantial degradation, loss of chemical stability, decomposition, and/or oxidation of a compound of Formula (I), a pharmaceutically acceptable salt, crystalline, solvate, polymorph, prodrug, metabolite, N-oxide, stereoisomer, or isomer thereof, or the at least one excipient after about 1 week, 2 weeks, 3 weeks, 4 weeks, 10 weeks, 20 weeks, 30 weeks, 40 weeks, one year, two years, or three years, or any time period therebetween, at about 25° C. when stored in otherwise atmospheric conditions. In certain embodiments, the storage conditions of the tablet pharmaceutical composition may include a relative humidity. In certain embodiments, the storage conditions of the tablet pharmaceutical composition may include a relative humidity of about 60% or about 75%.

In certain embodiments, a tablet pharmaceutical composition disclosed herein may not exhibit oxidation, loss of chemical stability, substantial degradation, and/or decomposition after about 1 week, 2 weeks, 3 weeks, 4 weeks, 10 weeks, 20 weeks, 30 weeks, 40 weeks, or one year, two years, or three years, or any time period therebetween, at about 40° C. when stored in otherwise atmospheric conditions. In certain embodiments, the storage conditions of the tablet pharmaceutical composition may include a relative humidity. In certain embodiments, the storage conditions of the tablet pharmaceutical composition may include a relative humidity of about 60% or about 75%. Storing a tablet pharmaceutical composition at temperatures above room temperature is used to approximate storage of a tablet over a longer period of time as the high temperature expedites instability of a tablet and/or accelerates undesirable chemical reactions.

In certain embodiments, a tablet pharmaceutical composition disclosed herein may not exhibit substantial degradation, loss of chemical stability, decomposition, and/or oxidation of a compound of Formula (I), a pharmaceutically acceptable salt, crystalline, solvate, polymorph, prodrug, metabolite, N-oxide, stereoisomer, or isomer thereof, or the at least one excipient after about 1 week, 2 weeks, 3 weeks, 4 weeks, 10 weeks, 20 weeks, 30 weeks, 40 weeks, one year, two years, or three years, or any time period therebetween, at about 40° C. when stored in otherwise atmospheric conditions. In certain embodiments, the storage conditions of the tablet pharmaceutical composition may include a relative humidity. In certain embodiments, the storage conditions of the tablet pharmaceutical composition may include a relative humidity of about 60% or about 75%. Storing a tablet pharmaceutical composition at temperatures above room temperature is used to approximate storage of a tablet over a longer period of time as the high temperature expedites instability of a tablet and/or accelerates undesirable chemical reactions.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 5 minutes when placed in an aqueous medium at about pH 7 and at 25° C. In certain embodiments, the aqueous medium may comprise water.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 10 minutes when placed in an aqueous medium at about pH7 and at 25° C. In certain embodiments, the aqueous medium may comprise water.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 20 minutes when placed in an aqueous medium at about pH7 and at 25° C. In certain embodiments, the aqueous medium may comprise water.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 30 minutes when placed in an aqueous medium at about pH7 and at 25° C. In certain embodiments, the aqueous medium may comprise water.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 40 minutes when placed in an aqueous medium at about pH7 and at 25° C. In certain embodiments, the aqueous medium may comprise water.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 50 minutes when placed in an aqueous medium at about pH7 and at 25° C. In certain embodiments, the aqueous medium may comprise water.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about one hour when placed in an aqueous medium at about pH7 and at 25° C. In certain embodiments, the aqueous medium may comprise water.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 5 minutes in simulated gastric fluid at 37° C., e.g., when tested in USP Apparatus Type II at 50 rpm in 900 ml of simulated gastric fluid at 37° C.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 10 minutes in simulated gastric fluid at 37° C., e.g., when tested in USP Apparatus Type II at 50 rpm in 900 ml of simulated gastric fluid at 37° C.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 20 minutes in simulated gastric fluid at 37° C., e.g., when tested in USP Apparatus Type II at 50 rpm in 900 ml of simulated gastric fluid at 37° C.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 30 minutes in simulated gastric fluid at 37° C., e.g., when tested in USP Apparatus Type II at 50 rpm in 900 ml of simulated gastric fluid at 37° C.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 40 minutes in simulated gastric fluid at 37° C., e.g., when tested in USP Apparatus Type II at 50 rpm in 900 ml of simulated gastric fluid at 37° C.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about 50 minutes in simulated gastric fluid at 37° C., e.g., when tested in USP Apparatus Type II at 50 rpm in 900 ml of simulated gastric fluid at 37° C.

In certain embodiments, at least about 5%, 5.50%, 6.00%, 6.50%, 7.00%, 7.50%, 8.00%, 8.50%, 9.00%, 9.50%, 10.00%, 10.50%, 11.00%, 11.50%, 12.00%, 12.50%, 13.00%, 13.50%, 14.00%, 14.50%, 15.00%, 15.50%, 16.00%, 16.50%, 17.00%, 17.50%, 18.00%, 18.50%, 19.00%, 19.50%, 20.00%, 20.50%, 21.00%, 21.50%, 22.00%, 22.50%, 23.00%, 23.50%, 24.00%, 24.50%, 25.00%, 25.50%, 26.00%, 26.50%, 27.00%, 27.50%, 28.00%, 28.50%, 29.00%, 29.50%, 30.00%, 30.50%, 31.00%, 31.50%, 32.00%, 32.50%, 33.00%, 33.50%, 34.00%, 34.50%, 35.00%, 35.50%, 36.00%, 36.50%, 37.00%, 37.50%, 38.00%, 38.50%, 39.00%, 39.50%, 40.00%, 40.50%, 41.00%, 41.50%, 42.00%, 42.50%, 43.00%, 43.50%, 44.00%, 44.50%, 45.00%, 45.50%, 46.00%, 46.50%, 47.00%, 47.50%, 48.00%, 48.50%, 49.00%, 49.50%, 50.00%, 50.50%, 51.00%, 51.50%, 52.00%, 52.50%, 53.00%, 53.50%, 54.00%, 54.50%, 55.00%, 55.50%, 56.00%, 56.50%, 57.00%, 57.50%, 58.00%, 58.50%, 59.00%, 59.50%, 60.00%, 60.50%, 61.00%, 61.50%, 62.00%, 62.50%, 63.00%, 63.50%, 64.00%, 64.50%, 65.00%, 65.50%, 66.00%, 66.50%, 67.00%, 67.50%, 68.00%, 68.50%, 69.00%, 69.50%, 70.00%, 70.50%, 71.00%, 71.50%, 72.00%, 72.50%, 73.00%, 73.50%, 74.00%, 74.50%, 75.00%, 75.50%, 76.00%, 76.50%, 77.00%, 77.50%, 78.00%, 78.50%, 79.00%, 79.50%, 80.00%, 80.50%, 81.00%, 81.50%, 82.00%, 82.50%, 83.00%, 83.50%, 84.00%, 84.50%, 85.00%, 85.50%, 86.00%, 86.50%, 87.00%, 87.50%, 88.00%, 88.50%, 89.00%, 89.50%, 90.00%, 90.50%, 91.00%, 91.50%, 92.00%, 92.50%, 93.00%, 93.50%, 94.00%, 94.50%, 95.00%, 95.50%, 96.00%, 96.50%, 97.00%, 97.50%, 98.00%, 98.50%, 99.00%, 99.50%, or about 100.00%, or any amount therebetween, of a tablet pharmaceutical composition disclosed herein by weight have dissociated or dissolved after about one hour in simulated gastric fluid at 37° C., e.g., when tested in USP Apparatus Type II at 50 rpm in 900 ml of simulated gastric fluid at 37° C.

In certain embodiments, the at least one excipient of a tablet pharmaceutical composition disclosed herein may be about 0.01% to about 99% by weight of the tablet pharmaceutical composition, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 1.10%, 1.20%, 1.30%, 1.40%, 1.50%, 1.60%, 1.70%, 1.80%, 1.90%, 2.00%, 2.10%, 2.20%, 2.30%, 2.40%, 2.50%, 2.60%, 2.70%, 2.80%, 2.90%, 3.00%, 3.10%, 3.20%, 3.30%, 3.40%, 3.50%, 3.60%, 3.70%, 3.80%, 3.90%, 4.00%, 4.10%, 4.20%, 4.30%, 4.40%, 4.50%, 4.60%, 4.70%, 4.80%, 4.90%, 5.00%, 5.10%, 5.20%, 5.30%, 5.40%, 5.50%, 5.60%, 5.70%, 5.80%, 5.90%, 6.00%, 6.10%, 6.20%, 6.30%, 6.40%, 6.50%, 6.60%, 6.70%, 6.80%, 6.90%, 7.00%, 7.10%, 7.20%, 7.30%, 7.40%, 7.50%, 7.60%, 7.70%, 7.80%, 7.90%, 8.00%, 8.10%, 8.20%, 8.30%, 8.40%, 8.50%, 8.60%, 8.70%, 8.80%, 8.90%, 9.00%, 9.10%, 9.20%, 9.30%, 9.40%, 9.50%, 9.60%, 9.70%, 9.80%, 9.90%, 10.00%, 11.00%, 12.00%, 13.00%, 14.00%, 15.00%, 16.00%, 17.00%, 18.00%, 19.00%, 20.00%, 21.00%, 22.00%, 23.00%, 24.00%, 25.00%, 26.00%, 27.00%, 28.00%, 29.00%, 30.00%, 31.00%, 32.00%, 33.00%, 34.00%, 35.00%, 36.00%, 37.00%, 38.00%, 39.00%, 40.00%, 41.00%, 42.00%, 43.00%, 44.00%, 45.00%, 46.00%, 47.00%, 48.00%, 49.00%, 50.00%, 51.00%, 52.00%, 53.00%, 54.00%, 55.00%, 56.00%, 57.00%, 58.00%, 59.00%, 60.00%, 61.00%, 62.00%, 63.00%, 64.00%, 65.00%, 66.00%, 67.00%, 68.00%, 69.00%, 70.00%, 71.00%, 72.00%, 73.00%, 74.00%, 75.00%, 76.00%, 77.00%, 78.00%, 79.00%, 80.00%, 81.00%, 82.00%, 83.00%, 84.00%, 85.00%, 86.00%, 87.00%, 88.00%, 89.00%, 90.00%, 91.00%, 92.00%, 93.00%, 94.00%, 95.00%, 96.00%, 97.00%, 98.00%, or 99.00%, or any percentage therebetween. In embodiments, the various percentages listed above may be applicable to a disintegrant, a dispersion polymer, a diluent, a binder, an anti-adherent, a filler, a sweetener, a wetting agent, a glidant, a lubricant, or a surfactant, or any combinations thereof. In embodiments, the at least one excipient of a tablet pharmaceutical composition disclosed herein may comprise about 43.25% of MCC, about 43.50% of LMH, about 6.00% of HPC, about 0.75% of CCNa, about 0.54% of CSD, and about 1.00% magnesium stearate, all by weight of the tablet pharmaceutical composition.

In certain embodiments, the at least one excipient of a tablet pharmaceutical composition disclosed herein may be from about 0.01% to about 99% by weight of the tablet pharmaceutical composition, e.g., about 0.01% to about 1%, about 0.01% to about 22%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.1% to about 3%, about 0.1% to about 4%, about 0.1% to about 5%, about 0.1% to about 6%, about 0.1% to about 77%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 22% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, about 90% to about 99%. In embodiments, the various ranges listed above may be applicable to a disintegrant, a dispersion polymer, a diluent, a binder, an anti-adherent, a filler, a sweetener, a wetting agent, a glidant, a lubricant, or a surfactant, or any combinations thereof.

In certain embodiments, the dispersion polymer is HPMC-AS. The HPMC-AS can be of any grade, including HPMC-AS, LF; HPMC-AS, M; HPMC-AS, H; HPMC-AS, HF; and HPMC-AS, HG. The HPMC-AS can also be of any viscosity, including low, normal, and high. In certain embodiments, the dispersion polymer comprises HPMC-AS, LF. In certain embodiments, the dispersion polymer comprises HPMC-AS, M. In certain embodiments, the dispersion polymer comprises HPMC-AS, HF. In certain embodiments, the dispersion polymer comprises HPMC-AS, HG. In some embodiments, the indicator “L,” “M,” or “H” refers to a low, medium, or high ratio of acetyl to succinyl substituents on the HPMC backbone, respectively. In some embodiments, the indicator “F” or “G” refers to either a fine or granular particle size, respectively.

In certain embodiments, a particular grade of HPMC-AS is specified, such as L, M, or H. In some embodiments, the grade refers to the ratio of acetyl to succinyl substituents on the HPMC backbone, with “L” grade being a low ratio, “M” grade being a medium ratio, and “H” grade being a high ratio. In some embodiments, HPMC-AS, L comprises an acetyl content of about 5-9% and a succinyl content of about 14-18%. In some embodiments, the HPMC-AS, L further comprises a methoxyl content of about 20-24% and a hydroxypropyl content of about 5-9%. In some embodiments, HPMC-AS, M comprises an acetyl content of about 7-11% and a succinyl content of about 10-14%. In some embodiments, the HPMC-AS, M further comprises a methoxyl content of about 21-25% and a hydroxypropyl content of about 5-9%. In some embodiments, HPMC-AS, H comprises an acetyl content of about 10-14% and a succinyl content of about 4-8%. In some embodiments, the HPMC-AS, H further comprises a methoxyl content of 22-26% and a hydroxypropyl content of about 60-10%. The percentages referred to in this paragraph refer to percentages by weight of the HPMC-AS composition.

In certain embodiments, the dispersion polymer is HPMC. In certain embodiments, the HPMC is an HPMC derivative. The HPMC or HPMC derivative can be of any grade, including low, normal, or high viscosity grades. Non-limiting examples of suitable HPMC or HPMC derivatives include Methocel™ K100M, K15M, F4M, E4M, K4M, K100LV, K3, E15LV, E15LN, E15CLV, E50, E5, E5LV, E3, and E3LV (available from Dow Chemical, Midland Mich.). In certain embodiments, the dispersion polymer is HPMC E3LV.

### Excipient

In certain embodiments, a tablet pharmaceutical composition disclosed herein may comprise at least one excipient. In certain embodiments, the at least one excipient may comprise a disintegrant, a dispersion polymer, a diluent, a binder, an anti-adherent, a filler, a sweetener, a wetting agent, a glidant, a lubricant, or a surfactant, or any combinations thereof.

### Fillers

In certain embodiments, a tablet pharmaceutical composition disclosed herein may further comprise a filler. Fillers may be any biocompatible substance that is unreactive with the compound of Formula (I). Non-limiting examples of fillers include lactose monohydrate, mannitol, sorbitol, cellulose, calcium phosphate, starch, sugar, cellulose, modified cellulose, sodium carboxymethyl cellulose, ethyl cellulose hydroxymethyl cellulose, hydroxypropylcellulose, cellulose acetate, microcrystalline cellulose, dibasic calcium phosphate, sucrose, lactose, corn starch, potato starch, or any combination thereof.

In certain embodiments, the filler comprises about 0.01-99% by weight of the pharmaceutical composition. In certain embodiments, the filler comprises about 10% by weight of the pharmaceutical composition. In certain embodiments, the filler comprises about 20% by weight of the pharmaceutical composition. In certain embodiments, the filler comprises about 30% by weight of the pharmaceutical composition. In certain embodiments, the filler comprises about 40% by weight of the pharmaceutical composition. In certain embodiments, the filler comprises about 50% by weight of the pharmaceutical composition. In certain embodiments, the filler comprises about 60% by weight of the pharmaceutical composition. In certain embodiments, the filler comprises about 70% by weight of the pharmaceutical composition. In certain embodiments, the filler comprises about 80% by weight of the pharmaceutical composition. In certain embodiments, the filler comprises about 90% by weight of the pharmaceutical composition.

In certain embodiments, the filler comprises up to about 10% by weight of the pharmaceutical composition. In certain embodiments, the filler comprises up to about 20% by weight of the pharmaceutical composition. In certain embodiments, the filler comprises up to about 30% by weight of the pharmaceutical composition. In certain embodiments, the filler comprises up to about 40% by weight of the pharmaceutical composition. In certain embodiments, the filler comprises up to about 50% by weight of the pharmaceutical composition. In certain embodiments, the filler comprises up to about 60% by weight of the pharmaceutical composition. In certain embodiments, the filler comprises up to about 70% by weight of the pharmaceutical composition. In certain embodiments, the filler comprises up to about 80% by weight of the pharmaceutical composition. In certain embodiments, the filler comprises up to about 90% by weight of the pharmaceutical composition.

In certain embodiments, the filler comprises at least about 10% by weight of the pharmaceutical composition. In certain embodiments, the filler comprises at least about 20% by weight of the pharmaceutical composition. In certain embodiments, the filler comprises at least about 30% by weight of the pharmaceutical composition. In certain embodiments, the filler comprises at least about 40% by weight of the pharmaceutical composition. In certain embodiments, the filler comprises at least about 50% by weight of the pharmaceutical composition. In certain embodiments, the filler comprises at least about 60% by weight of the pharmaceutical composition. In certain embodiments, the filler comprises at least about 70% by weight of the pharmaceutical composition. In certain embodiments, the filler comprises at least about 80% by weight of the pharmaceutical composition. In certain embodiments, the filler comprises at least about 90% by weight of the pharmaceutical composition.

### Sweeteners

In certain embodiments, a tablet pharmaceutical composition disclosed herein further comprises a sweetener. Non-limiting examples of sweeteners include water-soluble sweetening agents such as monosaccharides, disaccharides and polysaccharides such as xylose, ribose, glucose (dextrose), mannose, galactose, fructose (levulose), sucrose (sugar), high fructose corn syrup, maltose, invert sugar (a mixture of fructose and glucose derived from sucrose), partially hydrolyzed starch, corn syrup solids, and dihydrochalcones; water-soluble artificial sweeteners such as the soluble saccharin salts, i.e., sodium or calcium saccharin salts, cyclamate salts, the sodium, ammonium or calcium salt of 3,4-dihydro-6-methyl-1,2,3-oxathiazine-4-one-2,2-dioxide, the potassium salt of 3,4-dihydro-6-methyl-1,2,3-oxathiazine-4-one-2,2-dioxide (acesulfame-K), the free acid form of saccharin and the like; dipeptide based sweeteners, such as L-aspartic acid derived sweeteners, such as L-aspartyl-L-phenylalanine methyl ester (aspartame), L-alpha-aspartyl-N-(2,2,4,4-tetramethyl-3-thietanyl)-D-alaninamide hydrate, methyl esters of L-aspartyl-L-phenylglycerin and L-aspartyl-L-2,5,dihydrophenylglycine, L-aspartyl-2,5-dihydro-L-phenylalanine, L-aspartyl-L-(1-cyclohexyen)-alanine, and the like; and water-soluble sweeteners derived from naturally occurring water-soluble sweeteners, such as a chlorinated derivatives of ordinary sugar (sucrose), known, for example, as sucralose.

In certain embodiments, the sweetener comprises about 0.01-99% by weight of the pharmaceutical composition. In certain embodiments, the sweetener comprises about 10% by weight of the pharmaceutical composition. In certain embodiments, the sweetener comprises about 20% by weight of the pharmaceutical composition. In certain embodiments, the sweetener comprises about 30% by weight of the pharmaceutical composition. In certain embodiments, the sweetener comprises about 40% by weight of the pharmaceutical composition. In certain embodiments, the sweetener comprises about 50% by weight of the pharmaceutical composition. In certain embodiments, the sweetener comprises about 60% by weight of the pharmaceutical composition. In certain embodiments, the sweetener comprises about 70% by weight of the pharmaceutical composition. In certain embodiments, the sweetener comprises about 80% by weight of the pharmaceutical composition. In certain embodiments, the sweetener comprises about 90% by weight of the pharmaceutical composition.

In certain embodiments, the sweetener comprises up to about 10% by weight of the pharmaceutical composition. In certain embodiments, the sweetener comprises up to about 20% by weight of the pharmaceutical composition. In certain embodiments, the sweetener comprises up to about 300% by weight of the pharmaceutical composition. In certain embodiments, the sweetener comprises up to about 40% by weight of the pharmaceutical composition. In certain embodiments, the sweetener comprises up to about 50% by weight of the pharmaceutical composition. In certain embodiments, the sweetener comprises up to about 60% by weight of the pharmaceutical composition. In certain embodiments, the sweetener comprises up to about 70% by weight of the pharmaceutical composition. In certain embodiments, the sweetener comprises up to about 80% by weight of the pharmaceutical composition. In certain embodiments, the sweetener comprises up to about 90% by weight of the pharmaceutical composition.

In certain embodiments, the sweetener comprises at least about 10% by weight of the pharmaceutical composition. In certain embodiments, the sweetener comprises at least about 20% by weight of the pharmaceutical composition. In certain embodiments, the sweetener comprises at least about 30% by weight of the pharmaceutical composition. In certain embodiments, the sweetener comprises at least about 40% by weight of the pharmaceutical composition. In certain embodiments, the sweetener comprises at least about 50% by weight of the pharmaceutical composition. In certain embodiments, the sweetener comprises at least about 60% by weight of the pharmaceutical composition. In certain embodiments, the sweetener comprises at least about 70% by weight of the pharmaceutical composition. In certain embodiments, the sweetener comprises at least about 80% by weight of the pharmaceutical composition. In certain embodiments, the sweetener comprises at least about 90% by weight of the pharmaceutical composition.

### Disintegrants

In certain embodiments, a tablet pharmaceutical composition further comprises a disintegrant. Non-limiting examples of disintegrants include agar-agar, algins, calcium carbonate, carboxymethylcellulose, cellulose, hydroxypropylcellulose, low substituted hydroxypropylcellulose, clays, croscarmellose sodium, crospovidone, gums, magnesium aluminum silicate, methylcellulose, polacrilin potassium, sodium alginate, sodium starch glycolate, maize starch, potato starch, tapioca starch, or any combination thereof.

In certain embodiments, the disintegrant comprises about 0.01-99% by weight of the pharmaceutical composition. In certain embodiments, the disintegrant comprises about 10% by weight of the pharmaceutical composition. In certain embodiments, the disintegrant comprises about 20% by weight of the pharmaceutical composition. In certain embodiments, the disintegrant comprises about 30% by weight of the pharmaceutical composition. In certain embodiments, the disintegrant comprises about 40% by weight of the pharmaceutical composition. In certain embodiments, the disintegrant comprises about 50% by weight of the pharmaceutical composition. In certain embodiments, the disintegrant comprises about 60% by weight of the pharmaceutical composition. In certain embodiments, the disintegrant comprises about 70% by weight of the pharmaceutical composition. In certain embodiments, the disintegrant comprises about 80% by weight of the pharmaceutical composition. In certain embodiments, the disintegrant comprises about 90% by weight of the pharmaceutical composition.

In certain embodiments, the disintegrant comprises up to about 10% by weight of the pharmaceutical composition. In certain embodiments, the disintegrant comprises up to about 20% by weight of the pharmaceutical composition. In certain embodiments, the disintegrant comprises up to about 30% by weight of the pharmaceutical composition. In certain embodiments, the disintegrant comprises up to about 40% by weight of the pharmaceutical composition. In certain embodiments, the disintegrant comprises up to about 50% by weight of the pharmaceutical composition. In certain embodiments, the disintegrant comprises up to about 60% by weight of the pharmaceutical composition. In certain embodiments, the disintegrant comprises up to about 70% by weight of the pharmaceutical composition. In certain embodiments, the disintegrant comprises up to about 80% by weight of the pharmaceutical composition. In certain embodiments, the disintegrant comprises up to about 90% by weight of the pharmaceutical composition.

In certain embodiments, the disintegrant comprises at least about 10% by weight of the pharmaceutical composition. In certain embodiments, the disintegrant comprises at least about 20% by weight of the pharmaceutical composition. In certain embodiments, the disintegrant comprises at least about 30% by weight of the pharmaceutical composition. In certain embodiments, the disintegrant comprises at least about 40% by weight of the pharmaceutical composition. In certain embodiments, the disintegrant comprises at least about 50% by weight of the pharmaceutical composition. In certain embodiments, the disintegrant comprises at least about 60% by weight of the pharmaceutical composition. In certain embodiments, the disintegrant comprises at least about 70% by weight of the pharmaceutical composition. In certain embodiments, the disintegrant comprises at least about 80% by weight of the pharmaceutical composition. In certain embodiments, the disintegrant comprises at least about 90% by weight of the pharmaceutical composition.

### Dispersion Polymers

In certain embodiments, a tablet pharmaceutical composition further comprises a dispersion polymer. Non-limiting examples of dispersion polymers include hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, or combinations thereof.

In certain embodiments, the dispersion polymer comprises about 0.01-99% by weight of the pharmaceutical composition. In certain embodiments, the dispersion polymer comprises about 10% by weight of the pharmaceutical composition. In certain embodiments, the dispersion polymer comprises about 20% by weight of the pharmaceutical composition. In certain embodiments, the dispersion polymer comprises about 30% by weight of the pharmaceutical composition. In certain embodiments, the dispersion polymer comprises about 40% by weight of the pharmaceutical composition. In certain embodiments, the dispersion polymer comprises about 50% by weight of the pharmaceutical composition. In certain embodiments, the dispersion polymer comprises about 60% by weight of the pharmaceutical composition. In certain embodiments, the dispersion polymer comprises about 70% by weight of the pharmaceutical composition. In certain embodiments, the dispersion polymer comprises about 80% by weight of the pharmaceutical composition. In certain embodiments, the dispersion polymer comprises about 90% by weight of the pharmaceutical composition.

In certain embodiments, the dispersion polymer comprises up to about 10% by weight of the pharmaceutical composition. In certain embodiments, the dispersion polymer comprises up to about 20% by weight of the pharmaceutical composition. In certain embodiments, the dispersion polymer comprises up to about 30% by weight of the pharmaceutical composition. In certain embodiments, the dispersion polymer comprises up to about 40% by weight of the pharmaceutical composition. In certain embodiments, the dispersion polymer comprises up to about 50% by weight of the pharmaceutical composition. In certain embodiments, the dispersion polymer comprises up to about 60% by weight of the pharmaceutical composition. In certain embodiments, the dispersion polymer comprises up to about 70% by weight of the pharmaceutical composition. In certain embodiments, the dispersion polymer comprises up to about 80% by weight of the pharmaceutical composition. In certain embodiments, the dispersion polymer comprises up to about 90% by weight of the pharmaceutical composition.

In certain embodiments, the dispersion polymer comprises at least about 10% by weight of the pharmaceutical composition. In certain embodiments, the dispersion polymer comprises at least about 20% by weight of the pharmaceutical composition. In certain embodiments, the dispersion polymer comprises at least about 30% by weight of the pharmaceutical composition. In certain embodiments, the dispersion polymer comprises at least about 40% by weight of the pharmaceutical composition. In certain embodiments, the dispersion polymer comprises at least about 50% by weight of the pharmaceutical composition. In certain embodiments, the dispersion polymer comprises at least about 60% by weight of the pharmaceutical composition. In certain embodiments, the dispersion polymer comprises at least about 70% by weight of the pharmaceutical composition. In certain embodiments, the dispersion polymer comprises at least about 80% by weight of the pharmaceutical composition. In certain embodiments, the dispersion polymer comprises at least about 90% by weight of the pharmaceutical composition.

### Wetting Agents

In certain embodiments, a tablet pharmaceutical composition disclosed herein further comprises a wetting agent. Non-limiting examples of wetting agents include sodium lauryl sulfate, cetostearyl alcohol, cetomacrogol emulsifying wax, gelatin, casein, docusate sodium, benzalkonium chloride, calcium stearate, polyethylene glycols, phosphates, polyoxyethylene sorbitan fatty acid esters, gum acacia, cholesterol, tragacanth, polyoxyethylene 20 stearyl ethers, polyoxyethylene alkyl ethers, polyoxyethylene castor oil derivatives, PEGylated hydrogenated castor oils, sorbitan esters of fatty acids, Vitamin E or tocopherol derivatives, vitamin E TPGS, tocopheryl esters, lecithin, phospholipids and their derivatives, poloxamers, stearic acid, oleic acid, oleic alcohol, cetyl alcohol, mono and diglycerides, propylene glycol esters of fatty acids, glycerol esters of fatty acids, ethylene glycol palmitostearate, polyoxylglycerides, propylene glycol monocaprylate, propylene glycol monolaurate, alkyl aryl polyether alcohols and polyglyceryl oleate or combinations thereof.

In certain embodiments, the wetting agent comprises about 0.01-99% by weight of the pharmaceutical composition. In certain embodiments, the wetting agent comprises about 10% by weight of the pharmaceutical composition. In certain embodiments, the wetting agent comprises about 20% by weight of the pharmaceutical composition. In certain embodiments, the wetting agent comprises about 30% by weight of the pharmaceutical composition. In certain embodiments, the wetting agent comprises about 40% by weight of the pharmaceutical composition. In certain embodiments, the wetting agent comprises about 50% by weight of the pharmaceutical composition. In certain embodiments, the wetting agent comprises about 60% by weight of the pharmaceutical composition. In certain embodiments, the wetting agent comprises about 70% by weight of the pharmaceutical composition. In certain embodiments, the wetting agent comprises about 80% by weight of the pharmaceutical composition. In certain embodiments, the wetting agent comprises about 90% by weight of the pharmaceutical composition.

In certain embodiments, the wetting agent comprises up to about 10% by weight of the pharmaceutical composition. In certain embodiments, the wetting agent comprises up to about 20% by weight of the pharmaceutical composition. In certain embodiments, the wetting agent comprises up to about 30% by weight of the pharmaceutical composition. In certain embodiments, the wetting agent comprises up to about 40% by weight of the pharmaceutical composition. In certain embodiments, the wetting agent comprises up to about 50% by weight of the pharmaceutical composition. In certain embodiments, the wetting agent comprises up to about 60% by weight of the pharmaceutical composition. In certain embodiments, the wetting agent comprises up to about 70% by weight of the pharmaceutical composition. In certain embodiments, the wetting agent comprises up to about 80% by weight of the pharmaceutical composition. In certain embodiments, the wetting agent comprises up to about 90% by weight of the pharmaceutical composition.

In certain embodiments, the wetting agent comprises at least about 10% by weight of the pharmaceutical composition. In certain embodiments, the wetting agent comprises at least about 20% by weight of the pharmaceutical composition. In certain embodiments, the wetting agent comprises at least about 30% by weight of the pharmaceutical composition. In certain embodiments, the wetting agent comprises at least about 40% by weight of the pharmaceutical composition. In certain embodiments, the wetting agent comprises at least about 50% by weight of the pharmaceutical composition. In certain embodiments, the wetting agent comprises at least about 60% by weight of the pharmaceutical composition. In certain embodiments, the wetting agent comprises at least about 70% by weight of the pharmaceutical composition. In certain embodiments, the wetting agent comprises at least about 80% by weight of the pharmaceutical composition. In certain embodiments, the wetting agent comprises at least about 90% by weight of the pharmaceutical composition.

### Glidants

In certain embodiments, a tablet pharmaceutical composition disclosed herein further comprises a glidant. Non-limiting examples of glidants include colloidal silicon dioxide, talk, corn starch, metal silicates, higher fatty acid metal salts, metal oxides, alkaline earth metal salts, and metal hydroxides or any combination thereof.

In certain embodiments, the glidant comprises about 0.01-99% by weight of the pharmaceutical composition. In certain embodiments, the glidant comprises about 10% by weight of the pharmaceutical composition. In certain embodiments, the glidant comprises about 20% by weight of the pharmaceutical composition. In certain embodiments, the glidant comprises about 30% by weight of the pharmaceutical composition. In certain embodiments, the glidant comprises about 40% by weight of the pharmaceutical composition. In certain embodiments, the glidant comprises about 50% by weight of the pharmaceutical composition. In certain embodiments, the glidant comprises about 60% by weight of the pharmaceutical composition. In certain embodiments, the glidant comprises about 70% by weight of the pharmaceutical composition. In certain embodiments, the glidant comprises about 80% by weight of the pharmaceutical composition. In certain embodiments, the glidant comprises about 90% by weight of the pharmaceutical composition.

In certain embodiments, the glidant comprises up to about 10% by weight of the pharmaceutical composition. In certain embodiments, the glidant comprises up to about 20% by weight of the pharmaceutical composition. In certain embodiments, the glidant comprises up to about 30% by weight of the pharmaceutical composition. In certain embodiments, the glidant comprises up to about 40% by weight of the pharmaceutical composition. In certain embodiments, the glidant comprises up to about 50% by weight of the pharmaceutical composition. In certain embodiments, the glidant comprises up to about 60% by weight of the pharmaceutical composition. In certain embodiments, the glidant comprises up to about 70% by weight of the pharmaceutical composition. In certain embodiments, the glidant comprises up to about 80% by weight of the pharmaceutical composition. In certain embodiments, the glidant comprises up to about 90% by weight of the pharmaceutical composition.

In certain embodiments, the glidant comprises at least about 10% by weight of the pharmaceutical composition. In certain embodiments, the glidant comprises at least about 20% by weight of the pharmaceutical composition. In certain embodiments, the glidant comprises at least about 30% by weight of the pharmaceutical composition. In certain embodiments, the glidant comprises at least about 40% by weight of the pharmaceutical composition. In certain embodiments, the glidant comprises at least about 50% by weight of the pharmaceutical composition. In certain embodiments, the glidant comprises at least about 60% by weight of the pharmaceutical composition. In certain embodiments, the glidant comprises at least about 70% by weight of the pharmaceutical composition. In certain embodiments, the glidant comprises at least about 80% by weight of the pharmaceutical composition. In certain embodiments, the glidant comprises at least about 90% by weight of the pharmaceutical composition.

### Lubricants

In certain embodiments, a tablet pharmaceutical composition disclosed herein further comprises a lubricant. Non-limiting examples of lubricants include magnesium stearate, calcium stearate, zinc stearate, sodium stearate, stearic acid, aluminum stearate, leucine, glyceryl behenate, hydrogenated vegetable oil, sodium stearyl fumarate, or any combination thereof.

In certain embodiments, the lubricant comprises about 0.01-99% by weight of the pharmaceutical composition. In certain embodiments, the lubricant comprises about 10% by weight of the pharmaceutical composition. In certain embodiments, the lubricant comprises about 20% by weight of the pharmaceutical composition. In certain embodiments, the lubricant comprises about 30% by weight of the pharmaceutical composition. In certain embodiments, the lubricant comprises about 40% by weight of the pharmaceutical composition. In certain embodiments, the lubricant comprises about 50% by weight of the pharmaceutical composition. In certain embodiments, the lubricant comprises about 60% by weight of the pharmaceutical composition. In certain embodiments, the lubricant comprises about 70% by weight of the pharmaceutical composition. In certain embodiments, the lubricant comprises about 80% by weight of the pharmaceutical composition. In certain embodiments, the lubricant comprises about 90% by weight of the pharmaceutical composition.

In certain embodiments, the lubricant comprises up to about 10% by weight of the pharmaceutical composition. In certain embodiments, the lubricant comprises up to about 20% by weight of the pharmaceutical composition. In certain embodiments, the lubricant comprises up to about 30% by weight of the pharmaceutical composition. In certain embodiments, the lubricant comprises up to about 40% by weight of the pharmaceutical composition. In certain embodiments, the lubricant comprises up to about 50% by weight of the pharmaceutical composition. In certain embodiments, the lubricant comprises up to about 60% by weight of the pharmaceutical composition. In certain embodiments, the lubricant comprises up to about 70% by weight of the pharmaceutical composition. In certain embodiments, the lubricant comprises up to about 80% by weight of the pharmaceutical composition. In certain embodiments, the lubricant comprises up to about 90% by weight of the pharmaceutical composition.

In certain embodiments, the lubricant comprises at least about 10% by weight of the pharmaceutical composition. In certain embodiments, the lubricant comprises at least about 20% by weight of the pharmaceutical composition. In certain embodiments, the lubricant comprises at least about 30% by weight of the pharmaceutical composition. In certain embodiments, the lubricant comprises at least about 40% by weight of the pharmaceutical composition. In certain embodiments, the lubricant comprises at least about 50% by weight of the pharmaceutical composition. In certain embodiments, the lubricant comprises at least about 60% by weight of the pharmaceutical composition. In certain embodiments, the lubricant comprises at least about 70% by weight of the pharmaceutical composition. In certain embodiments, the lubricant comprises at least about 80% by weight of the pharmaceutical composition. In certain embodiments, the lubricant comprises at least about 90% by weight of the pharmaceutical composition.

### Surfactant

In certain embodiments, a tablet pharmaceutical composition further comprises a surfactant. Non limiting examples of surfactants include sodium dodecyl sulfate (SDS), sodium laurel sulfate (SLS), macroglycerol ricinoleate (Kolliphor EL® or Cremophor EL®), caprylocaproyl polyoxyl-8 glyceride (Labrasol®), lauroyl polyoxyl-6 glycerides (Labrafil® M 2130 CS), lauroyl polyoxyl-32 glyceride (Gelucire® 44/14), polyethylene glycol monostearate (Gelucire® 48/16), polyoxyethylene hydrogenated castor oil 60 (HCO-60), polysorbate 80 (Tween®-80), polyethylene glycol sorbitan monolaurate (Tween®-20), polyoxyethylene sorbitan trioleate (Tween®-85), polyoxyethylene glyceryl trioleate (tagot-TO), sorbitan monooleate (Span®-80), sorbitan monolaurate (Span®-20), or any combinations thereof.

In certain embodiments, the surfactant comprises about 0.01-99% by weight of the pharmaceutical composition. In certain embodiments, the surfactant comprises about 10% by weight of the pharmaceutical composition. In certain embodiments, the surfactant comprises about 20% by weight of the pharmaceutical composition. In certain embodiments, the surfactant comprises about 30% by weight of the pharmaceutical composition. In certain embodiments, the surfactant comprises about 40% by weight of the pharmaceutical composition. In certain embodiments, the surfactant comprises about 50% by weight of the pharmaceutical composition. In certain embodiments, the surfactant comprises about 60% by weight of the pharmaceutical composition. In certain embodiments, the surfactant comprises about 70% by weight of the pharmaceutical composition. In certain embodiments, the surfactant comprises about 80% by weight of the pharmaceutical composition. In certain embodiments, the surfactant comprises about 90% by weight of the pharmaceutical composition.

In certain embodiments, the surfactant comprises up to about 10% by weight of the pharmaceutical composition. In certain embodiments, the surfactant comprises up to about 20% by weight of the pharmaceutical composition. In certain embodiments, the surfactant comprises up to about 30% by weight of the pharmaceutical composition. In certain embodiments, the surfactant comprises up to about 40% by weight of the pharmaceutical composition. In certain embodiments, the surfactant comprises up to about 50% by weight of the pharmaceutical composition. In certain embodiments, the surfactant comprises up to about 60% by weight of the pharmaceutical composition. In certain embodiments, the surfactant comprises up to about 70% by weight of the pharmaceutical composition. In certain embodiments, the surfactant comprises up to about 80% by weight of the pharmaceutical composition. In certain embodiments, the surfactant comprises up to about 90% by weight of the pharmaceutical composition.

In certain embodiments, the surfactant comprises at least about 10% by weight of the pharmaceutical composition. In certain embodiments, the surfactant comprises at least about 20% by weight of the pharmaceutical composition. In certain embodiments, the surfactant comprises at least about 30% by weight of the pharmaceutical composition. In certain embodiments, the surfactant comprises at least about 40% by weight of the pharmaceutical composition. In certain embodiments, the surfactant comprises at least about 50% by weight of the pharmaceutical composition. In certain embodiments, the surfactant comprises at least about 60% by weight of the pharmaceutical composition. In certain embodiments, the surfactant comprises at least about 70% by weight of the pharmaceutical composition. In certain embodiments, the surfactant comprises at least about 80% by weight of the pharmaceutical composition. In certain embodiments, the surfactant comprises at least about 90% by weight of the pharmaceutical composition.

### Methods of Manufacturing a Tablet Pharmaceutical Composition

Described herein are methods of manufacturing a tablet pharmaceutical composition disclosed herein, such as those comprising a compound of Formula (I), a pharmaceutically acceptable salt, crystalline, solvate, polymorph, prodrug, metabolite, N-oxide, stereoisomer, or isomer thereof, and at least one excipient. The methods provided herein may be used to prepare a tablet pharmaceutical composition disclosed herein.

In certain embodiments, a method of manufacturing a tablet pharmaceutical composition disclosed herein may include a step (i) of co-sifting a compound of Formula (I), a pharmaceutically acceptable salt, crystalline, solvate, polymorph, prodrug, metabolite, N-oxide, stereoisomer, or isomer thereof, and at least one excipient through a 30-mesh screen. In certain embodiments, the at least one excipient may include a disintegrant, a dispersion polymer, two diluents, a glidant and a lubricant.

In certain embodiments, the disintegrant may be selected from the group including agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof.

In certain embodiments, the dispersion polymer may be selected from the group including hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, poly oxyethylene-poly oxypropylene block copolymers, and combinations thereof.

In certain embodiments, the disintegrant may include CCNa. In certain embodiments, the dispersion polymer may include HPC and/or HPMC. In certain embodiments, the two diluents may include MCC and LMH. In certain embodiments, the glidant may include CSD. In certain embodiments, the lubricant may include magnesium stearate. In certain embodiments, the disintegrant, dispersion polymer, two diluents, glidant and lubricant may include CCNa, HPC, MCC, LMH, CSD, and magnesium stearate. In certain embodiments, the lubricant may be intragranular and/or extragranular. In certain embodiments, at least one of the two diluents may function as a binder and/or disintegrant. In certain embodiments, the glidant may function as an anti-adherent. In certain embodiments, the lubricant may function as an anti-adherent.

In certain embodiments, each of the disintegrant, dispersion polymer, two diluents, glidant and lubricant may be about 0.01-99% by weight of a tablet pharmaceutical composition manufactured according to a method disclosed herein, e.g., about 0.010% to about 1%, about 0.01% to about 2%, about 0.01% to about 3%, about 0.01% to about 4%, about 0.01% to about 5%, about 0.01% to about 6%, about 0.01% to about 7%, about 0.01% to about 8%, about 0.01% to about 9%, about 0.01% to about 10%, about 0.01% to about 20%, about 0.01% to about 30%, about 0.01% to about 40%, about 0.01% to about 50%, about 0.01% to about 60%, about 0.01% to about 70%, about 0.01% to about 80%, about 0.01% to about 90%, about 0.01% to about 99%, about 0.1% to about 1%, about 0.1% to about 2%, about 0.1% to about 3%, about 0.1% to about 4%, about 0.1% to about 5%, about 0.1% to about 6%, about 0.1% to about 7%, about 0.1% to about 8%, about 0.1% to about 9%, about 0.1% to about 10%, about 0.1% to about 20%, about 0.1% to about 30%, about 0.1% to about 40%, about 0.1% to about 50%, about 0.1% to about 60%, about 0.1% to about 70%, about 0.1% to about 80%, about 0.1% to about 90%, about 0.1% to about 99%, about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 2% to about 10%, about 2% to about 20%, about 2% to about 30%, about 2% to about 40%, about 2% to about 50%, about 2% to about 60%, about 2% to about 70%, about 2% to about 80%, about 2% to about 90%, about 2% to about 99%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 5% to about 99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 20% to about 30%, about 20% to about 40%, about 20% to about 50%, about 20% to about 60%, about 20% to about 70%, about 20% to about 80%, about 20% to about 90%, about 20% to about 99%, about 30% to about 40%, about 30% to about 50%, about 30% to about 60%, about 30% to about 70%, about 30% to about 80%, about 30% to about 90%, about 30% to about 99%, about 40% to about 50%, about 40% to about 60%, about 40% to about 70%, about 40% to about 80%, about 40% to about 90%, about 40% to about 99%, about 50% to about 60%, about 50% to about 70%, about 50% to about 80%, about 50% to about 90%, about 50% to about 99%, about 60% to about 70%, about 60% to about 80%, about 60% to about 90%, about 60% to about 99%, about 70% to about 80%, about 70% to about 90%, about 70% to about 99%, about 80% to about 90%, about 80% to about 99%, or about 90% to about 99%.

In certain embodiments, the method of manufacturing a tablet pharmaceutical composition may further include a step (ii) of loading the sifted materials from step (i) to a blender and blending for a first period of time. The first period of time may be from about 0.5 to about 60 min, e.g., about 0.5 min, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, 10 min, 10.5 min, 11 min, 11.5 min, 12 min, 12.5 min, 13 min, 13.5 min, 14 min, 14.5 min, 15 min, 15.5 min, 16 min, 16.5 min, 17 min, 17.5 min, 18 min, 18.5 min, 19 min, 19.5 min, 20 min, 20.5 min, 21 min, 21.5 min, 22 min, 22.5 min, 23 min, 23.5 min, 24 min, 24.5 min, 25 min, 25.5 min, 26 min, 26.5 min, 27 min, 27.5 min, 28 min, 28.5 min, 29 min, 29.5 min, 30 min, 30.5 min, 31 min, 31.5 min, 32 min, 32.5 min, 33 min, 33.5 min, 34 min, 34.5 min, 35 min, 35.5 min, 36 min, 36.5 min, 37 min, 37.5 min, 38 min, 38.5 min, 39 min, 39.5 min, 40 min, 40.5 min, 41 min, 41.5 min, 42 min, 42.5 min, 43 min, 43.5 min, 44 min, 44.5 min, 45 min, 45.5 min, 46 min, 46.5 min, 47 min, 47.5 min, 48 min, 48.5 min, 49 min, 49.5 min, 50 min, 50.5 min, 51 min, 51.5 min, 52 min, 52.5 min, 53 min, 53.5 min, 54 min, 54.5 min, 55 min, 55.5 min, 56 min, 56.5 min, 57 min, 57.5 min, 58 min, 58.5 min, 59 min, 59.5 min, 60 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or about 60 min, or any duration therebetween. In certain embodiments, the first period of time may be about 15 min. In certain embodiments, the blending in step (ii) may be carried out for 15 min at 15 rpm.

In certain embodiments, the method of manufacturing a tablet pharmaceutical composition may further comprise a step (iii) of unloading the blended materials from step (ii) and sifting through a 30-mesh screen. In certain embodiments, the method may further comprise a step (iv) of adding the sifted materials from step (iii) to a blender and blending for a second period of time. The second period of time may be from about 0.5 to about 60 min, e.g., about 0.5 min, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, 10 min, 10.5 min, 11 min, 11.5 min, 12 min, 12.5 min, 13 min, 13.5 min, 14 min, 14.5 min, 15 min, 15.5 min, 16 min, 16.5 min, 17 min, 17.5 min, 18 min, 18.5 min, 19 min, 19.5 min, 20 min, 20.5 min, 21 min, 21.5 min, 22 min, 22.5 min, 23 min, 23.5 min, 24 min, 24.5 min, 25 min, 25.5 min, 26 min, 26.5 min, 27 min, 27.5 min, 28 min, 28.5 min, 29 min, 29.5 min, 30 min, 30.5 min, 31 min, 31.5 min, 32 min, 32.5 min, 33 min, 33.5 min, 34 min, 34.5 min, 35 min, 35.5 min, 36 min, 36.5 min, 37 min, 37.5 min, 38 min, 38.5 min, 39 min, 39.5 min, 40 min, 40.5 min, 41 min, 41.5 min, 42 min, 42.5 min, 43 min, 43.5 min, 44 min, 44.5 min, 45 min, 45.5 min, 46 min, 46.5 min, 47 min, 47.5 min, 48 min, 48.5 min, 49 min, 49.5 min, 50 min, 50.5 min, 51 min, 51.5 min, 52 min, 52.5 min, 53 min, 53.5 min, 54 min, 54.5 min, 55 min, 55.5 min, 56 min, 56.5 min, 57 min, 57.5 min, 58 min, 58.5 min, 59 min, 59.5 min, 60 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or about 60 min, or any duration therebetween. In certain embodiments, the second period of time may be about 15 min. In certain embodiments, the blending in step (iv) may be carried out for 15 min at 15 rpm.

In certain embodiments, the method of manufacturing a tablet pharmaceutical composition may further comprise a step (v) of co-sifting a glidant and the lubricant through a 60-mesh screen and lubricating the blended materials from the step (iv) with a blender for a third period of time. The third period of time may be from about 0.5 to about 60 min, e.g., about 0.5 min, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, 10 min, 10.5 min, 11 min, 11.5 min, 12 min, 12.5 min, 13 min, 13.5 min, 14 min, 14.5 min, 15 min, 15.5 min, 16 min, 16.5 min, 17 min, 17.5 min, 18 min, 18.5 min, 19 min, 19.5 min, 20 min, 20.5 min, 21 min, 21.5 min, 22 min, 22.5 min, 23 min, 23.5 min, 24 min, 24.5 min, 25 min, 25.5 min, 26 min, 26.5 min, 27 min, 27.5 min, 28 min, 28.5 min, 29 min, 29.5 min, 30 min, 30.5 min, 31 min, 31.5 min, 32 min, 32.5 min, 33 min, 33.5 min, 34 min, 34.5 min, 35 min, 35.5 min, 36 min, 36.5 min, 37 min, 37.5 min, 38 min, 38.5 min, 39 min, 39.5 min, 40 min, 40.5 min, 41 min, 41.5 min, 42 min, 42.5 min, 43 min, 43.5 min, 44 min, 44.5 min, 45 min, 45.5 min, 46 min, 46.5 min, 47 min, 47.5 min, 48 min, 48.5 min, 49 min, 49.5 min, 50 min, 50.5 min, 51 min, 51.5 min, 52 min, 52.5 min, 53 min, 53.5 min, 54 min, 54.5 min, 55 min, 55.5 min, 56 min, 56.5 min, 57 min, 57.5 min, 58 min, 58.5 min, 59 min, 59.5 min, 60 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or about 60 min, or any duration therebetween. In certain embodiments, the third period of time may be about 5 min. In certain embodiments, the blending in step (v) may be carried out for 5 min at 15 rpm. In certain embodiments, the lubricant used in the step (v) may be intragranular. In certain embodiments, the bulk density and tapped density (BD/TD) of the lubricated materials from the step (v) may be tested. In embodiments, the BD/TD of the lubricated materials form the step (v) may be from about 0.45 g/ml to about 0.60 g/ml. In certain embodiments, the BD/TD of the lubricated materials from the step (v) may be about 0.50 g/ml.

In certain embodiments, the method of manufacturing a tablet pharmaceutical composition may further comprise a step (vi) of granulating the lubricated materials from step (v) by roller compaction. In certain embodiments, the step (vi) may be carried out at a roll pressure from about 2 to about 5 MPa, e.g., about 2 MPa, 2.1 MPa, 2.2 MPa, 2.3 MPa, 2.4 MPa, 2.5 MPa, 2.6 MPa, 2.7 MPa, 2.8 MPa, 2.9 MPa, 3 MPa, 3.1 MPa, 3.2 MPa, 3.3 MPa, 3.4 MPa, 3.5 MPa, 3.6 MPa, 3.7 MPa, 3.8 MPa, 3.9 MPa, 4 MPa, 4.1 MPa, 4.2 MPa, 4.3 MPa, 4.4 MPa, 4.5 MPa, 4.6 MPa, 4.7 MPa, 4.8 MPa, 4.9 MPa, or 5 MPa, or any pressure therebetween. In certain embodiments, the step (vi) may be carried out at a roll pressure of about 3 MPa. In certain embodiments, the step (vi) may be carried out at a roll gap from about 0.3 to about 4 mm, e.g., about 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, or 4 mm, or any value therebetween. In certain embodiments, the step (vi) may be carried out at a roll gap of around 2.0 mm. In certain embodiments, the step (vi) may be carried out at a roll speed from about 3 to about 7 rpm, e.g., about 3 rpm, 3.1 rpm, 3.2 rpm, 3.3 rpm, 3.4 rpm, 3.5 rpm, 3.6 rpm, 3.7 rpm, 3.8 rpm, 3.9 rpm, 4 rpm, 4.1 rpm, 4.2 rpm, 4.3 rpm, 4.4 rpm, 4.5 rpm, 4.6 rpm, 4.7 rpm, 4.8 rpm, 4.9 rpm, 5 rpm, 5.1 rpm, 5.2 rpm, 5.3 rpm, 5.4 rpm, 5.5 rpm, 5.6 rpm, 5.7 rpm, 5.8 rpm, 5.9 rpm, 6 rpm, 6.1 rpm, 6.2 rpm, 6.3 rpm, 6.4 rpm, 6.5 rpm, 6.6 rpm, 6.7 rpm, 6.8 rpm, 6.9 rpm, or 7 rpm, or any speed therebetween. In certain embodiments, the step (vi) may be carried out at a roll speed of about 4.0 rpm. In certain embodiments, the step (vi) may be carried out at a feed screw speed from about 18 to about 81 rpm, e.g., about 18 rpm, 19 rpm, 20 rpm, 21 rpm, 22 rpm, 23 rpm, 24 rpm, 25 rpm, 26 rpm, 27 rpm, 28 rpm, 29 rpm, 30 rpm, 31 rpm, 32 rpm, 33 rpm, 34 rpm, 35 rpm, 36 rpm, 37 rpm, 38 rpm, 39 rpm, 40 rpm, 41 rpm, 42 rpm, 43 rpm, 44 rpm, 45 rpm, 46 rpm, 47 rpm, 48 rpm, 49 rpm, 50 rpm, 51 rpm, 52 rpm, 53 rpm, 54 rpm, 55 rpm, 56 rpm, 57 rpm, 58 rpm, 59 rpm, 60 rpm, 61 rpm, 62 rpm, 63 rpm, 64 rpm, 65 rpm, 66 rpm, 67 rpm, 68 rpm, 69 rpm, 70 rpm, 71 rpm, 72 rpm, 73 rpm, 74 rpm, 75 rpm, 76 rpm, 77 rpm, 78 rpm, 79 rpm, 80 rpm, or 81 rpm, or any speed therebetween. In certain embodiments, the step (vi) may be carried out at a feed screw speed of about 20 rpm. In certain embodiments, the step (vi) may be carried out at a roll pressure of about 3 MPa, a roll gap of about 2 mm, a roll speed of about 4 rpm, and a feed screw speed of about 20 rpm. In certain embodiments, the bulk density of the lubricated materials of step (vi) may be from about 0.45 to about 0.6 g/ml. In certain embodiments, the bulk density of the lubricated materials of step (vi) may be about 0.5 g/ml.

In certain embodiments, the method of manufacturing a tablet pharmaceutical composition may further comprise a step (vii) of sifting the lubricant through a 60-mesh screen and lubricating the granulated materials from the step (vi) for a fourth period of time. The fourth period of time may be from about 0.5 to about 60 min, e.g., about 0.5 min, 1 min, 1.5 min, 2 min, 2.5 min, 3 min, 3.5 min, 4 min, 4.5 min, 5 min, 5.5 min, 6 min, 6.5 min, 7 min, 7.5 min, 8 min, 8.5 min, 9 min, 9.5 min, 10 min, 10.5 min, 11 min, 11.5 min, 12 min, 12.5 min, 13 min, 13.5 min, 14 min, 14.5 min, 15 min, 15.5 min, 16 min, 16.5 min, 17 min, 17.5 min, 18 min, 18.5 min, 19 min, 19.5 min, 20 min, 20.5 min, 21 min, 21.5 min, 22 min, 22.5 min, 23 min, 23.5 min, 24 min, 24.5 min, 25 min, 25.5 min, 26 min, 26.5 min, 27 min, 27.5 min, 28 min, 28.5 min, 29 min, 29.5 min, 30 min, 30.5 min, 31 min, 31.5 min, 32 min, 32.5 min, 33 min, 33.5 min, 34 min, 34.5 min, 35 min, 35.5 min, 36 min, 36.5 min, 37 min, 37.5 min, 38 min, 38.5 min, 39 min, 39.5 min, 40 min, 40.5 min, 41 min, 41.5 min, 42 min, 42.5 min, 43 min, 43.5 min, 44 min, 44.5 min, 45 min, 45.5 min, 46 min, 46.5 min, 47 min, 47.5 min, 48 min, 48.5 min, 49 min, 49.5 min, 50 min, 50.5 min, 51 min, 51.5 min, 52 min, 52.5 min, 53 min, 53.5 min, 54 min, 54.5 min, 55 min, 55.5 min, 56 min, 56.5 min, 57 min, 57.5 min, 58 min, 58.5 min, 59 min, 59.5 min, 60 min, 31 min, 32 min, 33 min, 34 min, 35 min, 36 min, 37 min, 38 min, 39 min, 40 min, 41 min, 42 min, 43 min, 44 min, 45 min, 46 min, 47 min, 48 min, 49 min, 50 min, 51 min, 52 min, 53 min, 54 min, 55 min, 56 min, 57 min, 58 min, 59 min, or about 60 min, or any duration therebetween. In certain embodiments, the fourth period of time may be about 5 min. In certain embodiments, the lubricating in step (vii) may be carried out for 5 min at 15 rpm. In embodiments, the lubricant used in the step (vii) may be extragranular.

In certain embodiments, the method of manufacturing a tablet pharmaceutical composition may further comprise a step (viii) of compressing the materials from the step (vii) into the tablet pharmaceutical composition. In certain embodiments, the step (viii) may be carried out using a round-shaped punch of about 6.0 mm. In certain embodiments, the step (viii) may be carried out at a rotary speed from about 20 to about 30 rpm, e.g., about 20 rpm, 21 rpm, 22 rpm, 23 rpm, 24 rpm, 25 rpm, 26 rpm, 27 rpm, 28 rpm, 29 rpm, or 30 rpm, or any speed therebetween. In certain embodiments, the step (viii) may be carried out at a rotary speed of about 30 rpm. In certain embodiments, the step (viii) may be carried out using a thickness scale from about 1.0 to about 3.5 mm, e.g., about 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, or about 3.5 mm, or value therebetween. In certain embodiments, the step (viii) may be carried out using a thickness scale of about 2.0 mm. In certain embodiments, the step (viii) may be carried out using a fill depth scale from about 3.0 to about 10.0 mm, e.g., about 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, 6.1 mm, 6.2 mm, 6.3 mm, 6.4 mm, 6.5 mm, 6.6 mm, 6.7 mm, 6.8 mm, 6.9 mm, 7 mm, 7.1 mm, 7.2 mm, 7.3 mm, 7.4 mm, 7.5 mm, 7.6 mm, 7.7 mm, 7.8 mm, 7.9 mm, 8 mm, 8.1 mm, 8.2 mm, 8.3 mm, 8.4 mm, 8.5 mm, 8.6 mm, 8.7 mm, 8.8 mm, 8.9 mm, 9 mm, 9.1 mm, 9.2 mm, 9.3 mm, 9.4 mm, 9.5 mm, 9.6 mm, 9.7 mm, 9.8 mm, 9.9 mm, or about 10 mm, or any value therebetween. In certain embodiments, the step (viii) may be carried out using a fill depth scale of about 6.0 mm. In certain embodiments, the step (viii) may be carried out at a main compression force from about 3.0 to about 10.0 kN, e.g., about 3 kN, 3.1 kN, 3.2 kN, 3.3 kN, 3.4 kN, 3.5 kN, 3.6 kN, 3.7 kN, 3.8 kN, 3.9 kN, 4 kN, 4.1 kN, 4.2 kN, 4.3 kN, 4.4 kN, 4.5 kN, 4.6 kN, 4.7 kN, 4.8 kN, 4.9 kN, 5 kN, 5.1 kN, 5.2 kN, 5.3 kN, 5.4 kN, 5.5 kN, 5.6 kN, 5.7 kN, 5.8 kN, 5.9 kN, 6 kN, 6.1 kN, 6.2 kN, 6.3 kN, 6.4 kN, 6.5 kN, 6.6 kN, 6.7 kN, 6.8 kN, 6.9 kN, 7 kN, 7.1 kN, 7.2 kN, 7.3 kN, 7.4 kN, 7.5 kN, 7.6 kN, 7.7 kN, 7.8 kN, 7.9 kN, 8 kN, 8.1 kN, 8.2 kN, 8.3 kN, 8.4 kN, 8.5 kN, 8.6 kN, 8.7 kN, 8.8 kN, 8.9 kN, 9 kN, 9.1 kN, 9.2 kN, 9.3 kN, 9.4 kN, 9.5 kN, 9.6 kN, 9.7 kN, 9.8 kN, 9.9 kN, or about 10 kN, or any pressure therebetween. In certain embodiments, the step (viii) may be carried out at a main compression force of about 7.0 kN. In certain embodiments, the step (viii) may be carried out at a rotary speed of about 30 rpm, a thickness scale of about 2.0 mm, a fill depth scale of about 6.0 mm, and a main compression force of about 7.0 kN.

In certain embodiments, a tablet pharmaceutical composition manufactured according to a method disclosed herein may be from about 92.5 to about 107.5 mg. In certain embodiments, a tablet pharmaceutical composition manufactured according to a method disclosed herein may be about 100 mg. In certain embodiments, a tablet pharmaceutical composition manufactured according to a method disclosed herein may include a thickness from about 2.7 to about 3.3 mm, e.g., about 2.7 mm, 2.8 mm, 2.9 mm, 3.0 mm, 3.1 mm, 3.2 mm, or 3.3 mm, or any thickness therebetween. In certain embodiments, a tablet pharmaceutical composition manufactured according to a method disclosed herein may include a thickness of about 3.0 mm. In certain embodiments, a tablet pharmaceutical composition manufactured according to a method disclosed herein may include a hardness from about 45 to about 95 N, e.g., about 45 N, 46 N, 47 N, 48 N, 49 N, 50 N, 51 N, 52 N, 53 N, 54 N, 55 N, 56 N, 57 N, 58 N, 59 N, 60 N, 61 N, 62 N, 63 N, 64 N, 65 N, 66 N, 67 N, 68 N, 69 N, 70 N, 71 N, 72 N, 73 N, 74 N, 75 N, 76 N, 77 N, 78 N, 79 N, 80 N, 81 N, 82 N, 83 N, 84 N, 85 N, 86 N, 87 N, 88 N, 89 N, 90 N, 91 N, 92 N, 93 N, 94 N, or about 95 N, or any hardness therebetween. In certain embodiments, a tablet pharmaceutical composition manufactured according to a method disclosed herein may include a hardness of about 70 N. In embodiments, the measurement of hardness of a tablet pharmaceutical composition manufactured according to a method disclosed herein may be carried out using a standard USP protocol <1217>.

In certain embodiments, a tablet pharmaceutical composition manufactured according to a method disclosed herein may include a friability of no more than 1.0% by weight, e.g., about 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, or about 0.99%, or any percentage between 0% and 1.0%. In embodiments, the measurement of friability of a tablet pharmaceutical composition manufactured according to a method disclosed herein may be carried out using a standard USP protocol <1216>.

In certain embodiments, a tablet pharmaceutical composition manufactured according to a method disclosed herein may include a disintegration time of no more than 15 min, e.g., about 0.1 min, 0.2 min, 0.3 min, 0.4 min, 0.5 min, 0.6 min, 0.7 min, 0.8 min, 0.9 min, 1 min, 1.1 min, 1.2 min, 1.3 min, 1.4 min, 1.5 min, 1.6 min, 1.7 min, 1.8 min, 1.9 min, 2 min, 2.1 min, 2.2 min, 2.3 min, 2.4 min, 2.5 min, 2.6 min, 2.7 min, 2.8 min, 2.9 min, 3 min, 3.1 min, 3.2 min, 3.3 min, 3.4 min, 3.5 min, 3.6 min, 3.7 min, 3.8 min, 3.9 min, 4 min, 4.1 min, 4.2 min, 4.3 min, 4.4 min, 4.5 min, 4.6 min, 4.7 min, 4.8 min, 4.9 min, 5 min, 5.1 min, 5.2 min, 5.3 min, 5.4 min, 5.5 min, 5.6 min, 5.7 min, 5.8 min, 5.9 min, 6 min, 6.1 min, 6.2 min, 6.3 min, 6.4 min, 6.5 min, 6.6 min, 6.7 min, 6.8 min, 6.9 min, 7 min, 7.1 min, 7.2 min, 7.3 min, 7.4 min, 7.5 min, 7.6 min, 7.7 min, 7.8 min, 7.9 min, 8 min, 8.1 min, 8.2 min, 8.3 min, 8.4 min, 8.5 min, 8.6 min, 8.7 min, 8.8 min, 8.9 min, 9 min, 9.1 min, 9.2 min, 9.3 min, 9.4 min, 9.5 min, 9.6 min, 9.7 min, 9.8 min, 9.9 min, 10 min, 10.1 min, 10.2 min, 10.3 min, 10.4 min, 10.5 min, 10.6 min, 10.7 min, 10.8 min, 10.9 min, 11 min, 11.1 min, 11.2 min, 11.3 min, 11.4 min, 11.5 min, 11.6 min, 11.7 min, 11.8 min, 11.9 min, 12 min, 12.1 min, 12.2 min, 12.3 min, 12.4 min, 12.5 min, 12.6 min, 12.7 min, 12.8 min, 12.9 min, 13 min, 13.1 min, 13.2 min, 13.3 min, 13.4 min, 13.5 min, 13.6 min, 13.7 min, 13.8 min, 13.9 min, 14 min, 14.1 min, 14.2 min, 14.3 min, 14.4 min, 14.5 min, 14.6 min, 14.7 min, 14.8 min, 14.9 min, or about 14.99 min, or any duration between 0 and 15 min. In embodiments, the measurement of disintegration time of a tablet pharmaceutical composition manufactured according to a method disclosed herein may be carried out using a standard USP protocol <701>.

In certain embodiments, a method of manufacturing a tablet pharmaceutical composition disclosed herein may further comprise a step (ix) of packaging the manufactured tablet pharmaceutical composition from step (viii) into a bottle. In certain embodiments, the step (ix) may be carried out using a HDPE bottle of about 45 ml and the HDPE bottle contains 30 of the manufactured tablet pharmaceutical composition. In certain embodiments, at least one of the blenders used in steps (ii), (iv) and (v) of a method of manufacturing a tablet pharmaceutical composition disclosed herein may be a bin blender of about 100 L.

In certain embodiments, after the step (iv), a method of manufacturing a tablet pharmaceutical composition disclosed herein may further comprise a step (x) of taking a sample of the blended materials from each of about 10 locations of the blender. In certain embodiments, the sample taken in step (x) may be from about 98.5 to about 295.5 mg, e.g., about 98.5 mg, 99 min, 99.5 min, 100 min, 100.5 min, 101 min, 101.5 min, 102 min, 102.5 min, 103 min, 103.5 min, 104 min, 104.5 min, 105 min, 105.5 min, 106 min, 106.5 min, 107 min, 107.5 min, 108 min, 108.5 min, 109 min, 109.5 min, 110 min, 110.5 min, 111 min, 111.5 min, 112 min, 112.5 min, 113 min, 113.5 min, 114 min, 114.5 min, 115 min, 115.5 min, 116 min, 116.5 min, 117 min, 117.5 min, 118 min, 118.5 min, 119 min, 119.5 min, 120 min, 120.5 min, 121 min, 121.5 min, 122 min, 122.5 min, 123 min, 123.5 min, 124 min, 124.5 min, 125 min, 125.5 min, 126 min, 126.5 min, 127 min, 127.5 min, 128 min, 128.5 min, 129 min, 129.5 min, 130 min, 130.5 min, 131 min, 131.5 min, 132 min, 132.5 min, 133 min, 133.5 min, 134 min, 134.5 min, 135 min, 135.5 min, 136 min, 136.5 min, 137 min, 137.5 min, 138 min, 138.5 min, 139 min, 139.5 min, 140 min, 140.5 min, 141 min, 141.5 min, 142 min, 142.5 min, 143 min, 143.5 min, 144 min, 144.5 min, 145 min, 145.5 min, 146 min, 146.5 min, 147 min, 147.5 min, 148 min, 148.5 min, 149 min, 149.5 min, 150 min, 150.5 min, 151 min, 151.5 min, 152 min, 152.5 min, 153 min, 153.5 min, 154 min, 154.5 min, 155 min, 155.5 min, 156 min, 156.5 min, 157 min, 157.5 min, 158 min, 158.5 min, 159 min, 159.5 min, 160 min, 160.5 min, 161 min, 161.5 min, 162 min, 162.5 min, 163 min, 163.5 min, 164 min, 164.5 min, 165 min, 165.5 min, 166 min, 166.5 min, 167 min, 167.5 min, 168 min, 168.5 min, 169 min, 169.5 min, 170 min, 170.5 min, 171 min, 171.5 min, 172 min, 172.5 min, 173 min, 173.5 mg 174 mg, 174.5 mg, 175 mg, 175.5 mg, 176 mg, 176.5 mg, 177 mg, 177.5 mg, 178 mg, 178.5 mg 179 mg, 179.5 mg, 180 mg, 180.5 mg, 181 mg, 181.5 mg, 182 mg, 182.5 mg, 183 mg, 183.5 mg 184 mg, 184.5 mg, 185 mg, 185.5 mg, 186 mg, 186.5 mg, 187 mg, 187.5 mg, 188 mg, 188.5 mg 189 mg, 189.5 mg, 190 mg, 190.5 mg, 191 mg, 191.5 mg, 192 mg, 192.5 mg, 193 mg, 193.5 mg 194 mg, 194.5 mg, 195 mg, 195.5 mg, 196 mg, 196.5 mg, 197 mg, 197.5 mg, 198 mg, 198.5 mg 199 mg, 199.5 mg, 200 mg, 200.5 mg, 201 mg, 201.5 mg, 202 mg, 202.5 mg, 203 mg, 203.5 mg 204 mg, 204.5 mg, 205 mg, 205.5 mg, 206 mg, 206.5 mg, 207 mg, 207.5 mg, 208 mg, 208.5 mg 209 mg, 209.5 mg, 210 mg, 210.5 mg, 211 mg, 211.5 mg, 212 mg, 212.5 mg, 213 mg, 213.5 mg 214 mg, 214.5 mg, 215 mg, 215.5 mg, 216 mg, 216.5 mg, 217 mg, 217.5 mg, 218 mg, 218.5 mg 219 mg, 219.5 mg, 220 mg, 220.5 mg, 221 mg, 221.5 mg, 222 mg, 222.5 mg, 223 mg, 223.5 mg 224 mg, 224.5 mg, 225 mg, 225.5 mg, 226 mg, 226.5 mg, 227 mg, 227.5 mg, 228 mg, 228.5 mg 229 mg, 229.5 mg, 230 mg, 230.5 mg, 231 mg, 231.5 mg, 232 mg, 232.5 mg, 233 mg, 233.5 mg 234 mg, 234.5 mg, 235 mg, 235.5 mg, 236 mg, 236.5 mg, 237 mg, 237.5 mg, 238 mg, 238.5 mg 239 mg, 239.5 mg, 240 mg, 240.5 mg, 241 mg, 241.5 mg, 242 mg, 242.5 mg, 243 mg, 243.5 mg 244 mg, 244.5 mg, 245 mg, 245.5 mg, 246 mg, 246.5 mg, 247 mg, 247.5 mg, 248 mg, 248.5 mg 249 mg, 249.5 mg, 250 mg, 250.5 mg, 251 mg, 251.5 mg, 252 mg, 252.5 mg, 253 mg, 253.5 mg 254 mg, 254.5 mg, 255 mg, 255.5 mg, 256 mg, 256.5 mg, 257 mg, 257.5 mg, 258 mg, 258.5 mg 259 mg, 259.5 mg, 260 mg, 260.5 mg, 261 mg, 261.5 mg, 262 mg, 262.5 mg, 263 mg, 263.5 mg 264 mg, 264.5 mg, 265 mg, 265.5 mg, 266 mg, 266.5 mg, 267 mg, 267.5 mg, 268 mg, 268.5 mg 269 mg, 269.5 mg, 270 mg, 270.5 mg, 271 mg, 271.5 mg, 272 mg, 272.5 mg, 273 mg, 273.5 mg 274 mg, 274.5 mg, 275 mg, 275.5 mg, 276 mg, 276.5 mg, 277 mg, 277.5 mg, 278 mg, 278.5 mg 279 mg, 279.5 mg, 280 mg, 280.5 mg, 281 mg, 281.5 mg, 282 mg, 282.5 mg, 283 mg, 283.5 mg 284 mg, 284.5 mg, 285 mg, 285.5 mg, 286 mg, 286.5 mg, 287 mg, 287.5 mg, 288 mg, 288.5 mg 289 mg, 289.5 mg, 290 mg, 290.5 mg, 291 mg, 291.5 mg, 292 mg, 292.5 mg, 293 mg, 293.5 mg 294 mg, 294.5 mg, 295 mg, or about 295.5 mg, or any amount therebetween. In certain embodiments, the sample taken from each location of the blender in step (x) may be about 197.0 mg. In certain embodiments, the step (x) of taking a sample of the blended materials may be carried out so that all the individual assays are within mean±10% (absolute) and RSD % and the NMT is about 5%.

In certain embodiments, the compound of Formula (I) may be from about 1% to about 99.99% by weight of a tablet pharmaceutical composition manufactured according to a method disclosed herein, e.g., about 1% to about 10%, about 1% to about 20%, about 1% to about 30%, about 1% to about 40%, about 1% to about 50%, about 1% to about 60%, about 1% to about 70%, about 1% to about 80%, about 1% to about 90%, about 1% to about 99%, about 1% to about 99.99%, about 10% to about 20%, about 10% to about 30%, about 10% to about 40%, about 10% to about 50%, about 10% to about 60%, about 10% to about 70%, about 10% to about 80%, about 10% to about 90%, about 10% to about 99%, about 10% to about 99.99%, about 20% to about 30%, about 20% to about 40%, about 20% to about 50%, about 20% to about 60%, about 20% to about 70%, about 20% to about 80%, about 20% to about 90%, about 20% to about 99%, about 20% to about 99.99%, about 30% to about 40%, about 30% to about 50%, about 30% to about 60%, about 30% to about 70%, about 30% to about 80%, about 30% to about 90%, about 30% to about 99%, about 30% to about 99.99%, about 40% to about 50%, about 40% to about 60%, about 40% to about 70%, about 40% to about 80%, about 40% to about 90%, about 40% to about 99%, about 40% to about 99.99%, about 50% to about 60%, about 50% to about 70%, about 50% to about 80%, about 50% to about 90%, about 50% to about 99%, about 50% to about 99.99%, about 60% to about 70%, about 60% to about 80%, about 60% to about 90%, about 60% to about 99%, about 60% to about 99.99%, about 70% to about 80%, about 70% to about 90%, about 70% to about 99%, about 70% to about 99.99%, about 80% to about 90%, about 80% to about 99%, about 80% to about 99.99%, about 90% to about 99%, about 90% to about 99.99%, or about 99% to about 99.99%.

In certain embodiments, the dry weight of a tablet pharmaceutical composition manufactured according to a method disclosed herein may be from about 0.1 mg to about 400 mg, e.g., about 0.1 mg, 0.2 mg, 0.3 mg, 0.4 mg, 0.5 mg, 0.6 mg, 0.7 mg, 0.8 mg, 0.9 mg, 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 102 mg, 104 mg, 106 mg, 108 mg, 110 mg, 112 mg, 114 mg, 116 mg, 118 mg, 120 mg, 122 mg, 124 mg, 126 mg, 128 mg, 130 mg, 132 mg, 134 mg, 136 mg, 138 mg, 140 mg, 142 mg, 144 mg, 146 mg, 148 mg, 150 mg, 152 mg, 154 mg, 156 mg, 158 mg, 160 mg, 162 mg, 164 mg, 166 mg, 168 mg, 170 mg, 172 mg, 174 mg, 176 mg, 178 mg, 180 mg, 182 mg, 184 mg, 186 mg, 188 mg, 190 mg, 192 mg, 194 mg, 196 mg, 198 mg, 200 mg, 202 mg, 204 mg, 206 mg, 208 mg, 210 mg, 212 mg, 214 mg, 216 mg, 218 mg, 220 mg, 222 mg, 224 mg, 226 mg, 228 mg, 230 mg, 232 mg, 234 mg, 236 mg, 238 mg, 240 mg, 242 mg, 244 mg, 246 mg, 248 mg, 250 mg, 252 mg, 254 mg, 256 mg, 258 mg, 260 mg, 262 mg, 264 mg, 266 mg, 268 mg, 270 mg, 272 mg, 274 mg, 276 mg, 278 mg, 280 mg, 282 mg, 284 mg, 286 mg, 288 mg, 290 mg, 292 mg, 294 mg, 296 mg, 298 mg, 300 mg, 302 mg, 304 mg, 306 mg, 308 mg, 310 mg, 312 mg, 314 mg, 316 mg, 318 mg, 320 mg, 322 mg, 324 mg, 326 mg, 328 mg, 330 mg, 332 mg, 334 mg, 336 mg, 338 mg, 340 mg, 342 mg, 344 mg, 346 mg, 348 mg, 350 mg, 352 mg, 354 mg, 356 mg, 358 mg, 360 mg, 362 mg, 364 mg, 366 mg, 368 mg, 370 mg, 372 mg, 374 mg, 376 mg, 378 mg, 380 mg, 382 mg, 384 mg, 386 mg, 388 mg, 390 mg, 392 mg, 394 mg, 396 mg, 398 mg, or about 400 mg, or any amount therebetween. In certain embodiments, the dry weight of a tablet pharmaceutical composition manufactured according to a method disclosed herein may be about 100 mg.

In certain embodiments of methods of manufacturing a tablet pharmaceutical composition, the dispersion polymer may be selected from the group comprising hydroxypropyl methylcellulose (HPMC), hydroxypropyl methylcellulose-acetate succinate (HPMC-AS or HPMCAS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, polyoxyethylene-polyoxypropylene block copolymers, and derivatives or combinations thereof. In certain embodiments, the dispersion polymer is HPMC-AS. In certain embodiments, the dispersion polymer is HPMC. In some embodiments, the dispersion polymer is PVP.

In some embodiments, the dispersion polymer may be a particular grade of HPMC-AS. In some embodiments, the dispersion polymer is HPMC-AS, L. In some embodiments, the dispersion polymer is HPMC-AS, M. In some embodiments, the dispersion polymer is HPMC-AS, H. In some embodiments, the dispersion polymer is PVP. In some embodiments, the PVP has a molecular weight from about 7000 Daltons to about 11000 Daltons.

In certain embodiments, the compound of Formula (I) has the structure:

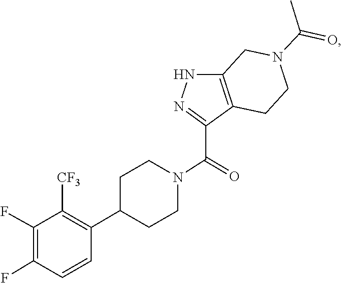 [SMILES: CC(=O)N1CCc2c(C(=O)N3CCC(c4ccc(F)c(F)c4C(F)(F)F)CC3)n[nH]c2C1]

or a pharmaceutically acceptable salt, solvate, polymorph, prodrug, metabolite, N-oxide, stereoisomer, or isomer thereof. In certain embodiments, the compound of Formula (I) has the structure:

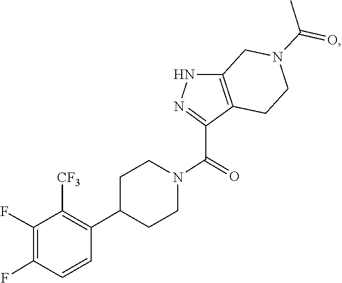 [SMILES: CC(=O)N1CCc2c(C(=O)N3CCC(c4ccc(F)c(F)c4C(F)(F)F)CC3)n[nH]c2C1]

or a pharmaceutically acceptable salt, solvate, polymorph, prodrug, metabolite, N-oxide, stereoisomer, or isomer thereof is formulated as a tablet pharmaceutical composition.

### Polymorphs of Compound 1

Provided herein is a new, unique polymorph of Compound 1. This polymorph, described herein as “Form C.” This polymorph was found to be readily prepared from a variety of methods provided in the Examples section. Form C is a crystalline solid having high thermodynamic stability, making it well suited for pharmaceutical formulations of Compound 1.

Provided herein is a polymorph of Compound 1 having an X-ray powder diffraction pattern that shows numerous peaks at many degrees two theta, including with limitation at approximately 6.7, 9.3, 14.1, 17.2, 23.5, 27.1, and/or 29.0. XRPD peak values in the application refer to those obtained using a copper source with a wavelength of 1.5406 angstrom unless otherwise noted.

In one aspect, provided herein, is polymorph of a compound of Formula (I) having the structure

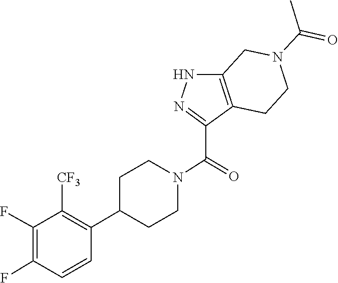 [SMILES: CC(=O)N1CCc2c(C(=O)N3CCC(c4ccc(F)c(F)c4C(F)(F)F)CC3)n[nH]c2C1]

wherein the compound of Formula (I) exhibits an x-ray powder diffraction pattern having at least three characteristic peaks expressed in degrees two theta (+/−0.5 degree theta) at 6.7, 9.3, 14.1, 17.2, 23.5, 27.1, and/or 29.0. In some embodiments, the polymorph exhibits an x-ray powder diffraction pattern having at least four characteristic peaks expressed in degrees two theta (+/−0.5 degree theta) at 6.7, 9.3, 14.1, 17.2, 23.5, 27.1, and/or 29.0. In some embodiments, the polymorph exhibits an x-ray powder diffraction pattern having at least five characteristic peaks expressed in degrees two theta (+/−0.5 degree theta) at 6.7, 9.3, 14.1, 17.2, 23.5, 27.1, and/or 29.0. In some embodiments, the polymorph exhibits an x-ray powder diffraction pattern having at least six characteristic peaks expressed in degrees two theta (+/−0.5 degree theta) at 6.7, 9.3, 14.1, 17.2, 23.5, 27.1, and/or 29.0. In some embodiments, the polymorph exhibits an x-ray powder diffraction pattern having characteristic peaks expressed in degrees two theta (+/−0.5 degree theta) at 6.7, 9.3, 14.1, 17.2, 23.5, 27.1, and/or 29.0.

In one aspect, provided herein, is polymorph of a compound of Formula (I) having the structure

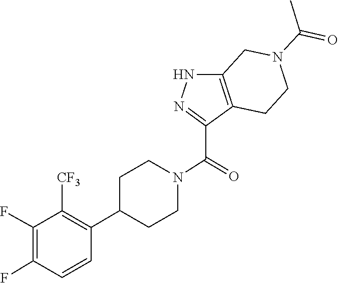 [SMILES: CC(=O)N1CCc2c(C(=O)N3CCC(c4ccc(F)c(F)c4C(F)(F)F)CC3)n[nH]c2C1]

wherein the polymorph exhibits an x-ray powder diffraction pattern having at least three characteristic peaks expressed in degrees two theta (+/−1.0 degree theta) at 6.7, 9.3, 14.1, 17.2, 23.5, 27.1, and/or 29.0. In some embodiments, the polymorph exhibits an x-ray powder diffraction pattern having at least four characteristic peaks expressed in degrees two theta (+/−1.0 degree theta) at 6.7, 9.3, 14.1, 17.2, 23.5, 27.1, and/or 29.0. In some embodiments, the polymorph exhibits an x-ray powder diffraction pattern having at least five characteristic peaks expressed in degrees two theta (+/−1.0 degree theta) at 6.7, 9.3, 14.1, 17.2, 23.5, 27.1, and/or 29.0. In some embodiments, the polymorph exhibits an x-ray powder diffraction pattern having at least six characteristic peaks expressed in degrees two theta (+/−1.0 degree theta) at 6.7, 9.3, 14.1, 17.2, 23.5, 27.1, and/or 29.0. In some embodiments, the polymorph exhibits an x-ray powder diffraction pattern having characteristic peaks expressed in degrees two theta (+/−1.0 degree theta) at 6.7, 9.3, 14.1, 17.2, 23.5, 27.1, and/or 29.0.

In one aspect, provided herein, is polymorph of a compound of Formula (I) having the structure

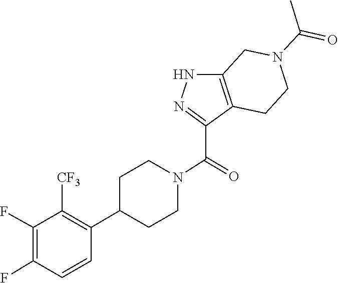 [SMILES: CC(=O)N1CCc2c(C(=O)N3CCC(c4ccc(F)c(F)c4C(F)(F)F)CC3)n[nH]c2C1]

wherein the polymorph exhibits an x-ray powder diffraction pattern having at least three characteristic peaks expressed in degrees two theta (+/−0.2 degree theta) at 6.7, 9.3, 14.1, 17.2, 23.5, 27.1, and/or 29.0. In some embodiments, the polymorph exhibits an x-ray powder diffraction pattern having at least four characteristic peaks expressed in degrees two theta (+/−0.2 degree theta) at 6.7, 9.3, 14.1, 17.2, 23.5, 27.1, and/or 29.0. In some embodiments, the polymorph exhibits an x-ray powder diffraction pattern having at least five characteristic peaks expressed in degrees two theta (+/−0.2 degree theta) at 6.7, 9.3, 14.1, 17.2, 23.5, 27.1, and/or 29.0. In some embodiments, the polymorph exhibits an x-ray powder diffraction pattern having at least six characteristic peaks expressed in degrees two theta (+/−0.2 degree theta) at 6.7, 9.3, 14.1, 17.2, 23.5, 27.1, and/or 29.0. In some embodiments, the polymorph exhibits an x-ray powder diffraction pattern having characteristic peaks expressed in degrees two theta (+/−0.2 degree theta) at 6.7, 9.3, 14.1, 17.2, 23.5, 27.1, and/or 29.0.

In some embodiments, the polymorph exhibits an x-ray powder diffraction pattern comprising characteristic peaks expressed in degrees two theta (+/−0.2 degree theta) at 6.7, 9.3, and 14.1. In some embodiments, the polymorph exhibits an x-ray powder diffraction pattern comprising characteristic peaks expressed in degrees two theta (+/−0.2 degree theta) at 6.7, 9.3, and 17.2. In some embodiments, the polymorph exhibits an x-ray powder diffraction pattern comprising characteristic peaks expressed in degrees two theta (+/−0.2 degree theta) at 6.7, 9.3, and 23.5. In some embodiments, the polymorph exhibits an x-ray powder diffraction pattern comprising characteristic peaks expressed in degrees two theta (+/−0.2 degree theta) at 6.7, 9.3, and 27.1. In some embodiments, the polymorph exhibits an x-ray powder diffraction pattern comprising characteristic peaks expressed in degrees two theta (+/−0.2 degree theta) at 6.7, 9.3, and 29.0.

In some embodiments, the polymorph exhibits an x-ray powder diffraction pattern comprising characteristic peaks expressed in degrees two theta (+/−0.2 degree theta) at 6.7, 14.1, and 17.2. In some embodiments, the polymorph exhibits an x-ray powder diffraction pattern comprising characteristic peaks expressed in degrees two theta (+/−0.2 degree theta) at 6.7, 14.1, and 23.5. In some embodiments, the polymorph exhibits an x-ray powder diffraction pattern comprising characteristic peaks expressed in degrees two theta (+/−0.2 degree theta) at 6.7, 14.1, and 27.1. In some embodiments, the polymorph exhibits an x-ray powder diffraction pattern comprising characteristic peaks expressed in degrees two theta (+/−0.2 degree theta) at 6.7, 14.1, and 29.0.

In some embodiments, the polymorph exhibits an x-ray powder diffraction pattern comprising characteristic peaks expressed in degrees two theta (+/−0.2 degree theta) at 9.3, 14.1, and 17.2. In some embodiments, the polymorph exhibits an x-ray powder diffraction pattern comprising characteristic peaks expressed in degrees two theta (+/−0.2 degree theta) at 9.3, 14.1, and 23.5. In some embodiments, the polymorph exhibits an x-ray powder diffraction pattern comprising characteristic peaks expressed in degrees two theta (+/−0.2 degree theta) at 9.3, 14.1, and 27.1. In some embodiments, the polymorph exhibits an x-ray powder diffraction pattern comprising characteristic peaks expressed in degrees two theta (+/−0.2 degree theta) at 9.3, 14.1, and 29.0.

In some embodiments, the polymorph exhibits an x-ray powder diffraction pattern comprising characteristic peaks expressed in degrees two theta (+/−0.2 degree theta) at 9.3, 14.1, and 17.2. In some embodiments, the polymorph exhibits an x-ray powder diffraction pattern comprising characteristic peaks expressed in degrees two theta (+/−0.2 degree theta) at 9.3, 14.1, and 23.5. In some embodiments, the polymorph exhibits an x-ray powder diffraction pattern comprising characteristic peaks expressed in degrees two theta (+/−0.2 degree theta) at 9.3, 14.1, and 27.1. In some embodiments, the polymorph exhibits an x-ray powder diffraction pattern comprising characteristic peaks expressed in degrees two theta (+/−0.2 degree theta) at 9.3, 14.1, and 29.0.

In some embodiments, the polymorph exhibits an x-ray powder diffraction pattern comprising characteristic peaks expressed in degrees two theta (+/−0.2 degree theta) at 14.1, 17.2, and 23.5. In some embodiments, the XRPD pattern further comprises a peak at 29.0 (+/−0.2 degree theta). In some embodiments, the XRPD pattern further comprises a peak at 27.1 (+/−0.2 degree theta). In some embodiments, the XRPD pattern further comprises a peak at 9.3 (+/−0.2 degree theta). In some embodiments, the XRPD pattern further comprises a peak at 6.7 (+/−0.2 degree theta).

In some embodiments, the polymorph exhibits an x-ray powder diffraction pattern comprising characteristic peaks expressed in degrees two theta (+/−0.2 degree theta) at 9.3, 17.2, 23.5, and 27.1. In some embodiments, the XRPD pattern further comprises a peak at 14.1 (+/−0.2 degree theta). In some embodiments, the XRPD pattern further comprises a peak at 29.0 (+/−0.2 degree theta). In some embodiments, the XRPD pattern further comprises a peak at 6.7 (+/−0.2 degree theta).

In some embodiments, the polymorph exhibits an x-ray powder diffraction pattern comprising characteristic peaks expressed in degrees two theta (+/−0.2 degree theta) at 6.7, 9.3, 17.2, and 23.5. In some embodiments, the XRPD pattern further comprises a peak at 14.1 (+/−0.2 degree theta). In some embodiments, the XRPD pattern further comprises a peak at 29.0 (+/−0.2 degree theta). In some embodiments, the XRPD pattern further comprises a peak at 27.1 (+/−0.2 degree theta).

In some embodiments, the polymorph exhibits an x-ray powder diffraction pattern comprising characteristic peaks expressed in degrees two theta (+/−0.2 degree theta) at 6.7, 9.3, 17.2, 23.5, and 27.1. In some embodiments, the XRPD pattern further comprises a peak at 14.1 (+/−0.2 degree theta). In some embodiments, the XRPD pattern further comprises a peak at 29.0 (+/−0.2 degree theta).

In some embodiments, the polymorph exhibits an x-ray powder diffraction pattern comprising characteristic peaks expressed in degrees two theta (+/−0.5 degree theta) at 6.7, 9.3, 17.2, 23.5, and 27.1. In some embodiments, the XRPD pattern further comprises a peak at 14.1 (+/−0.5 degree theta). In some embodiments, the XRPD pattern further comprises a peak at 29.0 (+/−0.5 degree theta).

In some embodiments, the polymorph exhibits Differential Scanning calorimetry (DSC) of an endotherm at about 228° C.

### Methods of Dosing and Treatment Regimens

The dose of a tablet pharmaceutical composition as described herein including a compound of Formula (I), a pharmaceutically acceptable salt, crystalline, solvate, polymorph, prodrug, metabolite, N-oxide, stereoisomer, or isomer thereof and at least one excipient differ, depending upon the patient's condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome), or a lessening of symptom severity. Optimal doses are generally determined using experimental models and/or clinical trials. The optimal dose depends upon the body mass, weight, or blood volume of the patient.

In one embodiment, a tablet pharmaceutical composition described herein, is used in the preparation of medicaments for the treatment of diseases or conditions in a mammal that would benefit from administration of any one of the tablet pharmaceutical compositions disclosed. Methods for treating any of the diseases or conditions described herein in a mammal in need of such treatment, involves administration of pharmaceutical compositions that include at least one compound described herein or a pharmaceutically acceptable salt, active metabolite, prodrug or pharmaceutically acceptable solvate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, in which the mammal previously experienced at least one symptom of the disease being treated and is currently in remission, a pharmaceutical composition comprising a compound described herein, or a pharmaceutically acceptable salt thereof, in order to prevent a return of the symptoms of the disease or condition.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

In another aspect of the present disclosure, a method for treating an eye disease is disclosed herein. The method includes administering a therapeutically effective amount of a tablet pharmaceutical composition disclosed herein to a subject in need thereof. In certain embodiments, a tablet pharmaceutical composition may be administered orally. In certain embodiments, the tablet pharmaceutical composition may include a compound of Formula (I), a pharmaceutically acceptable salt, crystalline, solvate, polymorph, prodrug, metabolite, N-oxide, stereoisomer, or isomer thereof disclosed herein and at least one excipient.

In another aspect of the present disclosure, a method for lowering the serum concentration of RBP4 in a subject is disclosed herein. The method includes administering to the subject a tablet pharmaceutical composition disclosed herein. In embodiments, the tablet pharmaceutical composition may include a therapeutically effective amount of a compound of Formula (I), or a pharmaceutically acceptable salt, crystalline, solvate, polymorph, prodrug, metabolite, N-oxide, stereoisomer, or isomer thereof and at least one excipient. In certain embodiments, the tablet pharmaceutical composition may include a therapeutically effective amount of a compound having the structure

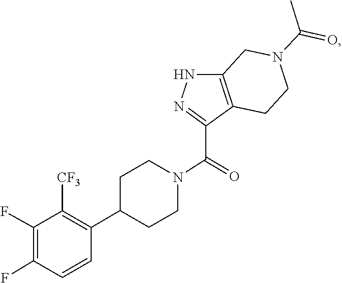 [SMILES: CC(=O)N1CCc2c(C(=O)N3CCC(c4ccc(F)c(F)c4C(F)(F)F)CC3)n[nH]c2C1]

or a pharmaceutically acceptable salt, crystalline, solvate, polymorph, prodrug, metabolite, N-oxide, stereoisomer, or isomer thereof and at least one excipient.

In certain embodiments, the eye disease may be a disease characterized by excessive lipofuscin accumulation in the retina. In certain embodiments, the disease characterized by excessive lipofuscin accumulation may be Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or an ABCA4 gene associated retinal disease.

In certain embodiments, a therapeutically effective amount of a tablet pharmaceutical composition disclosed herein may include about 0.1 mg to about 400 mg of the compound of Formula (I), e.g., about 0.1 mg, 0.2 mg, 0.3 mg, 0.4 mg, 0.5 mg, 0.6 mg, 0.7 mg, 0.8 mg, 0.9 mg 1 mg, 1.1 mg, 1.2 mg, 1.3 mg, 1.4 mg, 1.5 mg, 1.6 mg, 1.7 mg, 1.8 mg, 1.9 mg, 2 mg, 2.1 mg 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg 7 mg 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 102 mg, 104 mg, 106 mg, 108 mg, 110 mg, 112 mg 114 mg, 116 mg, 118 mg, 120 mg, 122 mg, 124 mg, 126 mg, 128 mg, 130 mg, 132 mg, 134 mg 136 mg, 138 mg, 140 mg, 142 mg, 144 mg, 146 mg, 148 mg, 150 mg, 152 mg, 154 mg, 156 mg 158 mg, 160 mg, 162 mg, 164 mg, 166 mg, 168 mg, 170 mg, 172 mg, 174 mg, 176 mg, 178 mg 180 mg, 182 mg, 184 mg, 186 mg, 188 mg, 190 mg, 192 mg, 194 mg, 196 mg, 198 mg, 200 mg 202 mg, 204 mg, 206 mg, 208 mg, 210 mg, 212 mg, 214 mg, 216 mg, 218 mg, 220 mg, 222 mg 224 mg, 226 mg, 228 mg, 230 mg, 232 mg, 234 mg, 236 mg, 238 mg, 240 mg, 242 mg, 244 mg, 246 mg, 248 mg, 250 mg, 252 mg, 254 mg, 256 mg, 258 mg, 260 mg, 262 mg, 264 mg, 266 mg 268 mg, 270 mg, 272 mg, 274 mg, 276 mg, 278 mg, 280 mg, 282 mg, 284 mg, 286 mg, 288 mg 290 mg, 292 mg, 294 mg, 296 mg, 298 mg, 300 mg, 302 mg, 304 mg, 306 mg, 308 mg, 310 mg 312 mg, 314 mg, 316 mg, 318 mg, 320 mg, 322 mg, 324 mg, 326 mg, 328 mg, 330 mg, 332 mg 334 mg, 336 mg, 338 mg, 340 mg, 342 mg, 344 mg, 346 mg, 348 mg, 350 mg, 352 mg, 354 mg 356 mg, 358 mg, 360 mg, 362 mg, 364 mg, 366 mg, 368 mg, 370 mg, 372 mg, 374 mg, 376 mg 378 mg, 380 mg, 382 mg, 384 mg, 386 mg, 388 mg, 390 mg, 392 mg, 394 mg, 396 mg, 398 mg or about 400 mg, or any amount therebetween.

In certain embodiments, a therapeutically effective amount of a tablet pharmaceutical composition disclosed herein may include about 0.1 mg to about 400 mg of the compound of Formula (I), e.g., about 0.1 to about 0.5 mg, about 0.1 to about 1 mg, about 0.1 to about 5 mg, about 0.1 to about 10 mg, about 0.1 to about 25 mg, about 0.1 to about 50 mg, about 0.1 to about 100 mg, about 0.1 to about 200 mg, about 0.1 to about 400 mg, about 0.5 to about 1 mg, about 0.5 to about 5 mg, about 0.5 to about 10 mg, about 0.5 to about 25 mg, about 0.5 to about 50 mg, about 0.5 to about 100 mg, about 0.5 to about 200 mg, about 0.5 to about 400 mg, about 1 to about 5 mg, about 1 to about 10 mg, about 1 to about 25 mg, about 1 to about 50 mg, about 1 to about 100 mg, about 1 to about 200 mg, about 1 to about 400 mg, about 5 to about 10 mg, about 5 to about 25 mg, about 5 to about 50 mg, about 5 to about 100 mg, about 5 to about 200 mg, about 5 to about 400 mg, about 10 to about 25 mg, about 10 to about 50 mg, about 10 to about 100 mg about 10 to about 200 mg, about 10 to about 400 mg, about 25 to about 50 mg, about 25 to about 100 mg, about 25 to about 200 mg, about 25 to about 400 mg, about 50 to about 100 mg, about 50 to about 200 mg, about 50 to about 400 mg, about 100 to about 200 mg, about 100 to about 400 mg, or about 200 to about 400 mg. In embodiments, a therapeutically effective amount of a tablet pharmaceutical composition disclosed herein may include about 0.1 mg, about 0.5 mg, about 1 mg, about 5 mg, about 10 mg, about 25 mg, about 50 mg, about 100 mg, about 200 mg or about 400 mg of the compound of Formula (I).

Oral doses of a tablet pharmaceutical composition may range from about 1.0 mg to about 1000 mg, one to four times, or more, per day. In certain embodiments, a tablet pharmaceutical composition disclosed herein may be administered one, two, three, or four times daily. In certain embodiments, a tablet pharmaceutical composition disclosed herein may be administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months.

In embodiment, administration of a tablet pharmaceutical composition disclosed herein may be one, two, three, four or more times daily for a time course of one day to several days, weeks, months, and even years, and may even be for the life of the patient. In embodiments, illustrative dosing regimens may last a period of at least about 30 mins, one hour, two hours, four hours, 8 hours, 16 hours, a day, a week, from about 1-4 weeks, from about 1-8 weeks, from 1-12 weeks, from 1-16 weeks, from 1-20 weeks, from 1-24 weeks, from 1-36 weeks, from 1-48 weeks, from 1-52 weeks, from 1-60 weeks, from 1-72 weeks, from 1-84 weeks, from 1-96 weeks, from 1 week to 1 year, from 1 week to 2 years, from 1 week to 3 years, from 1 week to 4 years, from 1 week to 5 years, or longer. In embodiments, the doses may be for a period of at least about 1, 2, 4, 6, 8, 12, 24, 36, 48, 60, 72, 84, or 96 weeks. In embodiments, the doses may be for a period of about 12, 24, 36, 48, 60, 72, 84, or 96 weeks. In embodiments, the doses may be for a period of at least about 1 year, 2 years, 3 years, 4 years, or 5 years. In embodiments, the doses may be for a period of about 1 year, 2 years, 3 years, 4 years, or 5 years. In certain embodiments, the serum RBP4 concentration of a subject treated with a method disclosed herein may be reduced to below 1 μM after treatment.

In general, however, doses employed for adult human treatment are typically in the range of 0.01 mg to 5000 mg per day. In certain embodiments, oral doses range from about 0.1 mg to about 20 mg per day. In certain embodiments, oral doses range from about 0.5 mg to about 50 mg per day. In certain embodiments, oral dosages range from about 1 mg to about 10 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg, about 0.5 mg, about 1 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg about 180 mg, about 185 mg, about 190 mg, about 195 mg, about 200 mg, about 225 mg, about 240 mg, about 250 mg, about 275 mg, about 300 mg, about 325 mg, about 350 mg, about 375 mg about 400 mg, about 425 mg, about 450 mg, about 475 mg, or about 500 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg, about 0.5 mg, about 1 mg, about 5 mg, about 10 mg, about 15 mg, or about 20 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to about 0.1 mg, up to about 0.5 mg up to about 1 mg, up to about 5 mg, up to about 10 mg, up to about 15 mg, up to about 20 mg, up to about 25 mg, up to about 30 mg, up to about 35 mg, up to about 40 mg, up to about 45 mg, up to about 50 mg, up to about 55 mg, up to about 60 mg, up to about 65 mg, up to about 70 mg, up to about 75 mg, up to about 80 mg, up to about 85 mg, up to about 90 mg, up to about 95 mg, up to about 100 mg, up to about 105 mg, up to about 110 mg, up to about 115 mg, up to about 120 mg up to about 125 mg, up to about 130 mg, up to about 135 mg, up to about 140 mg, up to about 145 mg, up to about 150 mg, up to about 155 mg, up to about 160 mg, up to about 165 mg, up to about 170 mg, up to about 175 mg, up to about 180 mg, up to about 185 mg, up to about 190 mg, up to about 195 mg, up to about 200 mg, up to about 225 mg, up to about 240 mg, up to about 250 mg up to about 275 mg, up to about 300 mg, up to about 325 mg, up to about 350 mg, up to about 375 mg, up to about 400 mg, up to about 425 mg, up to about 450 mg, up to about 475 mg, or up to about 500 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to about 0.1 mg, up to about 0.5 mg up to about 1 mg, up to about 5 mg, up to about 10 mg, up to about 15 mg, or up to about 20 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least 0.1 mg, least 0.5 mg, least 1 mg, at least 5 mg, at least 10 mg, at least 15 mg, at least 20 mg, at least 25 mg, at least 30 mg, at least 35 mg, at least 40 mg at least 45 mg, at least 50 mg, at least 55 mg, at least 60 mg, at least 65 mg, at least 70 mg, at least 75 mg, at least 80 mg, at least 85 mg, at least 90 mg, at least 95 mg, at least 100 mg, at least 105 mg, at least 110 mg, at least 115 mg, at least 120 mg, at least 125 mg, at least 130 mg, at least 135 mg, at least 140 mg, at least 145 mg, at least 150 mg, at least 155 mg, at least 160 mg, at least 165 mg, at least 170 mg, at least 175 mg, at least 180 mg, at least 185 mg, at least 190 mg, at least 195 mg, at least 200 mg, at least 225 mg, at least 240 mg, at least 250 mg, at least 275 mg, at least 300 mg, at least 325 mg, at least 350 mg, at least 375 mg, at least 400 mg, at least 425 mg, at least 450 mg, at least 475 mg, or at least 500 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least 0.1 mg, at least 0.5 mg, at least 1 mg, at least 5 mg, at least 10 mg, at least 15 mg, at least 20 mg.

In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg, about 0.5 mg, about 1 mg, about 5 mg, about 10 mg, about 25 mg, about 50 mg, about 75 mg, about 100 mg, about 150 mg, about 200 mg, or about 400 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg, about 0.5 mg, about 1 mg, about 5 mg, about 10 mg, about 15 mg, or about 20 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 0.1 mg, up to 0.5 mg, up to 1 mg, up to 5 mg, up to 10 mg, up to 25 mg, up to 50 mg, up to 100 mg or up to 200 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least 0.1 mg, at least 0.5 mg, at least 1 mg, at least 5 mg, at least 10 mg, at least 25 mg, at least 50 mg, at least 100 mg, or at least 200 mg.

In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg per day, about 0.5 mg per day, about 1 mg per day, about 5 mg per day, about 10 mg per day, about 15 mg per day, about 20 mg per day, about 25 mg per day, about 50 mg per day, about 75 mg per day, about 100 mg per day, about 150 mg per day, about 200 mg per day, or about 400 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg per day, about 0.5 mg per day, about 1 mg per day, about 5 mg per day, about 10 mg per day, about 25 mg per day, or about 50 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 0.1 mg per day, up to 0.5 mg per day, up to 1 mg per day, up to 5 mg per day, up to 10 mg per day, up to 25 mg per day, up to 50 mg per day, up to 100 mg per day, or up to 200 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least 0.1 mg per day, at least 0.5 mg per day, at least 1 mg per day, at least 5 mg per day, at least 10 mg per day, at least 25 mg per day, at least 50 mg per day, at least 100 mg per day, or at least 200 mg per day.

In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg to about 20 mg. In some embodiments, a compound of Formula (I), a pharmaceutically acceptable salt, solvate, polymorph, prodrug metabolite, N-oxide, stereoisomer, or isomer thereof is administered to a subject or patient in an amount of about 1 mg to about 10 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg to about 20 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.5 mg to about 50 mg. In some embodiments, a compound of Formula (I), a pharmaceutically acceptable salt, solvate, polymorph, prodrug metabolite, N-oxide, stereoisomer, or isomer thereof is administered to a subject or patient in an amount of about 0.1 mg to about 10 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg to about 100 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg to about 500 mg.

In some embodiments, a compound of Formula (I), a pharmaceutically acceptable salt, solvate, polymorph, prodrug, metabolite, N-oxide, stereoisomer, or isomer thereof is administered to a subject or patient in an amount of up to 400 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 200 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 150 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 100 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 75 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 50 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 25 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 10 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 5 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 1 mg In some embodiments, a compound of Formula (I), a pharmaceutically acceptable salt, solvate, polymorph, prodrug, metabolite, N-oxide, stereoisomer, or isomer thereof is administered to a subject or patient in an amount of up to 0.5 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 0.1 mg.

In some embodiments, a compound of Formula (I), a pharmaceutically acceptable salt, solvate, polymorph, prodrug, metabolite, N-oxide, stereoisomer, or isomer thereof is administered to a subject or patient in an amount of up to 400 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 200 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 150 mg per day. In some embodiments, a compound of Formula (I), a pharmaceutically acceptable salt, solvate, polymorph, prodrug metabolite, N-oxide, stereoisomer, or isomer thereof is administered to a subject or patient in an amount of up to 100 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 75 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 50 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 25 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 10 mg per day. In some embodiments, a compound of Formula (I), a pharmaceutically acceptable salt, solvate, polymorph, prodrug metabolite, N-oxide, stereoisomer, or isomer thereof is administered to a subject or patient in an amount of up to 5 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 1 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 0.5 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of up to 0.1 mg per day.

In some embodiments, a compound of Formula (I), a pharmaceutically acceptable salt, solvate, polymorph, prodrug, metabolite, N-oxide, stereoisomer, or isomer thereof is administered to a subject or patient in an amount of about 400 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 200 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 150 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 100 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 75 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 50 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 25 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 10 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 5 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.5 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg.

In some embodiments, a compound of Formula (I), a pharmaceutically acceptable salt, solvate, polymorph, prodrug, metabolite, N-oxide, stereoisomer, or isomer thereof is administered to a subject or patient in an amount of ab out 400 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 200 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 150 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 100 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 75 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 50 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 25 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 10 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 5 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.5 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg per day.

In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 400 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 200 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 150 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 100 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 75 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 50 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 25 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 10 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 5 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 1 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 0.5 mg. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 0.1 mg.

In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 400 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 200 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 150 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 100 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 75 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 50 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 25 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 10 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 5 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 1 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 0.5 mg per day. In some embodiments, a compound of Formula (I), a pharmaceutically acceptable salt, solvate, polymorph, prodrug, metabolite, N-oxide, stereoisomer, or isomer thereof is administered to a subject or patient in an amount of at least about 0.1 mg per day.

In one embodiment, the daily dosages appropriate for the compound of Formula (I) described herein, or a pharmaceutically acceptable salt thereof, are from about 0.01 to about 50 mg/kg per body weight. In certain embodiments the daily dosages are from about 0.01 to about 25, about 0.01 to about 1, about 0.1 to about 5, about 1 to about 10, about 1 to about 5, about 0.5 to about 5 or about 5 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED50 with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal. In some embodiments, a compound of Formula (I), a pharmaceutically acceptable salt, solvate, polymorph, prodrug, metabolite, N-oxide, stereoisomer, or isomer thereof is administered orally or parenterally to the subject in need thereof. Parenteral administration, as used herein, include intravenous, intraarterial, intraperitoneal, intrathecal, intraventricular, intraurethral, intrasternal, intracranial, intramuscular, intrasynovial, intravesical, and subcutaneous administration. In some embodiments, a compound of Formula (I), a pharmaceutically acceptable salt, solvate, polymorph, prodrug, metabolite, N-oxide, stereoisomer, or isomer thereof is administered orally or intravenously to a subject in need thereof. In some embodiments, a compound of Formula (I), a pharmaceutically acceptable salt, solvate, polymorph, prodrug, metabolite, N-oxide, stereoisomer, or isomer thereof is administered orally to a subject in need thereof. In some embodiments, a compound of Formula (I), a pharmaceutically acceptable salt, solvate, polymorph, prodrug, metabolite, N-oxide, stereoisomer, or isomer thereof is administered intravenously to a subject in need thereof.

Tablet formulations may result in increased systemic exposure of a compound of Formula I). In some instances, the Cmax is at least 20, 30, 40, 50, 60, 70, 80, 90, or at least 100 ng/mL. In some instances, the Cmax is 20-150, 20-200, 20-100, 20-75, 50-300, 50-200, 50-150, 50-250, 75-200, 75-300, 100-200, 100-300, 100-250, 100-150, 150-300, 150-250, or 200-300 ng/mL. In some instances the AUC₀₋ₗₐₛₜ is at least 200, 500, 750, 1000, 1250, 1500, 1750, 2000, 2250, 2500,2750, or at least 3000 ng h/mL. In some instances the AUC₀₋ₗₐₛₜ is 200-3000, 200-2000, 200-1500, 200-1000, 200-750, 200-500, 500-3000, 500-2500, 500-2000, 500-1000, 750-3000, 750-2000, 1000-4000, 1000-5000, 1000-3000, 1000-2500, 1500-3000, 1500-2500, 2000-5000, 2000-3000, or 2000-4000 ng h/mL.

In any of the aforementioned aspects are further embodiments comprising single administrations of the effective amount of the compound, including further embodiments in which (i) the compound is administered once a day; or (ii) the compound is administered to the mammal multiple times over the span of one day, e.g., two, three, four or more times daily. In some embodiments, the compounds of Formula (I) described herein are administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months. In some embodiments, the heterocyclic RBP4 inhibitory compounds described herein, or a pharmaceutically acceptable salt, solvate, polymorph, prodrug, metabolite, N-oxide, stereoisomer, or isomer thereof, are administered daily.

In any of the aforementioned aspects are further embodiments comprising multiple administrations of the effective amount of the compound, including further embodiments in which (i) the compound is administered continuously or intermittently: as in a single dose; (ii) the time between multiple administrations is every 6 hours; (iii) the compound is administered to the mammal every 8 hours; (iv) the compound is administered to the mammal every 12 hours; (v) the compound is administered to the mammal every 24 hours.

In certain embodiments wherein a patient's status does improve, the dose of drug being administered is temporarily reduced or temporarily suspended for a certain length of time (e.g., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In further or alternative embodiments, the method comprises a drug holiday, wherein the administration of the compound is temporarily suspended or the dose of the compound being administered is temporarily reduced; at the end of the drug holiday, dosing of the compound is resumed. In one embodiment, the length of the drug holiday varies from 2 days to 7 days. In one embodiment, the length of the drug holiday is 7 days. In one embodiment, the length of the drug holiday is 14 days. In one embodiment, the length of the drug holiday is 28 days.

In some embodiments, a compound of Formula (I) is administered as a pharmaceutical composition. In any of the aforementioned aspects are further embodiments wherein a compound of Formula (I) is administered to a subject as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg, about 0.5 mg, about 1 mg, about 5 mg, about 10 mg, about 25 mg, about 50 mg, about 75 mg, about 100 mg, about 150 mg about 200 mg, or about 400 mg as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg, about 0.5 mg about 1 mg, about 5 mg, about 10 mg, about 15 mg, or about 20 mg as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of up to 0.1 mg, up to 0.5 mg, up to 1 mg, up to 5 mg, up to 10 mg, up to 25 mg, up to 50 mg, up to 100 mg, or up to 200 mg as part of a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of at least 0.1 mg, at least 0.5 mg, at least 1 mg at least 5 mg, at least 10 mg, at least 25 mg, at least 50 mg, at least 100 mg, or at least 200 mg as a pharmaceutical composition comprising a tablet pharmaceutical composition comprising the compound of Formula (I).

In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg as a pharmaceutical composition comprising a tablet pharmaceutical composition comprising a compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.5 mg as a pharmaceutical composition comprising a tablet pharmaceutical composition comprising a compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg as a pharmaceutical composition comprising a tablet pharmaceutical composition comprising a compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 5 mg as a pharmaceutical composition comprising a tablet pharmaceutical composition comprising a compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 10 mg as a pharmaceutical composition comprising a tablet pharmaceutical composition comprising a compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 15 mg as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 20 mg as a pharmaceutical composition comprising a tablet pharmaceutical composition comprising the compound of Formula (I).

In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg to about 20 mg as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg to about 100 mg as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 1 mg to about 500 mg as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg to about 1000 mg as a pharmaceutical composition comprising a tablet pharmaceutical composition comprising the compound of Formula (I). In some embodiments, a compound of Formula (I), a pharmaceutically acceptable salt, solvate, polymorph, prodrug, metabolite, N-oxide, stereoisomer, or isomer thereof is administered to a subject or patient in an amount of about 0.1 mg to about 20 mg as a pharmaceutical composition comprising a tablet pharmaceutical composition comprising the compound of Formula (I).

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Pharmacokinetics (PK) and Pharmacodynamics (PD) of Compound I in Capsule and Tablet Formulations

This example illustrates the pharmacokinetics (PK) and pharmacodynamics (PD) of Compound 1 in an API-in-Capsule formulation and five types of tablet formulations following a single oral (PO) administration in male cynomolgus monkeys. The concentrations of Compound 1 (test article) and retinol binding protein 4 (RBP4) were monitored in plasma for up to 24 h following each dose.

Table 2 lists the information of the compound of Formula (I) tested.

TABLE 2
Information of the Tested Compound 1

| | | | | | | Stor- |
|---|---|---|---|---|---|---|
| | | | | | | age |
| Test | | | | | | Con- |
| Article | Batch No. | M.W. | F.W. | Formula | Purity | dition |
| Com- | CR- | 456.42 | 456.42 | C₂₁H₂₁F₅N₄O₂ | 97.22% | RT |
| pound | C18031554- | | | | | |
| 1^{a} | F19801M | | | | | |

^{a}For API-in-Capsule formulation, the Compound was hand filled into Size 0. HPMC capsules on site at the testing facility.
RT: room temperature.

TABLE 3
Information of the Tested Compound 1 Tablet
Formulations and the Corresponding Batch No.

| | | Storage |
|---|---|---|
| Tablet Formulations* | Batch No. | Condition |
| 3% CCNa⁽¹⁾ | D-2010FP283501-02 | RT |
| 0.75% CCNa⁽²⁾ | D-2011FP283501-01 | RT |
| 0.75% CCNa + 6% HPC⁽³⁾ | D-2011FP283501-03 | RT |
| 0.75% CCNa + 10% HPMC⁽⁴⁾ | D-2011FP283501-04 | RT |
| 0.75% CCNa + 20% HPMC⁽⁵⁾ | D-2011FP283501-05 | RT |

RT: room temperature; CCNa: croscarmellose sodium; HPC: hydroxypropyl cellulose; HPMC: Hydroxypropylmethylcellulose.
*Besides the ingredients listed in Table 3, The five tablet formulations listed in Table 3 contain the following additional ingredients:
⁽¹⁾45.625% of MCC, 45.625% of LMH, 0.25% of CSD, 0.5% of magnesium stearate, 5% of compound 1;
⁽²⁾46.625% of MCC, 46.625% of LMH, 0.25% of CSD, 0.75% of magnesium stearate, 5% of compound 1;
⁽³⁾43.5% of MCC, 43.5% of LMH, 0.25% of CSD, 1.00% of magnesium stearate, 5% of compound 1;
⁽⁴⁾41.5% of MCC, 41.5% of LMH, 0.25% of CSD, 1.00% of magnesium stearate, 5% of compound 1;
⁽⁵⁾36.5% of MCC, 36.5% of LMH, 0.25% of CSD, 1.00% of magnesium stearate, 5% of compound 1. All the above percentages are based on the weight of the tablet formulation.

The test system and study design were as follows. A total of 4 male non-naïve cynomolgus monkeys were assigned to the study (age: >2 years old and body weight: 2.5-4 kg). Each animal received a single dose of capsule or tablet at the start of each phase and there were 6 phases in total. The same animals, Animal Nos. C1001, C1002, C1003 and C1004, were used for each phase with a minimum 5-day washout period between dosing for each phase, except for Animal No. C1002, Animal Tattoo No. C1708339, which was replaced by another non-naïve cynomolgus monkey, Animal Tattoo No. C1703319, due to dosing failure that happened in Phase 3. As a result, Animal Tattoo No. C1708339 was used in Phase 1 and 2, Animal Tattoo No. C1703319 was used in Phases 3 to 6. All animals were fasted overnight prior to dosing, food was returned at 4 hours post-dose. Details for the study design are shown in Table 4 and PK/PD sample collection timepoints are shown in Table 5.

TABLE 4
Design of the Compound 1 Tablet Formulation PK/PD Study

| Phase | Test | | Dose Level | Dosing | |
|---|---|---|---|---|---|
| No. | Article | Formulation* | (mg/animal) | Route | Remarks |
| P1 | Compound 1 | Micronized API in Size | 5 | PO | 1 capsule/animal |
| | | 0 HPMC Capsule | | | |
| P2 | Compound 1 | 3% CCNa | 5 | PO | 1 tablet/animal |
| P3 | Compound 1 | 0.75% CCNa | 5 | PO | 1 tablet/animal |
| P4 | Compound 1 | 0.75% CCNa + 6% HPC | 5 | PO | 1 tablet/animal |
| P5 | Compound 1 | 0.75% CCNa + 10% HPMC | 5 | PO | 1 tablet/animal |
| P6 | Compound 1 | 0.75% CCNa + 20% HPMC | 5 | PO | 1 tablet/animal |

Note:
4 male non-naïve cynomolgus monkeys were used in each of the 6 phases.
*For the full list of ingredients of the five tablet formulations, please refer to Table 3.

TABLE 5
PK/PD Sample Collection
Timepoints for each Phase of the Study

| | | PK Sampling | | | | | | | | PD Sampling | | |
|---|---|---|---|---|---|---|---|---|---|---|---|---|
| | Number | Timepoints (h) | | | | | | | | Timepoint (h) | | |
| Phase | of | Pre- | | | | | | | | Pre- | | |
| No. | Animals | dose | 0.5 | 1 | 2 | 4 | 8 | 12 | 24 | dose | 8 | 24 |
| P1 | 4 | P | P | P | P | P | P | P | P | Se | Se | Se |
| P2 | 4 | P | P | P | P | P | P | P | P | Se | Se | Se |
| P3 | 4 | P | P | P | P | P | P | P | P | Se | Se | Se |
| P4 | 4 | P | P | P | P | P | P | P | P | Se | Se | Se |
| P5 | 4 | P | P | P | P | P | P | P | P | Se | Se | Se |
| P6 | 4 | P | P | P | P | P | P | P | P | Se | Se | Se |

Note:
4 male non-naive cynomolgus monkeys were used in each of the 6 phases.
P: Plasma;
Se: Serum.

Animals were physically examined prior to study initiation to confirm their health. Clinical observations were performed twice daily (approximately 9:30 a.m. and 4:00 p.m.). Cage-side observations for general health and appearance were also performed. On dosing day, animals were also observed before and after each sample collection time point. General condition, behavior, activity, excretion, respiration or other unusual observations noted throughout the study were recorded.

The route of administration was oral via capsule or tablet. Dose formulations were administered according to the following steps: (1) putting on leather protective gloves and opening the animal's mouth; (2) using a pill gun to place one capsule or tablet beyond the root of the tongue; (3) closing the animal's jaw to assist with swallowing; (4) injecting 10 mL water into the mouth via a syringe; (5) facilitating swallowing by rubbing the animal's neck; and (6) after administering water, check the animal's mouth to ensure the capsule/tablet had been swallowed.

For the PK study, blood was collected from peripheral vessel at the designated sampling time points, as shown in Table 5. Approximately 0.5 mL blood was collected into tubes containing K2-EDTA as an anti-coagulant at each time point. All blood samples were placed on wet ice and processed for plasma. Samples were centrifuged at 3,200×g for 10 min at 2-8° C. within one hour of collection. ˜0.2 mL plasma was transferred into labeled polypropylene micro-centrifuge tubes and stored frozen in a freezer (−60° C. or lower) until the liquid chromatography/tandem mass spectrometry (LC-MS/MS) analysis

For PD study, blood was collected from peripheral vessel at the designed sampling time point as shown in Table 5. Approximately 1 mL blood was collected into a serum separator tube and allowed to clot for 30 mins. Samples were centrifuged at 1000×g for 15 mins. Duplicates of ˜0.2 mL serum were transferred into labeled polypropylene micro-centrifuge tubes and stored frozen in a freezer (−60° C. or lower) until PD analysis.

For PK data analysis, individual plasma concentration data of the Compound 1 was subjected to non-compartmental pharmacokinetic analysis using Phoenix WinNonlin™ (version 6.3, Pharsight, Mountain View, CA). Peak plasma concentrations (Cm,) and the corresponding peak times (Tₘₐₓ) were taken directly from the plasma concentration/time profiles for each phase. Terminal half-life (T_{1/2}), mean residence time (MRT) from time zero to the last quantifiable concentration (MRT₀₋ₗₐₛₜ) and from time zero to infinity (MRT_{0-inf}), the area under the plasma concentration-time curve (AUC) from time zero to the last quantifiable concentration (AUC₀₋ₗₐₛₜ) and AUC from time zero extrapolated to infinity (AUC_{0-inf}) were calculated using the linear/log trapezoidal rule. All the values were reported to three significant figures and mean to three significant figures. Nominal sampling times were used to calculate all pharmacokinetic parameters, since in no situation was there a deviation larger than 5% between the actual and nominal sampling times.

For PD data analysis, individual serum retinol binding protein 4 (RBP4) concentration was obtained and the mean values were calculated for each phase. To elucidate the pharmacological effects between the different formulations, the level of RBP4 reduction from baseline (i.e., pre-dose level) was calculated. To account for the different baseline levels, all data were normalized and represented as percentage (%) RBP4 reduction from baseline, mean value was also calculated for each phase.

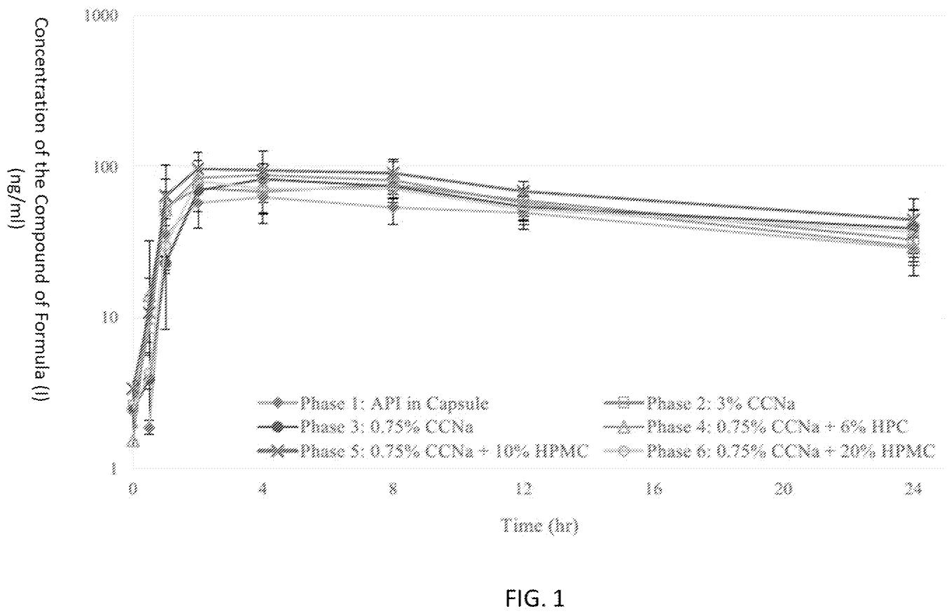

All animals had tolerated the test Compound 1 in the in-life study. No adverse effects were observed during the in-life phase of the study. In regard to PK in monkeys, following oral administration of the Compound 1 in different tablet formulations or in capsule to male cynomolgus monkey, mean plasma concentrations of the Compound 1 for all six phases were calculated and listed in Table 6. The selected pharmacokinetic parameters of the six phases are shown in Table 7. The mean plasma concentration of the Compound 1 vs. time pharmacokinetic profiles for each of the tablet formulations in comparison to API-in-capsule are illustrated in FIG. 1.

TABLE 6
Mean Plasma Concentration of the Compound 1 (ng/mL) for Phases 1-6 at
Designated PK Sampling Timepoints

| | | | | | | | Phase 4 | | Phase 5 | | Phase 6 | |
|---|---|---|---|---|---|---|---|---|---|---|---|---|
| For- | | | | | | | Tablet | | Tablet | | Tablet | |
| mu- | Phase 1 | | Phase 2 | | Phase 3 | | 0.75% | | 0.75% | | 0.75% | |
| lation | API-in- | | Tablet 3% | | Tablet 0.75% | | CCNa + | | CCNa + | | CCNa + | |
| Time | Capsule | | CCNa | | CCNa | | 6% HPC | | 10% HPMC | | 20% HPMC | |
| (h) | Mean | SD | Mean | SD | Mean | SD | Mean | SD | Mean | SD | Mean | SD |
| 0.00 | BQL | ND | 2.64 | 0.750 | 2.46 | 0.535 | 1.53 | ND | 3.38 | 0.431 | 2.48 | 1.07 |
| 0.500 | 1.86 | ND | 8.50 | 2.71 | 3.89 | 1.79 | 14.0 | 18.3 | 10.7 | 7.38 | 4.28 | 2.60 |
| 1.00 | 35.5 | 10.0 | 54.5 | 13.5 | 22.9 | 14.5 | 51.9 | 312 | 63.8 | 38.6 | 30.0 | 10.5 |
| 2.00 | 57.5 | 18.2 | 71.8 | 21.9 | 69.4 | 10.8 | 84.4 | 25.0 | 96.7 | 28.0 | 80.3 | 14.0 |
| 4.00 | 62.7 | 20.3 | 68.3 | 19.6 | 82.7 | 21.5 | 87.9 | 38.3 | 94.6 | 31.6 | 71.1 | 12.6 |
| 8.00 | 53.5 | 12.4 | 75.5 | 14.0 | 74.1 | 15.7 | 81.2 | 26.7 | 90.6 | 21.3 | 71.0 | 8.94 |
| 12.0 | 49.4 | 10.7 | 59.7 | 12.5 | 54.2 | 9.74 | 57.4 | 14.1 | 68.6 | 11.1 | 52.5 | 11.1 |
| 24.0 | 28.8 | 5.25 | 32.8 | 7.80 | 39.0 | 12.4 | 29.5 | 10.8 | 44.4 | 16.7 | 37.0 | 14.8 |

BQL: Below the Lower Limit of Quantitation
ND: not determined

TABLE 7
Calculated PK Parameters for each of the Compound 1
Tablet/Capsule Formulations (Phase 1-6)
Phase No./Formulation

| | | | | | Phase | Phase |
|---|---|---|---|---|---|---|
| | | | | | 5 | 6 |
| | Phase | Phase | Phase | Phase 4 | Tablet | Tablet |
| | 1 | 2 | 3 | Tablet | 0.75% | 0.75% |
| | API in | Tablet | Tablet | 0.75% | CCNa + | CCNa + |
| PK | Cap- | 3% | 0.75% | CCNa + | 10% | 20% |
| Parameters | sule | CCNa | CCNa | 6% HPC | HPMC | HPMC |
| Rsq_adj | — | — | — | — | — | — |
| No. points | ND | 3.00 | ND | ND | ND | ND |
| used for T_{1/2} | | | | | | |
| Cmax (ng/ml) | 66.2 | 83.5 | 92.9 | 108 | 116 | 82.4 |
| Tₘₐₓ (h) | 4.00 | 6.50 | 4.50 | 3.75 | 3.75 | 5.00 |
| T_{1/2} (h) | 26.6 | 13.6 | 35.4 | 18.4 | 20.5 | 14.8 |
| Tₗₐₛₜ (h) | 24.0 | 24.0 | 24.0 | 24.0 | 24.0 | 24.0 |
| AUC₀₋ₗₐₛₜ | 1074 | 1315 | 1325 | 1368 | 1638 | 1272 |
| (ng · h/mL) | | | | | | |
| AUC_{0-inf} | 2228 | 1967 | 3690 | 2271 | 3193 | 1926 |
| (ng · h/mL) | | | | | | |
| MRT₀₋ₗₐₛₜ (h) | 11.1 | 10.9 | 11.0 | 10.2 | 10.8 | 10.9 |
| MRT_{0-inf} (h) | 39.3 | 21.6 | 52.1 | 27.4 | 31.4 | 22.6 |
| AUC_{Extra} (%) | 48.1 | 32.5 | 51.1 | 33.3 | 40.2 | 33.0 |
| AUMC_{Extra} (%) | 79.0 | 65.4 | 79.3 | 61.7 | 69.9 | 65.4 |

ND: not determined

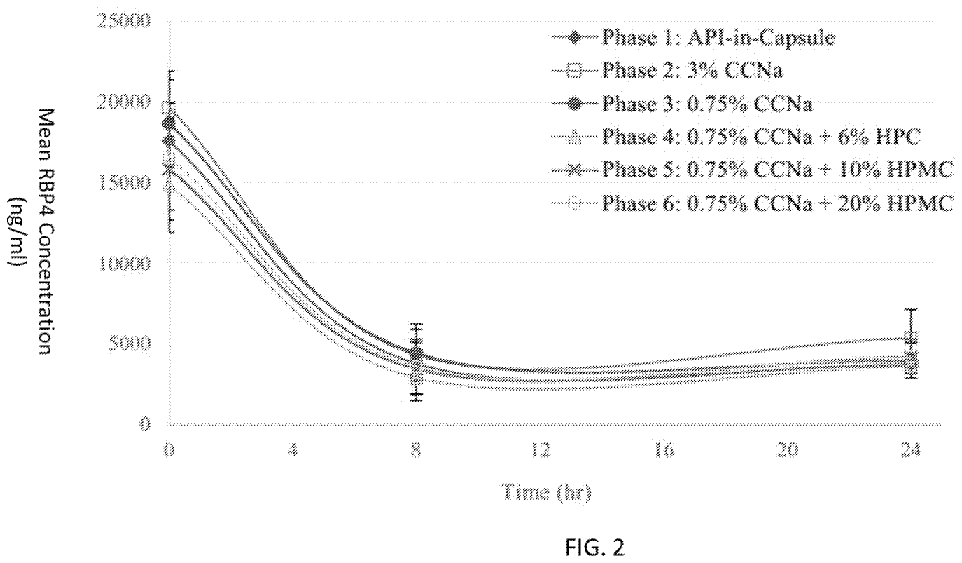

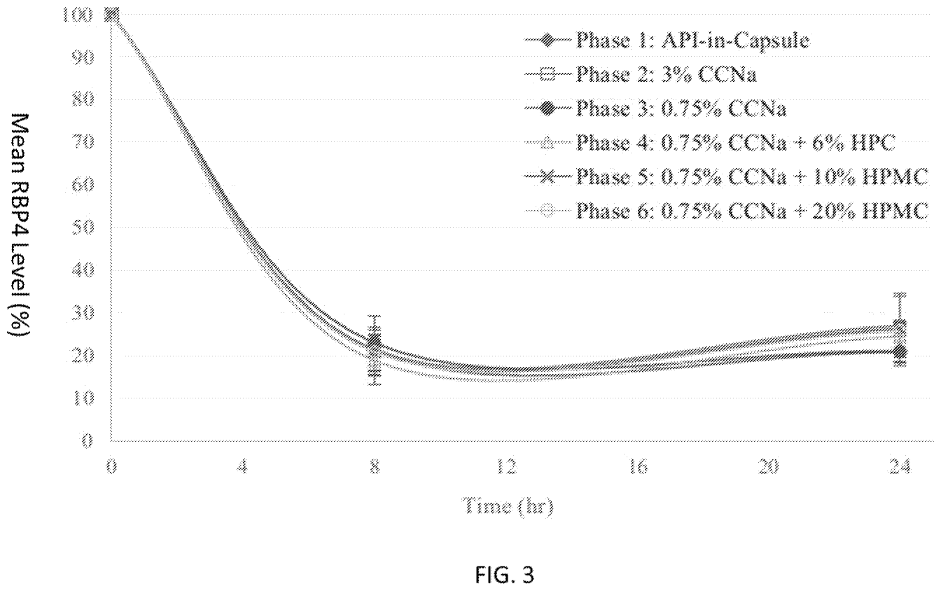

The pharmacodynamics (PK) of the Compound 1 were also determined for the different formulations studied. The mean concentration of the biomarker RBP4 at predose and 8- and 24-hours post-dose across the six phases were determined. Results are summarized in Table 8 and the mean RBP4 concentration vs. time pharmacodynamic profile for each of the tablet formulation in comparison to API-in-capsule is illustrated in FIG. 2. The mean percentage of RBP4 inhibition across the different phases was calculated. Results are shown in Table 9 and the RBP4 reduction profile up to 24 hours post-dose is illustrated in FIG. 3.

TABLE 8
Mean Serum RBP4 Concentration (ng/mL) for Phase 1-6 at
Designated PD Sampling Timepoints

| | | | | | | | Phase 4 | | Phase 5 | | Phase 6 | |
|---|---|---|---|---|---|---|---|---|---|---|---|---|
| For- | | | | | Phase 3 | | Tablet | | Tablet | | Tablet | |
| mu- | Phase 1 | | Phase 2 | | Tablet | | 0.75% | | 0.75% | | 0.75% | |
| lation | API-in- | | Tablet 3% | | 0.75% | | CCNa + | | CCNa + | | CCNa + | |
| Time | Capsule | | CCNa | | CCNa | | 6% HPC | | 10% HPMC | | 20% HPMC | |
| (h) | Mean | SD | Mean | SD | Mean | SD | Mean | SD | Mean | SD | Mean | SD |
| 0.00 | 17600 | 1639 | 19650 | 2317 | 18675 | 2699 | 14850 | 3001 | 15850 | 3154 | 16600 | 3314 |
| 8.00 | 3760 | 841 | 4288 | 1561 | 4410 | 1841 | 2890 | 1426 | 3428 | 1644 | 3583 | 1661 |
| 24.0 | 3695 | 775 | 5358 | 1760 | 3903 | 441 | 3618 | 693 | 4195 | 1085 | 4148 | 936 |

Lower Limit of Quantitation (LLOQ): 1.56 ng/ml;
Higher Limit of Quantitation (HLOQ): 100 ng/ml;
Minimum Required Dilution (MRD): 1000-fold.

TABLE 9
Mean Serum RBP4 Reduction (%) from Baseline for each of the Compound 1
Tablet/Capsule Formulations (Phase 1-6) at Designated PD Sampling Timepoints

| | | | | | | | Phase 4 | | Phase 5 | | Phase 6 | |
|---|---|---|---|---|---|---|---|---|---|---|---|---|
| For- | | | Phase 2 | | Phase 3 | | Tablet | | Tablet | | Tablet | |
| mu- | Phase 1 | | Tablet | | Tablet | | 0.75% | | 0.75% | | 0.75% | |
| lation | API-in- | | 3% | | 0.75% | | CCNa + | | CCNa + | | CCNa + | |
| Time | Capsule | | CCNa | | CCNa | | 6% HPC | | 10% HPMC | | 20% HPMC | |
| (h) | Mean | SD | Mean | SD | Mean | SD | Mean | SD | Mean | SD | Mean | SD |
| 0.00 | Base- | N/A | Base- | N/A | Base- | N/A | Base- | N/A | Base- | N/A | Base- | N/A |
| | line^{a} | | line^{a} | | line^{a} | | line^{a} | | line^{a} | | line^{a} | |
| 8.00 | 78.76 | 3.67 | 78.57 | 5.13 | 77.01 | 6.28 | 81.17 | 5.69 | 79.08 | 5.58 | 79.18 | 5.27 |
| 24.0 | 79.16 | 2.54 | 72.99 | 7.48 | 78.95 | 2.32 | 75.49 | 3.26 | 73.73 | 1.71 | 74.28 | 8.14 |

^{a}Baseline is defined as 100% compare for each formulation.

This example demonstrates the PK and PD of Compound 1 in API-in-Capsule formulation and in the five types of tablet formulations following a single oral administration in male cynomolgus monkeys. Five tablet formulations were investigated and compared to API-in-Capsule formulation in a total of 6-phase study. The PK results indicated no significant differences in systemic exposure of Compound 1 under the different formulations (i.e., at different phases).

Although there were no significant changes between the different formulations, the API-in-Capsule formulation (phase 1) exhibited the lowest systemic exposure (Cmax: 66.2±16.4 ng/mL and AUC₀₋ₗₐₛₜ: 1074±227 ng h/mL). Phases 2, 3 and 6, representing tablet formulations 3% CCNa, 0.75% CCNa and 0.75% CCNa+20% HPMC respectively, showed higher systemic exposure compared to phase 1 but lower compared to phases 4 (0.75% CCNa+6% HPC) and 5 (0.75% CCNa+10% HPMC). Phase 4 demonstrated comparable Cₘₐₓ to phase 5 and a slightly lower exposure level (AUC₀₋ₗₐₛₜ) without any significant difference. The AUC level for phase 4 was comparable to phases 2, 3 and 6 (AUC₀₋ₗₐₛₜ: 1368±330 ng h/mL vs. 1315±238, 1325±218 and 1272±198 ng h/mL).

The PD of Compound 1 in different tablet formulations were also evaluated by determining the serum concentration of RBP4 (ng/mL) at predose and 8- and 24-hours post-dose for all phases. To elucidate the pharmacological effects between the different tablet formulations, the mean level of RBP4 reduction was calculated and compared with that of API-in-Capsule formulation. To account for different baseline serum RBP4 concentrations, all data were normalized and were calculated as percentage (%) RBP4 reduction from baseline. Although Phase 1 API-in-Capsule indicated the highest % serum RBP4 reduction at 24 hours post-dose (79.16±2.54%) and Phase 2 showed the lowest % serum RBP4 reduction (72.99±7.48%), all five tablet formulation phases showed comparable RBP4 reduction profile to that of API-in-Capsule formulation. No significant differences were observed across the six phases studied.

### Example 2: Dissolution Data of GMP Batch of Tablets

This example illustrates the dissolution profile of the GMP batch of tablet pharmaceutical compositions containing Compound 1. This batch of tablets was prepared according to methods disclosed herein and contain the following ingredients: 5% Compound 1, 43.25% MCC, 43.5% LMH, 6% HPC, 0.75 CCNa, 0.5% CSD and 1.00% magnesium stearate, by weight of the tablet formulation. These tablets were assessed for their dissolution profile at 5 min, 10 min, 15 min, 30 min, 45 min, 60 min and 90 min time points when tested±5% (Table 10). The dissolution test of Table 11 was conducted under the long term condition (1 month) at the temperature of 25±2° C. and relative humidity of 60±5%. The dissolution test of Table 12 was conducted under accelerated storage condition (1 month) at the temperature of 40±2° C. and relative humidity of 75±5%.

TABLE 10
Dissolution Profile of Tablets (25 ± 2° C./60 ± 5%/initial/dissolution (HPLC))

| Time | | | | | | Vessel | Vessel | Vessel | Vessel | Vessel | Vessel |
|---|---|---|---|---|---|---|---|---|---|---|---|
| Point | Max | Mean | Min | RSD | SD | 1 | 2 | 3 | 4 | 5 | 6 |
| 05 Min | 51% | 46% | 40% | 9% | 4% | 47% | 40% | 43% | 46% | 51% | 50% |
| 10 Min | 72% | 65% | 61% | 6% | 4% | 67% | 61% | 61% | 64% | 67% | 72% |
| 15 Min | 82% | 75% | 70% | 6% | 4% | 75% | 72% | 70% | 74% | 76% | 82% |
| 30 Min | 93% | 87% | 82% | 4% | 4% | 86% | 84% | 82% | 86% | 89% | 93% |
| 45 Min | 96% | 92% | 88% | 4% | 3% | 91% | 90% | 88% | 92% | 96% | 96% |
| 60 Min | 98% | 95% | 91% | 3% | 3% | 94% | 94% | 91% | 95% | 98% | 98% |
| 90 Min | 100% | 98% | 97% | 1% | 1% | 98% | 98% | 98% | 97% | 100% | 99% |

RSD: relative standard deviation;
SD: standard deviation

TABLE 11
Dissolution Profile of Tablets (25 ± 2° C./60 ± 5%/1 M/dissolution (HPLC))

| Time | | | | | | Vessel | Vessel | Vessel | Vessel | Vessel | Vessel |
|---|---|---|---|---|---|---|---|---|---|---|---|
| Point | Max | Mean | Min | RSD | SD | 1 | 2 | 3 | 4 | 5 | 6 |
| 05 Min | 60% | 53% | 49% | 7% | 4% | 51% | 60% | 49% | 52% | 53% | 54% |
| 10 Min | 81% | 75% | 72% | 4% | 3% | 74% | 81% | 73% | 76% | 72% | 74% |
| 15 Min | 89% | 84% | 82% | 4% | 3% | 82% | 89% | 85% | 86% | 82% | 83% |
| 30 Min | 97% | 93% | 91% | 3% | 2% | 92% | 97% | 95% | 94% | 91% | 91% |
| 45 Min | 99% | 97% | 95% | 2% | 2% | 97% | 99% | 99% | 97% | 96% | 95% |
| 60 Min | 101% | 99% | 98% | 1% | 1% | 100% | 100% | 101% | 99% | 98% | 98% |
| 90 Min | 102% | 101% | 99% | 1% | 1% | 100% | 100% | 102% | 99% | 99% | 101% |

RSD: relative standard deviation;
SD: standard deviation

TABLE 12
Dissolution Profile of Tablets (40 ± 2° C./75 ± 5%/1 M/dissolution (HPLC))

| Time | | | | | | Vessel | Vessel | Vessel | Vessel | Vessel | Vessel |
|---|---|---|---|---|---|---|---|---|---|---|---|
| Point | Max | Mean | Min | RSD | SD | 1 | 2 | 3 | 4 | 5 | 6 |
| 05 Min | 61% | 55% | 50% | 7% | 4% | 51% | 56% | 61% | 54% | 55% | 50% |
| 10 Min | 80% | 77% | 73% | 4% | 3% | 74% | 79% | 80% | 77% | 78% | 73% |
| 15 Min | 87% | 85% | 83% | 2% | 2% | 83% | 87% | 87% | 85% | 86% | 83% |
| 30 Min | 96% | 94% | 92% | 1% | 1% | 93% | 96% | 95% | 94% | 95% | 92% |
| 45 Min | 99% | 97% | 96% | 1% | 1% | 96% | 99% | 98% | 98% | 98% | 96% |
| 60 Min | 101% | 99% | 98% | 1% | 1% | 98% | 101% | 99% | 99% | 99% | 98% |
| 90 Min | 103% | 100% | 99% | 1% | 1% | 99% | 103% | 100% | 101% | 100% | 100% |

RSD: relative standard deviation;
SD: standard deviation

### Example 3: Dissolution Data of Non-GMP Batch of Tablets

This example illustrates the dissolution profile of non-GMP batch of tablet pharmaceutical compositions comprising Compound 1. This batch of tablets was prepared according to methods disclosed herein containing the same ingredients as the tablets in Example 2 and assessed for their dissolution profile at 5 min, 10 min, 15 min, 30 min, 45 min, 60 min and 90 min time points when tested in USP<711> type II apparatus (paddle) at 60 rpm in 900 ml sodium acetate (NaAc) buffer solution containing 0.2500 Sodium dodecyl sulfate SDS with a pH of 4.5. The tests were conducted at 25±2° C. with a relative humidity of 60±5% for the tablets in Tables 14, 15, 17 and 18, and at 40±2° C. with a relative humidity of 75±5% for the tablets in Tables 16 and 19. Tablets of Tables 14 and 17 were tested with desiccant and tablets of Tables 15, 1,1 and 19 were tested without desiccant. The tests were conducted with desiccant for tablets in Tables 14 and 17 and without desiccant for tablets in Tables 15, 16,18 and 19. Data for percent of tablet release at each time point (e.g., 1M=1 month, 3M=3 months, etc.) are shown in Tables 13-19.

TABLE 13
Dissolution Profile of Tablets

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 53 | 75 | 85 | 93 | 97 | 99 | 100 | 101.08 |
| 2 | 57 | 76 | 85 | 95 | 99 | 100 | 100 | 102.05 |
| 3 | 55 | 71 | 80 | 91 | 97 | 99 | 100 | 100.11 |
| 4 | 48 | 68 | 77 | 87 | 93 | 96 | 101 | 102.42 |
| 5 | 57 | 76 | 84 | 94 | 98 | 99 | 100 | 102.41 |
| 6 | 55 | 79 | 88 | 95 | 98 | 98 | 99 | 100.82 |
| Mean % | 54 | 74 | 83 | 93 | 97 | 99 | 100 | N/A |
| Max. % | 57 | 79 | 88 | 95 | 99 | 100 | 101 | |
| Min. % | 48 | 68 | 77 | 87 | 93 | 96 | 99 | |
| SD % | 3 | 4 | 4 | 3 | 2 | 1 | 1 | |
| RSD % | 6 | 5 | 5 | 3 | 2 | 1 | 1 | |

RSD: relative standard deviation;
SD: standard deviation

TABLE 14
Dissolution Profile of Tablets
(1 M 25 ± 2° C./60 ± 5% RH (with desiccant)

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 60 | 79 | 87 | 93 | 96 | 98 | 99 | 101.20 |
| 2 | 47 | 67 | 77 | 86 | 91 | 94 | 100 | 100.94 |
| 3 | 60 | 79 | 87 | 95 | 98 | 100 | 101 | 101.17 |
| 4 | 53 | 71 | 79 | 87 | 90 | 92 | 98 | 99.39 |
| 5 | 53 | 71 | 79 | 88 | 92 | 94 | 97 | 99.57 |
| 6 | 63 | 85 | 91 | 96 | 97 | 97 | 98 | 99.21 |
| Mean % | 56 | 75 | 83 | 91 | 94 | 96 | 99 | N/A |
| Max. % | 63 | 85 | 91 | 96 | 98 | 100 | 101 | |
| Min. % | 47 | 67 | 77 | 86 | 90 | 92 | 97 | |
| SD % | 6 | 7 | 6 | 4 | 3 | 3 | 2 | |
| RSD % | 10 | 9 | 7 | 5 | 4 | 3 | 2 | |

RSD: relative standard deviation;
SD: standard deviation

TABLE 15
Dissolution Profile of Tablets
(1 M 25 ± 2° C./60 ± 5% RH (without desiccant)

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 55 | 77 | 86 | 93 | 96 | 98 | 98 | 101/44 |
| 2 | 52 | 72 | 79 | 88 | 91 | 94 | 98 | 100.76 |
| 3 | 56 | 78 | 86 | 96 | 99 | 99 | 100 | 102.12 |
| 4 | 56 | 76 | 82 | 91 | 95 | 97 | 99 | 103.07 |
| 5 | 55 | 72 | 78 | 88 | 93 | 95 | 97 | 101.54 |
| 6 | 48 | 79 | 89 | 97 | 100 | 100 | 101 | 104.30 |
| Mean % | 54 | 75 | 83 | 92 | 96 | 97 | 99 | N/A |
| Max. % | 56 | 79 | 89 | 97 | 100 | 100 | 101 | |
| Min. % | 48 | 72 | 78 | 88 | 91 | 94 | 97 | |
| SD % | 3 | 3 | 4 | 4 | 3 | 2 | 1 | |
| RSD % | 6 | 4 | 5 | 4 | 3 | 2 | 1 | |

RSD: relative standard deviation;
SD: standard deviation

TABLE 16
Dissolution Profile of Tablets (1M 40 ± 2° C./
75 ± 5% RH (without desiccant))

| | | | | | | | 90 min | |
|---|---|---|---|---|---|---|---|---|
| Tablets | | | | | | | (Infinity, | Tablet |
| No.\ | 5 | 10 | 15 | 30 | 45 | 60 | 250 rpm/ | Weight |
| % Release | min | min | min | min | min | min | 30 min) | (mg) |
| 1 | 52 | 72 | 80 | 90 | 97 | 99 | 100 | 105.59 |
| 2 | 51 | 76 | 86 | 95 | 98 | 98 | 99 | 102.81 |
| 3 | 49 | 74 | 83 | 93 | 96 | 97 | 98 | 101.20 |
| 4 | 56 | 80 | 88 | 96 | 98 | 99 | 99 | 102.95 |
| 5 | 47 | 67 | 77 | 88 | 94 | 97 | 99 | 102.77 |
| 6 | 54 | 79 | 89 | 97 | 99 | 100 | 100 | 103.54 |
| Mean % | 51 | 74 | 84 | 93 | 97 | 98 | 99 | N/A |
| Max. % | 56 | 80 | 89 | 97 | 99 | 100 | 100 | |
| Min. % | 47 | 67 | 77 | 88 | 94 | 97 | 98 | |
| SD % | 3 | 5 | 5 | 4 | 2 | 1 | 1 | |
| RSD % | 6 | 6 | 6 | 4 | 2 | 1 | 1 | |

RSD: relative standard deviation; SD: standard deviation

TABLE 17
Dissolution Profile of Tablets (3M 25 ±
2° C./60 ± 5% RH (with desiccant))

| | | | | | | | 90 min | |
|---|---|---|---|---|---|---|---|---|
| Tablets | | | | | | | (Infinity, | Tablet |
| No.\ | 5 | 10 | 15 | 30 | 45 | 60 | 250 rpm/ | Weight |
| % Release | min | min | min | min | min | min | 30 min) | (mg) |
| 1 | 54 | 72 | 80 | 87 | 90 | 92 | 99 | 102.63 |
| 2 | 54 | 72 | 79 | 86 | 91 | 93 | 98 | 103.02 |
| 3 | 57 | 75 | 82 | 91 | 94 | 96 | 98 | 102.38 |
| 4 | 55 | 73 | 80 | 87 | 90 | 92 | 94 | 98.13 |
| 5 | 53 | 70 | 77 | 85 | 89 | 91 | 97 | 102.05 |
| 6 | 47 | 73 | 83 | 92 | 96 | 98 | 99 | 103.59 |
| Mean % | 53 | 72 | 80 | 88 | 92 | 94 | 98 | N/A |
| Max. % | 57 | 75 | 83 | 92 | 96 | 98 | 99 | |
| Min. % | 47 | 70 | 77 | 85 | 89 | 91 | 94 | |
| SD % | 3 | 2 | 2 | 3 | 3 | 3 | 2 | |
| RSD % | 6 | 2 | 3 | 3 | 3 | 3 | 2 | |

RSD: relative standard deviation; SD: standard deviation

TABLE 18
Dissolution Profile of Tablets (3M 25 ± 2° C./
60 ± 5% RH (without desiccant))

| | | | | | | | 90 min | |
|---|---|---|---|---|---|---|---|---|
| Tablets | | | | | | | (Infinity, | Tablet |
| No.\ | 5 | 10 | 15 | 30 | 45 | 60 | 250 rpm/ | Weight |
| % Release | min | min | min | min | min | min | 30 min) | (mg) |
| 1 | 53 | 76 | 86 | 95 | 98 | 98 | 99 | 103.21 |
| 2 | 48 | 64 | 71 | 80 | 85 | 88 | 94 | 101.50 |
| 3 | 53 | 70 | 78 | 90 | 97 | 99 | 99 | 104.14 |
| 4 | 44 | 64 | 74 | 85 | 90 | 92 | 95 | 102.89 |
| 5 | 47 | 63 | 71 | 84 | 91 | 95 | 97 | 102.46 |
| 6 | 52 | 73 | 83 | 91 | 95 | 98 | 99 | 102.42 |
| Mean % | 49 | 68 | 77 | 88 | 92 | 95 | 97 | N/A |
| Max. % | 53 | 76 | 86 | 95 | 98 | 99 | 99 | |
| Min. % | 44 | 63 | 71 | 80 | 85 | 88 | 94 | |
| SD % | 3 | 6 | 6 | 6 | 5 | 4 | 2 | |
| RSD % | 7 | 8 | 8 | 6 | 5 | 4 | 2 | |

RSD: relative standard deviation; SD: standard deviation

TABLE 19
Dissolution Profile of Tablets (3M 40 ± 2° C./
75 ± 5% RH (without desiccant))

| | | | | | | | 90 min | |
|---|---|---|---|---|---|---|---|---|
| Tablets | | | | | | | (Infinity, | Tablet |
| No.\ | 5 | 10 | 15 | 30 | 45 | 60 | 250 rpm/ | Weight |
| % Release | min | min | min | min | min | min | 30 min) | (mg) |
| 1 | 47 | 71 | 84 | 92 | 94 | 96 | 97 | 102.67 |
| 2 | 56 | 74 | 84 | 94 | 97 | 97 | 97 | 102.90 |
| 3 | 45 | 64 | 74 | 86 | 92 | 94 | 96 | 100.76 |
| 4 | 48 | 67 | 77 | 87 | 93 | 95 | 97 | 101.53 |
| 5 | 45 | 65 | 77 | 89 | 96 | 100 | 103 | 103.61 |
| 6 | 56 | 79 | 88 | 95 | 97 | 98 | 99 | 104.64 |
| Mean % | 49 | 70 | 80 | 90 | 95 | 97 | 98 | N/A |
| Max. % | 56 | 79 | 88 | 95 | 97 | 100 | 103 | |
| Min. % | 45 | 64 | 74 | 86 | 92 | 94 | 96 | |
| SD % | 5 | 6 | 5 | 4 | 2 | 2 | 2 | |
| RSD % | 11 | 8 | 7 | 4 | 2 | 2 | 2 | |

RSD: relative standard deviation; SD: standard deviation

### Example 4: Stability Data of Capsules and Tablets

This example illustrates the chemical stability of capsules comprising Compound 1 and tablets comprising Compound 1. Tables 20 to 25 are test results of capsules and Tables 26 to 29 are test results of tablets.

TABLE 20
Results from the In-Use Stability Study of 25 mg Capsules

| | | | Storage | | |
|---|---|---|---|---|---|
| Attribute | Specification | Initial | Condition | 2 Weeks | 4 Weeks |
| Appearance | White capsules | Conforms | 25° C./60% RH | Conforms | Conforms |
| (TM.795) | free from | | 40° C./75% RH | Conforms | Conforms |
| | physical defects | | | | |
| | containing an | | | | |
| | off-white to | | | | |
| | white powder | | | | |
| Assay by UPLC | 90.0-110.0% | 96.5% | 25° C./60% RH | 96.4% | 96.2% |
| (TM.05064) | | | 40° C./75% RH | 96.0% | 95.7% |
| Purity by UPLC | ≥95.0% | 97.1% | 25° C./60% RH | 97.1% | 97.1% |
| (TM.05064) | | | 40° C./75% RH | 97.1% | 97.1% |
| Total Impurities | ≤5.0% | 2.8% | 25° C./60% RH | 2.8% | 2.8% |
| (TM.05064) | | | 40° C./75% RH | 2.8% | 2.8% |
| Disintegration | Report results | 3.56, 4.36, 6.44, | 25° C./60% RH | 2.57, 3.09, 3.26, | 4.21, 4.05, 3.36, |
| (TM.05056) | (min) | 4.11, 10.05, 7.04 | 40° C./75% RH | 3.49, 3.38, 3.58 | 4.44, 3.41, 3.58 |
| | Mean (n = 6) | Mean: 6.06 min | | Mean: 3.30 min | Mean: 4.04 min |
| | | | | 3.12, 4.11, 4.12, | 3.51, 4.21, 4.19, |
| | | | | 3.15, 3.18, 4.01 | 4.01, 4.38, 4.26 |
| | | | | Mean: 3.42 min | Mean: 4.16 min |
| Water Content | ≤5.0% | 0.19% | 25° C./60% RH | 0.13% | 0.13% |
| (Karl Fischer) | | | 40° C./75% RH | 0.13% | 0.12% |
| XRPD | Crystalline | Conforms | 25° C./60% RH | Conforms | Conforms |
| (TM.60) | Form C | | 40° C./75% RH | Conforms | Conforms |

RH = relative humidity; TM = test method; UPLC = ultra-performance liquid chromatography; XRPD = x-ray powder diffraction.

TABLE 21
Results for Impurities from the In-Use Stability Study of the 25 mg Capsules

| | | | | 2 Weeks | | 4 Weeks | |
|---|---|---|---|---|---|---|---|
| | | | | 25° C./ | 40° C./ | 25° C./ | 40° C./ |
| Attributes | Specification | RRT | Initial | 60% RH | 75% RH | 60% RH | 75% RH |
| Report | Report all | 0.68 | 1.2% | 1.2% | 1.2% | 1.2% | 1.2% |
| Im purities | results ≥0.05% | 0.92 | 0.05% | <0.05% | <0.05% | <0.05% | <0.05% |
| (TM.05064) | | 0.94 | 0.95% | 0.95% | 0.95% | 0.95% | 0.95% |
| | | 0.97 | 0.61% | 0.65% | 0.65% | 0.62% | 0.62% |

RH = relative humidity; RRT = relative retention time; TM = test method.

TABLE 22
Results from the In-Use Stability Study of 100 mg Capsules

| | | | Storage | | |
|---|---|---|---|---|---|
| Attributes | Specification | Initial | Condition | 2 Weeks | 4 Weeks |
| Appearance | White capsules | Conforms | 25° C./60% RH | Conforms | Conforms |
| (TM.795) | free from | | 40° C./75% RH | Conforms | Conforms |
| | physical defects | | | | |
| | containing an | | | | |
| | off-white to | | | | |
| | white powder | | | | |
| Assay by UPLC | 90.0-110.0% | 95.8% | 25° C./60% RH | 96.4% | 95.8% |
| (TM.05064) | | | 40° C./75% RH | 96.5% | 95.8% |
| Purity by UPLC | ≥95.0% | 97.1% | 25° C./60% RH | 97.1% | 97.1% |
| (TM.05064) | | | 40° C./75% RH | 97.1% | 97.1% |
| Total Impurities | ≤5.0% | 2.8% | 25° C./60% RH | 2.8% | 2.8% |
| (TM.05064) | | | 40° C./75% RH | 2.8% | 2.8% |
| Disintegration | Report results | 4.38, 3.59, 5.02, | 25° C./60% RH | 3.41, 3.55, 4.26, | 3.19, 4.13, 4.29, |
| (TM.05056) | (min) | 3.05, 5.10, 4.48 | 40° C./75% RH | 4.17, 4.04, 3.51 | 4.19, 3.51, 4.11 |
| | Mean (n = 6) | Mean: 4.27 min | | Mean: 4.02 min | Mean: 4.04 min |
| | | | | 3.07, 3.23, 3.02, | 3.47, 3.55, 3.41, |
| | | | | 3.44, 3.23, 3.56 | 4.11, 4.01, 4.44 |
| | | | | Mean: 3.26 min | Mean: 4.03 min |
| Water Content | ≤5.0% | 0.18% | 25° C./60% RH | 0.13% | 0.13% |
| (Karl Fischer) | | | 40° C./75% RH | 0.15% | 0.13% |
| XRPD | Crystalline | Conforms | 25° C./60% RH | Conforms | Conforms |
| (TM.60) | Form C | | 40° C./75% RH | Conforms | Conforms |

RH = relative humidity; TM = test method; UPLC = ultra-performance liquid chromatography; XRPD = x-ray powder diffraction.

TABLE 23
Results for Impurities from the In-Use Stability Study of 100 mg Capsules

| | | | | 2 Weeks | | 4 Weeks | |
|---|---|---|---|---|---|---|---|
| | | | | 25° C./ | 40° C./ | 25° C./ | 40° C./ |
| Attribute | Specification | RRT | Initial | 60% RH | 75% RH | 60% RH | 75% RH |
| Report | Report all | 0.68 | 1.2% | 1.2% | 1.2% | 1.2% | 1.2% |
| Impurities | results ≥0.05% | 0.94 | 0.95% | 0.95% | 0.96% | 0.95% | 0.95% |
| (TM.05064) | | 0.97 | 0.62% | 0.65% | 0.65% | 0.62% | 0.62% |

Abbreviations: RH = relative humidity; RRT = relative retentiontime; TM = test method.

TABLE 24
Results from the In-Use Stability Study of the 200 mg Capsules

| | | | Storage | | |
|---|---|---|---|---|---|
| Attributes | Specification | Initial | Condition | 2 Weeks | 4 Weeks |
| Appearance | White capsules | Conforms | 25° C./60% RH | Conforms | Conforms |
| (TM.795) | free from | | 40° C./75% RH | Conforms | Conforms |
| | physical defects | | | | |
| | containing an | | | | |
| | off-white to | | | | |
| | white powder | | | | |
| Assay by UPLC | 90.0-110.0% | 96.4% | 25° C./60% RH | 96.6% | 95.8% |
| (TM.05064) | | | 40° C.75% RH | 96.0% | 95.8% |
| Purity by UPLC | ≥95.0% | 97.1% | 25° C./60% RH | 97.1% | 97.1% |
| (TM.05064) | | | 40° C./75% RH | 97.1% | 97.1% |
| Total Impurities | ≤5.0% | 2.8% | 25° C./60% RH | 2.8% | 2.8% |
| (TM.05064) | | | 40° C./75% RH | 2.8% | 2.8% |
| Disintegration | Report results | 5.40, 7.54, 4.15, | 25° C./60% RH | 4.01, 4.31, 4.21, | 3.41, 3.56, 4.58, |
| (TM.05056) | (min) | 3.21, 9.07, 6.36 | 40° C./75% RH | 4.51, 3.59, 3.46 | 4.23, 4.50, 3.54 |
| | Mean (n = 6) | Mean: 6.08 min | | Mean: 4.15 min | Mean: 4.17 min |
| | | | | 3.47, 3.55, 4.11, | 4.12, 3.46, 4.23, |
| | | | | 3.49, 3.38, 4.21 | 4.19, 3.52, 4.02 |
| | | | | Mean: 3.57 min | Mean: 4.05 min |
| Water Content | ≤5.0% | 0.17% | 25° C./60% RH | 0.12% | 0.14% |
| (Karl Fischer) | | | 40° C./75% RH | 0.14% | 0.12% |
| XRPD | Crystalline | Conforms | 25° C./60% RH | Conforms | Conforms |
| (TM.60) | Form C | | 40° C./75% RH | Conforms | Conforms |

RH = relative humidity; TM = test method; UPLC = ultra-performance liquid chromatography; XRPD = x-ray powder diffraction.

TABLE 25
Results for Impurities from the In-Use Stability Study of 200 mg Capsules

| | | | | 2 Weeks | | 4 Weeks | |
|---|---|---|---|---|---|---|---|
| | | | | 25° C./ | 40° C./ | 25° C./ | 40° C./ |
| Attribute | Specification | RRT | Initial | 60% RH | 75% RH | 60% RH | 75% RH |
| Report | Report all | 0.68 | 1.2% | 1.2% | 1.2% | 1.2% | 1.2% |
| Impurities | results ≥0.05% | 0.94 | 0.95% | 0.95% | 0.95% | 0.95% | 0.95% |
| (TM.05064) | | 0.97 | 0.61% | 0.65% | 0.65% | 0.62% | 0.63% |

RH = relative humidity; RRT = relative retention time; TM = test method.

TABLE 26
Results for Stability Study of the Non-GMP Active Tablets

| | | Storage | | |
|---|---|---|---|---|
| Attributes | Initial | Condition | 1M | 3M |
| Appearance | White, round, | 25 ± 2° C./ | White, round, | White, round, |
| | uncoated tablet, | 60 ± 5% RH | uncoated tablet, | uncoated tablet, |
| | free from visible | (with desiccant) | free from visible | free from visible |
| | defects or | | defects or | defects or |
| | discoloration | | discoloration | discoloration |
| Assay | 100.9% | | 100.2% | 100.6% |
| Total Impurities | 2.75% | | 2.71% | 2.67% |
| Dissolution | 97% released | | 94% released | 92% released |
| | at 45 min | | at 45 min | at 45 min |
| Water Content | 5.3% | | 4.5% | 4.2% |
| Disintegration | Max: 2 min | | Max: 3 min | Max: 2 min |
| | Min: 1 min | | Min: 2 min | Min: 2 min |
| | Mean: 2 min | | Mean: 3 min | Mean: 2 min |
| Hardness | Max: 58N | | Max: 65N | Max: 67N |
| | Min: 43N | | Min: 53N | Min: 50N |
| | Mean: 50N | | Mean: 60N | Mean: 60N |
| Friability | 0.1% | | 0.1% | — |
| XRPD | Form A not | | Form A not | — |
| | detected in DP | | detected in DP | |
| Appearance | White, round, | 25 ± 2° C./ | White, round, | White, round, |
| | uncoated tablet, | 60 ± 5% RH | uncoated tablet, | uncoated tablet, |
| | free from visible | (without | free from visible | free from visible |
| | defects or | desiccant) | defects or | defects or |
| | discoloration | | discoloration | discoloration |
| Assay | 100.9% | | 100.5% | 100.6% |
| Total Impurities | 2.75% | | 2.73% | 2.68% |
| Dissolution | 97% released | | 96% released | 92% released |
| | at 45 min | | at 45 min | at 45 min |
| Water Content | 5.3% | | 5.2% | 4.9% |
| Disintegration | Max: 2 min | | Max: 2 min | Max: 2 min |
| | Min: 1 min | | Min: 2 min | Min: 1 min |
| | Mean: 2 min | | Mean: 2 min | Mean: 2 min |
| Hardness | Max: 58N | | Max: 71N | Max: 59N |
| | Min: 43N | | Min: 53N | Min: 51N |
| | Mean: 50N | | Mean: 59N | Mean: 54N |
| Friability | 0.1% | | 0.1% | — |
| XRPD | Form A not | | Form A not | — |
| | detected in DP | | detected in DP | |
| Appearance | White, round, | 40 ± 2° C./ | White, round, | White, round, |
| | uncoated tablet, | 75 ± 5% RH | uncoated tablet, | uncoated tablet, |
| | free from visible | (without | free from visible | free from visible |
| | defects or | desiccant) | defects or | defects or |
| | discoloration | | discoloration | discoloration |
| Assay | 100.9% | | 101.1% | 100.3% |
| Total Impurities | 2.75% | | 2.69% | 2.63% |
| Dissolution | 97% released | | 97% released | 95% released |
| | at 45 min | | at 45 min | at 45 min |
| Water Content | 5.3% | | 4.7% | 5.1% |
| Disintegration | Max: 2 min | | Max: 2 min | Max: 3 min |
| | Min: 1 min | | Min: 2 min | Min: 2 min |
| | Mean: 2 min | | Mean: 2 min | Mean: 2 min |
| Hardness | Max: 58N | | Max: 64N | Max: 61N |
| | Min: 43N | | Min: 48N | Min: 48N |
| | Mean: 50N | | Mean: 54N | Mean: 56N |
| Friability | 0.1% | | 0.1% | — |
| XRPD | Form A not | | Form A not | Form A not |
| | detected in DP | | detected in DP | detected in DP |

DP, drug product;
XRPD, x-ray powder diffraction

TABLE 27
Results for Individual Impurities from Stability Study of the Non-GMP Active Tablets

| | | Storage | | |
|---|---|---|---|---|
| Attributes | Initial | Condition | 1M | 3M |
| Individual | RRT 0.682 | 25 ± 2° C./ | RRT 0.679 | RRT 0.683 |
| Impurities | (C18031554-E): | 60 ± 5% RH | (C18031554-E): | (C18031554-E): |
| | 1.18% | (with desiccant) | 1.17% | 1.17% |
| | RRT 0.940: 0.22% | | RRT 0.940: 0.23% | RRT 0.940: 0.23% |
| | RRT 0.966: 0.16% | | RRT 0.966: 0.16% | RRT 0.966: 0.16% |
| | RRT 0.972: 1.00% | | RRT 0.972: 1.00% | RRT 0.972: 1.00% |
| | RRT 0.980: 0.18% | | RRT 0.980: 0.15% | RRT 0.979: 0.12% |
| Individual | RRT 0.682 | 25 ± 2° C./ | RRT 0.680 | RRT 0.683 |
| Impurities | (C18031554-E): | 60 ± 5% RH | (C18031554-E): | (C18031554-E): |
| | 1.18% | (without | 1.17% | 1.17% |
| | RRT 0.940: 0.22% | desiccant) | RRT 0.940: 0.23% | RRT 0.940: 0.23% |
| | RRT 0.966: 0.16% | | RRT 0.966: 0.16% | RRT 0.966: 0.16% |
| | RRT 0.972: 1.00% | | RRT 0.972: 1.00% | RRT 0.972: 1.01% |
| | RRT 0.980: 0.18% | | RRT 0.980: 0.17% | RRT 0.980: 0.12% |
| Individual | RRT 0.682 | 40 ± 2° C./ | RRT 0.680 | RRT 0.683 |
| Impurities | (C18031554-E): | 75 ± 5% RH | (C18031554-E): | (C18031554-E): |
| | 1.18% | (without | 1.13% | 1.10% |
| | RRT 0.940: 0.22% | desiccant) | RRT 0.940: 0.22% | RRT 0.940: 0.23% |
| | RRT 0.966: 0.16% | | RRT 0.966: 0.16% | RRT 0.966: 0.16% |
| | RRT 0.972: 1.00% | | RRT 0.972: 1.00% | RRT 0.972: 1.02% |
| | RRT 0.980: 0.18% | | RRT 0.980: 0.17% | RRT 0.980: 0.13% |

RRT, relative retention time

TABLE 28
Results for Stability Study of the GMP Active Tablets

| | | Storage | |
|---|---|---|---|
| Attributes | Initial | Condition | 1M |
| Appearance | White, round, uncoated | 25 ± 2° C./ | White, round, uncoated |
| | tablet, free from visible | 60 ± 5% RH | tablet, free from visible |
| | defects or discoloration | | defects or discoloration |
| Assay | 102.2% | | 101.3% |
| Total Impurities | 1.56% | | 1.47% |
| Dissolution | 92% released at 45 min | | 97% released at 45 min |
| Water Content | 5.3% | | 5.1% |
| Disintegration | Max: 2 min | | Max: 2 min |
| | Min: 1 min | | Min: 1 min |
| | Mean: 1 min | | Mean: 2 min |
| Hardness | Max: 58N | | — |
| | Min: 49N | | |
| | Mean: 52N | | |
| Friability | 0.0% | | — |
| XRPD | Form A not detected in DP | | Form A not detected in DP |
| Microbial Limits | TAMC: <50 cfu/g | | |
| | TYMC: <50 cfu/g | | |
| | E. coli: Absence/g | | |
| Appearance | White, round, uncoated | 40 ± 2° C./ | White, round, uncoated |
| | tablet, free from visible | 75 ± 5% RH | tablet, free from visible |
| | defects or discoloration | | defects or discoloration |
| Assay | 102.2% | | 101.2% |
| Total Impurities | 1.56% | | 1.35% |
| Dissolution | 92% released at 45 min | | 97% released at 45 min |
| Water Content | 5.3% | | 4.9% |
| Disintegration | Max: 2 min | | Max: 2 min |
| | Min: 1 min | | Min: 1 min |
| | Mean: 1 min | | Mean: 1 min |
| Hardness | Max: 58N | | — |
| | Min: 49N | | |
| | Mean: 52N | | |
| Friability | 0.0% | | — |
| XRPD | Form A not detected in DP | | Form A not detected in DP |
| Microbial Limits | TAMC: <50 cfu/g | | — |
| | TYMC: <50 cfu/g | | — |
| | E. coli: Absence/g | | — |

DP = drug product; XRPD, x-ray powder diffraction; TAMC, total aerobic microbial count; TYMC, total yeast and mold count; E. coli, Escherichia coli.

TABLE 29
Results for Individual Impurities from the
Stability Study of GMP Active Tablets

| | | Storage | |
|---|---|---|---|
| Attributes | Initial | Condition | 1M |
| Individual | RRT 0.679 | 25 ± 2° C./ | RRT 0.693 |
| Impurities | (C18031554-E): | 60 ± 5% RH | (C18031554-E): |
| | 0.55% | | 0.53% |
| | RRT 0.940: 0.73% | | RRT 0.943: 0.67% |
| | RRT 0.973: 0.16% | | RRT 0.974: 0.16% |
| | RRT 0.979: 0.11% | | RRT 0.979: 0.10% |
| Individual | RRT 0.679 | 40 ± 2° C./ | RRT 0.693 |
| Impurities | (C18031554-E): | 75 ± 5% RH | (C18031554-E): |
| | 0.55% | | 0.52% |
| | RRT 0.940: 0.73% | | RRT 0.943: 0.67% |
| | RRT 0.973: 0.16% | | RRT 0.974: 0.16% |
| | RRT 0.979: 0.11% | | |

RRT, Relative retention time

### Example 5: Manufacturing Process for a Tablet

This example illustrates a manufacturing process executed for a tablet formulation disclosed herein. The tablet manufactured from the method contained Compound 1. Step (i): co-sifting Compound 1, MCC, LMH, HPC and CCNa through a 30-mesh screen. Step (ii): loading the sifted materials from step (i) to a 100 L blender and blending for 15 min at 15 rpm. Step (iii): unloading the blended materials from step (ii) and sifting through a 30-mesh screen. Step (iv): adding the sifted materials from step (iii) to a 100 L blender and blending for 15 min at 15 rpm. Step (v): co-sifting CSD and intragranular magnesium stearate through a 60-mesh screen and lubricating the blended materials from the step (iv) with a 100 L blender for 5 min at 15 rpm. Step (vi): granulating the lubricated materials from step (v) by roller compaction at a roll pressure of 3 MPa, roll gap of 2.0 mm, roll speed of 4.0 rpm, feed screw speed of 20 rpm and producing a bulk density of 0.5 g/ml. Step (vii): sifting intragranular magnesium stearate through a 60-mesh screen and lubricating with granulated materials from the step (vi) for 5 min at 15 rpm. Step (viii): compressing the materials from the step (vii) into the tablet pharmaceutical composition using a 6.0 mm round shaped punch at a rotary speed of 30 rpm, thickness scale of 2.0 mm, fill depth scale of 6.0 mm and main compression force of 7.0 kN. The tablet produced from step (viii) was 100 mg and had a thickness of 3.00 mm, hardness of 70 N, friability of no more than 1.0% by weight, and disintegration time of less than 15 min. Step (ix): packaging the manufactured tablet into a 45 HDPE and fill the bottle with 30 tablets. After the step (iv), sampling was carried out by taking a sample from each of 10 locations of the blender. A sample from each location was about 197 mg and the sampling was carried out so that all the individual assays are within mean±10% (absolute) and RSD % and the NMT is about 5%. After the step (v), BD/TD of the lubricated blend was tested by taking approximately 30 g of the lubricated granule blend from the bin to test the bulk density (BD), tapped density (TD) and compressibility index parameters. The acceptable density is around 0.50 g/ml or in the range of 0.45 to 0.60 g/ml. The standard protocol USP <701> was used to determine disintegration. The standard protocol USP <1217> was used to determine hardness. The standard protocol USP <1216> was used to determine friability.

### Example 6: Formula of a Tablet

The following is a formula of a tablet containing Compound 1 manufactured according to a method disclosed herein as shown in Table 30.

TABLE 30
Tablet formulation

| | Percentage by weight |
|---|---|
| Ingredients | of the Tablet |
| Compound 1 | 5.00 |
| CCNa | 0.75 |
| HPC | 6.00 |
| MCC | 43.25 |
| LMH | 43.50 |
| CSD | 0.50 |
| Magnesium Stearate | 1.00 |
| Total | 100% |

This example illustrates the dissolution profile of a batch of tablet pharmaceutical compositions comprising 2 mg of Compound 1 according to the formulation in Table 30. This batch of tablets was prepared according to methods disclosed herein containing the same ingredients as the tablets in Example 6 and assessed for their dissolution profile at 5 min, 10 min, 15 min, 30 m, 45 mi, 60 m and 90 mi time points when tested in USP <711> type II apparatus (paddle) at 60 rpm in 900 ml sodium acetate (NaAc) buffer solution containing 0.2500 Sodium dodecyl sulfate (SDS) with a pH of 4.5. The tests were conducted at 25±2° C. with a relative humidity of 60±5% for the tablets in Tables 31,32,33,34, 35,36,37, 38,39, 40, and 41, and at 40±2° C. with a relative humidity of 75±5% for the tablets in Tables 42, 43, and 44. Tests were conducted at 30±2° C. with a relative humidity of 65±50% for the tablets in Table 45. Tablets of Tables 32, 33, 34, 35, and 36 were tested with desiccant and tablets of Tables 37, 38, 39, 40, and 4l were tested without desiccant. The tests were conducted with desiccant for tablets in Tables 32, 33, 34, 35, and 36 and without desiccant for tablets in Tables 37, 38, 39, 40, and 41. Tablets of Tables 42, 43, 44, and 45 were tested without desiccant. Data for percent of tablet release at each time point are shown in Tables 31-45. Tablets stored for 1 month (1M), 3 months (3M), 6 months (6M), 9 months (9M), and 12 months (12M) were tested. Tablets containing 2.5 mg and 5 mg of compound 1 were also tested and gave similar profiles.

TABLE 31
Dissolution Profile of Tablets

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 51 | 74 | 85 | 97 | 101 | 102 | 102 | 41.36 |
| 2 | 59 | 86 | 93 | 100 | 102 | 102 | 102 | 41.46 |
| 3 | 44 | 78 | 91 | 101 | 103 | 104 | 105 | 42.21 |
| 4 | 51 | 82 | 91 | 99 | 101 | 102 | 103 | 41.58 |
| 5 | 55 | 83 | 93 | 100 | 102 | 102 | 102 | 41.48 |
| 6 | 52 | 80 | 90 | 97 | 100 | 100 | 100 | 40.91 |
| Mean % | 52 | 80 | 90 | 99 | 101 | 102 | 102 | N/A |
| Max. % | 59 | 86 | 93 | 101 | 103 | 104 | 105 | |
| Min. % | 44 | 74 | 85 | 97 | 100 | 100 | 100 | |
| SD % | 5 | 4 | 3 | 2 | 1 | 1 | 1 | |
| RSD % | 10 | 5 | 4 | 3 | 2 | 1 | 1 | |

RSD: relative standard deviation; SD: standard deviation

TABLE 32
Dissolution Profile of Tablets (25 ± 2° C./
60 ± 5% RH/1M/dissolution (HPLC))

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 61 | 82 | 89 | 96 | 98 | 98 | 98 | 41.97 |
| 2 | 59 | 84 | 91 | 96 | 98 | 99 | 99 | 41.96 |
| 3 | 60 | 89 | 94 | 99 | 100 | 101 | 101 | 41.91 |
| 4 | 57 | 86 | 92 | 97 | 98 | 99 | 99 | 41.22 |
| 5 | 49 | 79 | 89 | 99 | 101 | 101 | 102 | 42.70 |
| 6 | 53 | 84 | 92 | 99 | 101 | 102 | 102 | 42.82 |
| Mean % | 57 | 84 | 91 | 98 | 99 | 100 | 100 | N/A |
| Max. % | 61 | 89 | 94 | 99 | 101 | 102 | 102 | |
| Min. % | 49 | 79 | 89 | 96 | 98 | 98 | 98 | |
| SD % | 5 | 3 | 2 | 1 | 1 | 2 | 2 | |
| RSD % | 8 | 4 | 2 | 1 | 1 | 2 | 2 | |

TABLE 33
Dissolution Profile of Tablets (25 ± 2° C./
60 ± 5% RH/3M/dissolution (HPLC))

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 48 | 77 | 90 | 98 | 100 | 100 | 101 | 42.33 |
| 2 | 58 | 80 | 88 | 97 | 99 | 99 | 99 | 42.09 |
| 3 | 44 | 70 | 81 | 91 | 95 | 96 | 97 | 40.91 |
| 4 | 45 | 68 | 79 | 89 | 92 | 95 | 98 | 41.79 |
| 5 | 47 | 68 | 78 | 89 | 93 | 97 | 98 | 41.59 |
| 6 | 59 | 85 | 92 | 97 | 98 | 98 | 99 | 41.42 |
| Mean % | 50 | 75 | 85 | 93 | 96 | 98 | 99 | N/A |
| Max. % | 59 | 85 | 92 | 96 | 100 | 100 | 101 | |
| Min. % | 44 | 68 | 78 | 89 | 92 | 95 | 97 | |
| SD % | 6 | 7 | 6 | 4 | 3 | 2 | 1 | |
| RSD % | 13 | 10 | 7 | 5 | 3 | 2 | 1 | |

TABLE 34
Dissolution Profile of Tablets (25 ± 2° C./
60 ± 5% RH/6M/dissolution (HPLC))

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 64 | 88 | 95 | 100 | 102 | 103 | 103 | 42.26 |
| 2 | 55 | 76 | 87 | 97 | 101 | 102 | 103 | 43.68 |
| 3 | 54 | 72 | 83 | 98 | 103 | 104 | 104 | 43.02 |
| 4 | 54 | 76 | 86 | 96 | 100 | 102 | 102 | 42.46 |
| 5 | 48 | 73 | 84 | 94 | 98 | 100 | 100 | 41.43 |
| 6 | 46 | 70 | 81 | 92 | 99 | 101 | 102 | 42.53 |
| Mean % | 54 | 76 | 86 | 96 | 101 | 102 | 102 | N/A |
| Max. % | 64 | 88 | 95 | 100 | 102 | 104 | 104 | |
| Min. % | 46 | 70 | 81 | 92 | 98 | 100 | 100 | |
| SD % | 6 | 6 | 5 | 3 | 2 | 1 | 1 | |
| RSD % | 12 | 8 | 5 | 3 | 2 | 1 | 1 | |

TABLE 35
Dissolution Profile of Tablets (25 ± 2° C./
60 ± 5% RH/9M/dissolution (HPLC))

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 31 | 65 | 79 | 91 | 97 | 100 | 102 | 42.61 |
| 2 | 53 | 82 | 93 | 99 | 100 | 101 | 101 | 42.08 |
| 3 | 46 | 72 | 85 | 95 | 98 | 99 | 99 | 41.30 |
| 4 | 47 | 73 | 84 | 96 | 98 | 99 | 99 | 41.35 |
| 5 | 39 | 64 | 76 | 91 | 97 | 98 | 99 | 41.05 |
| 6 | 51 | 79 | 90 | 96 | 98 | 99 | 99 | 41.14 |
| Mean % | 45 | 73 | 84 | 95 | 98 | 99 | 100 | N/A |
| Max. % | 53 | 82 | 93 | 99 | 100 | 101 | 102 | |
| Min. % | 31 | 64 | 76 | 91 | 97 | 98 | 99 | |
| SD % | 8 | 7 | 6 | 3 | 1 | 1 | 1 | |
| RSD % | 18 | 10 | 7 | 3 | 1 | 1 | 1 | |

TABLE 36
Dissolution Profile of Tablets (25 ± 2° C./
60 ± 5% RH/12M/dissolution (HPLC))

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 68 | 90 | 95 | 99 | 100 | 101 | 101 | 40.49 |
| 2 | 47 | 71 | 80 | 93 | 98 | 100 | 101 | 40.40 |
| 3 | 58 | 85 | 94 | 102 | 104 | 105 | 105 | 42.20 |
| 4 | 62 | 80 | 87 | 99 | 102 | 103 | 103 | 41.40 |
| 5 | 54 | 75 | 85 | 98 | 102 | 103 | 104 | 41.43 |
| 6 | 53 | 80 | 91 | 98 | 101 | 102 | 102 | 41.01 |
| Mean % | 57 | 80 | 89 | 98 | 101 | 102 | 103 | N/A |
| Max. % | 68 | 90 | 95 | 102 | 104 | 105 | 105 | |
| Min. % | 47 | 71 | 80 | 93 | 98 | 100 | 101 | |
| SD % | 7 | 7 | 6 | 3 | 2 | 2 | 2 | |
| RSD % | 13 | 8 | 6 | 3 | 2 | 2 | 2 | |

TABLE 37
Dissolution Profile of Tablets (25 ± 2° C./
60 ± 5% RH/1M/dissolution (HPLC))

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 48 | 78 | 88 | 97 | 100 | 100 | 101 | 41.55 |
| 2 | 61 | 85 | 92 | 99 | 102 | 103 | 103 | 42.32 |
| 3 | 62 | 87 | 94 | 100 | 102 | 102 | 102 | 42.24 |
| 4 | 48 | 80 | 90 | 98 | 101 | 102 | 102 | 42.10 |
| 5 | 46 | 76 | 85 | 94 | 98 | 99 | 100 | 41.20 |
| 6 | 50 | 80 | 90 | 99 | 102 | 103 | 104 | 43.58 |
| Mean % | 52 | 81 | 90 | 98 | 101 | 102 | 102 | N/A |
| Max. % | 26 | 87 | 94 | 100 | 102 | 103 | 104 | |
| Min. % | 46 | 76 | 85 | 94 | 98 | 99 | 100 | |
| SD % | 7 | 4 | 3 | 2 | 2 | 2 | 2 | |
| RSD % | 13 | 5 | 3 | 2 | 2 | 2 | 2 | |

TABLE 38
Dissolution Profile of Tablets (25 ± 2° C./
60 ± 5% RH/3M/dissolution (HPLC))

| Tablets | | | | | | | 90 min | |
|---|---|---|---|---|---|---|---|---|
| No.\ | | | | | | | (Infinity, | Tablet |
| % | 5 | 10 | 15 | 30 | 45 | 60 | 250 rpm/ | Weight |
| Release | min | min | min | min | min | min | 30 min) | (mg) |
| 1 | 49 | 70 | 80 | 91 | 95 | 96 | 96 | 41.23 |
| 2 | 61 | 82 | 89 | 96 | 97 | 98 | 98 | 42.22 |
| 3 | 54 | 82 | 90 | 94 | 99 | 99 | 100 | 42.63 |
| 4 | 60 | 81 | 89 | 94 | 98 | 99 | 99 | 42.50 |
| 5 | 52 | 76 | 48 | 94 | 97 | 98 | 98 | 42.35 |
| 6 | 65 | 86 | 92 | 96 | 98 | 98 | 98 | 42.28 |
| Mean % | 57 | 80 | 87 | 95 | 97 | 98 | 98 | N/A |
| Max. % | 65 | 86 | 92 | 96 | 99 | 99 | 100 | |
| Min. % | 49 | 70 | 80 | 91 | 95 | 96 | 96 | |
| SD % | 6 | 6 | 4 | 2 | 1 | 1 | 1 | |
| RSD % | 11 | 7 | 5 | 2 | 1 | 1 | 1 | |

TABLE 39
Dissolution Profile of Tablets
(25 ± 2° C./60 ± 5% RH/6 M/dissolution (HPLC))

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 51 | 78 | 88 | 96 | 99 | 100 | 101 | 42.61 |
| 2 | 49 | 76 | 89 | 99 | 102 | 104 | 104 | 43.26 |
| 3 | 46 | 72 | 83 | 95 | 99 | 101 | 102 | 43.49 |
| 4 | 50 | 68 | 78 | 95 | 100 | 101 | 102 | 43.41 |
| 5 | 43 | 68 | 80 | 94 | 100 | 102 | 103 | 43.84 |
| 6 | 57 | 82 | 91 | 99 | 101 | 102 | 102 | 44.41 |
| Mean % | 49 | 74 | 85 | 96 | 100 | 102 | 102 | N/A |
| Max. % | 57 | 82 | 91 | 99 | 102 | 104 | 104 | |
| Min. % | 43 | 68 | 78 | 94 | 99 | 100 | 101 | |
| SD % | 5 | 6 | 5 | 2 | 1 | 1 | 1 | |
| RSD % | 10 | 8 | 6 | 2 | 1 | 1 | 1 | |

TABLE 40
Dissolution Profile of Tablets
(25 ± 2° C./60 ± 5% RH/9 M/dissolution (HPLC))

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 53 | 79 | 88 | 95 | 97 | 98 | 99 | 41.93 |
| 2 | 37 | 66 | 80 | 94 | 97 | 100 | 102 | 43.01 |
| 3 | 45 | 72 | 81 | 93 | 97 | 98 | 99 | 42.01 |
| 4 | 44 | 72 | 83 | 94 | 98 | 99 | 100 | 42.49 |
| 5 | 48 | 68 | 80 | 97 | 100 | 101 | 101 | 43.12 |
| 6 | 48 | 74 | 84 | 9.3 | 95 | 96 | 96 | 41.04 |
| Mean % | 46 | 72 | 83 | 94 | 97 | 99 | 99 | N/A |
| Max. % | 53 | 79 | 88 | 97 | 100 | 101 | 102 | |
| Min. % | 37 | 66 | 80 | 93 | 95 | 96 | 96 | |
| SD % | 5 | 5 | 3 | 1 | 1 | 2 | 2 | |
| RSD % | 11 | 6 | 4 | 1 | 1 | 2 | 2 | |

TABLE 41
Dissolution Profile of Tablets
(25 ± 2° C./60 ± 5% RH/12 M/dissolution (HPLC))

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 57 | 84 | 91 | 98 | 100 | 100 | 100 | 41.19 |
| 2 | 51 | 77 | 87 | 96 | 99 | 99 | 99 | 40.99 |
| 3 | 44 | 68 | 80 | 93 | 99 | 101 | 102 | 42.14 |
| 4 | 49 | 73 | 82 | 93 | 98 | 99 | 99 | 40.89 |
| 5 | 47 | 70 | 79 | 91 | 94 | 98 | 100 | 41.61 |
| 6 | 62 | 84 | 91 | 96 | 98 | 98 | 98 | 40.42 |
| Mean % | 53 | 76 | 85 | 95 | 98 | 99 | 100 | N/A |
| Max. % | 62 | 84 | 91 | 98 | 100 | 101 | 102 | |
| Min. % | 44 | 68 | 79 | 91 | 96 | 98 | 98 | |
| SD % | 7 | 7 | 5 | 3 | 1 | 1 | 1 | |
| RSD % | 13 | 9 | 6 | 3 | 1 | 1 | 1 | |

TABLE 42
Dissolution Profile of Tablets
(40 ± 2° C./75 ± 5% RH/1 M/dissolution (HPLC))

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 53 | 80 | 90 | 99 | 101 | 102 | 103 | 42.58 |
| 2 | 57 | 81 | 90 | 99 | 102 | 104 | 105 | 44.05 |
| 3 | 55 | 81 | 90 | 98 | 100 | 102 | 102 | 42.11 |
| 4 | 43 | 78 | 88 | 98 | 101 | 103 | 103 | 43.04 |
| 5 | 46 | 71 | 83 | 100 | 104 | 105 | 106 | 44.98 |
| 6 | 50 | 79 | 88 | 97 | 102 | 103 | 104 | 44.23 |
| Mean % | 50 | 78 | 88 | 99 | 102 | 103 | 104 | N/A |
| Max. % | 57 | 81 | 90 | 100 | 104 | 105 | 106 | |
| Min. % | 43 | 71 | 83 | 97 | 100 | 102 | 102 | |
| SD % | 5 | 4 | 3 | 1 | 1 | 1 | 2 | |
| RSD % | 11 | 5 | 3 | 2 | 1 | 1 | 2 | |

TABLE 43
Dissolution Profile of Tablets
(40 ± 2° C./75 ± 5% RH/6 M/dissolution (HPLC))

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 57 | 80 | 88 | 95 | 97 | 97 | 98 | 42.59 |
| 2 | 53 | 75 | 83 | 91 | 95 | 96 | 98 | 41.71 |
| 3 | 47 | 72 | 82 | 91 | 94 | 95 | 96 | 41.59 |
| 4 | 35 | 71 | 82 | 93 | 98 | 100 | 102 | 43.71 |
| 5 | 46 | 70 | 78 | 88 | 94 | 96 | 100 | 42.93 |
| 6 | 48 | 70 | 78 | 88 | 94 | 96 | 100 | 41.79 |
| Mean % | 48 | 73 | 82 | 91 | 95 | 97 | 98 | N/A |
| Max. % | 57 | 80 | 88 | 95 | 98 | 100 | 102 | |
| Min. % | 35 | 70 | 78 | 88 | 93 | 95 | 96 | |
| SD % | 8 | 4 | 3 | 3 | 2 | 2 | 2 | |
| RSD % | 16 | 6 | 4 | 3 | 2 | 2 | 2 | |

TABLE 44
Dissolution Profile of Tablets
(40 ± 2° C./75 ± 5% RH/12 M/dissolution (HPLC))

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 30 | 74 | 86 | 97 | 101 | 103 | 104 | 44.88 |
| 2 | 43 | 75 | 85 | 94 | 98 | 99 | 101 | 43.81 |
| 3 | 40 | 68 | 80 | 93 | 97 | 99 | 101 | 44.06 |
| 4 | 39 | 69 | 80 | 91 | 96 | 97 | 99 | 42.73 |
| 5 | 34 | 68 | 80 | 91 | 97 | 99 | 102 | 44.01 |
| 6 | 36 | 66 | 79 | 92 | 98 | 101 | 103 | 45.30 |
| Mean % | 37 | 70 | 82 | 93 | 98 | 100 | 102 | N/A |
| Max. % | 43 | 75 | 86 | 97 | 101 | 103 | 104 | |
| Min. % | 30 | 66 | 79 | 91 | 96 | 98 | 99 | |
| SD % | 4 | 4 | 3 | 2 | 2 | 2 | 2 | |
| RSD % | 12 | 5 | 4 | 2 | 2 | 2 | 2 | |

TABLE 45
Dissolution Profile of Tablets
(30 ± 2° C./65 ± 5% RH/12 M/dissolution (HPLC))

| | | | | | | | 90 min | |
|---|---|---|---|---|---|---|---|---|
| Tablets | | | | | | | (Infinity, | Tablet |
| No.\% | 5 | 10 | 15 | 30 | 45 | 60 | 250 rpm/ | Weight |
| Release | min | min | min | min | min | min | 30 min) | (mg) |
| 1 | 58 | 83 | 92 | 99 | 102 | 102 | 103 | 42.12 |
| 2 | 37 | 65 | 77 | 92 | 98 | 100 | 102 | 41.52 |
| 3 | 47 | 76 | 87 | 97 | 100 | 100 | 101 | 41.29 |
| 4 | 39 | 64 | 74 | 87 | 94 | 98 | 101 | 41.40 |
| 5 | 46 | 71 | 82 | 95 | 99 | 100 | 101 | 41.26 |
| 6 | 45 | 72 | 86 | 95 | 98 | 100 | 101 | 41.02 |
| Mean % | 45 | 72 | 83 | 96 | 98 | 100 | 101 | N/A |
| Max. % | 58 | 83 | 92 | 99 | 102 | 102 | 103 | |
| Min. % | 37 | 64 | 74 | 87 | 94 | 98 | 101 | |
| SD % | 7 | 6 | 4 | 3 | 2 | 1 | 1 | |
| RSD % | 16 | 10 | 8 | 4 | 3 | 1 | 1 | |

### Example 7: Treatment of STGD1 Pediatric Patients

10 patients aged 10-20 years old with symptoms associated with autosomal recessive Stargardt disease (STGD1), including at least two pathogenic mutations of the ABCA4 gene and a defined lesion size are treated with a tablet pharmaceutical composition of a compound of Formula (I) (e.g., Compound 1) or placebo, for up to 18 months. Primary outcomes are PK/PD and RBP4 reduction. The results are analyzed following methods well known to the skilled artisan.

### Example 8: Treatment of STGD1 Patients

120 patients aged 12-40 years old with symptoms associated with autosomal recessive Stargardt disease (STGD1), including at least two pathogenic mutations of the ABCA4 gene and a defined lesion size are randomly assigned to receive a tablet pharmaceutical composition of a compound of Formula (I) (e.g., Compound 1) or placebo, for up to 18 months. Primary outcomes are PK/PD, RBP4 reduction, and the mean rate of change in the area of ellipsoid zone defect measured by en face SD-OCT, as measured by spectral Domain-Optical Coherence Tomography (SD-OCT). The results are analyzed following methods well known to the skilled artisan.

### Example 9: Treatment of (Dry) Age-Related Macular Degeneration (AMD) in Humans

100 patients at least 18 years of age with symptoms associated with dry AMD are randomly assigned to receive a tablet pharmaceutical composition of a compound of Formula (I) (e.g., Compound 1) or the standard of care, for up to 48 weeks. Primary outcomes are change from baseline of geographical atrophy (GA, mm²) size, change from baseline of retinal drusen volume (mm³), change in macular sensitivity (DB), change in monocular reading speed from baseline (words/min). The results are analyzed following methods well known to the skilled artisan.

### Example 10: Treatment of (Wet) Age-Related Macular Degeneration (AMD) in Humans

100 patients at least 50 years of age with symptoms associated with neovascular/wet AMD, including active primary or recurrent subfoveal choroidal neovascularization (CNV) secondary to age-related macular degeneration (AMD) are randomly assigned to receive a tablet pharmaceutical composition of a compound of Formula (I) (e.g., Compound 1) or the standard of care, for up to 48 weeks. Primary outcomes are change in the best corrected visual acuity score measured using the Early Treatment Diabetic Retinopathy Study (ETDRS) protocol by Week 16. Secondary outcomes are percent of subjects gaining >/=15 letters in the best corrected visual acuity score at 16 weeks compared to baseline, as measured using the ETDRS protocol; mean change from Baseline over time (16 weeks) in the best corrected visual acuity score, as measured using the ETDRS protocol; incidence and severity of ocular adverse events identified by ophthalmic examination and or spontaneously reported (at 48 weeks); change from baseline to weeks 4,8, 12, and 16 in retinal central subfield thickness and retinal lesion thickness assessed by OCT; incidence and severity of systemic adverse events identified by physical examination, changes in vital signs, clinical laboratory abnormalities and or spontaneously reported (at 48 weeks); and change from baseline in lesion size on FFA at Week 16. The results are analyzed following methods well known to the skilled artisan.

### Example 11: Treatment of Best Disease in Humans

50 patients at least 18 years of age with symptoms associated with Best disease or other eye disease caused by mutations in the gene BEST1 are randomly assigned to receive a tablet pharmaceutical composition of a compound of Formula (I) (e.g., Compound 1) or the standard of care, for up to 24 months. Primary outcomes are mean change in central retinal thickness as measured by OCT at month 12 compared to baseline and change in leakage area seen during fluorescein angiography at month 12 as compared with baseline. The results are analyzed following methods well known to the skilled artisan.

### Example 12: Treatment of Adult Vitelliform Maculopathy in Humans

50 patients at least 18 years of age with symptoms associated with Adult Vitelliform Maculopathy are randomly assigned to receive a tablet pharmaceutical composition of a compound of Formula (I) (e.g., Compound 1) or the standard of care, for up to 24 months. Primary outcomes are mean change in central retinal thickness as measured by OCT at month 12 compared to baseline and change in leakage area seen during fluorescein angiography at month 12 as compared with baseline. The results are analyzed following methods well known to the skilled artisan.

### Example 13: Treatment of Diabetic Retinopathy in Humans

50 patients 45-75 years of age with symptoms associated with diabetic retinopathy are randomly assigned to receive a tablet pharmaceutical composition of a compound of Formula (I) (e.g., Compound 1) or the standard of care, for up to 24 months. Primary outcomes are Best Corrected Visual Acuity (BCVA) assessed by ETDRS (Early Treatment Diabetic Retinopathy Study) scale; Retinal Nerve Fiber Layer (RNFL), Ganglion Cell Layer (GCL), Retinal thickness, and central Retinal thickness assessed by Spectral Domain Optical Coherence Tomography (SD-OCT); and microaneurysm turnover assessed by Colour Fundus Photography. Outcomes are assessed at 0, 6, 12, 18, and 24 months. The results are analyzed following methods well known to the skilled artisan.

### Example 14: Treatment of Geographic Atrophy in Humans

50 patients at least 50 years of age with symptoms associated with geographic atrophy are randomly assigned to receive a tablet pharmaceutical composition of a compound of Formula (I) (e.g., Compound 1) or the standard of care, for up to 12 months. Primary outcomes are the change in square root geographic atrophy (GA) lesion size from baseline at week 24 as measured by FAF (Fundus Autofluorescence). A positive change from baseline indicates an increase in size of geographic atrophy lesion area (worsening; disease progression). The results are analyzed following methods well known to the skilled artisan.

### Example 15: Formulas for a Tablet

The following are formulas of a tablet containing Compound 1 which are manufactured according to a method disclosed herein as shown in Table 46.

TABLE 46
Tablet formulations

| | Ingredient % weight in Tablet | | | | | | |
|---|---|---|---|---|---|---|---|
| | Com- | | | | | | Mag- |
| | pound | | | | | | nesium |
| Formula | 1 | CCNa | HPC | MCC | LMH | CSD | Stearate |
| 1 | 5 | 0.75 | 6 | 43.25 | 43.5 | 0.5 | 1 |
| 2 | 7 | 0.75 | 3 | 44.25 | 43.5 | 0.5 | 1 |
| 3 | 10 | 0.75 | 3 | 43.25 | 41.5 | 0.5 | 1 |
| 4 | 15 | 0.75 | 3 | 39.5 | 40.25 | 0.5 | 1 |
| 5 | 5 | 0 | 6 | 45 | 42.5 | 0.5 | 1 |
| 6 | 7 | 0 | 6 | 43 | 42.5 | 0.5 | 1 |
| 7 | 5 | 0 | 6 | 43.75 | 43.75 | 0.5 | 1 |
| 8 | 7 | 0 | 6 | 42.75 | 42.75 | 0.5 | 1 |
| 9 | 5 | 0 | 6 | 43.75 | 43.75 | 1.5 | 0 |
| 10 | 7 | 0 | 6 | 42.75 | 42.75 | 1.5 | 0 |
| 11 | 5 | 0 | 6 | 43.75 | 43.75 | 1 | 0.5 |
| 12 | 7 | 0 | 6 | 42.75 | 42.75 | 1 | 0.5 |
| 13 | 5 | 0.75 | 6 | 43.75 | 43.75 | 0.75 | 0 |
| 14 | 7 | 0.75 | 6 | 42.75 | 42.75 | 0.75 | 0 |
| 15 | 5 | 0.75 | 3 | 45.75 | 44.75 | 0.75 | 0 |
| 16 | 5 | 0.75 | 4 | 44.75 | 44.75 | 0.75 | 0 |
| 17 | 5 | 0.75 | 8 | 42.75 | 42.75 | 0.75 | 0 |
| 18 | 5 | 0.75 | 0 | 46.75 | 46.75 | 0.75 | 0 |
| 19 | 5 | 0.75 | 6 | 43.75 | 43.75 | 0.5 | 0.25 |
| 20 | 7 | 0.75 | 6 | 42.75 | 42.75 | 0.5 | 0.25 |
| 21 | 5 | 0.75 | 3 | 45.75 | 44.75 | 0.5 | 0.25 |
| 22 | 5 | 0.75 | 4 | 44.75 | 44.75 | 0.5 | 0.25 |
| 23 | 5 | 0.75 | 8 | 42.75 | 42.75 | 0.5 | 0.25 |
| 24 | 5 | 0.75 | 0 | 46.75 | 46.75 | 0.5 | 0.25 |
| 25 | 5 | 0.75 | 3 | 43.25 | 43.25 | 0.5 | 2 |
| 26 | 7 | 0.75 | 3 | 43.25 | 43.25 | 0.75 | 2 |
| 27 | 10 | 0.75 | 3 | 42 | 42 | 0.25 | 2 |
| 28 | 15 | 0.75 | 3 | 39 | 39.75 | 0.5 | 2 |
| 29 | 5 | 1 | 6 | 43.25 | 43.25 | 0.5 | 1 |
| 30 | 7 | 1 | 6 | 43.25 | 41.25 | 0.5 | 1 |

The following are formulas of a tablet containing Compound 1 are manufactured according to a method disclosed herein as shown in Table 47.

TABLE 47
Tablet formulations

| | Ingredient % weight in Tablet | | | | | | |
|---|---|---|---|---|---|---|---|
| | Com- | | | | | | Mag- |
| | pound | | | | | | nesium |
| Formula | 1 | CCNa | HPMC | MCC | LMH | CSD | Stearate |
| 31 | 5 | 0.75 | 6 | 43.25 | 43.5 | 0.5 | 1 |
| 32 | 7 | 0.75 | 3 | 44.25 | 43.5 | 0.5 | 1 |
| 33 | 10 | 0.75 | 3 | 43.25 | 41.5 | 0.5 | 1 |
| 34 | 15 | 0.75 | 3 | 39.5 | 40.25 | 0.5 | 1 |
| 35 | 5 | 0 | 6 | 45 | 42.5 | 0.5 | 1 |
| 36 | 7 | 0 | 6 | 43 | 42.5 | 0.5 | 1 |
| 37 | 5 | 0 | 6 | 43.75 | 43.75 | 0.5 | 1 |
| 38 | 7 | 0 | 6 | 42.75 | 42.75 | 0.5 | 1 |
| 39 | 5 | 0 | 6 | 43.75 | 43.75 | 1.5 | 0 |
| 40 | 7 | 0 | 6 | 42.75 | 42.75 | 1.5 | 0 |
| 41 | 5 | 0 | 6 | 43.75 | 43.75 | 1 | 0.5 |
| 42 | 7 | 0 | 6 | 42.75 | 42.75 | 1 | 0.5 |
| 43 | 5 | 0.75 | 6 | 43.75 | 43.75 | 0.75 | 0 |
| 44 | 7 | 0.75 | 6 | 42.75 | 42.75 | 0.75 | 0 |
| 45 | 5 | 0.75 | 3 | 45.75 | 44.75 | 0.75 | 0 |
| 46 | 5 | 0.75 | 4 | 44.75 | 44.75 | 0.75 | 0 |
| 47 | 5 | 0.75 | 8 | 42.75 | 42.75 | 0.75 | 0 |
| 48 | 5 | 0.75 | 0 | 46.75 | 46.75 | 0.75 | 0 |
| 49 | 5 | 0.75 | 6 | 43.75 | 43.75 | 0.5 | 0.25 |
| 50 | 7 | 0.75 | 6 | 42.75 | 42.75 | 0.5 | 0.25 |
| 51 | 5 | 0.75 | 3 | 45.75 | 44.75 | 0.5 | 0.25 |
| 52 | 5 | 0.75 | 4 | 44.75 | 44.75 | 0.5 | 0.25 |
| 53 | 5 | 0.75 | 8 | 42.75 | 42.75 | 0.5 | 0.25 |
| 54 | 5 | 0.75 | 0 | 46.75 | 46.75 | 0.5 | 0.25 |
| 55 | 5 | 0.75 | 3 | 43.25 | 43.25 | 0.5 | 2 |
| 56 | 7 | 0.75 | 3 | 43.25 | 43.25 | 0.75 | 2 |
| 57 | 10 | 0.75 | 3 | 42 | 42 | 0.25 | 2 |
| 58 | 15 | 0.75 | 3 | 39 | 39.75 | 0.5 | 2 |
| 59 | 5 | 1 | 6 | 43.25 | 43.25 | 0.5 | 1 |
| 60 | 7 | 1 | 6 | 43.25 | 41.25 | 0.5 | 1 |

The following are formulas of a tablet containing Compound 1 are manufactured according to a method disclosed herein in Table 48.

TABLE 48
Tablet formulations

| | Ingredient % weight in Tablet | | | | | | |
|---|---|---|---|---|---|---|---|
| | Com- | | | | | | Mag- |
| | pound | | | | | | nesium |
| Formula | 1 | CMC | HPC | MCC | LMH | CSD | Stearate |
| 61 | 5 | 0.75 | 6 | 43.25 | 43.5 | 0.5 | 1 |
| 62 | 7 | 0.75 | 3 | 44.25 | 43.5 | 0.5 | 1 |
| 63 | 10 | 0.75 | 3 | 43.25 | 41.5 | 0.5 | 1 |
| 64 | 15 | 0.75 | 3 | 39.5 | 40.25 | 0.5 | 1 |
| 65 | 5 | 0 | 6 | 45 | 42.5 | 0.5 | 1 |
| 66 | 7 | 0 | 6 | 43 | 42.5 | 0.5 | 1 |
| 67 | 5 | 0 | 6 | 43.75 | 43.75 | 0.5 | 1 |
| 68 | 7 | 0 | 6 | 42.75 | 42.75 | 0.5 | 1 |
| 69 | 5 | 0 | 6 | 43.75 | 43.75 | 1.5 | 0 |
| 70 | 7 | 0 | 6 | 42.75 | 42.75 | 1.5 | 0 |
| 71 | 5 | 0 | 6 | 43.75 | 43.75 | 1 | 0.5 |
| 72 | 7 | 0 | 6 | 42.75 | 42.75 | 1 | 0.5 |
| 73 | 5 | 0.75 | 6 | 43.75 | 43.75 | 0.75 | 0 |
| 74 | 7 | 0.75 | 6 | 42.75 | 42.75 | 0.75 | 0 |
| 75 | 5 | 0.75 | 3 | 45.75 | 44.75 | 0.75 | 0 |
| 76 | 5 | 0.75 | 4 | 44.75 | 44.75 | 0.75 | 0 |
| 77 | 5 | 0.75 | 8 | 42.75 | 42.75 | 0.75 | 0 |
| 78 | 5 | 0.75 | 0 | 46.75 | 46.75 | 0.75 | 0 |
| 79 | 5 | 0.75 | 6 | 43.75 | 43.75 | 0.5 | 0.25 |
| 80 | 7 | 0.75 | 6 | 42.75 | 42.75 | 0.5 | 0.25 |
| 81 | 5 | 0.75 | 3 | 45.75 | 44.75 | 0.5 | 0.25 |
| 82 | 5 | 0.75 | 4 | 44.75 | 44.75 | 0.5 | 0.25 |
| 83 | 5 | 0.75 | 8 | 42.75 | 42.75 | 0.5 | 0.25 |
| 84 | 5 | 0.75 | 0 | 46.75 | 46.75 | 0.5 | 0.25 |
| 85 | 5 | 0.75 | 3 | 43.25 | 43.25 | 0.5 | 2 |
| 86 | 7 | 0.75 | 3 | 43.25 | 43.25 | 0.75 | 2 |
| 87 | 10 | 0.75 | 3 | 42 | 42 | 0.25 | 2 |
| 88 | 15 | 0.75 | 3 | 39 | 39.75 | 0.5 | 2 |
| 89 | 5 | 1 | 6 | 43.25 | 43.25 | 0.5 | 1 |
| 90 | 7 | 1 | 6 | 43.25 | 41.25 | 0.5 | 1 |

The following are formulas of a tablet containing Compound 1 are manufactured according to a method disclosed herein in Table 49.

TABLE 49
Tablet formulations

| | Ingredient % weight in Tablet | | | | | | |
|---|---|---|---|---|---|---|---|
| | Compound | sodium | | | | | Zinc |
| Formula | 1 | alginate | PVP | MCC | LMH | CSD | Stearate |
| 91 | 5 | 0.75 | 6 | 43.25 | 43.5 | 0.5 | 1 |
| 92 | 7 | 0.75 | 3 | 44.25 | 43.5 | 0.5 | 1 |
| 93 | 10 | 0.75 | 3 | 43.25 | 41.5 | 0.5 | 1 |
| 94 | 15 | 0.75 | 3 | 39.5 | 40.25 | 0.5 | 1 |
| 95 | 5 | 0 | 6 | 45 | 42.5 | 0.5 | 1 |
| 96 | 7 | 0 | 6 | 43 | 42.5 | 0.5 | 1 |
| 97 | 5 | 0 | 6 | 43.75 | 43.75 | 0.5 | 1 |
| 98 | 7 | 0 | 6 | 42.75 | 42.75 | 0.5 | 1 |
| 99 | 5 | 0 | 6 | 43.75 | 43.75 | 1.5 | 0 |
| 100 | 7 | 0 | 6 | 42.75 | 42.75 | 1.5 | 0 |
| 101 | 5 | 0 | 6 | 43.75 | 43.75 | 1 | 0.5 |
| 102 | 7 | 0 | 6 | 42.75 | 42.75 | 1 | 0.5 |
| 103 | 5 | 0.75 | 6 | 43.75 | 43.75 | 0.75 | 0 |
| 104 | 7 | 0.75 | 6 | 42.75 | 42.75 | 0.75 | 0 |
| 105 | 5 | 0.75 | 3 | 45.75 | 44.75 | 0.75 | 0 |
| 106 | 5 | 0.75 | 4 | 44.75 | 44.75 | 0.75 | 0 |
| 107 | 5 | 0.75 | 8 | 42.75 | 42.75 | 0.75 | 0 |
| 108 | 5 | 0.75 | 0 | 46.75 | 46.75 | 0.75 | 0 |
| 109 | 5 | 0.75 | 6 | 43.75 | 43.75 | 0.5 | 0.25 |
| 110 | 7 | 0.75 | 6 | 42.75 | 42.75 | 0.5 | 0.25 |
| 111 | 5 | 0.75 | 3 | 45.75 | 44.75 | 0.5 | 0.25 |
| 112 | 5 | 0.75 | 4 | 44.75 | 44.75 | 0.5 | 0.25 |
| 113 | 5 | 0.75 | 8 | 42.75 | 42.75 | 0.5 | 0.25 |
| 114 | 5 | 0.75 | 0 | 46.75 | 46.75 | 0.5 | 0.25 |
| 115 | 5 | 0.75 | 3 | 43.25 | 43.25 | 0.5 | 2 |
| 116 | 7 | 0.75 | 3 | 43.25 | 43.25 | 0.75 | 2 |
| 117 | 10 | 0.75 | 3 | 42 | 42 | 0.25 | 2 |
| 118 | 15 | 0.75 | 3 | 39 | 39.75 | 0.5 | 2 |
| 119 | 5 | 1 | 6 | 43.25 | 43.25 | 0.5 | 1 |
| 120 | 7 | 1 | 6 | 43.25 | 41.25 | 0.5 | 1 |

The following are formulas of a tablet containing Compound 1 are manufactured according to a method disclosed herein in Table 50.

TABLE 50
Tablet formulations

| | Ingredient % weight in Tablet | | | | | | |
|---|---|---|---|---|---|---|---|
| | Com- | | | | | | |
| | pound | | | | | | Aluminum |
| Formula | 1 | CCNa | HPC | MCC | LMH | CSD | Stearate |
| 121 | 5 | 0.75 | 6 | 43.25 | 43.5 | 0.5 | 1 |
| 122 | 7 | 0.75 | 3 | 44.25 | 43.5 | 0.5 | 1 |
| 123 | 10 | 0.75 | 3 | 43.25 | 41.5 | 0.5 | 1 |
| 124 | 15 | 0.75 | 3 | 39.5 | 40.25 | 0.5 | 1 |
| 125 | 5 | 0 | 6 | 45 | 42.5 | 0.5 | 1 |
| 126 | 7 | 0 | 6 | 43 | 42.5 | 0.5 | 1 |
| 127 | 5 | 0 | 6 | 43.75 | 43.75 | 0.5 | 1 |
| 128 | 7 | 0 | 6 | 42.75 | 42.75 | 0.5 | 1 |
| 129 | 5 | 0 | 6 | 43.75 | 43.75 | 1.5 | 0 |
| 130 | 7 | 0 | 6 | 42.75 | 42.75 | 1.5 | 0 |
| 131 | 5 | 0 | 6 | 43.75 | 43.75 | 1 | 0.5 |
| 132 | 7 | 0 | 6 | 42.75 | 42.75 | 1 | 0.5 |
| 133 | 5 | 0.75 | 6 | 43.75 | 43.75 | 0.75 | 0 |
| 134 | 7 | 0.75 | 6 | 42.75 | 42.75 | 0.75 | 0 |
| 135 | 5 | 0.75 | 3 | 45.75 | 44.75 | 0.75 | 0 |
| 136 | 5 | 0.75 | 4 | 44.75 | 44.75 | 0.75 | 0 |
| 137 | 5 | 0.75 | 8 | 42.75 | 42.75 | 0.75 | 0 |
| 138 | 5 | 0.75 | 0 | 46.75 | 46.75 | 0.75 | 0 |
| 139 | 5 | 0.75 | 6 | 43.75 | 43.75 | 0.5 | 0.25 |
| 140 | 7 | 0.75 | 6 | 42.75 | 42.75 | 0.5 | 0.25 |
| 141 | 5 | 0.75 | 3 | 45.75 | 44.75 | 0.5 | 0.25 |
| 142 | 5 | 0.75 | 4 | 44.75 | 44.75 | 0.5 | 0.25 |
| 143 | 5 | 0.75 | 8 | 42.75 | 42.75 | 0.5 | 0.25 |
| 144 | 5 | 0.75 | 0 | 46.75 | 46.75 | 0.5 | 0.25 |
| 145 | 5 | 0.75 | 3 | 43.25 | 43.25 | 0.5 | 2 |
| 146 | 7 | 0.75 | 3 | 43.25 | 43.25 | 0.75 | 2 |
| 147 | 10 | 0.75 | 3 | 42 | 42 | 0.25 | 2 |
| 148 | 15 | 0.75 | 3 | 39 | 39.75 | 0.5 | 2 |
| 149 | 5 | 1 | 6 | 43.25 | 43.25 | 0.5 | 1 |
| 150 | 7 | 1 | 6 | 43.25 | 41.25 | 0.5 | 1 |

The following are formulas of a tablet containing Compound 1 are manufactured according to a method disclosed herein as shown in Table 51.

TABLE 51
Tablet formulations

| | Ingredient % weight in Tablet | | | | | | |
|---|---|---|---|---|---|---|---|
| | Com- | | | | | | |
| | pound | agar- | | cros- | | | Zinc |
| Formula | 1 | agar | HPMC | povidone | LMH | CSD | Stearate |
| 151 | 5 | 0.75 | 6 | 43.25 | 43.5 | 0.5 | 1 |
| 152 | 7 | 0.75 | 3 | 44.25 | 43.5 | 0.5 | 1 |
| 153 | 10 | 0.75 | 3 | 43.25 | 41.5 | 0.5 | 1 |
| 154 | 15 | 0.75 | 3 | 39.5 | 40.25 | 0.5 | 1 |
| 155 | 5 | 0 | 6 | 45 | 42.5 | 0.5 | 1 |
| 156 | 7 | 0 | 6 | 43 | 42.5 | 0.5 | 1 |
| 157 | 5 | 0 | 6 | 43.75 | 43.75 | 0.5 | 1 |
| 158 | 7 | 0 | 6 | 42.75 | 42.75 | 0.5 | 1 |
| 159 | 5 | 0 | 6 | 43.75 | 43.75 | 1.5 | 0 |
| 160 | 7 | 0 | 6 | 42.75 | 42.75 | 1.5 | 0 |
| 161 | 5 | 0 | 6 | 43.75 | 43.75 | 1 | 0.5 |
| 162 | 7 | 0 | 6 | 42.75 | 42.75 | 1 | 0.5 |
| 163 | 5 | 0.75 | 6 | 43.75 | 43.75 | 0.75 | 0 |
| 164 | 7 | 0.75 | 6 | 42.75 | 42.75 | 0.75 | 0 |
| 165 | 5 | 0.75 | 3 | 45.75 | 44.75 | 0.75 | 0 |
| 166 | 5 | 0.75 | 4 | 44.75 | 44.75 | 0.75 | 0 |
| 167 | 5 | 0.75 | 8 | 42.75 | 42.75 | 0.75 | 0 |
| 168 | 5 | 0.75 | 0 | 46.75 | 46.75 | 0.75 | 0 |
| 169 | 5 | 0.75 | 6 | 43.75 | 43.75 | 0.5 | 0.25 |
| 170 | 7 | 0.75 | 6 | 42.75 | 42.75 | 0.5 | 0.25 |
| 171 | 5 | 0.75 | 3 | 45.75 | 44.75 | 0.5 | 0.25 |
| 172 | 5 | 0.75 | 4 | 44.75 | 44.75 | 0.5 | 0.25 |
| 173 | 5 | 0.75 | 8 | 42.75 | 42.75 | 0.5 | 0.25 |
| 174 | 5 | 0.75 | 0 | 46.75 | 46.75 | 0.5 | 0.25 |
| 175 | 5 | 0.75 | 3 | 43.25 | 43.25 | 0.5 | 2 |
| 176 | 7 | 0.75 | 3 | 43.25 | 43.25 | 0.75 | 2 |
| 177 | 10 | 0.75 | 3 | 42 | 42 | 0.25 | 2 |
| 178 | 15 | 0.75 | 3 | 39 | 39.75 | 0.5 | 2 |
| 179 | 5 | 1 | 6 | 43.25 | 43.25 | 0.5 | 1 |
| 180 | 7 | 1 | 6 | 43.25 | 41.25 | 0.5 | 1 |

The following are formulas of a tablet containing Compound 1 are manufactured according to a method disclosed herein as shown in Table 52.

TABLE 52
Tablet formulations

| | Ingredient % weight in Tablet | | | | | | |
|---|---|---|---|---|---|---|---|
| | Com- | magnesium | | | | | Mag- |
| | pound | aluminum | | | | | nesium |
| Formula | 1 | silicate | HPC | MCC | LMH | CSD | Stearate |
| 181 | 5 | 0.75 | 6 | 43.25 | 43.5 | 0.5 | 1 |
| 182 | 7 | 0.75 | 3 | 44.25 | 43.5 | 0.5 | 1 |
| 183 | 10 | 0.75 | 3 | 43.25 | 41.5 | 0.5 | 1 |
| 184 | 15 | 0.75 | 3 | 39.5 | 40.25 | 0.5 | 1 |
| 185 | 5 | 0 | 6 | 45 | 42.5 | 0.5 | 1 |
| 186 | 7 | 0 | 6 | 43 | 42.5 | 0.5 | 1 |
| 187 | 5 | 0 | 6 | 43.75 | 43.75 | 0.5 | 1 |
| 188 | 7 | 0 | 6 | 42.75 | 42.75 | 0.5 | 1 |
| 189 | 5 | 0 | 6 | 43.75 | 43.75 | 1.5 | 0 |
| 190 | 7 | 0 | 6 | 42.75 | 42.75 | 1.5 | 0 |
| 191 | 5 | 0 | 6 | 43.75 | 43.75 | 1 | 0.5 |
| 192 | 7 | 0 | 6 | 42.75 | 42.75 | 1 | 0.5 |
| 193 | 5 | 0.75 | 6 | 43.75 | 43.75 | 0.75 | 0 |
| 194 | 7 | 0.75 | 6 | 42.75 | 42.75 | 0.75 | 0 |
| 195 | 5 | 0.75 | 3 | 45.75 | 44.75 | 0.75 | 0 |
| 196 | 5 | 0.75 | 4 | 44.75 | 44.75 | 0.75 | 0 |
| 197 | 5 | 0.75 | 8 | 42.75 | 42.75 | 0.75 | 0 |
| 198 | 5 | 0.75 | 0 | 46.75 | 46.75 | 0.75 | 0 |
| 199 | 5 | 0.75 | 6 | 43.75 | 43.75 | 0.5 | 0.25 |
| 200 | 7 | 0.75 | 6 | 42.75 | 42.75 | 0.5 | 0.25 |
| 201 | 5 | 0.75 | 3 | 45.75 | 44.75 | 0.5 | 0.25 |
| 202 | 5 | 0.75 | 4 | 44.75 | 44.75 | 0.5 | 0.25 |
| 203 | 5 | 0.75 | 8 | 42.75 | 42.75 | 0.5 | 0.25 |
| 204 | 5 | 0.75 | 0 | 46.75 | 46.75 | 0.5 | 0.25 |
| 205 | 5 | 0.75 | 3 | 43.25 | 43.25 | 0.5 | 2 |
| 206 | 7 | 0.75 | 3 | 43.25 | 43.25 | 0.75 | 2 |
| 207 | 10 | 0.75 | 3 | 42 | 42 | 0.25 | 2 |
| 208 | 15 | 0.75 | 3 | 39 | 39.75 | 0.5 | 2 |
| 209 | 5 | 1 | 6 | 43.25 | 43.25 | 0.5 | 1 |
| 210 | 7 | 1 | 6 | 43.25 | 41.25 | 0.5 | 1 |

The following are formulas of a tablet containing Compound 1 are manufactured according to a method disclosed herein as shown in Table 53.

TABLE 53
Tablet formulations

| | Ingredient % weight in Tablet | | | | | | |
|---|---|---|---|---|---|---|---|
| | Com- | | | | | | Mag- |
| | pound | calcium | methyl | | | | nesium |
| Formula | 1 | carbonate | cellulose | MCC | LMH | CSD | Stearate |
| 211 | 5 | 0.75 | 6 | 43.25 | 43.5 | 0.5 | 1 |
| 212 | 7 | 0.75 | 3 | 44.25 | 43.5 | 0.5 | 1 |
| 213 | 10 | 0.75 | 3 | 43.25 | 41.5 | 0.5 | 1 |
| 214 | 15 | 0.75 | 3 | 39.5 | 40.25 | 0.5 | 1 |
| 215 | 5 | 0 | 6 | 45 | 42.5 | 0.5 | 1 |
| 216 | 7 | 0 | 6 | 43 | 42.5 | 0.5 | 1 |
| 217 | 5 | 0 | 6 | 43.75 | 43.75 | 0.5 | 1 |
| 218 | 7 | 0 | 6 | 42.75 | 42.75 | 0.5 | 1 |
| 219 | 5 | 0 | 6 | 43.75 | 43.75 | 1.5 | 0 |
| 220 | 7 | 0 | 6 | 42.75 | 42.75 | 1.5 | 0 |
| 221 | 5 | 0 | 6 | 43.75 | 43.75 | 1 | 0.5 |
| 222 | 7 | 0 | 6 | 42.75 | 42.75 | 1 | 0.5 |
| 223 | 5 | 0.75 | 6 | 43.75 | 43.75 | 0.75 | 0 |
| 224 | 7 | 0.75 | 6 | 42.75 | 42.75 | 0.75 | 0 |
| 225 | 5 | 0.75 | 3 | 45.75 | 44.75 | 0.75 | 0 |
| 226 | 5 | 0.75 | 4 | 44.75 | 44.75 | 0.75 | 0 |
| 227 | 5 | 0.75 | 8 | 42.75 | 42.75 | 0.75 | 0 |
| 228 | 5 | 0.75 | 0 | 46.75 | 46.75 | 0.75 | 0 |
| 229 | 5 | 0.75 | 6 | 43.75 | 43.75 | 0.5 | 0.25 |
| 230 | 7 | 0.75 | 6 | 42.75 | 42.75 | 0.5 | 0.25 |
| 231 | 5 | 0.75 | 3 | 45.75 | 44.75 | 0.5 | 0.25 |
| 232 | 5 | 0.75 | 4 | 44.75 | 44.75 | 0.5 | 0.25 |
| 233 | 5 | 0.75 | 8 | 42.75 | 42.75 | 0.5 | 0.25 |
| 234 | 5 | 0.75 | 0 | 46.75 | 46.75 | 0.5 | 0.25 |
| 235 | 5 | 0.75 | 3 | 43.25 | 43.25 | 0.5 | 2 |
| 236 | 7 | 0.75 | 3 | 43.25 | 43.25 | 0.75 | 2 |
| 237 | 10 | 0.75 | 3 | 42 | 42 | 0.25 | 2 |
| 238 | 15 | 0.75 | 3 | 39 | 39.75 | 0.5 | 2 |
| 239 | 5 | 1 | 6 | 43.25 | 43.25 | 0.5 | 1 |
| 240 | 7 | 1 | 6 | 43.25 | 41.25 | 0.5 | 1 |

We claim:

1. A tablet pharmaceutical composition comprising:
(i) a compound of Formula (I), a pharmaceutically acceptable salt,
crystalline, solvate, polymorph, prodrug, metabolite, N-oxide, stereoisomer, or isomer thereof: 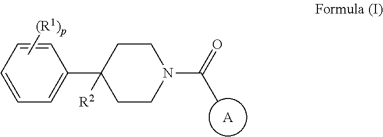
wherein:
each R¹ is independently halogen, haloalkyl, or alkyl;
R² is —H, —OH, or halogen;
p is 0, 1, 2, 3, 4, or 5;
A has the structure: 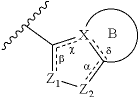
wherein:
α, β, χ, and δ are each independently absent or present, and when present each is a bond;
X is C;
Z₁ is S, O, or N;
Z₂ is S, O, N, or NR³;
R³ is H, C₁-C₄ alkyl, or oxetane; and
B is a substituted or unsubstituted fused 5-, 6-, or 7-membered ring structure; and
(ii) at least one excipient, wherein the excipient comprises one or more of a disintegrant, dispersion polymer, diluent, glidant, and lubricant.

2. The pharmaceutical composition of claim 1, wherein A has the structure 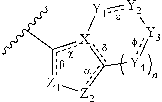
wherein:
n is 0, 1, or 2;
α, β, χ, δ, ε, and ϕ are each independently absent or present, and when present each is a bond;
Z₁ is S, O, or N;
Z₂ is S, O, N or NR³,
wherein R³ is H, C₁-C₄ alkyl, or oxetane;
X is C;
Y₁, Y₂, Y₃ and each occurrence of Y₄ are each independently CR⁴, C(R⁵)₂, NR⁶, O, N, SO₂, or —(C=O)—, wherein:
R⁴ is H, halogen, C₁-C₁₀ alkyl, C₁-C₁₀ cycloalkyl, —O(C₁-C₁₀ alkyl), —C(O)OH, —C(O)O(C₁-C₁₀ alkyl), —C(O)NH₂, —C(O)NH (C₁-C₄ alkyl), —C(O)N(C₁-C₄ alkyl)₂, —NHC(O)NH(C₁-C₁₀ alkyl), —NHC(O)N(C₁-C₄ alkyl)₂, —SO₂NH(C₁-C₁₀ alkyl), —SO₂N(C₁-C₁₀ alkyl)₂, —CN, or —CF₃;
R⁵ is H or C₁-C₁₀ alkyl; and
R⁶ is H, C₁-C₁₀ alkyl, C₃-C₆ cycloalkyl, —(C₁-C₁₀ alkylene)CF₃, —(C₁-C₁₀ alkylene)OCH₃, —(C₁-C₁₀ alkylene)-halogen, —SO₂(C₁-C₁₀ alkyl), —SO₂(C₁-C₁₀ alkylene)-CF₃, —SO₂(C₁-C₁₀ alkylene)OCH₃, —SO₂(C₁-C₁₀ alkylene)-halogen, —C(O)(C₁-C₁₀ alkyl), —C(O)(C₁-C₁₀ alkylene)CF₃, —C(O)(C₁-C₁₀ alkylene)OCH₃, —C(O)(C₁-C₁₀ alkylene)-halogen, —C(O)NH(C₁-C₁₀ alkyl), —C(O)N(C₁-C₁₀ alkyl)₂, —(C₁-C₁₀ alkyl)C(O)OH, —C(O)NH₂, or oxetane.

3. The pharmaceutical composition of claim 1, wherein A has the structure 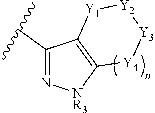
wherein:
n is 0;
R³ is H, C₁-C₄ alkyl, or oxetane;
Y₁ and Y₃ are each CH₂ or C(CH₃)₂;
Y₂ is O, SO₂, or NR⁶; and
R⁶ is H, C₁-C₄ alkyl, C₃-C₆ cycloalkyl, —(C₁-C₄ alkylene)CF₃, —(C₁-C₄ alkylene)OCH₃, —(C₁-C₄ alkylene)-halogen, —SO₂(C₁-C₄ alkyl), —SO₂(C₁-C₄ alkylene)CF₃, —SO₂(C₁-C₄ alkylene)OCH₃, —SO₂(C₁-C₄ alkylene)-halogen, —C(O)(C₁-C₄ alkyl), —C(O)(C₁-C₄ alkylene)CF₃, —C(O)(C₁-C₄ alkylene)OCH₃, —C(O)(C₁-C₄ alkylene)-halogen, —C(O)NH(C₁-C₄ alkyl), —C(O)N(C₁-C₄ alkyl)₂, —(C₁-C₄ alkylene)C(O)OH, —C(O)NH₂, or oxetane.

4. The pharmaceutical composition of claim 1, wherein the compound of Formula (I) has the structure: 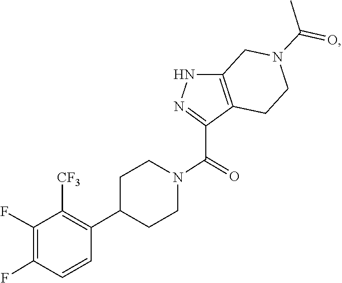 [SMILES: CC(=O)N1CCc2c(C(=O)N3CCC(c4ccc(F)c(F)c4C(F)(F)F)CC3)n[nH]c2C1]
or a pharmaceutically acceptable salt, crystalline, solvate, polymorph, prodrug, metabolite, N-oxide, stereoisomer, or isomer thereof.

5. The pharmaceutical composition of any one of the preceding claims, wherein the compound of Formula (I) is effective to reduce RBP4 concentrations in dosage forms of about 1 to about 200 mg or about 5 to about 25 mg.

6. The pharmaceutical composition of any one of the preceding claims, wherein the compound of Formula (I) is a micronized crystalline.

7. The pharmaceutical composition of any one of the preceding claims, wherein the compound of Formula (I) is a polymorph exhibiting an x-ray powder diffraction pattern having at least three characteristic peaks expressed in degrees two theta (+/−0.5 degree theta) at 6.7, 9.3, 14.1, 17.2, 23.5, 27.1, and/or 29.0.

8. The pharmaceutical composition of any one of the preceding claims, wherein the compound of Formula (I) is amorphous.

9. The pharmaceutical composition of any one of the preceding claims, wherein the at least one excipient comprises a disintegrant.

10. The pharmaceutical composition of claim 9, wherein the disintegrant is selected from the group comprising agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof.

11. The pharmaceutical composition of claim 9 or 10, wherein the disintegrant comprises CCNa, MCC, crospovidone, tapioca starch, or a combination thereof.

12. The pharmaceutical composition of claims 9 to 11, wherein the disintegrant is about 0.01-99% by weight, 1-50% by weight, or 2-20% by weight of the pharmaceutical composition.

13. The pharmaceutical composition of claim 11, wherein the CCNa or MCC is about 0.75% or 3% by weight of the pharmaceutical composition.

14. The pharmaceutical composition of any one of the preceding claims, wherein the at least one excipient comprises a dispersion polymer.

15. The pharmaceutical composition of claim 14, wherein the dispersion polymer is selected from the group comprising hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, poly oxyethylene-polyoxypropylene block copolymers, and combinations thereof.

16. The pharmaceutical composition of claim 14 or 15, wherein the compound of Formula (I) is amorphous and/or molecularly dispersed in the dispersion polymer.

17. The pharmaceutical composition of claim 14 or 15, wherein the dispersion polymer comprises HPC, HPMC, or both.

18. The pharmaceutical composition of claim 14 or 15, wherein the dispersion polymer comprises HPMC, HPMC-AS, PVP, or a combination thereof.

19. The pharmaceutical composition of any one of claims 14 to 18, wherein the dispersion polymer is about 0.01-99% by weight, 1-50% by weight, or 2-20% by weight of the pharmaceutical composition.

20. The pharmaceutical composition of claim 17, wherein the HPC or HPMC is about 6% by weight of the pharmaceutical composition.

21. The pharmaceutical composition of claim 18, wherein the HPMC, HPMC-AS, or PVP is about 10% or 20% by weight of the pharmaceutical composition.

22. The pharmaceutical composition of any one of claims 1 to 16, wherein the at least one excipient comprises about 0.75% of CCNa, MCC, crospovidone, or tapioca starch by weight of the pharmaceutical composition and about 6% of HPC by weight of the of the pharmaceutical composition.

23. The pharmaceutical composition of any one of claims 1 to 16, wherein the at least one excipient comprises about 0.75% of CCNa, MCC, crospovidone, or tapioca starch by weight of the pharmaceutical composition and about 10% of HPMC by weight of the pharmaceutical composition.

24. The pharmaceutical composition of any one of claims 1 to 16, wherein the at least one excipient comprises about 0.75% of CCNa, MCC, crospovidone, or tapioca starch by weight of the pharmaceutical composition and about 20% of HPMC by weight of the pharmaceutical composition.

25. The pharmaceutical composition of any one of claims 14-24, wherein the compound of Formula (I) is 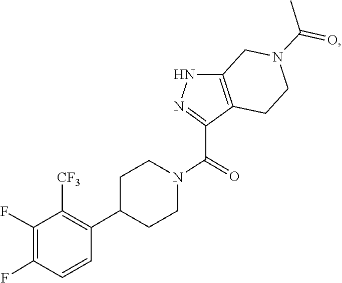 [SMILES: CC(=O)N1CCc2c(C(=O)N3CCC(c4ccc(F)c(F)c4C(F)(F)F)CC3)n[nH]c2C1]
or a pharmaceutically acceptable salt, crystalline solvate, polymorph, prodrug, metabolite, N-oxide, stereoisomer, or isomer thereof.

26. The pharmaceutical composition of any one of the preceding claims, wherein the at least one excipient comprises a diluent, a binder, an anti-adherent, a filler, a sweetener, a wetting agent, a glidant, a lubricant, or a surfactant, or any combinations thereof.

27. The pharmaceutical composition of any one of the preceding claims, wherein the compound of Formula (I) is not formulated for delayed release.

28. The pharmaceutical composition of any one of the preceding claims, wherein the at least one excipient is not formulated for delayed release.

29. The pharmaceutical composition of any one of the preceding claims, further comprising an external coating.

30. The pharmaceutical composition of claim 29, wherein the external coating is about 0.01-99% by weight, 1-50% by weight, or 2-20% by weight of the pharmaceutical composition.

31. The pharmaceutical composition of claim 29 or 30, wherein the external coating does not cause delayed release of the compound of Formula (I) and/or the at least one excipient.

32. The pharmaceutical composition of any one of claims 26 to 31, wherein the diluent comprises MCC, lactose monohydrate (LMH), or both.

33. The pharmaceutical composition of any one of claims 26 to 31, wherein the binder comprises MCC, HPC, or both.

34. The pharmaceutical composition of any one of claims 26 to 31, wherein the anti-adherent comprises colloidal silicon dioxide (CSD), magnesium stearate, or both.

35. The pharmaceutical composition of any one of claims 26 to 31, wherein the glidant comprise CSD.

36. The pharmaceutical composition of any one of claims 26 to 31, wherein the lubricant comprises magnesium stearate.

37. The pharmaceutical composition of any one of claims 26 to 36, wherein the at least one excipient comprises one or more of MCC, LMH, HPC, CCNa, CSD and magnesium stearate.

38. The pharmaceutical composition of claim 37, wherein the at least one excipient comprises two or more of MCC, LMH, HPC, CCNa, CSD and magnesium stearate.

39. The pharmaceutical composition of any one of claims 26 to 38, wherein the diluent functions as a binder and/or a disintegrant.

40. The pharmaceutical composition of any one of claims 26 to 39, wherein the anti-adherent functions as a gliant or a lubricant.

41. The pharmaceutical composition of any one of the preceding claims, wherein the dry weight of the pharmaceutical composition is from about 0.1 mg to about 400 mg.

42. The pharmaceutical composition of any one of the preceding claims, wherein the compound of Formula (I) is about 1-99.99% by weight, about 10-80% by weight, or about 20-60% by weight of the pharmaceutical composition.

43. The pharmaceutical composition of any one of claims 1 to 42, wherein the pharmaceutical composition does not exhibit substantial loss of chemical stability, degradation, and/or decomposition after about 1 week, 2 weeks, 3 weeks, 4 weeks, 10 weeks, 20 weeks, 30 weeks, 40 weeks, one year, two years, or three years at 25° C. when stored in otherwise atmospheric conditions.

44. The pharmaceutical composition of any one of claims 1 to 42, wherein the pharmaceutical composition does not exhibit substantial loss of chemical stability, degradation, and/or decomposition of the compound of Formula (I), a pharmaceutically acceptable salt, crystalline, solvate, polymorph, prodrug, metabolite, N-oxide, stereoisomer, or isomer thereof, or the at least one excipient after about 1 week, 2 weeks, 3 weeks, 4 weeks, 10 weeks, 20 weeks, 30 weeks, 40 weeks, one year, two years, or three years at 25° C. when stored in otherwise atmospheric conditions.

45. The pharmaceutical composition of any one of claims 1 to 42, wherein the pharmaceutical composition does not exhibit substantial loss of chemical stability, degradation, and/or decomposition after about 1 week, 2 weeks, 3 weeks, 4 weeks, 10 weeks, 20 weeks, 30 weeks, 40 weeks, or one year at 40° C. when stored in otherwise atmospheric conditions.

46. The pharmaceutical composition of any one of claims 1 to 42, wherein the pharmaceutical composition does not exhibit substantial loss of chemical stability, degradation, and/or decomposition of the compound of Formula (I), a pharmaceutically acceptable salt, crystalline, solvate, polymorph, prodrug, metabolite, N-oxide, stereoisomer, or isomer thereof, or the at least one excipient after about 1 week, 2 weeks, 3 weeks, 4 weeks, 10 weeks, 20 weeks, 30 weeks, 40 weeks, or one year at 40° C. when stored in otherwise atmospheric conditions.

47. The pharmaceutical composition of any one of the preceding claims, wherein at least about 5%, 10%, 25%, 50%, 75%, 80%, 90%, 95%, or 100% of the pharmaceutical composition by weight has dissociated or dissolved after about 5 minutes, 10 minutes, 20 minutes, 30 minutes, 40 minutes, 50 minutes, or one hour when placed in an aqueous medium at about pH 7 and at 25° C.

48. The pharmaceutical composition of any one of the preceding claims, wherein at least about 5%, 10%, 25%, 50%, 75%, 80%, 90%, 95%, or 100% of the pharmaceutical composition by weight has dissociated or dissolved after about 5 minutes, 10 minutes, 20 minutes, 30 minutes, 40 minutes, 50 minutes, or one hour when placed in a simulated gastric fluid at 37° C.

49. The pharmaceutical composition of claim 47, wherein the aqueous medium comprises water.

50. The pharmaceutical composition of claim 48, wherein the pharmaceutical composition is placed in USP Apparatus Type II at 50 rpm in 900 mL of simulated gastric fluid medium at 37° C.

51. The pharmaceutical composition of claims 1 to 50, wherein the at least one excipient is about 0.01-99% by weight, 1-50% by weight, or 2-20% by weight of the pharmaceutical composition.

52. A method of treating an eye disease comprising administering a therapeutically effective amount of a pharmaceutical composition of any one of the preceding claims to a subject in need thereof.

53. The method of claim 52, wherein the eye disease is a disease characterized by excessive lipofuscin accumulation in the retina.

54. The method of claim 53, wherein the disease characterized by excessive lipofuscin accumulation comprises Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or an ABCA4 gene associated retinal disease.

55. A method for lowering the serum or plasma concentration of RBP4 in a subject, comprising administering to the subject a therapeutically effective amount of a pharmaceutical composition of any one of claims 1-51.

56. The method of any one of claims 52-55, wherein the therapeutically effective amount of the pharmaceutical composition comprises about 0.1 mg to about 400 mg of the compound of Formula (I).

57. The method of any one of claims 52-56, wherein the therapeutically effective amount of the pharmaceutical composition comprises about 0.5 mg to about 50 mg of the compound of Formula (I).

58. The method of any one of claims 52-57, wherein the therapeutically effective amount of the pharmaceutical composition comprises about 0.1 mg, about 0.5 mg, about 1 mg, about 5 mg, about 10 mg, about 25 mg, about 50 mg, about 100 mg, about 200 mg, or about 400 mg of the compound of Formula (I).

59. The method of any one of claims 52-58, wherein the pharmaceutical composition is administered one, two, three, or four times daily.

60. The method of any one of claims 52-58, wherein the pharmaceutical composition is administered daily, every other day, every other day 3 times a week, every 2 weeks, every 3 weeks, every 4 weeks, every 5 weeks, every 3 days, every 4 days, every 5 days, every 6 days, weekly, bi-weekly, 3 times a week, 4 times a week, 5 times a week, 6 times a week, once a month, twice a month, 3 times a month, once every 2 months, once every 3 months, once every 4 months, once every 5 months, or once every 6 months.

61. The method of any one of claims 52-58, wherein the pharmaceutical composition is administered once daily.

62. The method of any one of claims 52-61, wherein the pharmaceutical composition is administered orally.

63. The method of any one of claims 52-62, wherein the serum or plasma RBP4 concentration of the subject is reduced to below 1 μM after treatment.

64. A method of manufacturing a tablet pharmaceutical composition comprising the steps:
(i) co-sifting a compound of Formula (I) of any one of claims 1-4 or its pharmaceutically acceptable salt, a disintegrant, a dispersion polymer, two diluents, a glidant and a lubricant through a 30-mesh screen;
(ii) loading the sifted materials from step (i) to a blender and blending for a first period of time;
(iii) unloading the blended materials from step (ii) and sifting through a 30-mesh screen;
(iv) adding the sifted materials from step (iii) to a blender and blending for a second period of time;
(v) co-sifting a glidant and the lubricant through a 60-mesh screen and lubricating the blended materials from the step (iv) with a blender for a third period of time;
(vi) granulating the lubricated materials from step (v) by roller compaction;
(vii) sifting the lubricant through a 60-mesh screen and lubricating with granulated materials from the step (vi) for a fourth period of time; and
(viii) compressing the materials from the step (vii) into the tablet pharmaceutical composition.

65. The method of claim 64, further comprising packaging the manufactured tablet pharmaceutical composition into a bottle.

66. The method of claim 64, wherein the disintegrant comprises agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, or combination thereof.

67. The method of claim 64, wherein the dispersion polymer comprises hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, poly oxyethylene-poly oxypropylene block copolymers, or combination thereof.

68. The method of claim 64, wherein the diluent comprises CCNa, HPC, MCC, LMH, CSD, magnesium stearate, or a combination thereof.

69. The method of claim 64, wherein the glidant comprises colloidal silicon dioxide, talk, corn starch, metal silicates, higher fatty acid metal salts, metal oxides, alkaline earth metal salts, metal hydroxides, or combination thereof.

70. The method of claim 64, wherein the lubricant comprises magnesium stearate, calcium stearate, zinc stearate, sodium stearate, stearic acid, aluminum stearate, leucine, glyceryl behenate, hydrogenated vegetable oil, sodium stearyl fumarate, or any combination thereof.

71. The method of claim 64, wherein the blending in step (ii) is carried out for 15 min at 15 rpm.

72. The method of claim 64, wherein the blending in step (iv) is carried out for 15 min at 15 rpm.

73. The method of claim 64, wherein the blending in step (v) is carried out for 5 min at 15 rpm.

74. The method of claim 64, wherein the blending in step (vii) is carried out for 5 min at 15 rpm.

75. The method of claim 64, wherein the step (vi) is carried out at a roll pressure from about 2 to about 5 MPa, a roll gap from about 0.3 to about 4 mm, a roll speed from about 3 to about 7 rpm, and/or a feed screw speed from about 18 to about 81 rpm.

76. The method of claim 64, wherein the step (vi) is carried out at a roll pressure of about 3 MPa, a roll gap of about 2 mm, a roll speed of about 4 rpm, and/or a feed screw speed of about 20 rpm.

77. The method of claim 64, wherein the step (viii) is carried out using a round-shaped punch of about 6.0 mm at a rotary speed from about 20 to about 30 rpm, a thickness scale from about 1.0 to about 3.5 mm, a fill depth scale from about 3.0 to about 10.0 mm, and/or a main compression force from about 3.0 to about 10.0 kN.

78. The method of claim 64, wherein the step (viii) is carried out at a rotary speed of about 30 rpm, a thickness scale of about 2.0 mm, a fill depth scale of about 6.0 mm, and/or a main compression force of about 7.0 kN.

79. The method of claim 64, wherein the bulk density of the lubricated materials of step (vi) is from about 0.45 to about 0.6 g/ml.

80. The method of claim 64, wherein the bulk density of the lubricated materials of step (vi) is from about 0.5 g/ml.

81. The method of claim 64, wherein the tablet pharmaceutical composition manufactured according to the method is from about 92.5 to about 107.5 mg.

82. The method of claim 64, wherein the tablet pharmaceutical composition manufactured according to the method is about 100 mg.

83. The method of claim 64, wherein the tablet pharmaceutical composition manufactured according to the method comprises a thickness from about 2.7 to about 3.3 mm.

84. The method of claim 64, wherein the tablet pharmaceutical composition manufactured according to the method comprises a thickness of about 3.0 mm.

85. The method of claim 64, wherein the tablet pharmaceutical composition manufactured according to the method comprises a hardness from about 45 to about 95N.

86. The method of claim 64, wherein the tablet pharmaceutical composition manufactured according to the method comprises a hardness of about 70N.

87. The method of claim 64, wherein the tablet pharmaceutical composition manufactured according to the method comprises a friability of no more than 1.0% by weight.

88. The method of claim 64, wherein the tablet pharmaceutical composition manufactured according to the method comprises a disintegration time of no more than 15 min.

89. The method of claim 64, wherein at least one of the two diluent functions as a binder and/or disintegrant.

90. The method of claim 64, wherein the glidant functions as an anti-adherent.

91. The method of claim 64, wherein the lubricant functions as an anti-adherent.

92. The method of claim 64, wherein at least one of the blenders used in steps (ii), (iv) and (v) is a bin blender of about 100 L.

93. The method of claim 65, wherein the step of packaging is carried out using a HDPE bottle of about 45 ml and the HDPE bottle contains 30 of the manufactured tablet pharmaceutical composition.

94. The method of claim 64, further comprising after step (iv), taking a sample of the blended materials from about 98.5 to about 295.5 mg from each of about 10 locations of the blender.

95. The method of claim 94, wherein the sample is about 197 mg.

96. The method of claim 94 or 95, wherein the taking is carried out so that all the individual assays are within mean±10% (absolute) and RSD % and the NMT is about 5%.

97. The method of claim 64, further comprising after step (v) testing BD and/or TD of the lubricated materials from step (v) so that the resulting density is around 0.5 g/ml or from about 0.45 to about 0.6 g/ml.

98. The method of claim 64, wherein the lubricant used in step (v) is intragranular.

99. The method of claim 64, wherein the lubricant used in step (vii) is extragranular.

100. The method of claim 64, wherein the disintegrant is selected from the group comprising agar-agar, algins, calcium carbonate, carboxymethylcellulose, cellulose, hydroxypropylcellulose, low substituted hydroxypropylcellulose, clays, croscarmellose sodium (CCNa), crospovidone, gums, magnesium aluminum silicate, methylcellulose, microcrystalline cellulose (MCC), polacrilin potassium, sodium alginate, sodium starch glycolate, maize starch, potato starch, tapioca starch, and combinations thereof.

101. The method of claim 64, wherein the dispersion polymer selected from the group comprising hydroxypropyl methylcellulose (HPMC), hydroxypropyl methylcellulose-acetate succinate (HPMC-AS), hydroxypropyl cellulose (HPC), methyl cellulose, hydroxyethyl methyl cellulose, hydroxyethyl cellulose acetate, hydroxyethyl ethyl cellulose, polyvinyl alcohol polyvinyl acetate copolymers, polyethylene glycol, polyethylene glycol polypropylene glycol copolymers, polyvinylpyrrolidone (PVP), polyethylene polyvinyl alcohol copolymers, poly oxyethylene-polyoxypropylene block copolymers, and combinations thereof.

102. The method of any of one of claims 64-101, wherein the compound of Formula (I) has the structure: 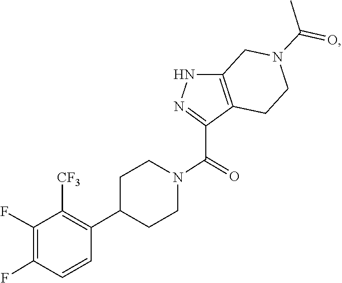 [SMILES: CC(=O)N1CCc2c(C(=O)N3CCC(c4ccc(F)c(F)c4C(F)(F)F)CC3)n[nH]c2C1]
or a pharmaceutically acceptable salt, crystalline solvate, polymorph, prodrug, metabolite, N-oxide, stereoisomer, or isomer thereof.

103. The method of any one of claims 64 to 102, wherein each of the disintegrant, dispersion polymer, two diluents, glidant and lubricant is about 0.01-99% by weight, 1-50% by weight, or 2-20% by weight of the tablet pharmaceutical composition.

104. The method of any one of claims 64 to 103, wherein the compound of Formula (I) is about 1-99.99% by weight, about 10-80% by weight, or about 20-60% by weight of the tablet pharmaceutical composition.

105. The method of any one of claims 64 to 104, wherein the dry weight of the tablet pharmaceutical composition is from about 0.1 mg to about 400 mg.

106. A method for lowering the serum or plasma concentration of RBP4 in a subject, comprising administering to the subject a tablet pharmaceutical composition comprising a therapeutically effective amount of a compound having the structure 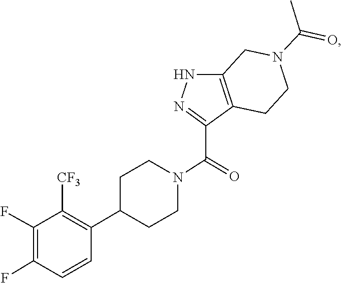 [SMILES: CC(=O)N1CCc2c(C(=O)N3CCC(c4ccc(F)c(F)c4C(F)(F)F)CC3)n[nH]c2C1]
or a pharmaceutically acceptable salt, crystalline, solvate, polymorph, prodrug, metabolite, N-oxide, stereoisomer, or isomer thereof and at least one excipient,
wherein the pharmaceutical composition is administered in an amount of about 5 mg;
wherein the pharmaceutical composition is administered daily;
wherein the serum or plasma levels of RBP4 of the subject are reduced to below 1 μM.

107. A pharmaceutical composition comprising any one of formulas 1-140 from Tables 31-38.

108. A method for lowering the serum or plasma concentration of RBP4 in a subject, comprising administering to the subject a tablet pharmaceutical of claim 107.

109. A method for treating Age-Related Macular Degeneration, dry (atrophic) Age-Related Macular Degeneration, Juvenile Macular Degeneration (Stargardt Disease), Best disease, adult vitelliform maculopathy, Geographic Atrophy, Stargardt-like macular dystrophy, diabetic retinopathy, or ABCA4 gene associated retinal diseases in a subject, comprising administering to the subject a tablet pharmaceutical of claim 107.
